(19) United States
(12) Patent Application Publication
(10) Pub. No.: US 2026/0226426 A1
(43) Pub. Date: Aug. 6, 2026

(54) Oncolytic Viruses as Therapeutic Agents for Treating Cancer

(71) Applicant: ImmVirX Pty Ltd, New Lambton Heights, NSW (AU)

(72) Inventor: Darren Shafren, New Lambton Heights, NSW (AU)

(21) Appl. No.: 19/153,164

(22) PCT Filed: Feb. 8, 2024
(86) PCT No.: PCT/AU2024/050082

§ 371 Date: Aug. 1, 2025

(30) Foreign Application Priority Data
Feb. 8, 2023 (AU) 2023900306

(51) Int. Cl. C12N 7/00; A61K 35/768; A61K 38/17; A61P 35/00; C07K 14/005

(52) U.S. Cl. CPC C12N 7/00; A61K 35/768; A61K 38/1774; A61P 35/00; C07K 14/005; C12N 2770/32021; C12N 2770/32022; C12N 2770/32032

(57) ABSTRACT

The present invention relates to compositions and methods for treating cancer utilising oncolytic viruses, their components, and/or derivatives thereof. In particular, the invention relates to a modified Picornavirus comprising change(s) in one or more of its capsid proteins conferring enhanced binding capacity to cancer cells.

### TECHNICAL FIELD

The present invention relates generally to the field of cancer therapeutics. More specifically, the present invention relates to compositions and methods for treating cancer utilising oncolytic viruses, their components, and/or derivatives thereof. The compositions and methods may optionally include one or more additional anticancer agents, or be combined with one or more anticancer treatments, including but not limited to immune checkpoint inhibitors, CAR-T cells, natural Killer (NK) cells, chemotherapy, and/or radiotherapy.

### BACKGROUND

Viral oncolytic therapy is emerging as a promising treatment for a number of human and animal cancers.

The Picornaviridae is one of the largest families of viruses named using the Greek ‘pico’ (very small), and ‘RNA’ after their ribonucleic acid genome. The family contains a number of clinically significant human and animal pathogens including poliovirus, rhinovirus and hepatitis A. The Picornaviridae family is divided into nine genera based on physical virion properties, RNA sequence similarities and viral RNA genomic organization (Stanway, G., et al., Molecular and Biological Basis of Picornavirus Taxonomy, in Molecular Biology of Picornaviruses, in B. Semler and E. Wimmer, Editors. 2002, ASM Press: Washington DC. p. 17-24). They are small, non-enveloped, icosahedral viruses, and their capsid contains sixty copies each of four viral proteins, VP1, VP2, VP3, and VP4, that form an icosahedral shell (approximately 300 ångströms in diameter) housing a single-stranded, positive-sense RNA genome. A distinctive feature of the capsid surface is a depression (“canyon”) around the 5-fold axes of symmetry which is the site of receptor binding for many Picornaviruses including echoviruses which utilize β-integrins. Receptor molecules that bind in the canyon have been identified as immunoglobulin superfamily members, which upon binding dislodge a factor within a pocket immediately below the canyon surface. When a receptor binds within the canyon it depresses the canyon floor which corresponds to the roof of the pocket. Similarly, when an antiviral compound or lipid binds to the pocket it expands the pocket roof which corresponds to the canyon floor. Hence, receptors that bind to the pocket factor and the canyon compete with each other to bind to the virus. An absence of the hydrophobic pocket factor destabilises the virus and initiates transition to an altered form which is a prelude to uncoating of the virion. Examples of cell receptors that bind to the canyon include immunoglobulin superfamily receptors (e.g. VCAM-1, ICAM-1, PVR, and CAR).

A number of receptors bind to Picornaviruses without using the canyon. For example, a subgroup of human rhinoviruses (HRV) bind to the low-density-lipoprotein receptor family. Other Picornaviruses, including certain coxsackieviruses and echoviruses, use decay-accelerating factor (DAF) as a cellular receptor, usually in combination with another receptor. DAF is a member of a complement activation protein family that bind to and accelerate the decay of both classical and alternative pathway C3 and C5 convertases, the central amplification enzymes of the complement cascade. DAF is expressed on virtually all cell surfaces and protects host cells from the immune system by rapidly dissociating any convertases that assemble, thereby halting the progression of a complement attack directed at the self-cell. The functional region of DAF consists of four short consensus repeats (SCR1-4) each containing about 60 residues and folded into a β structure stabilised by disulphide bridges. The four SCR domains form a relatively rigid extended rod. Closely related Picornaviruses have adapted to bind to DAF at different sites on the receptor surface.

Oncolytic virus therapy is based on the capacity of viruses to infect and kill tumour cells without destroying the normal tissue. While some viruses have a natural preference for tumour cells, most require at least some modification of their receptor tropism to specifically enter and replicate in cancerous cells. Oncolytic Picornaviridae have been successfully used in the treatment of certain cancers. For example, Coxsackievirus A21 (CVA21) is a naturally occurring Picornavirus that has the capacity to preferentially infect and destroy malignant cells bearing the virus-cell entry receptor intercellular adhesion molecule-1 (ICAM-1). The efficacy of CVA21 against melanoma cells has been demonstrated in a range of pre-clinical xenograft models using immune-deficient mice, and against other forms of cancer. There are a number of other cell receptor targets offering potential avenues for the design of effective oncolytic Picornaviruses.

The recent emergence of immune checkpoint inhibitor therapies has transformed cancer treatment in a wide range of tumour types. Effective and durable clinical responses in difficult-to-treat cancer histologies have been observed. However, despite these promising long-term responses, many patients fail to respond to immune checkpoint blockade, demonstrating primary resistance. Additionally, many patients that initially respond to treatment experience relapse secondary to acquired resistance. Both primary and acquired resistance are a result of complex and constantly evolving interactions between the immune system and cancer cells (see, for example, Fares et al., American Society of Clinical Oncology Educational Book 39, May 17, 2019, 147-164).

There remains a need for improved compositions and methods for the treatment, alleviation, or prevention of cancer, including cancer types that are poorly responsive to immune checkpoint inhibitor therapies.

### SUMMARY OF THE INVENTION

The present inventors have generated modified Picornaviridae family viruses with enhanced capacity to bind to decay accelerating factor (DAF/CD55) on the surface of cancer cells, as compared to wild-type counterpart viruses. These are demonstrated to be effective oncolytic agents against cancer cells.

Without any limitation to specific forms of cancer, the compositions and methods described herein may be effective in treating, alleviating and/or preventing cancer, including cancer types that are poorly responsive to immune checkpoint therapies.

The compositions and methods described herein may be used in conjunction with other anticancer agents or treatments, including but not limited to immune checkpoint inhibitors/immune checkpoint inhibitor therapy, CAR-T cells/CAR-T cell therapy, natural Killer (NK) cells, chemotherapeutic agents/chemotherapy, radiotherapeutic agents/radiotherapy, and the like.

Without limitation, the present invention relates at least to the following listed embodiments 1-71:
Embodiment 1. A modified Picornavirus comprising change(s) in any one or more of capsid proteins: VP1, VP2, VP3; compared to a wild-type strain of the virus which confer enhanced binding capacity to decay accelerating factor (DAF/CD55) and/or Neonatal Fc Receptor (FcRn), compared to the wild-type strain.
Embodiment 2. The modified Picornavirus of embodiment 1, comprising said change(s) in each of the VP2 and VP3 capsid proteins.
Embodiment 3. The modified Picornavirus of embodiment 1, comprising said change(s) in each of the VP1, VP2 and VP3 capsid proteins.
Embodiment 4. The modified Picornavirus of any one of embodiments 1 to 3, further comprising change(s) in any one or more of non-structural proteins: 2A, 3A, 3C, 3D; compared to the wild-type strain of the virus which confer enhanced binding capacity to decay accelerating factor (DAF/CD55) compared to the wild-type strain.
Embodiment 5. The modified Picornavirus of any one of embodiments 1 to 4, wherein the wild-type strain and the modified Picornavirus have an identical nucleotide sequence and/or an identical amino acid sequence, apart from:
(i) the change(s) in any one or more of capsid proteins VP1, VP2, VP3; and optionally
(ii) the change(s) in any one or more of non-structural proteins: 2A, 3A, 3C, 3D.
Embodiment 6. The modified Picornavirus of any one of embodiments 1 to 5, wherein the modified Picornavirus is an Enterovirus.
Embodiment 7. The modified Picornavirus of embodiment 6, wherein the enterovirus is selected from the group consisting of Echovirus, Poliovirus, unclassified Enteroviruses, Rhinovirus, Paraechovirus, Hepatovirus, and Cardiovirus.
Embodiment 8. The modified Picornavirus of any one of embodiments 1 to 7, wherein the modified Picornavirus is not a Coxsackievirus.
Embodiment 9. The modified Picornavirus of any one of embodiments 1 to 8, wherein the modified Picornavirus is an Echovirus, Enterovirus B85, Coxsackievirus A9, Coxsackievirus A13, Coxsackievirus 15 or Coxsackievirus 21.
Embodiment 10. The modified Picornavirus of any one of embodiments 1 to 9, wherein the modified Picornavirus is an Echovirus 1, Echovirus 3, Echovirus 6, Echovirus 7, Echovirus 9, Echovirus 11, Echovirus 12 (E12), Echovirus 12, Echovirus 13, Echovirus 14, Echovirus 15, Echovirus 17, Echovirus 25, Echovirus 26, Echovirus 29, or Echovirus 30.
Embodiment 11. The modified Picornavirus of any one of embodiments 1 to 10, wherein the change(s) in capsid protein VP2 comprise or consist of an asparagine to threonine change at residue 142.
Embodiment 12. The modified Picornavirus of any one of embodiments 1 to 11, wherein the change(s) in capsid protein VP3 comprise or consist of an alanine to valine change at residue 206.
Embodiment 13. The modified Picornavirus of any one of embodiments 1 to 12, wherein the change(s) in capsid protein VP2 comprise or consist of a histidine to tyrosine change at residue 154, and/or a serine to asparagine change at residue 168.
Embodiment 14. The modified Picornavirus of any one of embodiments 1 to 13, wherein the change(s) in capsid protein VP1 comprise or consist of a tyrosine to histidine change at residue 230.
Embodiment 15. The modified Picornavirus of any one of embodiments 1 to 14, wherein the change(s) in capsid protein VP1 comprise or consist of a phenylalanine to tyrosine change at residue 210.
Embodiment 16. The modified Picornavirus of any one of embodiments 1 to 15, wherein the change(s) in capsid protein VP1 comprise or consist of a glutamine to arginine change at residue 132.
Embodiment 17. The modified Picornavirus of embodiment 16, wherein the change confers enhanced binding to Neonatal Fc Receptor (FcRn).
Embodiment 18. The modified Picornavirus of any one of embodiments 1 to 17, wherein the change(s) in the non-structural proteins: 2A, 3A, 3C, 3D; comprise or consist of any one or more of:
a phenylalanine to tyrosine change at residue 47 of the non-structural 2A protein;
an isoleucine to tyrosine change at residue 6 of the non-structural 3A protein;
a histidine to arginine change at residue 39 of the non-structural 3C protein;
an isoleucine to leucine change at residue 123 of the non-structural 3D protein.
Embodiment 19. The modified Picornavirus of any one of embodiments 1 to 18, wherein the wild-type strain is Echovirus 12 strain Travis.
Embodiment 20. The modified Picornavirus of any one of embodiments 1 to 19, wherein the wild-type strain is Echovirus 12 strain Travis comprising or consisting of an amino acid sequence as defined in SEQ ID NO: 46.
Embodiment 21. The modified Picornavirus of any one of embodiments 1 to 20, further comprising at least one nucleic acid sequence encoding an exogenous protein or component thereof.
Embodiment 22. The modified Picornavirus of embodiment 21, wherein the exogenous protein is an immunostimulatory protein, or an agent (e.g. an antibody) capable of binding to and inhibiting the biological activity of an immune checkpoint molecule or a ligand of the immune checkpoint molecule.
Embodiment 23. The modified Picornavirus of embodiment 22, wherein the immunostimulatory protein is selected from any one or more of: an interleukin, including but not limited to interleukin-2 (IL-2), interleukin-12 (IL-12), interleukin-15 (IL-15), interleukin-21 (IL-21), RANTES, GM-CSF, an interferon, including but not limited to IFN-γ, TNF-α, a pro-drug converting enzyme, biologically active component(s) thereof, combinations thereof.
Embodiment 24. The modified Picornavirus of embodiment 22, wherein the immune checkpoint molecule is selected from any one or more of: PD-1, PD-L1, PD-L2, CTLA-4, CD134, CD134L, CD137, CD137L, CD80, CD86, B7-H3, B7-H4, B7RP1, LAG-3, ICOS, TIM3, GAL9, CD28 OX-40, and TIGIT, biologically active component(s) thereof, combinations thereof.
Embodiment 25. The modified Picornavirus of any one of embodiments 1 to 24, further comprising one or more components for expression of a tissue-specific miRNA capable of inhibiting replication of the modified Picornavirus in a tissue-specific manner.
Embodiment 26. The modified Picornavirus of any one of embodiments 1 to 25, comprising enhanced oncolytic activity compared to the wild-type strain.
Embodiment 27. The modified Picornavirus of embodiment 26, wherein the enhanced oncolytic activity is against any one or more of ovarian cancer cells, colorectal cancer cells, gastric cancer cells, liver cancer cells, pancreatic cancer cells, head and neck cancer cells, stomach cancer cells, breast cancer cells, sarcoma cells, lymphoma cells, brain cancer cells.
Embodiment 28. The modified Picornavirus of embodiment 27, wherein the cancer cells express decay accelerating factor (DAF/CD55) and/or Neonatal Fc Receptor (FcRn).
Embodiment 29. The modified Picornavirus of any one of embodiments 1 to 28, wherein the Picornavirus comprises:
(i) a VP1 capsid protein amino acid sequence comprising any one of SEQ ID NOs: 54-57, 74, 86, 98, 110, 122, 134, 146 or 158;
(ii) a VP2 capsid protein amino acid sequence comprising any one of SEQ ID NOs: 58-61, 75, 87, 99, 111, 123, 135, 147 or 159;
(iii) a VP3 capsid protein amino acid sequence comprising any one of SEQ ID NOs: 62, 63, 76, 88, 100, 112, 124, 136, 148 or 160.
(iv) a non-structural protein 2A sequence comprising any one of SEQ ID NOs: 64, 65, 77, 89, 101, 113, 125, 137, 149 or 161.
(v) a non-structural protein 3A sequence comprising any one of SEQ ID NOs: 66, 67, 78, 90, 102, 114, 126, 138, 150 or 162.
(vi) a non-structural protein 3C sequence comprising any one of SEQ ID NOs: 68, 69, 79, 91, 103, 115, 127, 139, 151 or 163.
(vii) a non-structural protein 3D sequence comprising any one of SEQ ID NOs: 70, 71, 80, 92, 104, 116, 128, 140, 152 or 164.
Embodiment 30. The modified Picornavirus of any one of embodiments 1 to 29, comprising or consisting of an amino acid sequence as defined in SEQ ID NO: 42, SEQ ID NO: 52, SEQ ID NO: 53, SEQ ID NO: 73, SEQ ID NO: 85, SEQ ID NO: 97, SEQ ID NO: 109, SEQ ID NO: 121, SEQ ID NO: 133, SEQ ID NO: 145 or SEQ ID NO: 157 or a variant thereof having at least: 80%, 85%, 90%, 95%, 96%, 97%, 98% or 99% sequence identity with SEQ ID NO: 42, SEQ ID NO: 52, SEQ ID NO: 53, SEQ ID NO: 73, SEQ ID NO: 85, SEQ ID NO: 97, SEQ ID NO: 109, SEQ ID NO: 121, SEQ ID NO: 133, SEQ ID NO: 145 or SEQ ID NO: 157.
Embodiment 31. The modified Picornavirus of any one of embodiments 1 to 30, encoded by a nucleotide sequence comprising or consisting of an RNA or cDNA/DNA sequence defined in SEQ ID NO: 41, SEQ ID NO: 50, SEQ ID NO: 51, SEQ ID NO: 72, SEQ ID NO: 84, SEQ ID NO: 96, SEQ ID NO: 108, SEQ ID NO: 120, SEQ ID NO: 132, SEQ ID NO: 144 or SEQ ID NO: 156, or a variant thereof having at least: 80%, 85%, 90%, 95%, 96%, 97%, 98% or 99% sequence identity with an RNA or cDNA/DNA sequence defined in SEQ ID NO: 41, SEQ ID NO: 50, SEQ ID NO: 51, SEQ ID NO: 72, SEQ ID NO: 84, SEQ ID NO: 96, SEQ ID NO: 108, SEQ ID NO: 120, SEQ ID NO: 132, SEQ ID NO: 144 or SEQ ID NO: 156.
Embodiment 32. The modified Picornavirus of any one of embodiments 1 to 31, synthesised using recombinant means.
Embodiment 33. A pharmaceutical composition comprising any one or more of:
the modified Picornavirus of any one of embodiments 1 to 32,
RNA of the modified Picornavirus of any one of embodiments 1 to 32,
complementary DNA (cDNA) encoding all or a portion of the genome of the modified Picornavirus of any one of embodiments 1 to 32;
and a pharmaceutically acceptable carrier, excipient or diluent.
Embodiment 34. The pharmaceutical composition of embodiment 33 wherein the RNA of the modified Picornavirus (e.g. synthetic RNA) and/or complementary DNA (cDNA) encoding all or a portion of the genome of the modified Picornavirus is provided within a nanoparticle (e.g. a lipid nanoparticle).
Embodiment 35. The pharmaceutical composition of embodiment 33 or embodiment 34, further comprising CAR-T cells, natural Killer (NK) cells, an immunostimulatory protein, and/or an agent (e.g. an antibody) capable of binding to an immune checkpoint molecule or a ligand of the immune checkpoint molecule.
Embodiment 36. The pharmaceutical composition of embodiment 35, wherein the CAR-T cells are engineered to bind to any one or more of: EGFRVIII, interleukin 13Ra2, FAP, GD2, EpCam, CD133, CD70, Her2, CEA, GAP, CD5, CD38, Muc1, GPC3, BCMA, Meso, PSCA, CD33, PSMA, ROR1, Muc1, EphA2, CD123, CD19, Claudin 18.2; on the surface of a cancerous cell.
Embodiment 37. The pharmaceutical composition of embodiment 35 or embodiment 36, wherein the immunostimulatory protein is selected from any one or more of: an interleukin, including but not limited to interleukin-2 (IL-2), interleukin-12 (IL-12), interleukin-15 (IL-15), interleukin-21 (IL-21), RANTES, GM-CSF, an interferon, including but not limited to IFN-γ, TNF-α, a pro-drug converting enzyme, biologically active component(s) thereof, combinations thereof.
Embodiment 38. The pharmaceutical composition of any one of embodiments 35 to 37, wherein the immune checkpoint molecule is selected from any one or more of: PD-1, PD-L1, PD-L2, CTLA-4, CD134, CD134L, CD137, CD137L, CD80, CD86, B7-H3, B7-H4, B7RP1, LAG-3, ICOS, TIM3, GAL9, CD28, OX-40, and TIGIT, biologically active component(s) thereof, combinations thereof.
Embodiment 39. The pharmaceutical composition of any one of embodiments 33 to 38, wherein the composition comprises Picornaviruses at between about 10⁸ and about 10¹⁵ viral particles per mL, between about 10⁸ and 10¹² viral particles per mL, or between about 10⁸ and 10¹⁰ viral particles per mL.
Embodiment 40. The pharmaceutical composition of any one of embodiments 33 to 39, wherein the composition comprises a particle to infectivity ratio of less than or equal to about 3000:1, less than or equal to about 300:1, in the range of about 200:1 to about 5:1, in the range of about 50:1 to about 10:1, or less than about 20:1.
Embodiment 41. The pharmaceutical composition of any one of embodiments 33 to 40, wherein the composition comprises a level of host cell DNA less than or equal to about 200 ng per mL, in the range of 1 to about 100 ng per mL, in the range of about 1 to about 50 ng per mL, or in the range of about 1 to about 10 ng per mL.
Embodiment 42. A method for treating cancer in a subject, the method comprising administering to the subject a therapeutically effective amount of any one or more of:
the modified Picornavirus of any one of embodiments 1 to 32;
RNA of the modified Picornavirus of any one of embodiments 1 to 32;
complementary DNA (cDNA) encoding all or a portion of a genome of the modified Picornavirus of any one of embodiments 1 to 32;
the pharmaceutical composition of any one of embodiments 33 to 41.
Embodiment 43. The method of embodiment 42, wherein the modified Picornavirus is bioselected for any one or more of: enhanced migration between cancer cells, enhanced DAF and/or FcRn binding, enhanced replicative capacity at mammalian body temperatures (33° C.-39° C.), enhanced lytic activity of cancer cells expressing any one or more of: PD-1, PD-L1, PD-L2, CTLA-4, CD134, CD134L, CD137, CD137L, CD80, CD86, B7-H3, B7-H4, B7RP1, ICOS, TIM3, LAG-3, GAL9, CD28 and/or OX-40.
Embodiment 44. The method of embodiment 43, wherein the modified Picornavirus is bioselected in vivo using a tumour xenograph.
Embodiment 45. The method of embodiment 44, wherein the tumour is from the subject.
Embodiment 46. The method of any one of embodiments 42 to 45, wherein the therapeutically effective amount is administered to the subject by parenteral, intravenous, intravesical, subcutaneous, oral, intratumoural, ocular, topical or systemic administration.
Embodiment 47. The method of any one of embodiments 42 to 46, wherein the therapeutically effective amount is co-administered with CAR-T cells and/or natural killer (NK) cells.
Embodiment 48. The method of embodiment 47, wherein the CAR-T cells are engineered to bind to any one or more of: EGFRVIII, interleukin 13Ra2, FAP, GD2, EpCam, CD133, CD70, Her2, CEA, GAP, CD5, CD38, Muc1, GPC3, BCMA, Meso, PSCA, CD33, PSMA, ROR1, Muc1, EphA2, CD123, CD19, Claudin 18.2; on the surface of a cancerous cell.
Embodiment 49. The method of embodiment 47 or embodiment 48, wherein the CAR-T cells and/or natural killer (NK) cells are co-administered prior to or following the administration of the therapeutically effective amount to the subject.
Embodiment 50. The method of any one of embodiments 42 to 49, wherein the therapeutically effective amount is co-administered with an immunostimulatory protein, and/or an agent (e.g. an antibody) capable of binding to an immune checkpoint molecule or a ligand of the immune checkpoint molecule.
Embodiment 51. The method of embodiment 50, wherein the immunostimulatory protein is selected from any one or more of: an interleukin, including but not limited to interleukin-2 (IL-2), interleukin-12 (IL-12), interleukin-15 (IL-15), interleukin-21 (IL-21), RANTES, GM-CSF, an interferon, including but not limited to IFN-γ, TNF-α, a pro-drug converting enzyme, biologically active component(s) thereof, combinations thereof.
Embodiment 52. The method of embodiment 50 or embodiment 51, wherein the immune checkpoint molecule is selected from the group consisting of: PD-1, PD-L1, PD-L2, CTLA-4, CD134, CD134L, CD137, CD137L, CD80, CD86, B7-H3, B7-H4, B7RP1, ICOS, LAG-3, TIM3, GAL9, CD28, OX-40, and TIGIT.
Embodiment 53. The method of any one of embodiments 50 to 52, wherein the agent is a monoclonal antibody.
Embodiment 54. The method of any one of embodiments 50 to 53, wherein the immuno-stimulatory protein and/or agent is co-administered prior to or following administration of the therapeutically effective amount to the subject.
Embodiment 55. The method of any one of embodiments 42 to 54, wherein the cancer is classified as a cancer that is resistant to immune checkpoint therapy.
Embodiment 56. The method of any one of embodiments 42 to 55, wherein the cancer is ovarian cancer, colorectal cancer, gastric cancer, liver cancer, pancreatic cancer, head and neck cancer, stomach cancer, breast cancer, sarcoma, lymphoma, or brain cancer.
Embodiment 57. The method of embodiment 56, wherein the cancer comprises cancerous cells which express decay accelerating factor (DAF/CD55) and/or Neonatal Fc Receptor (FcRn).
Embodiment 58. Use of any one or more of:
the modified Picornavirus of any one of embodiments 1 to 32;
RNA of the Picornavirus of any one of embodiments 1 to 32;
complementary DNA (cDNA) encoding all or a portion of a genome of the Picornavirus of any one of embodiments 1 to 32;
the pharmaceutical composition of any one of embodiments 33 to 41;
in the manufacture of a medicament for treating cancer in a subject.
Embodiment 59. The use of embodiment 58, wherein the medicament further comprises CAR-T cells, natural Killer (NK) cells, an immuno-stimulatory protein, and/or an agent (e.g. an antibody) capable of binding to an immune checkpoint molecule or a ligand of the immune checkpoint molecule.
Embodiment 60. Modified Picornavirus of any one of embodiments 1 to 32, RNA of the Picornavirus of any one of embodiments 1 to 32, complementary DNA (cDNA) encoding all or a portion of a genome of the Picornavirus of any one of embodiments 1 to 32, and/or pharmaceutical composition of any one of embodiments 33 to 41, for use in the treatment of cancer.
Embodiment 61. The modified Picornavirus, RNA and/or cDNA of embodiment 60, for use in treating cancer in combination with CAR-T cells, and/or natural killer (NK) cells, and/or an immuno-stimulatory agent.
Embodiment 62. The use of embodiment 59, or the modified Picornavirus, RNA and/or cDNA of embodiment 61, wherein the CAR-T cells are engineered to bind to any one or more of: EGFRVIII, interleukin 13Ra2, FAP, GD2, EpCam, CD133, CD70, Her2, CEA, GAP, CD5, CD38, Muc1, GPC3, BCMA, Meso, PSCA, CD33, PSMA, ROR1, Muc1, EphA2, CD123, CD19, Claudin 18.2; on the surface of a cancerous cell.
Embodiment 63. The use of embodiment 59 or embodiment 62, or the modified Picornavirus, RNA and/or cDNA of embodiment 60 or embodiment 61, wherein the immuno-stimulatory protein is selected from any one or more of: an interleukin, including but not limited to interleukin-2 (IL-2), interleukin-12 (IL-12), interleukin-15 (IL-15), interleukin-21 (IL-21), RANTES, GM-CSF, an interferon, including but not limited to IFN-γ, TNF-α, a pro-drug converting enzyme, biologically active component(s) thereof, combinations thereof.
Embodiment 64. The use of embodiment 63, or the modified Picornavirus, RNA and/or cDNA of embodiment 84, wherein the immune checkpoint molecule is selected from the group consisting of: PD-1, PD-L1, PD-L2, CTLA-4, CD134, CD134L, CD137, CD137L, CD80, CD86, B7-H3, B7-H4, LAG-3, B7RP1, ICOS, TIM3, GAL9, CD28, OX-40, and TIGIT.
Embodiment 65. The use of any one of embodiments 58, 59 or 62 to 64, or the modified Picornavirus, RNA and/or cDNA of any one of embodiments 60 to 64, wherein the modified Picornavirus is bioselected for any one or more of: enhanced migration between cancer cells, enhanced DAF and/or FcRn binding, enhanced replicative capacity at mammalian body temperatures (37° C.-39° C.), enhanced lytic activity of cancer cells expressing any one or more of: PD-1, PD-L1, PD-L2, CTLA-4, CD134, CD134L, CD137, CD137L, CD80, CD86, B7-H3, B7-H4, B7RP1, LAG-3, ICOS, TIM3, GAL9, CD28 and/or OX-40.
Embodiment 66. The use of any one of embodiments 58, 59 or 62 to 65, or the modified Picornavirus, RNA and/or cDNA of any one of embodiments 60 to 65, wherein the modified Picornavirus is bioselected in vivo using a tumour xenograph.
Embodiment 67. The use of embodiment 66, or the modified Picornavirus, RNA and/or cDNA of embodiment 66, wherein the tumour is from the subject.
Embodiment 68. The use of any one of embodiments 58, 59 or 62 to 67, or the modified Picornavirus, RNA and/or cDNA of any one of embodiments 60 to 67, wherein the cancer is ovarian cancer, colorectal cancer, gastric cancer, liver cancer, pancreatic cancer, head and neck cancer, stomach cancer, breast cancer, sarcoma, lymphoma, or brain cancer.
Embodiment 69. The use of embodiment 68, or the modified picornavirus RNA and/or cDNA of embodiment 68, wherein the cancer comprises cancerous cells which express decay accelerating factor (DAF/CD55) and/or Neonatal Fc Receptor (FcRn).
Embodiment 70. The use of any one of embodiments 58, 59 or 62 to 69, or the modified Picornavirus, RNA and/or cDNA of any one of embodiments 60 to 69, wherein the modified Picornavirus comprises or consists of an amino acid sequence as defined in SEQ ID NO: 42, SEQ ID NO: 52, SEQ ID NO: 53, SEQ ID NO: 73, SEQ ID NO: 85, SEQ ID NO: 97, SEQ ID NO: 109, SEQ ID NO: 121, SEQ ID NO: 133, SEQ ID NO: 145 or SEQ ID NO: 157 or a variant thereof having at least: 80%, 85%, 90%, 95%, 96%, 97%, 98% or 99% sequence identity with SEQ ID NO: 42, SEQ ID NO: 52, SEQ ID NO: 53, SEQ ID NO: 73, SEQ ID NO: 85, SEQ ID NO: 97, SEQ ID NO: 109, SEQ ID NO: 121, SEQ ID NO: 133, SEQ ID NO: 145 or SEQ ID NO: 157.
Embodiment 71. The use of any one of embodiments 58, 59 or 62 to 70, or the modified Picornavirus, RNA and/or cDNA of any one of embodiments 60 to 70, wherein the modified Picornavirus is encoded by a nucleotide sequence comprising or consisting of an RNA or cDNA/DNA sequence defined in SEQ ID NO: 41, SEQ ID NO: 50, SEQ ID NO: 51, SEQ ID NO: 72, SEQ ID NO: 84, SEQ ID NO: 96, SEQ ID NO: 108, SEQ ID NO: 120, SEQ ID NO: 132, SEQ ID NO: 144 or SEQ ID NO: 156, or a variant thereof having at least: 80%, 85%, 90%, 95%, 96%, 97%, 98% or 99% sequence identity with an RNA or cDNA/DNA sequence defined in SEQ ID NO: 41, SEQ ID NO: 50, SEQ ID NO: 51, SEQ ID NO: 72, SEQ ID NO: 84, SEQ ID NO: 96, SEQ ID NO: 108, SEQ ID NO: 120, SEQ ID NO: 132, SEQ ID NO: 144 or SEQ ID NO: 156.

### Definitions

The following are some definitions that may be helpful in understanding the description of the present invention. These are intended as general definitions which do not limit the scope of the present invention to those terms alone but are put forth for a better understanding of the following description.

As used in this application, the singular form “a”, “an” and “the” include plural references unless the context clearly dictates otherwise. For example, the term “Rhinovirus” includes a plurality of Rhinoviruses and/or modified forms thereof. Thus, in the context of this specification, the singular encompasses also the plural except where the specific context clearly indicates otherwise. For example, where it is stated that the invention includes methods for treatment of cancer by administration of an oncolytic virus or oncolytic viral RNA, it will be understood that this encompasses the administration of one or more such viruses or viral RNAs. Similarly, unless the context requires otherwise or specifically stated to the contrary, integers, steps, or elements of the invention recited herein as singular integers, steps or elements clearly encompass both singular and plural forms of the recited integers, steps or elements.

As used herein, the addition of “(s)” to a given term will be understood to convey an option of singular or plural. For example, “change(s)” will be understood to include a single change or multiple changes.

In the context of this specification, where a numerical range is provided it will be understood to encompass the stated end points of the range and all values between those end points, including any sub-ranges within those endpoints.

As used herein, the term “comprising” means “including” in a non-exhaustive sense. Variations of the word “comprising”, such as “comprise” and “comprises,” have correspondingly varied meanings. Thus, for example, a composition “comprising” Picornaviruses may consist exclusively of Picornaviruses or may include one or more additional components (e.g. a pharmaceutically acceptable excipient, carrier or diluent).

As used herein, the term “therapeutically effective amount” includes within its meaning a non-toxic but sufficient amount of a given agent or agents (e.g. an oncolytic Picornavirus or viral RNA from an oncolytic Picornavirus) of the present invention to provide the desired therapeutic effect. The exact amount of agent(s) required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

As used herein a nucleic acid or nucleic acid sequence “derived from” a Picornavirus will be understood to include viral RNA directly isolated from a Picornavirus, synthetic viral RNA, and cDNA encoding the viral genome or a component thereof corresponding to the isolated sequence. Also included are synthetic polynucleotide sequences comprising one or more mutations in the sequence compared to a wild-type sequence or parental sequence including, for example, mutations in capsid protein(s).

As used herein a “wild-type” strain, virus, sequence or organism will be understood to be a strain, virus, sequence or organism as existing in nature or as isolated from a naturally occurring source, and also includes forms that are artificially synthesised but otherwise identical to those existing in nature. In some embodiments they may be representative of a form most frequently observed in a natural population. A wild-type strain, virus, sequence or organism as referred to herein may serve as a base strain, virus, sequence or organism into which change(s) is/are introduced. They may thus be used as a reference when comparing a modified strain, virus, sequence or organism described herein.

The term “polynucleotide” as used herein refers to a single- or double-stranded polymer of deoxyribonucleotide, ribonucleotide bases or known analogues or natural nucleotides, or mixtures thereof.

As used herein, the term the term “treatment” and related terms such as “treating”, “treated”, and treat” refer to any and all uses which remedy or alleviate a disease state or symptoms, prevent the establishment of disease, or otherwise prevent, hinder, retard, or reverse the progression of disease or other undesirable symptoms in any way whatsoever. For the avoidance of misunderstanding it is noted that “treatment” and related terms as used herein does not require complete cure or remission of the disease being treated.

As used herein, the term “subject” or “patient” includes humans and individuals of any species of social, economic or research importance including but not limited to members of the genus ovine, bovine, equine, porcine, feline, canine, primates, rodents. The subject or patient may be mammalian, such as a human.

As used herein, the term “kit” refers to any delivery system for delivering materials. Such delivery systems include systems that allow for the storage, transport, or delivery of reaction reagents (for example labels, reference samples, supporting material, etc. in the appropriate containers) and/or supporting materials (for example, buffers, written instructions for performing an assay etc.) from one location to another. For example, kits may include one or more enclosures, such as boxes, containing the relevant reaction reagents and/or supporting materials. The term “kit” includes both fragmented and combined kits. A “fragmented kit” refers to a delivery system comprising two or more separate containers that each contains a sub-portion of the total kit components. The containers may be delivered to the intended recipient together or separately. Any delivery system comprising two or more separate containers that each contains a sub-portion of the total kit components are included within the meaning of the term “fragmented kit”. A “combined kit” refers to a delivery system containing all of the components of a reaction assay in a single container (e.g. in a single box housing each of the desired components).

Any description of prior art documents herein, or statements herein derived from or based on those documents, is not an admission that the documents or derived statements are part of the common general knowledge of the relevant art.

For the purposes of description, all documents referred to herein are hereby incorporated by reference in their entirety unless otherwise stated.

### BRIEF DESCRIPTION OF THE FIGURES

Non-limiting embodiments of the present invention are described below by way of example only, with reference to the accompanying figures wherein:

FIG. 1 depicts novel capsid changes in the DAF-binding footprint that enhance oncolytic activity of an IVX037 strain of the invention compared to prototype strains. Residues are coloured according to their contribution to the total contact area with DAF: yellow (<5%), orange (<9%), and red (>9%).

FIG. 2 shows a three-dimensional structure (front view) of a modified Echovirus 12 (E12) IVX037 strain according to an embodiment of the present invention.

FIG. 3 shows a three-dimensional structure (side view) of a modified E12 IVX037 strain according to an embodiment of the present invention.

FIG. 4 provides a series of graphs (A-C) comparing the oncolytic activity of modified E12 strains according to an embodiment of the present invention with other pre-existing E12 strains.

FIG. 5 shows an IVX 037 strain according to an embodiment of the present invention induced up-regulation of DDX58 (RIG-I), CD274 (PD-L1) and IFN-g inducible protein 10 (CXCL10) and PD-L1 in in vitro cultures of human MSS-colorectal and ovarian epithelial adenocarcinoma cells 10 hours post-infection

FIG. 6 shows in vivo anti-tumour activity and Oncolytic activity of an intratumoral IVX 037 strain according to an embodiment of the present invention in human MSS-Colorectal cancer (WiDr) xenografts.

FIG. 7 shows an IVX 037 strain according to an embodiment of the present invention induced up-regulation of DDX58 (RIG-I), CD274 (PD-L1) and IFN-g inducible protein 10 (CXCL10) and PD-L1 within human MSS-Colorectal (WiDr) xenografts.

FIG. 8 shows an IVX-037 OVO strain according to an embodiment of the present invention infectivity data on CHO cells transfected with human FcRn using the forward (A) and reverse (B) transfection method.

FIG. 9 shows an IVX-037 OVO strain according to an embodiment of the present invention infectivity data on CHO cells transfected with either the human FcRn plasmid or the pIND control plasmid (*p<0.05, **p<0.01, ***p<0.001, ****p<0.0001).

FIG. 10 shows the average (A) and individual (B) tumour volumes of mice treated with an IVX037 OVO strain of the invention (*p<0.05, **p<0.01, ***p<0.001, ****p<0.0001).

FIG. 11 shows the average (A) and individual (B) tumour volumes of mice treated with an IVX-037 OVO strain according to an embodiment of the present invention (*p<0.05, **p<0.01, ***p<0.001, ****p<0.0001).

FIG. 12 shows the individual tumour volumes of mice treated with an IVX037 strain of the invention.

FIG. 13 shows the average (A) and individual (B) tumour volumes of mice treated with an IVX-037 OVO strain according to an embodiment of the present invention (*p<0.05, **p<0.01, ***p<0.001, ****p<0.0001).

FIG. 14 shows that IVX037 infects and replicates in human colorectal cancer xenografts resulting in tumour growth inhibition. (A) A single injection of IVX037 (1×10⁸ TCID₅₀) was administered intratumourally (i.t) into WiDr tumour xenografts. (B) IVX037 at escalating doses (1×10⁴, 1×10⁶, 1×10⁸ TCID₅₀) was injected intratumourally to the WiDr tumour xenografts. (C) Mice bearing WiDr tumour xenografts were treated with four intravenous (i.v) infusions of IVX037 spaced three to four days apart. (D) A single injection of IVX037 (1×10⁸ TCID₅₀) was administered intratumourally into Caco-2 tumour xenografts. Tumour volumes were expressed as the mean±SD (mm³). ****P<0.0001.

FIG. 15 expression of RIG-I, CD274 and CXCL10 at 1-, 2- and 3-day post treatment. SCID mice were inoculated with WiDr cells. When tumours reached to an average size (approximately 50 mm3), mice were treated with virus IVX037 (n=8 mice) or control formulation buffer (n=6 mice) on the day 0. Mice were sacrificed at 1, 2, and 3-day post treatment. Tumours were collected and RNA was extracted. Expression of RIG-I, CD274 and CXCL10 was measured using RT-PCR and calculated as a fold change.

FIG. 16 (A) Patient serum levels of IVX037 viral RNA. IVX037 viral levels were determined using semi-quantitative RT-qPCR. (B) Patient serum levels of neutralizing antibodies (nAbs) post IVX037 administration. (C) Patient serum CXCL10 levels. CXCL10 levels were determined using a multi-plex flow cytometry assay. Statistical analysis utilised a paired sample “t-test” technique.

FIG. 17 shows Phase 1a/b trial schema.

FIG. 18 shows serum level of CXCL10 in patients on study. P value was calculated a T-test using Graphpad Prism. CXCL10 was expressed as pg/mL.

FIG. 19 shows dose response curves of IVX037 on a panel of susceptible liver cancer cell lines. Cell death induced by dose dependent IVX037 infection is graphed as a percentage of cell survival. A normalised dose response (variable slope) was used as the curve of best fit, analysed using GraphPad Prism. Cell survival data were generated using the XTT cell viability assay. IVX037 dose is defined as MOI expressed as TCID₅₀/cell. Photomicrographs on display show the cytopathic effects of IVX037 on these cell lines.

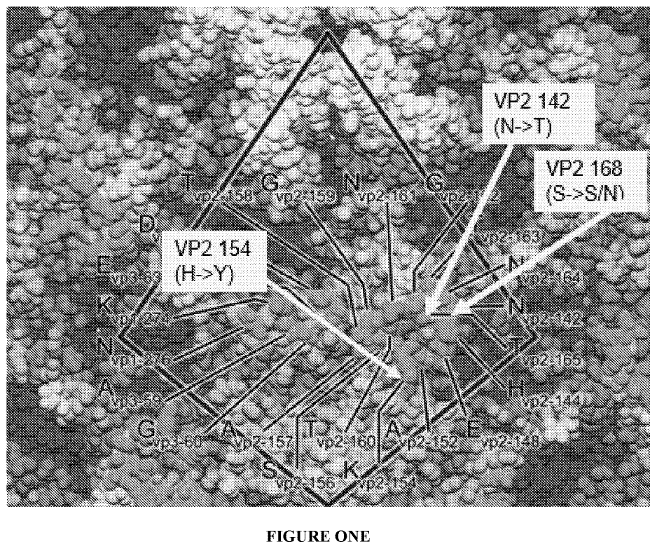

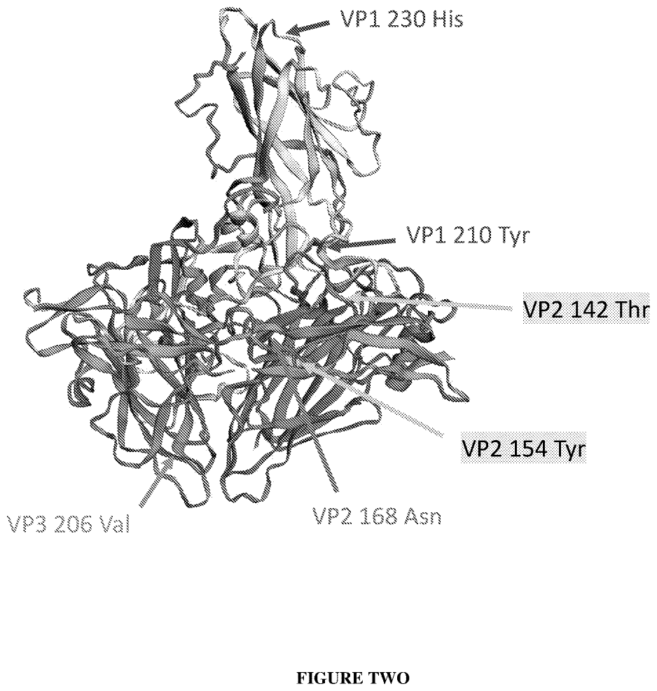

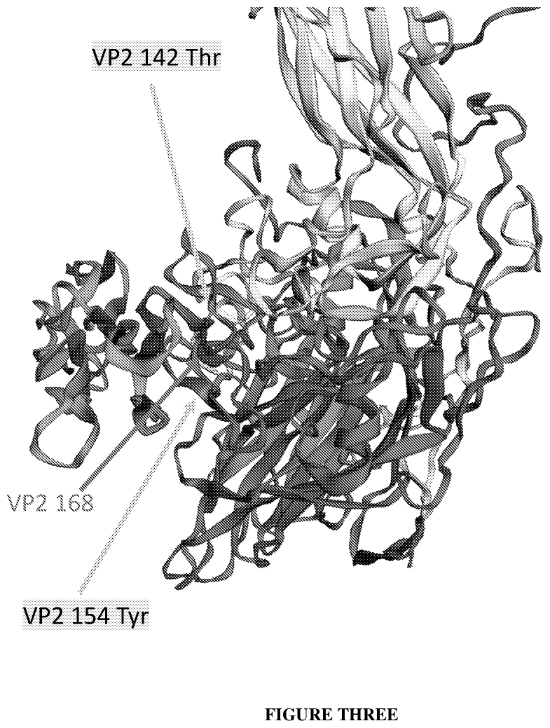

### DETAILED DESCRIPTION

The invention will now be described in more detail, including, by way of illustration only, with respect to the examples which follow.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention relates to Picornaviridae family viruses and modified forms thereof which bind to decay-accelerating factor (DAF) receptors on the surface of cancer cells. This binding facilitates lytic infection of the cancer cells by the viruses, which are consequently effective oncolytic agents in the treatment, alleviation, and/or prevention of cancer.

The oncolytic Picornaviruses of the present invention may be modified so as to enhance their binding capacity to DAF as compared to unmodified or wild-type Picornavirus strains. The increased capacity to bind DAF may facilitate more effective binding to cancer cells, thereby enhancing the effectiveness of the modified Picornavirus strains in the treatment, alleviation, and/or prevention of cancer.

Additionally or alternatively, the oncolytic Picornaviruses of the present invention may be modified by other mean(s) to enhance their effectiveness in the treatment, alleviation, and/or prevention of cancer. By way of non-limiting example, the viruses may be subjected to one or various bioselection procedures to increase their capacity to target and/or lyse cancer cells. Additionally or alternatively, and again by way of non-limiting example, the viruses may be subjected to recombinant modification or incorporate exogenous nucleic acids encoding proteins that increase their capacity to target and/or lyse cancer cells.

The oncolytic Picornaviruses and modified forms thereof of the present invention may be administered in combination with other anticancer agents such as, by way of non-limiting example, immune checkpoint inhibitors, CAR-T cells, natural Killer (NK) cells, chemotherapeutic agents, and the like.

Without any limitation to specific forms of cancer, the oncolytic Picornaviruses and modified forms thereof may be effective in treating, alleviating and/or preventing cancer types that are poorly responsive to immune checkpoint therapies such as, for example, cancers with objective clinical response rates <25%), non-limiting examples of which include liver cancer, pancreatic cancer, head and neck cancer, stomach cancer, colorectal cancer, gastric cancer, breast cancer, sarcoma, lymphoma, brain cancer and ovarian cancer. The cancer may be characterised by cancerous cells which express decay accelerating factor (DAF/CD55) and/or Neonatal Fc Receptor (FcRn).

Picornaviruses of the present invention may be any Picornavirus including known and classified Picornavirus and yet to be classified Picornavirus. The Picornaviruses may be selected from prototype and clinically isolated strains. Representative types of human Picornaviruses include Enteroviruses, Coxsackieviruses, Echoviruses, Polioviruses, unclassified Enteroviruses, Rhinoviruses, Paraechoviruses, Hepatoviruses, and Cardioviruses. In some embodiments the Picornaviruses may include Enteroviruses including Coxsackieviruses, Echoviruses, Polioviruses, unclassified Enteroviruses, or viruses from other genera(s) of Picornaviruses which may include Rhinoviruses, Paraechoviruses, Hepatoviruses, Cardioviruses, Aphthoviruses, Erboviruses, Koboviruses and Teschoviruses. Modified forms of these viruses are also included withion the scope of the present invention.

In certain embodiments, the Picornaviruses and modified forms thereof may be Picornaviruses that are capable of binding to decay-accelerating factor (DAF, also known as CD55) receptors on the surface of cancer cells such as, for example, Enteroviruses (e.g. Coxsackieviruses or Echoviruses). Non-limiting examples include Enterovirus 68, 70, Coxsackie B virus serotypes 1, 3, and 5, and Echovirus serotypes 6, 7, 12, 20, 21, Coxsackievirus A21. The Picornaviruses and modified forms thereof may use the DAF receptor to mediate viral adhesion to cancerous cells and/or to mediate viral entry into cancerous cells.

Additionally or alternatively, the Picornaviruses and modified forms thereof may be capable of binding to one or more other receptor type(s) on the surface of cancer cells such as, for example, Intercellular Adhesion Molecule 1 (ICAM-1), Intercellular Adhesion Molecule 1 (ICAM-5), Integrin α2 β1, Integrin αV β3, Integrin αV β6, Neonatal Fc receptor (FcRn), Coxsackievirus and adenovirus receptor (CAR/CXADR), Sialic acids (e.g. alpha 2,3 Sialic acids), Lysosomal integral membrane protein 2 (LIMP-2/SCARB2), P-Selectin Glycoprotein Ligand (SELPLG), low-density lipoprotein receptor (LDLR), Poliovirus receptor (PVR), and/or cadherin-related family member 3 (CDHR3).

Picornaviruses used in the compositions and methods of the present invention may be naturally-occurring or modified forms thereof.

A Picornavirus is “naturally-occurring” when it can be isolated from a source in nature and has not been intentionally modified by humans in the laboratory. For example, the Picornavirus may be obtained from a “field source”, such as, from a human patient.

A Picornavirus may be “modified” by altering one or more characteristics as compared to a naturally-occurring Picornavirus.

For example, the Picornavirus may be a recombinant Picornavirus derived from two or more types of Picornaviruses with differing pathogenic phenotypes such that it contains different antigenic determinants thereby reducing or preventing an immune response by a mammal previously exposed to a Picornavirus subtype. Such recombinant virions can be generated by co-infection of mammalian cells with different subtypes of Picornavirus with the resultant resorting and incorporation of different subtype coat proteins into the resulting virion capsids.

Additionally or alternatively, the Picornavirus may be modified by making change(s) to structural proteins, such as for example one or more of VP1, VP2, VP3 and/or VP4. The structural proteins(s) may exposed on the outer surface of the capsid (e.g. one or more of VP1, VP2, VP3). Non-limiting examples of changes in the VP1, VP2, and/or VP3 protein(s) include those set out in Table 1 below:

TABLE 1
Non-limiting VP1, VP2, VP3 amino acid changes

| | Exemplary Amino Acid | Amino Acid Identity |
|---|---|---|
| Protein/Residue | Identity in Wild-Type | in Modified |
| Number¹ | Strain | Picornavirus Strain |
| VP1 (132) | Gln | Arg |
| VP1 (210) | Phe | Tyr |
| VP1 (230) | Tyr | His |
| VP2 (142) | Asn | Thr |
| VP2 (154) | His | Tyr |
| VP2 (168) | Ser | Asn |
| VP3 (206) | Ala | Val |

¹Residue numbering in accordance with Echovirus 12 Travis strain

Without limitation, the Picornavirus may comprise any one or more of: a VP1 capsid protein amino acid sequence comprising any one SEQ ID NOs: 54-57, 74, 86, 98, 110, 122, 134, 146 or 158, a VP2 capsid protein amino acid sequence comprising any one of SEQ ID NOs: 58-61, 75, 87, 99, 111, 123, 135, 147 or 159, and/or a VP3 capsid protein amino acid sequence comprising any one of SEQ ID NOs: 62, 63, 76, 88, 100, 112, 124, 136, 148 or 160.

Additionally or alternatively, the Picornavirus may be modified by making change(s) to non-structural viral proteins such as, for example, one or more of 2A, 2B, 2C, 2BC, 3A, 3B, 3AB, 3C, 3D, and 3CD.

TABLE 2
Non-limiting amino acid changes in other Picornavirus proteins

| | Exemplary Amino Acid | Exemplary Amino Acid |
|---|---|---|
| Protein/Residue | Identity in Wild-Type | Identity in Modified |
| Number¹ | Strain | Picornavirus Strain |
| 2A (47) | Phe | Tyr |
| 3A (6) | Ile | Tyr |
| 3C (39) | His | Arg |
| 3D (123) | Ile | Leu |

¹Residue numbering in accordance with Echovirus 12 Travis strain

Without limitation, the Picornavirus may comprise any one or more of: a non-structural protein 2A sequence comprising any one of SEQ ID NOs: 64, 65, 77, 89, 101, 113, 125, 137, 149 or 161, a non-structural protein 3A sequence comprising any one of SEQ ID NOs: 66, 67, 78, 90, 102, 114, 126, 138, 150 or 162, a non-structural protein 3C sequence comprising any one of SEQ ID NOs: 68, 69, 79, 91, 103, 115, 127, 139, 151 or 163, and/or a non-structural protein 3D sequence comprising any one of SEQ ID NOs: 70, 71, 80, 92, 104, 116, 128, 140, 152 or 164.

It will be understood in relation to any amino acid sequence or nucleotide sequence changes described herein, including the amino acid changes noted in Tables 1 and 2 above, that the length of an amino acid sequence of a given individual protein may vary in different Picornavirus strains. Thus, reference herein to an amino acid change at a given residue position in a Picornavirus protein sequence ‘A’ will be understood to encompass that same amino acid change at an equivalent position in the same Picornavirus protein sequence ‘B’, which although differing in length to the sequence of ‘A’ is still representative of the same Picornavirus protein. Equivalent amino acid positions in amino acid sequences of different length but representative of the same Picornavirus protein can be routinely identified using standard methods of sequence alignment known in the art.

The Picornavirus proteins may be changed by replacement, insertion or deletion of amino acids. This may be achieved using any suitable technique including, but not limited to, recombinant methods. Replacement includes the insertion of different amino acids in place of the native amino acids. Insertions include the insertion of additional amino acid residues into the protein at one or more locations. Deletions include deletions of one or more amino acid residues in the protein. Changes of this nature may be generated by methods known in the art. For example, oligonucleotide site directed mutagenesis of gene(s) encoding one or more of the protein(s) (e.g. capsid protein(s)) could result in the generation of the desired altered capsid protein(s). Expression of the altered protein(s) in Picornavirus-infected mammalian cells in vitro can result in the incorporation of the altered protein into the Picornavirus virion particle.

In some embodiments, the Picornaviruses may be modified to reduce or eliminate an immune reaction to the Picornavirus. Such modified Picornaviruses are termed “immunoprotected Picornaviruses”. Suitable modifications could include packaging of the Picornavirus in a liposome, a micelle or other vehicle to mask the Picornavirus from the subject's immune system. Alternatively, the outer capsid of the Picornavirus virion particle may be removed or altered since the proteins present in the outer capsid are the major determinant of the host humoral and cellular responses.

In some embodiments, the Picornaviruses may be modified by making changes to viral nucleic acid sequences, including untranslated regions (UTRs), regulatory regions and the like.

The Picornaviruses may further be modified by insertion of exogenous nucleic acids encoding protein(s) of interest or components thereof. Exogenous nucleic acids may be inserted into RNA using standard methods known to persons skilled in the art (see, for example, Ferran and Skuse (Eds), “Recombinant Virus Vaccines Methods and Protocols”, 2017, Springer Protocols; Green and Joseph. (2012), Molecular cloning: a laboratory manual, fourth edition. Cold Spring Harbor, N.Y.: Cold Spring Harbor Laboratory Press; Ausubel et al. (1987-2016), Current Protocols in Molecular Biology. New York, NY, John Wiley & Sons). Non-limiting examples of exogenous nucleic acids that may be inserted into Picornaviruses of the present invention include those encoding cytokines (e.g. interleukins, interferons), chemokines, immune checkpoint inhibitors (e.g. PD-1, PD-L1, PD-L2, CTLA-4, CD134, CD134L, CD137, CD137L, CD80, CD86, B7-H3, B7-H4, B7RP1, ICOS, LAG-3, TIM3, GAL9, CD28, OX-40, TIGIT), and the like.

Additionally or alternatively, the Picornavirus may be modified via bioselection (also known in the art as directed evolution) for certain traits. For example, the Picornaviruses may be bioselected for enhanced infectivity, enhanced safety, enhanced lytic capability, enhanced migration between cancer cells, enhanced DAF and/or FcRn binding, enhanced replicative capacity at mammalian body temperatures (33° C.-39° C., 35° C.-39° C., 37° C.-39° C.), enhanced lytic activity of cancer cells expressing PD-L1, and the like. Methods for bioselecting viruses with target traits are well known to those of skill in the art (see, for example, Zainutdinov et al. “Directed evolution as a tool for the selection of oncolytic RNA viruses with desired phenotypes”, Oncolytic Virotherapy; Macclesfield Vol. 8, (2019): 9-26; Svyatchenko et al. “Bioselection of coxsackievirus B6 strain variants with altered tropism to human cancer cell lines”, Arch Virol (2017) 162:3355-3362; Yan et al. “Developing Novel Oncolytic Adenoviruses through Bioselection”, J. Virol, 2003, p. 2640-2650).

The present invention includes isolated nucleic acid molecules from Picornaviruses. In some embodiments the Picornaviruses are capable of binding at least to DAF receptors on the surface of cancer cells. In some embodiments the nucleic acid molecule may be derived from the Picornavirus and may be single-stranded RNA, or synthetically produced viral RNA or complementary DNA from the viruses. It will be appreciated that a nucleic acid sequence of the invention includes a nucleic acid sequence that has been derived from a Picornavirus including, for example, a nucleic acid sequence encoding the viral genome or a sufficient sequence thereof to permit generation of the virus or to be capable of eliciting a lytic infection in a cell. For example, the nucleic acid molecule may comprise a single viral RNA or DNA molecule, such as a complementary DNA molecule, or a plurality of such molecules encoding different viral sequences.

It is to be understood that in the context of the specification the term “derived” from thus includes that the sequence may be viral RNA directly isolated from a Picornavirus, synthetic viral RNA, or cDNA encoding the viral genome or a component thereof corresponding to the isolated sequence. The term also includes synthetic polynucleotide sequences comprising one or more changes in the sequence compared to wild-type sequence or parental sequence, including, for example changes in the capsid proteins.

Any suitable method for isolation of viral RNA may be used, including methods based on the use of phenol/chloroform extraction, such as provided in commercial kit form for isolation of viral RNA, such as Trizol® LS reagent (GIBCO BRL, Life Technologies Grand Island, NY, USA), isolation methods which utilise magnetic bead-based isolation, such as Ambion MagMax™ viral RNA isolation kits. Methods for the isolation of viral RNA are generally described in, for example Ausubel et al. (1987-2016), Current Protocols in Molecular Biology. New York, NY, John Wiley & Sons; and in Sambrook et al., (1989), Molecular Cloning: A Laboratory Manual, Second Ed., Cold Spring Harbour Laboratory Press, New York.

It will be appreciated that the invention does not require the nucleic acid sequence, such as viral RNA, whether it be directly isolated from virus, synthesised, presented as a plasmid molecule or generated in vitro such as from cDNA templates (e.g. those encoding the viral genome or a component thereof) using bacteriophage T7 RNA polymerase, to be devoid of contaminant material, such as cell debris, to be considered “isolated” in the context of this specification. Thus, in the context of the specification RNA will be considered isolated when non-RNA components from the source material, such as cellular proteins, have been partially or completely removed from the RNA. For example, the RNA will be considered “isolated” when greater than 50% of non-RNA material has been removed. It is preferred that greater than 60% of the non-RNA material be removed, more preferably greater than 70%, 80% or 90% of the non-RNA material will be removed. Typically, the RNA will contain less than 10% contaminant material, more typically less than 5% contaminant material. Thus, the RNA will preferably be greater than 95% pure for viral RNA, even more preferably greater than 97% pure or greater than 99% pure.

The nucleic acid molecule may comprise a nucleic acid sequence comprising or consisting of SEQ ID NO: 41, SEQ ID NO: 50, SEQ ID NO: 51, SEQ ID NO: 72, SEQ ID NO: 84, SEQ ID NO: 96, SEQ ID NO: 108, SEQ ID NO: 120, SEQ ID NO: 132, SEQ ID NO: 144 or SEQ ID NO: 156. Those skilled in the art will recognise that, in view of the degeneracy of the genetic code considerable sequence variation is possible among these polynucleotide molecules.

The present invention also provides isolated polynucleotide sequences that are substantially similar to the polynucleotides disclosed herein, for example to SEQ ID NO: 41, SEQ ID NO: 50, SEQ ID NO: 51, SEQ ID NO: 72, SEQ ID NO: 84, SEQ ID NO: 96, SEQ ID NO: 108, SEQ ID NO: 120, SEQ ID NO: 132, SEQ ID NO: 144 or SEQ ID NO: 156, where such sequences comprise or provide a Picornavirus with the capability of binding a DAF receptor and optionally additional cell receptor(s), and lytically infecting a cancerous cell. The polynucleotide sequence variants possess qualitative biological activity in common with the base sequences (for example, SEQ ID NO: 41, SEQ ID NO: 50, SEQ ID NO: 51, SEQ ID NO: 72, SEQ ID NO: 84, SEQ ID NO: 96, SEQ ID NO: 108, SEQ ID NO: 120, SEQ ID NO: 132, SEQ ID NO: 144 or SEQ ID NO: 156), and have at least about 60%, at least 70%, at least 80%, at least 90%, at least 95%, at least 96%, at least 97%, at least 98%, or at least 99%, sequence identity to the base sequences (for example, SEQ ID NO: 41, SEQ ID NO: 50, SEQ ID NO: 51, SEQ ID NO: 72, SEQ ID NO: 84, SEQ ID NO: 96, SEQ ID NO: 108, SEQ ID NO: 120, SEQ ID NO: 132, SEQ ID NO: 144 or SEQ ID NO: 156). As used herein “sequence identity” refers to the residues in two sequences that are the same when aligned for maximum correspondence over a specified window of comparison by means of computer programs known in the art such as GAP provided in the GCG program package (Program Manual for the Wisconsin Package, Version 8, August 1996, Genetics Computer Group, 575 Science Drive, Madison, Wisconsin, USA 53711) (Needleman, S. B. and Wunsch, C. D., (1970), Journal of Molecular Biology, 48, 443-453).

Picornaviruses, modified forms thereof, and picornavirus RNA/cDNA of the present invention can be manufactured using standard techniques well known in the art (see, for example, Chen et al. 2019, “Physical, chemical, and synthetic virology: Reprogramming viruses as controllable nanodevices, Wiley Interdiscip Rev Nanomed Nanobiotechnol. 11(3):e1545. doi:10.1002/wnan.1545; Geunther et al 2014, “Synthetic virology: engineering viruses for gene delivery.” Wiley interdisciplinary reviews. Nanomedicine and nanobiotechnology vol. 6,6: 548-58. doi:10.1002/wnan.1287. Non-limiting examples of modifications to the viruses and/or their RNA/cDNA include insertion of tissue specific miRNAs and/or nucleic acids encoding immune checkpoint inhibitors such as, for example, monoclonal antibodies binding to immune check-point targets, (e.g. PD-1, PD-L1, CTLA-4, IDO, TIM-3, LAG-3, TIGIT and the like).

Picornaviruses and/or modified forms thereof of the present invention may be administered to subjects in the form of a pharmaceutical composition comprising virus and a pharmaceutically acceptable carrier. The composition may comprise the virus at any suitable concentration, such as in a concentration range of about 10⁵ viral particles per mL to about 10¹⁵ viral particles per mL, or about 10⁶ viral particles per mL, or about 10⁷ viral particles per mL or about 10⁸ viral particles per mL, or about 10⁹ viral particles per mL, or about 10¹⁰ viral particles per mL, or about 10¹¹ viral particles per mL, or about 10¹² viral particles per mL, about 10¹³ viral particles per mL, or about 10¹⁴ viral particles per mL, or about 10¹⁵ viral particles per mL.

A stock of the virus composition may be diluted to an appropriate volume suitable for dosing, for example to achieve the desired dose of viral particles administered in a desired volume. For example, a subject may be administered a dose of virus comprising about 10⁵ viral particles to about 10¹⁵ viral particles, or about 10⁶ viral particles, or about 10⁷ viral particles, or about 10⁸ viral particles, or about 10⁹ viral particles, or about 10¹⁰ viral particles, or about 10¹¹ viral particles, or about 10¹² viral particles, or about 10¹³ viral particles, or about 10¹⁴ viral particles, or about 10¹⁵ viral particles. The volume in which the virus is administered will be influenced by the manner of administration. For example, administration of the virus by injection would typically be in a smaller volume, for example about 0.5 mL to about 10 mL. As a further example, intravenous administration of virus may typically use about 100 mL to about 500 mL of virus diluted in normal saline, infused by an automatic pump over approximately 30 minutes.

The pharmaceutical compositions provided herein may comprise Picornaviruses at between about 10⁸ and about 10¹⁵ viral particles per mL, between about 10⁸ and 10¹² viral particles per mL, or between about 10⁸ and 10¹⁰ viral particles per mL.

The pharmaceutical compositions provided herein may comprise Picornaviruses at a particle to infectivity ratio less than or equal to about 3000:1, less than or equal to about 300:1, in the range of about 200:1 to about 5:1, in the range of about 50:1 to about 10:1, or less than about 20:1.

The pharmaceutical compositions provided herein may further comprise a level of host cell DNA less than or equal to about 200 ng per mL, in the range of 1 to about 100 ng per mL, in the range of about 1 to about 50 ng per mL, or in the range of about 1 to about 10 ng per mL.

Pharmaceutical compositions comprising Picornaviruses and/or modified forms thereof of the present invention may additionally include a pharmaceutically acceptable diluent, excipient and/or adjuvant. The carriers, diluents, excipients and adjuvants must be “acceptable” in terms of being compatible with the other ingredients of the composition, and not unacceptably deleterious to the recipient subject.

The virus may be administered as naked viral RNA encoding the virus, rather than viral particles, as described for example in PCT/AU2006/000051 entitled “Methods and composition for the treatment of neoplasms”, filed 17 Jan. 2006, published as WO2006/074526, the entire contents of which are incorporated herein by reference. In such embodiments the viral RNA may be administered in the form of liposomes. Liposomes are generally derived from phospholipids or other lipid substances and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which is incorporated herein by reference.

Alternatively, the viral RNA may be administered in the form of lipid nanoparticles (LNPs). Lipid nanoparticles (LNPs) are typically composed of four main types of lipids: ionizable cationic lipids, which are crucial for encapsulating nucleic acids and aiding endosomal release; phospholipids, which contribute to the structural integrity of the nanoparticle; cholesterol, which enhances stability and fluidity; and polyethylene glycol (PEG)-lipids, which extend circulation time in vivo by forming a steric barrier. However, other non-toxic, physiologically acceptable and metabolisable lipids capable of forming LNPs can be used as understood by those skilled in the art. The formation of LNPs generally involves microfluidic mixing or ethanol dilution, where the lipids and the therapeutic payload are rapidly mixed in an aqueous environment. However, other methods to form LNPs are known in the art.

Administration routes for Picornaviruses and modified forms thereof according to the present invention, and pharmaceutical compositions comprising them, to a given subject include, but are not limited to intratumoural, intravenous, intravesical, subcutaneous, oral, intravesicular, dermal, isolated hepatic perfusion, intrahepatic, ocular and topical administration routes, and any combination thereof.

Picornaviruses and/or modified forms thereof of the present invention may be administered to subjects by any appropriate means, such as by injection. The injection may, for example, be systemic, parenteral, or directly into the cancer. Intralesional injection of a tumour may be performed by any appropriate means known to the skilled person, taking into account factors such as the type of tumour being treated, the size and location of the tumour, accessibility of the tumour to direct injection. Injection techniques which increase or maximise the distribution of the virus throughout the tumour may offer improved therapeutic outcomes. For example, in the treatment of melanoma and other solid tumours, multiple lesions may be injected in a dose hyper-fraction pattern, starting with the largest lesion(s) (2.0 mL injected into tumours >2.5 cm, 1.0 mL into 1.5 to 2.5 cm; 0.5 mL into 0.5 to 1.5 cm) to a 4.0 mL maximum. Following initial injection with of Picornaviruses described herein, any injected lesion that reduces in diameter to <0.5 cm may be injected with 0.1 mL of the Picornaviruses and/or modified forms thereof as per the stated treatment schedule until the lesion completely resolves.

In one embodiment of the invention, in the treatment of colorectal cancer and other solid tumours having specified lengths, single and multiple lesions may be treated using the following dosage regimen:
1. For tumours having a length ≥50 mm:
   Up to 10 mL IVX037 injected for a single lesion.
   If multiple lesions are injected, at least 5.0 mL is allocated to the largest lesion, and the remaining 5.0 mL divided among the smaller lesions based on their size.
2. For tumours having a length ≥25 mm and <50 mm:
   3.0 mL IVX037 injected for a single lesion.
   If multiple lesions are to be injected, up to an additional 3.0 mL can be injected, distributed based on size.
3. For tumours having a length ≥10 mm and <25 mm:
   1.0 mL IVX037 injected for a single lesion.
   If multiple lesions are to be injected (up to 4), the dose may be distributed among the target lesions, each receiving 1 mL and 25% of the total dose.
4. For tumours having a length ≥5 mm and <10 mm:
   If treated lesions regress to <10 mm, the volume of IVX037 injected is 0.5 mL until disappearance.

Maximizing the number of cancer cells and regions throughout the tumour that are initially infected theoretically will increase the amount of cancer cells destroyed. It will also increase the amount of viral progeny produced by the tumour, and therefore increase the chance of ongoing viremia for the seeding of remote tumours. Any appropriate means to achieve desired distribution of the administered virus through the tumour may be used and will be apparent to the skilled addressee.

Picornaviruses and/or modified forms thereof of the present invention may be administered to a subject, tumour or cancer cells with other agents, either simultaneously or sequentially.

For example, they may be administered in combination with immuno-stimulatory agent(s). The immuno-stimulatory agent(s) may be selected from any appropriate agents. In the context of the present invention an immuno-stimulatory agent will be understood as any agent capable of stimulating an immune response to tumour/cancer cells when administered to an individual. The immuno-stimulatory agent may be any agent that interacts with an immune checkpoint molecule to block, diminish or counteract the ability of that immune checkpoint molecule, or a complex comprising that immune checkpoint molecule, in reducing the innate immune-based anti-tumour responses of the individual. Hence, an immuno-stimulatory agent reduces the “handbrake” effect that the immune checkpoint molecules have on the anti-tumour response. For example, the immuno-stimulatory agent may be any agent that targets an immune checkpoint molecule selected from the group consisting of PD-1, PD-L1, PD-L2, CTLA-4, CD134, CD134L, CD137, CD137L, CD80, CD86, B7-H3, B7-H4, B7RP1, ICOS, TIM3, GAL9, CD28, OX-40, and TIGIT. It will also be understood that the term immuno-stimulatory agent as used herein may also be referred to as immune checkpoint inhibitors, when the immuno-stimulatory agent targets an immune checkpoint.

In some embodiments the immuno-stimulatory agent is an antibody, such as a monoclonal antibody. Preparation of antibodies for use in the present invention may be carried out by methods well known in the art, including preparing monoclonal antibodies using well known techniques and screening for high affinity antibodies, or by first identifying a monoclonal antibody having reasonably high affinity and then improving the affinity using well known methods (see, for example, Huse et al., Internat'l Rev. Immunol. 10:129-137 (1993); Yelton, et al., J. Immunol. 155:1994-2004 (1995); W, et al., Proc. Natl. Acad. Sci. (USA) 95:6037-6042 (1998); Crameri et al., Nature Medicine 2:100-103 (1996); Stemmer, Proc. Natl. Acad. Sci. (USA) 91:10747-10751 (1994); Stemmer, Nature 370:389-391 (1994); sections of these documents describing the preparation of antibodies are incorporated herein by reference). Alternatively, the antibodies may be obtained rather than prepared. Examples of antibodies targeting checkpoint inhibitor molecules include Nivolumab (BMS-936558, MDX-1106, ONO-4538), a fully human Immunoglobulin G4 (IgG4) monoclonal PD-1 antibody which was the first of its class to be tested in a phase I trial of 10⁷ patients with metastatic melanoma (see Sosman et al. 2012b), Lambrolizumab (MK-3475), a humanized monoclonal IgG4 PD-1 antibody, which was studied in a phase I trial that included 132 patients with metastatic melanoma (see Iannone et al. 2012), and BMS-936559, a fully human IgG4 PD-L1 antibody, was tested in 55 patients with metastatic melanoma as part of the phase I trial (see Brahmer et al. 2012).

Additionally or alternatively, Picornaviruses and/or modified forms thereof of the present invention may be administered to a subject, tumour or cancer cells in combination (either simultaneously or sequentially) with CAR-T cells and/or natural killer (NK) cells. Methods for the generation of CAR-T cells are well known to those skilled in the art (see, for example, Zhao et al. “Universal CARs, universal T cells, and universal CAR T cells”, Journal of Hematology & Oncology, volume 11, Article number: 132 (2018); Wang and Rivière, “Clinical manufacturing of CAR T cells: foundation of a promising therapy”, Molecular Therapy-Oncolytics (2016) 3, 16015; Vormittag et al. “A Guide to Manufacturing CAR-T Cell Therapies”, Curr Opin Biotechnol. 2018 October; 53:164-181). By way of non-limiting example, the CAR-T cells may be engineered to bind to any one or more of: EGFRVIII, interleukin 13Ra2, FAP, GD2, EpCam, CD133, CD70, Her2, CEA, GAP, CD5, CD38, Muc1, GPC3, BCMA, Meso, PSCA, CD33, PSMA, ROR1, Muc1, EphA2, CD123, CD19, Claudin 18.2; on the surface of a cancerous cell.

Additionally or alternatively, Picornaviruses and/or modified forms thereof of the present invention may be administered to a subject, tumour or cancer cells in combination (either simultaneously or sequentially) with chemotherapeutic agent(s). By way of non-limiting example, suitable chemotherapeutic agents include ABVD, AC chemotherapy, Amsacrine, (Amsidine), Asparaginase, Azacitidine, (Vidaza®), BEACOPP chemotherapy, BEAM chemotherapy, Bendamustine, (Levact®), BEP chemotherapy, Bleomycin, Busulfan, (Busilvex®, Myleran®), Cabazitaxel, (Jevtana®), Capecetabine and docetaxel, Capecitabine, (Xeloda®), Carboplatin, Carboplatin and etoposide chemotherapy, Carmustine, (BiCNU®), CAV chemotherapy, Chlorambucil, (Leukeran®), ChIVPP chemotherapy, CHOP chemotherapy, Cisplatin, Cisplatin and fluorouracil chemotherapy, 5FU, Cisplatin and topotecan chemotherapy, (CT), Cisplatin, capecitabine and trastuzumab, (HCX), Cladribine, (Leustat®, LITAK®), Clofarabine, (Evoltra®), CMF chemotherapy, CODOX-M chemotherapy, Crisantaspase, (Erwinase®, asparaginase or L-asparaginase), CTD chemotherapy, CVP chemotherapy, Cyclophosphamide, Cytarabine, Dacarbazine, (DTIC), Dactinomycin, (Cosmegen Lyovac®), Daunorubicin, De Gramont and modified de Gramont chemotherapy, DHAP Chemotherapy, Docetaxel, (Taxotere®), Docetaxel and carboplatin chemotherapy, (Taxotere®), Docetaxel and cisplatin chemotherapy, (Taxotere®), Doxorubicin, (Adriamycin@), Doxorubicin and ifosfamide chemotherapy, EC chemotherapy, ECF chemotherapy, E-CMF chemotherapy, (Epi-CMF), ECX chemotherapy, EOX chemotherapy, Epirubicin, (Pharmorubicin®), Eribulin, (Halaven®), ESHAP chemotherapy, Etoposide, (Etopophos®, Vepesid®), Etoposide and cisplatin chemotherapy, (EP/PE), FCR chemotherapy, FEC chemotherapy, FEC-T chemotherapy, FLAG-Ida, FLOT chemotherapy, Fludarabine, (Fludara®), Fluorouracil, (5FU), FOLFIRINOX chemotherapy, GemCarbo chemotherapy, Gemcitabine, (Gemzar®), Gemcitabine and capecitabine (GemCap), Gemcitabine and cisplatin chemotherapy, (GemCis or GemCisplat), Gliadel® wafers, (Carmustine), Hydroxycarbamide, (Hydrea®, hydroxyurea), Hyper-CVAD chemotherapy, Idarubicin, (Zavedos®), Ifosfamide, (Mitoxana®), Ifosfamide, carboplatin, etoposide-ICE chemotherapy, Irinotecan, (Campto®), Irinotecan with fluorouracil and folinic acid, (5FU) and (FOLFIRI), Leucovorin, (Folinic acid), Liposomal daunorubicin, (DaunoXome®), Liposomal doxorubicin, (Caelyx®, Myocet®), Lomustine, Melphalan, (Alkeran®), Mercaptopurine, (Puri-Nethol®), Mesna, (Uromitexan®), Methotrexate, Mitomycin, (Mitomycin C Kyowa®), Mitomycin and fluorouracil, (5FU), Mitotane, (Lysodren®), Mitoxantrone, MPT chemotherapy, MPT chemotherapy, MVAC chemotherapy, Nab-paclitaxel, (Abraxane®), Oxaliplatin, (Eloxatin®), Oxaliplatin with fluorouracil and folinic acid chemotherapy, (5FU) and (FOLFOX or OxMdG), Paclitaxel, (Taxol®), Paclitaxel and carboplatin chemotherapy, (Taxol/Carbo), PCV chemotherapy, Pemetrexed, Pemetrexed and carboplatin, Pemetrexed and cisplatin chemotherapy, Pentostatin, (Nipent®), Pertuzumab, trastuzumab and docetaxel, PMitCEBO chemotherapy, Procarbazine, Raltitrexed, (Tomudex®), Rasburicase, (Fasturtec®), R-CHOP chemotherapy, R-CVP, R-DHAP chemotherapy, R-ICE chemotherapy, Streptozocin, (Zanosar®), TAC chemotherapy, TC (Taxotere and cyclophosphamide) chemotherapy, Temozolomide, (Temodal®), Thiotepa, Tioguanine, (Lanvis®), TIP chemotherapy, Topotecan, (Hycamtin®), Trabectedin, (Yondelis®), Treosulfan, Trifluridine-tipiracil hydrochloride, (Lonsurf®), Vinblastine, (Velbe®), Vincristine, (Oncovin®), Vinorelbine, (Navelbine®), Vinorelbine and carboplatin chemotherapy, (VP), Vinorelbine and cisplatin chemotherapy, (VP), XELOX (or CAPOX), XELOX (or CAPOX).

The methods of the present invention may be used in combination with surgical treatment of the cancer. For example tumour resection may be followed by treatment of the subject using methods of the present invention. It is anticipated that this may prevent or reduce recurrence of the tumour.

Additionally or alternatively, the methods of the present invention may be used in combination with neoadjuvant therapy. For example, neoadjuvant therapy may be followed by treatment of the subject using methods of the present invention. It is anticipated that this may prevent or reduce recurrence of the tumour.

Additionally or alternatively, the methods of the present invention may be used in combination with radiation therapy (e.g. X-rays, protons, and/or other particles). For example, radiation therapy may be followed by treatment of the subject using methods of the present invention. It is anticipated that this may prevent or reduce recurrence of the tumour.

The methods may comprise single or multiple doses of any one or more of the Picornaviruses, immuno-stimulatory agents, CAR-T cells, natural Killer (NK) cells, chemotherapeutic agents, and/or radiotherapeutic agents. The methods of the present invention comprise administering the Picornaviruses and/or modified forms thereof described herein and/or their RNA and/or cDNA produced from the RNA to a subject, tumour or cancer cell. While there is no particular limitation to the type of cancer that can be treated, alleviated or prevented according to the methods described herein, in some embodiments the cancer is one that is recognised, either in the general sense or in the case of a specific subject, tumour or cancer cell, to have resistance (i.e. reduced response levels) to treatment with immune checkpoint inhibitors. Non-limiting examples of such cancers include liver cancer, pancreatic cancer, head and neck cancer, stomach cancer, colorectal cancer, gastric cancer, breast cancer, sarcoma, lymphoma, brain cancer and ovarian cancer. The cancer may be characterised by cancerous cells which express decay accelerating factor (DAF/CD55) and/or Neonatal Fc Receptor (FcRn).

The invention also relates to kits for use in the methods of the invention. In a basic form, the kit may comprise a pharmaceutical composition comprising Picornaviruses and/or modified forms thereof of the present invention, a pharmaceutically acceptable carrier, and instructions for the use of the composition. The kit may additionally comprise any one or more of: immune checkpoint inhibitors, CAR-T cells, natural Killer (NK) cells, chemotherapeutic agents, radiotherapeutic agents. The composition may be provided in any suitable container, such as for example a vial, ampoule or syringe. The composition may be provided lyophilised, freeze-dried, in liquid form or frozen state.

The kit may comprise any number of other additional components. By way of non-limiting example, additional components may include (i) one or more anti-viral agents, such as Plecornil; (ii) one or more additional pharmaceutical compositions comprising an oncolytic virus; (iii) one or more additional pharmaceutical compositions comprising oncolytic viral RNA; (iv) one or more additional therapeutic agents useful in the treatment of cancer in a patient. The kit may also comprise of the composition being contained in a single-use vial, a pre-loaded syringe for direct human administration, diluted in a physiological solution for intravenous infusion or in a concentrated form enabling suitable dilution with physiological solutions. Such solutions may be, for example, phosphate buffered saline or physiological concentrations of NaCl₂.

It will be appreciated by persons of ordinary skill in the art that numerous variations and/or modifications can be made to the present invention as disclosed in the specific embodiments without departing from the spirit or scope of the present invention as broadly described. The present embodiments are, therefore, to be considered in all respects as illustrative and not restrictive.

### EXAMPLES

The present invention will now be described with reference to the following Examples, which should not be construed as in any way limiting.

### Example One: Novel Echovirus 12 Strain of IVX037 with Enhanced DAF-binding Capacity

The complete genomic sequences of the novel Echovirus 12 (E12) strains designated IVX037 (internal designation) with high affinity DAF binding were determined.

### Materials and Methods

Viral RNA was extracted from IVX037 viral stock (IVX037 RD PLC 15.06.2020) using QIAamp Viral RNA Mini Kit (SOP LAB-PROC 018) and reverse transcribed and amplified using one step reverse transcription polymerase chain reaction (LAB-PROC 027) using virus-specific primers (see Table 3).

TABLE 3
List of virus-specific primers used for sequence
analysis

| Primer name | Primer sequence 5′-3′ |
|---|---|
| EVP-1 | GCCACGTGTGGAGTCTTGTGC |
| | (SEQ ID NO: 1) |
| EVP-2 | GGTAGTGTTTATTCCTCGATGC |
| | (SEQ ID NO: 2) |
| EVP-3 | CAGTATAGGCAACGCGTATAGC |
| | (SEQ ID NO: 3) |
| EVP-4 | TCTGACTGTGCTCGTAATTGC |
| | (SEQ ID NO: 4) |
| EVP-5 | CAGCAGGATGAATATACGAGTGC |
| | (SEQ ID NO: 5) |
| EVP-6 | CTTCTACCAGACCTGGTCCTT |
| | (SEQ ID NO: 6) |
| EVP-7 | TTAAAACAGCCTGTGGGTTG |
| | (SEQ ID NO: 7) |
| EVP-8 | TGAATGCGGCTAATCCTAAC |
| | (SEQ ID NO: 8) |
| EVP-9 | AACAGCAGAAGAATGTGGGT |
| | (SEQ ID NO: 9) |
| EVP-10 | AAAGCATAGTGACAAACGCC |
| | (SEQ ID NO: 10) |
| EVP-11 | GCAACCAACTGTCAACTCAA |
| | (SEQ ID NO: 11) |
| EVP-12 | AGGAGGCCGTGAACAGAG |
| | (SEQ ID NO: 12) |
| EVP-13 | CTAGCATCTTCTGGACGGAG |
| | (SEQ ID NO: 13) |
| EVP-14 | CACAGCTCATGGGTGTGAC |
| | (SEQ ID NO: 14) |
| EVP-15 | CACTGTAACTGCTACTCTTGCC |
| | (SEQ ID NO: 15) |
| EVP-16 | GGAGCAGGGAAATCAGTTG |
| | (SEQ ID NO: 16) |
| EVP-17 | GAAGAACCCAAGTCAGATATTCA |
| | (SEQ ID NO: 17) |
| EVP-18 | CACCATGCTCGGCATCTAT |
| | (SEQ ID NO: 18) |
| EVP-19 | ATTGAGAGCTCAAAGGATGC |
| | (SEQ ID NO: 19) |
| EVP-20 | AATCAAGACTCATTGAAGCATCT |
| | (SEQ ID NO: 20) |
| EVP-21 | ACTACTTGCTGAAGCTGGGA |
| | (SEQ ID NO: 21) |
| EVP-22 | AAAGTAGTCGGTTCCGCTG |
| | (SEQ ID NO: 22) |
| EVP-23 | ACCACATTGGCACACTCC |
| | (SEQ ID NO: 23) |
| EVP-24 | AATAGTCGCGCTGTTGTTG |
| | (SEQ ID NO: 24) |
| EVP-25 | TATCTCACCTAGTAATGTCCTTTTGA |
| | (SEQ ID NO: 25) |
| EVP-26 | GTTTCTGCTGCTGTTAGTGC |
| | (SEQ ID NO: 26) |
| EVP-27 | TGAAATGTGACCAACCATCA |
| | (SEQ ID NO: 27) |
| EVP-28 | TCCTTCAAAACTGACTGGGT |
| | (SEQ ID NO: 28) |
| EVP-29 | TGGGTATCCCGTAGTATTGTG |
| | (SEQ ID NO: 29) |
| EVP-30 | GATCTGGGTCTGGTGGTAAG |
| | (SEQ ID NO: 30) |
| EVP-31 | GAACTGAATAGCCTTGCCAC |
| | (SEQ ID NO: 31) |
| EVP-32 | CCGATCTCCTGATCATTCAT |
| | (SEQ ID NO: 32) |
| EVP-33 | ATTGCCTTCAAACACCTGAT |
| | (SEQ ID NO: 33) |
| EVP-34 | CGGGTTCAGATGGAATGTT |
| | (SEQ ID NO: 34) |
| EVP-35 | CCAGGTAACCTCATTGAAGC |
| | (SEQ ID NO: 35) |
| EVP-36 | TTCCGCACCGAATGCGGAG |
| | (SEQ ID NO: 36) |
| EVP-37 | AAACAGCCTGTGGGTTGTC |
| | (SEQ ID NO: 37) |
| EVP-38 | CGCCATGGAACAAGGAGT |
| | (SEQ ID NO: 38) |
| 3′ Anchor primer | TTTTTTTTTTTTTTTTTTTT |
| | TTTTTCCGCACC |
| | (SEQ ID NO: 39) |
| Notdt25 | ATAAGAATGCGGCCGCTTTT |
| | TTTTTTTTTTTTTTTTTTTTT |
| | (SEQ ID NO: 40) |

Amplified DNA was analysed on agarose gels (LAB PROC 014) and purified using QIAquick PCR purification kit (LAB-PROC 016) or QIAquick Gel Extraction Kit (LAB-PROC 015). The complete viral genomic sequence was determined using Sanger sequencing and virus-specific primers (LAB-PROC 012) at the AGRF, Westmead NSW, Australia. The genomic sequence was analysed using Sequencher version 5.4.6 and compared to the wild-type E12 prototype strain Travis viral sequence (GenBank accession number X79047.01). Pairwise sequence alignments were performed using Emboss needle global alignment tool (EMBL-EBI).

### Results

In some embodiments, the complete genomic sequences of the IVX 037 strains isolated were 7423 nucleotides (nt) excluding the poly(A) tail (for example, SEQ ID NO: 41, SEQ ID NO: 50, SEQ ID NO: 51, SEQ ID NO: 72, SEQ ID NO: 84, SEQ ID NO: 96, SEQ ID NO: 108, SEQ ID NO: 120, SEQ ID NO: 132, SEQ ID NO: 144 or SEQ ID NO: 156). In some embodiments, the open reading frame (for example SEQ ID NO: 43, SEQ ID NO: 81, SEQ ID NO: 93, SEQ ID NO: 105, SEQ ID NO: 117, SEQ ID NO: 129, SEQ ID NO: 141, SEQ ID NO: 153 or SEQ ID NO: 165) encodes a polyprotein of 2193 amino acids (for example SEQ ID NO: 42, SEQ ID NO: 52, SEQ ID NO: 53, SEQ ID NO: 73, SEQ ID NO: 85, SEQ ID NO: 97, SEQ ID NO: 109, SEQ ID NO: 121, SEQ ID NO: 133, SEQ ID NO: 145 or SEQ ID NO: 157) and is proceeded by a 741 nucleotide long 5′ untranslated region (UTR) (for example, SEQ ID NO: 44, SEQ ID NO: 82, SEQ ID NO: 94, SEQ ID NO: 106, SEQ ID NO: 118, SEQ ID NO: 130, SEQ ID NO: 142, SEQ ID NO: 154 or SEQ ID NO: 166) and followed by a 103 nt long 3′UTR (for example, SEQ ID NO: 45, SEQ ID NO: 83, SEQ ID NO: 95, SEQ ID NO: 107, SEQ ID NO: 119, SEQ ID NO: 131, SEQ ID NO: 143, SEQ ID NO: 155 or SEQ ID NO: 167) and a poly(A) tail.

The viral genome of the IVX037 strains of the invention is a positive-sense single-stranded RNA virus and due to the nature of the genome, the first 20 nucleotides in the extreme 5′ end are not experimentally determined, but these regions are well conserved amongst enteroviruses and is therefore highly likely that these sequences represent the true sequence of IVX037 (see Polacek et al., (2001). Genetic characterization if the Coxsackievirus B2 3′ untranslated region. Journal of General Virology 82, 1339; Mahud et al. (2019). Structure of the 5′ Untranslated Region of Enteroviral Genomic RNA. Journal of Virology 93, e01288). The average sequence coverage for an exemplary IVX037 genome of the invention is >5-fold and the vast majority of the genome was determined with at least one sequence of at high quality in forward direction and once in reverse direction (high and medium quality values have predicted probabilities of error of ≤0.01% and ≤1%, respectively.

In one embodiment, one ambiguous position was identified in the genome at nucleotide position 3074 where the virus sequence was identified to possess a mix of U (Uracil) and A (Adenine). This position was sequenced in both directions using two separate PCR amplicons (see Table 4) with the same result (mix of U/A).

TABLE 4
Detailed sequence analysis of ambiguous nt mix at position 3074.

| | | | Reading | | |
|---|---|---|---|---|---|
| | | | length | | |
| | | | from | Sequence | |
| Sequence | | | 5′end of | quality of | |
| reaction | Sequence | Target PCR | sequence | position | |
| number | direction | product | reaction | 3074² | Sequence result¹ |
| AGBD20 | [symbol] | PCR 3 17.06.2020 | 1075 | Low | T |
| | | | | | (traces of A) |
| AGBD19 | [symbol] | PCR 3 17.06.2020 | 763 | High | A |
| AGBD16 | [symbol] | PCR 3 17.06.2020 | 686 | High | A |
| | | | | | (some traces of T) |
| AGBD21 | [symbol] | PCR 3 17.06.2020 | 572 | Medium | A |
| | | | | | (some traces of T) |
| AGBD5 | [symbol] | PCR 5 17.06.2020 | 88 | Low | A |
| | | | | | (some traces of T, |
| | | | | | ~40%) |
| AGBD24 | [symbol] | PCR 5 17.06.2020 | 18 | Low | A |
| | | | | | (some traces of T) |
| AGBD18 | [symbol] | PCR 3 17.06.2020 | 40 | Medium | A/T |
| AGBD17 | [symbol] | PCR 3 17.06.2020 | 425 | Low | A/T |
| AGBD4 | [symbol] | PCR 3 17.06.2020 | 432 | Low | A/T |
| AGBD25 | [symbol] | PCR 5 17.06.2020 | 439 | Low | A/T |
| AGBD28 | [symbol] | PCR 5 17.06.2020 | 936 | Low | A/T |

¹Note that the sequence results are presented here with T (Thymidine) as determined during Sanger sequencing while the RNA sequence of IVX037 is U (Uracil).
²The sequence quality scores are a measure of the uncertainty of base calls and due to the presence of both T and A signals at this position, the generated quality scores are lower than in surrounding regions.

This ambiguity is therefore not likely to be a proof-reading error introduced during the RT-PCR amplification, but this position is in fact comprise a mix of both these nucleotides (shown as ‘W’ in the genomic sequence in SEQ ID NO: 41). This sequence ambiguity results in a mix of two amino acids being encoded at amino acid 210 in capsid protein VP1 where U encodes Phenylalanine and A Tyrosine, respectively (see Table 5).

During the analysis of the IVX037 viral genomes of the invention, a number of positions were identified where the sequence of the IVX037 genome differs from the E12 Travis prototype strain, including a number of both silent and coding nucleotide changes in the non-structural proteins (see Table 5). In some embodiments, a change is also found at nucleotide position 108 in the 5′UTR and based on primary and secondary sequence similarities between IVX037 viruses of the invention and the closely related Coxsackievirus B3 (CVB3), this nucleotide is predicted to be located in a loop of domain II of the 5′UTR and this change is not likely to affect the secondary structure of the IVX037 viral proteins.

In some embodiments, a number of coding changes were also identified in the capsid proteins responsible for cellular binding (Table 5). In some embodiments, a further amino acid change was identified in capsid protein VP3 at amino acid 206. In some embodiments, in VP1, one amino acid change was identified at amino acid 230 and a mix of amino acids are encoded at amino acid VP1 210 (see nucleotide position 3074 in Table 5). Interestingly, in some embodiments, two amino acid changes were detected in VP2 (amino acids 142 and 154) and both of these amino acids are predicted to be located in the hypervariable puff region of VP2, identified as the DAF-binding area of E12.

In addition to the complete genomic sequence of the E12 prototype strain Travis, there are three complete genomic sequences of three E12 strains (isolated from the stools of three healthy children in China in 2013) available in GenBank (Hongbu et al., (2018), Molecular characterization of echovirus 12 strains isolated from healthy children in China. Scientific Reports 8, 11716). These sequences are shown in SEQ ID NOs: 47-49 and were used for further comparative sequence analysis of the IVX037 genomic sequences of the invention. Of the changes in the capsid coding region, three changes (nucleotide 1373 [VP2 amino acid 142, 2348 [VP3 206] and 3074 [VP1 210]) were identified in some embodiments as being unique amongst the IVX037 sequences, E12 prototype strain Travis (SEQ ID NO: 46) and the three Chinese isolates (see Table 5). These changes are all coding changes and of special interest is amino acid VP2 141 (N->T) which is located in the hypervariable PUFF region of VP2, implicated in viral binding to DAF.

TABLE 5
Comparison of genomic differences between exemplary IVX037 sequences of the invention, E12 Travis
and three E12 strains isolated from three healthy children in China, in 2013. Nucleotide differences in
the capsid region which are unique for the IVX037 sequences amongst these five viral sequences
(IVX037, E12 Travis and three Chinese isolates) are highlighted in aqua.

| | | | | | | IVX037 | | |
|---|---|---|---|---|---|---|---|---|
| | | | | | IVX037 | OVO | | |
| | | | | | OVO | (3074 A) | IVX037 | |
| Position | Genomic | E12 Travis | E12 | IVX037 | (3074 | BDS | COLO | |
| (nt) | region¹ | (X79047.01) | isolates² | (3074 W) | T/A) | 11.08.2020 | (3074 U) | Comment |
| 108 | 5′UTR | C | T | T | T | T | T | Predicted to be located in loop structure of |
| | | | | | | | | IRES domain II. Not likely to change the |
| | | | | | | | | secondary structure of 5′UTR. |
| 1373 | VP2 | A (Asn) | A (Asn) | C (Thr) | C (Thr) | C (Thr) | C (Thr) | Coding change (VP2 142 T), unique IVX037, |
| | (142) | | | | | | | IVX037 OVO and COLO change. |
| | | | | | | | | Both amino acids (N & T) have polar neutral |
| | | | | | | | | side chains |
| 1408 | VP2 | C (His) | T (Tyr) | T (Tyr) | T (Tyr) | T (Tyr) | T (Tyr) | Coding change (VP2 154 Y) |
| | (154) | | | | | | | His [H] Electrically Charged Side Chains- |
| | | | | | | | | Basic |
| | | | | | | | | Tyr [Y] Hydrophobic Side Chain-Aromatic |
| 1451 | VP2 | G (Ser) | A (Asn) | G (Ser) | G/A | G/A | G (Ser) | Coding change in OVO strain (mix of aa), in |
| | (168) | | | | (Ser/Asn) | (Ser/Asn) | | VP2 PUFF region. |
| | | | | | | | | Both aa have polar Neutral Side Chains |
| 2348 | VP3 | C (Ala) | C (Ala) | T (Val) | T (Val) | T (Val) | T (Val) | Coding change (VP3 206 V), unique IVX037, |
| | (206) | | | | | | | IVX037 OVO and COLO change. |
| | | | | | | | | Both aa Hydrophobic Side Chain-Aliphatic |
| 2840 | VP1 | A (Gln) | A (Gln) | A (Gln) | A (Gln) | A (Gln) | G (Arg) | Coding change (VP1 132 R) unique change for |
| | (132) | | | | | | | COLO strain |
| | | | | | | | | Gln [Q]-Polar Neutral Side Chain |
| | | | | | | | | Arg [R] Electrically Charged Side Chains- |
| | | | | | | | | Basic |
| 3074 | VP1 | T (Phe) | T (Phe) | T/A | T/A | A (Tyr) | T (Phe) | Coding change (mix of amino acids; VP1 210 |
| | (210) | | | (Phe/Tyr) | (Phe/Tyr) | | | Y), unique change IVX037 and OVO strains |
| | | | | | | | | Both amino acids (F & Y) have Hydrophobic |
| | | | | | | | | Side Chain-Aromatic |
| 3133 | VP1 | T (Tyr) | C (His) | C (His) | C (His) | C (His) | C (His) | Coding change (VP1 230 H) |
| | (230) | | | | | | | Tyr [Y] Hydrophobic Side Chain-Aromatic |
| | | | | | | | | His [H] Electrically Charged Side Chains- |
| | | | | | | | | Basic |
| 3545 | 2A (47) | T (Phe) | T (Phe) | T (Phe) | T (Phe) | | T/A | Coding change in COLO strains (mix of |
| | | | | | | | (Phe/Tyr) | amino acids) conserved change |
| | | | | | | | | Both aa (F and Y) have hydrophobic side chain |
| | | | | | | | | aromatic |
| 3846 | 2B (26) | C | C | C | C | | C/T | Silent |
| 4101 | 2C (13) | T | C | C | C | | C | Silent |
| 4764 | 2C (234) | G | A | A | A | | A | Silent |
| 4881 | 2C (273) | G | G | A | A | | A | Silent |
| 5065- | 3A (6) | AT (Ile) | TA (Tyr) | TA (Tyr) | TA (Tyr) | | TA (Tyr) | Coding change (3A 6 I→Y) in IVX037, OVO |
| 5066 | | | | | | | | and COLO strains |
| | | | | | | | | Ile [I] Hydrophobic Side Chain-Aliphatic |
| | | | | | | | | Tyr [Y] Hydrophobic Side Chain-Aromatic |
| 5501 | 3C (39) | A (His) | G (Arg) | G (Arg) | G (Arg) | | G (Arg) | Coding change (3C 39 H→R) conserved |
| | | | | | | | | change |
| | | | | | | | | Both aa Electrically Charged Side Chains- |
| | | | | | | | | Basic |
| 6300- | 3D (123) | CA (Ile) | CC (Leu) | AC (Leu) | AC (Leu) | | AC (Leu) | Coding change (3D 123 I→L) conserved |
| 6301 | | | | | | | | change |
| | | | | | | | | Both aa Hydrophobic Side Chain-Aliphatic |
| 7423 | 3′UTR | GAA | * | GGA | GGA | | GGA | *Extreme 3′UTR seq is not experimentally |
| | | | | | | | | determined (primer-derived) for clinical E12 |
| | | | | | | | | isolates |

BDS: Bulk drug substance

### DISCUSSION

The complete genomic sequences of the IVX037 viruses of the invention, which were selected on cells expressing high levels of the attachment receptor DAF, were identified to have a number of changes compared to E12 Travis prototype strain. Changes in the capsid proteins are of particular interest as these may affect viral binding to the receptor on the cellular surface. Interestingly, no unique changes were identified in the non-structural proteins, while a number of changes were identified in the capsid coding region when the IVX037 sequences were compared to the E12 Travis prototype strain. In some embodiments, two amino acid changes located to the DAF-binding region of E12 VP2 protein (VP2 141 N->T and VP2 153 H->Y) were identified.

Of these two changes, VP2 142 T is unique amongst the E12 sequences analysed (E12 prototype strain Travis and three Chinese isolates from healthy children).

A sequence alignment showing exemplary IVX037 sequences of the invention including structural features as compared to E12 prototype strain Travis and three Chinese isolates from healthy children is presented below.

### CLUSTAL O(1.2.4) Multiple Sequence Alignment

```
Key:
VP1, VP2, VP3, VP4, 2A, 2B, 2C, 3A, 3B, 3C, 3D

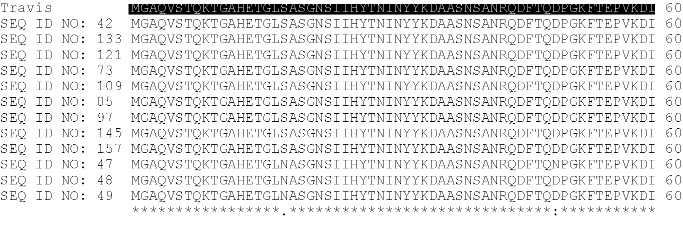

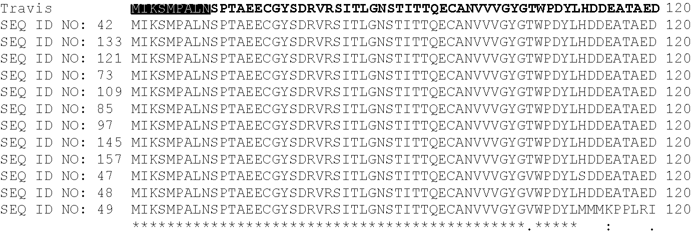


Travis QPTQPDVATCRFYTLESIQWQKTSDGWWWKFPEALKDMGLFGQNMHYHYLGRSGYTIHVQ 180
SEQ ID NO: 42 QPTQPDVATCRFYTLESIQWQKTSDGWWWKFPEALKDMGLFGQNMHYHYLGRSGYTIHVQ 180
SEQ ID NO: 133 QPTQPDVATCRFYTLESIQWQKTSDGWWWKFPEALKDMGLFGQNMHYHYLGRSGYTIHVQ 180
SEQ ID NO: 121 QPTQPDVATCRFYTLESIQWQKTSDGWWWKFPEALKDMGLFGQNMHYHYLGRSGYTIHVQ 180
SEQ ID NO: 73 QPTQPDVATCRFYTLESIQWQKTSDGWWWKFPEALKDMGLFGQNMHYHYLGRSGYTIHVQ 180
SEQ ID NO: 109 QPTQPDVATCRFYTLESIQWQKTSDGWWWKFPEALKDMGLFGQNMHYHYLGRSGYTIHVQ 180
SEQ ID NO: 85 QPTQPDVATCRFYTLESIQWQKTSDGWWWKFPEALKDMGLFGQNMHYHYLGRSGYTIHVQ 180
SEQ ID NO: 97 QPTQPDVATCRFYTLESIQWQKTSDGWWWKFPEALKDMGLFGQNMHYHYLGRSGYTIHVQ 180
SEQ ID NO: 145 QPTQPDVATCRFYTLESIQWQKTSDGWWWKFPEALKDMGLFGQNMHYHYLGRSGYTIHVQ 180
SEQ ID NO: 157 QPTQPDVATCRFYTLESIQWQKTSDGWWWKFPEALKDMGLFGQNMHYHYLGRSGYTIHVQ 180
SEQ ID NO: 47 QPTQPDVATCRFYTLESIQWQKTSDGWWWKFPEALKDMGLFGQNMHYHYLGRSGYTIHVQ 180
SEQ ID NO: 48 QPTQPDVATCRFYTLESIQWQKTSDGWWWKFPEALKDMGLFGQNMHYHYLGRSGYTIHVQ 180
SEQ ID NO: 49 NRHKPDVATCRFYTLESIQWQKTSDGWWWKFPEALKDMGLFGQNMHYHYLGRSGYTIHVQ 180
                              : :********************************************************
Travis CNASKFHQGCLLVVCVPEAEMGCATVANEVNAAALSSGETAKYFAKTSATGTHTVQSIVT 240
SEQ ID NO: 42 CNASKFHQGCLLVVCVPEAEMGCATVANEVTAAALSSGETAKYFAKTGATGTHTVQSIVT 240
SEQ ID NO: 133 CNASKFHQGCLLVVCVPEAEMGCATVANEVTAAALSSGETAKYFAKTGATGTHTVQNIVT 240
SEQ ID NO: 121 CNASKFHQGCLLVVCVPEAEMGCATVANEVTAAALSSGETAKYFAKTGATGTHTVQNIVT 240
SEQ ID NO: 73 CNASKFHQGCLLVVCVPEAEMGCATVANEVTAAALSSGETAKYFAKTGATGTHTVQSIVT 240
SEQ ID NO: 109 CNASKFHQGCLLVVCVPEAEMGCATVANEVTAAALSSGETAKYFAKTGATGTHTVQSIVT 240
SEQ ID NO: 85 CNASKFHQGCLLVVCVPEAEMGCATVANEVTAAALSSGETAKYFAKTGATGTHTVQSIVT 240
SEQ ID NO: 97 CNASKFHQGCLLVVCVPEAEMGCATVANEVTAAALSSGETAKYFAKTGATGTHTVQSIVT 240
SEQ ID NO: 145 CNASKFHQGCLLVVCVPEAEMGCATVANEVTAAALSSGETAKYFAKTGATGTHTVQSIVT 240
SEQ ID NO: 157 CNASKFHQGCLLVVCVPEAEMGCATVANEVTAAALSSGETAKYFAKTGATGTHTVQSIVT 240
SEQ ID NO: 47 CNASKFHQGCLLVVCVPEAEMGCATVANEVNAAALSSGEVAKYFAKTGATGTNTVQNIVT 240
SEQ ID NO: 48 CNASKFHQGCLLVVCVPEAEMGCATVANEVNAAALSSGEVAKYFAKTGATGTNTVQNIVT 240
SEQ ID NO: 49 CNASKFHQGCLLVVCVPEAEMGCATVANEVNAAALSSGEVAKYFAKTGATGTNTVQNIVT 240
                              ******************************.********.*******.****:***.***
Travis NAGMGVGVGNLTIFPHQWINLRTNNSATIVMPYINSVPMDNMFRHYNFTLMIIPFVPLDF 300
SEQ ID NO: 42 NAGMGVGVGNLTIFPHQWINLRTNNSATIVMPYINSVPMDNMFRHYNFTLMIIPFVPLDF 300
SEQ ID NO: 133 NAGMGVGVGNLTIFPHQWINLRTNNSATIVMPYINSVPMDNMFRHYNFTLMIIPFVPLDF 300
SEQ ID NO: 121 NAGMGVGVGNLTIFPHQWINLRTNNSATIVMPYINSVPMDNMFRHYNFTLMIIPFVPLDF 300
SEQ ID NO: 73 NAGMGVGVGNLTIFPHQWINLRTNNSATIVMPYINSVPMDNMFRHYNFTLMIIPFVPLDF 300
SEQ ID NO: 109 NAGMGVGVGNLTIFPHQWINLRTNNSATIVMPYINSVPMDNMFRHYNFTLMIIPFVPLDF 300
SEQ ID NO: 85 NAGMGVGVGNLTIFPHQWINLRTNNSATIVMPYINSVPMDNMFRHYNFTLMIIPFVPLDF 300
SEQ ID NO: 97 NAGMGVGVGNLTIFPHQWINLRTNNSATIVMPYINSVPMDNMFRHYNFTLMIIPFVPLDF 300
SEQ ID NO: 145 NAGMGVGVGNLTIFPHQWINLRTNNSATIVMPYINSVPMDNMFRHYNFTLMIIPFVPLDF 300
SEQ ID NO: 157 NAGMGVGVGNLTIFPHQWINLRTNNSATIVMPYINSVPMDNMFRHYNFTLMIIPFVPLDF 300
SEQ ID NO: 47 NAGMGVGVGNLTIFPHQWINLRTNNSATIVMPYINSVPMDNMFRHYNFTLMIIPFVPLDF 300
SEQ ID NO: 48 NAGMGVGVGNLTIFPHQWINLRTNNSATIVMPYINSVPMDNMFRHYNFTLMIIPFVPLDF 300
SEQ ID NO: 49 NAGMGVGVGNLTIFPHQWINLRTNNSATIVMPYINSVPMDNMFRHYNFTLMIIPFVPLDF 300
                              ************************************************************

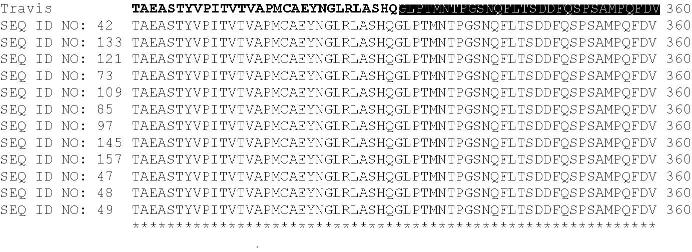

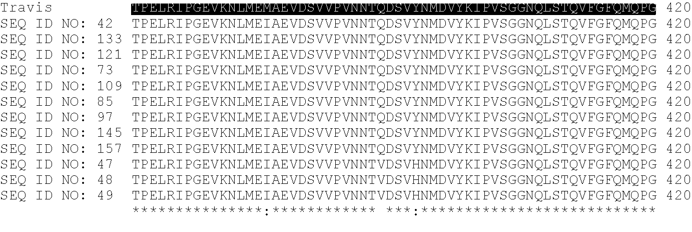

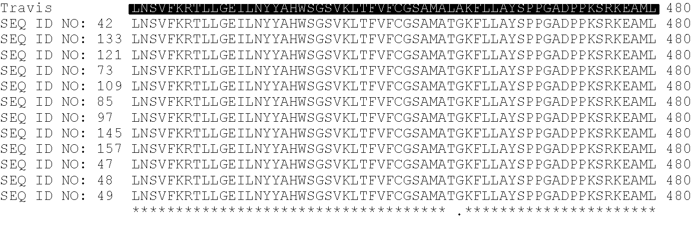

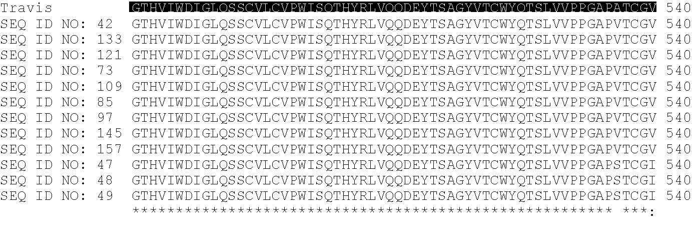

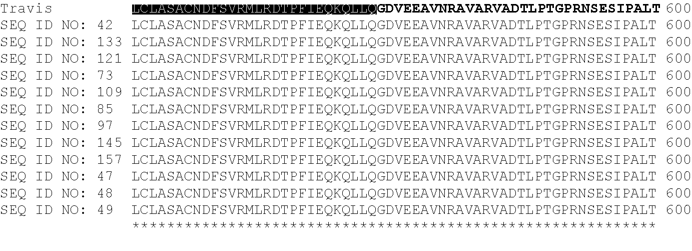





Travis AAETGHTSQVVPGDTMQTRHVKNYHSRTESSVENFLCRAACVCITKYKTKDSDPVQRYAN 660
SEQ ID NO: 42 AAETGHTSQVVPGDTMQTRHVKNYHSRTESSVENFLCRAACVYITKYKTKDSDPVQRYAN 660
SEQ ID NO: 133 AAETGHTSQVVPGDTMQTRHVKNYHSRTESSVENFLCRAACVYITKYKTKDSDPVQRYAN 660
SEQ ID NO: 121 AAETGHTSQVVPGDTMQTRHVKNYHSRTESSVENFLCRAACVYITKYKTKDSDPVQRYAN 660
SEQ ID NO: 73 AAETGHTSQVVPGDTMQTRHVKNYHSRTESSVENFLCRAACVYITKYKTKDSDPVQRYAN 660
SEQ ID NO: 109 AAETGHTSQVVPGDTMQTRHVKNYHSRTESSVENFLCRAACVYITKYKTKDSDPVQRYAN 660
SEQ ID NO: 85 AAETGHTSQVVPGDTMQTRHVKNYHSRTESSVENFLCRAACVYITKYKTKDSDPVQRYAN 660
SEQ ID NO: 97 AAETGHTSQVVPGDTMQTRHVKNYHSRTESSVENFLCRAACVYITKYKTKDSDPVQRYAN 660
SEQ ID NO: 145 AAETGHTSQVVPGDTMQTRHVKNYHSRTESSVENFLCRAACVYITKYKTKDSDPVQRYAN 660
SEQ ID NO: 157 AAETGHTSQVVPGDTMQTRHVKNYHSRTESSVENFLCRAACVYITKYKTKDSDPVQRYAN 660
SEQ ID NO: 47 AAETGHTSQVVPGDTMQTRHVKNYHSRTESSVENFLCRAACVYITKYKTKDSDPVQRYAN 660
SEQ ID NO: 48 AAETGHTSQVVPGDTMQTRHVKNYHSRTESSVENFLCRAACVYITKYKTKDSDPVQRYAN 660
SEQ ID NO: 49 AAETGHTSQVVPGDTMQTRHVKNYHSRTESSVENFLCRAACVYITKYKTKDSDPVQRYAN 660
                              ****************************************** *****************
Travis WRINTRQMAQLRRKFELFTYLRFDMEVTFVITSSQDDGTQLAQDMPVLTHQVMYIPPGGP 720
SEQ ID NO: 42 WRINTRQMVQLRRKFELFTYLRFDMEVTFVITSSQDDGTQLAQDMPVLTHQVMYIPPGGP 720
SEQ ID NO: 133 WRINTRQMVQLRRKFELFTYLRFDMEVTFVITSSQDDGTQLAQDMPVLTHQVMYIPPGGP 720
SEQ ID NO: 121 WRINTRQMVQLRRKFELFTYLRFDMEVTFVITSSQDDGTQLAQDMPVLTHQVMYIPPGGP 720
SEQ ID NO: 73 WRINTRQMVQLRRKFELFTYLRFDMEVTFVITSSQDDGTQLAQDMPVLTHQVMYIPPGGP 720
SEQ ID NO: 109 WRINTRQMVQLRRKFELFTYLRFDMEVTFVITSSQDDGTQLAQDMPVLTHQVMYIPPGGP 720
SEQ ID NO: 85 WRINTRQMVQLRRKFELFTYLRFDMEVTFVITSSQDDGTQLAQDMPVLTHQVMYIPPGGP 720
SEQ ID NO: 97 WRINTRQMVQLRRKFELFTYLRFDMEVTFVITSSQDDGTQLAQDMPVLTHQVMYIPPGGP 720
SEQ ID NO: 145 WRINTRQMVQLRRKFELFTYLRFDMEVTFVITSSQDDGTRLAQDMPVLTHQVMYIPPGGP 720
SEQ ID NO: 157 WRINTRQMVQLRRKFELFTYLRFDMEVTFVITSSQDDGTRLAQDMPVLTHQVMYIPPGGP 720
SEQ ID NO: 47 WRINTRQMVQLRRKFELFTYLRFDMEVTFVITSSQDDGTQLAQDMPVLTHQVMYIPPGGP 720
SEQ ID NO: 48 WRINTRQMVQLRRKFELFTYLRFDMEVTFVITSSQDDGTQLAQDMPVLTHQVMYIPPGGP 720
SEQ ID NO: 49 WRINTRQMVQLRRKFELFTYLRFDMEVTFVITSSQDDGTQLAQDMPVLTHQVMYIPPGGP 720
                              ********.******************************:********************
Travis VPNSATDFAWQSSTNPSIFWTEGNAPARMSIPFISIGNAYSNFYDGWSHFTQDGVYGFNS 780
SEQ ID NO: 42 VPNSVTDFAWQSSTNPSIFWTEGNAPARMSIPFISIGNAYSNFYDGWSHFTQDGVYGXNS 780
SEQ ID NO: 133 VPNSVTDFAWQSSTNPSIFWTEGNAPARMSIPFISIGNAYSNFYDGWSHFTQDGVYGFNS 780
SEQ ID NO: 121 VPNSVTDFAWQSSTNPSIFWTEGNAPARMSIPFISIGNAYSNFYDGWSHFTQDGVYGYNS 780
SEQ ID NO: 73 VPNSVTDFAWQSSTNPSIFWTEGNAPARMSIPFISIGNAYSNFYDGWSHFTQDGVYGYNS 780
SEQ ID NO: 109 VPNSVTDFAWQSSTNPSIFWTEGNAPARMSIPFISIGNAYSNFYDGWSHFTQDGVYGYNS 780
SEQ ID NO: 85 VPNSVTDFAWQSSTNPSIFWTEGNAPARMSIPFISIGNAYSNFYDGWSHFTQDGVYGFNS 780
SEQ ID NO: 97 VPNSVTDFAWQSSTNPSIFWTEGNAPARMSIPFISIGNAYSNFYDGWSHFTQDGVYGFNS 780
SEQ ID NO: 145 VPNSVTDFAWQSSTNPSIFWTEGNAPARMSIPFISIGNAYSNFYDGWSHFTQDGVYGFNS 780
SEQ ID NO: 157 VPNSVTDFAWQSSTNPSIFWTEGNAPARMSIPFISIGNAYSNFYDGWSHFTQDGVYGFNS 780
SEQ ID NO: 47 VPNSVTDFAWQSSTNPSIFWTEGNAPARMSIPFISIGNAYSNFYDGWSHFTQDGVYGFNS 780
SEQ ID NO: 48 VPNSVTDFAWQSSTNPSIFWTEGNAPARMSIPFISIGNAYSNFYDGWSHFTQDGVYGFNS 780
SEQ ID NO: 49 VPNSVTDFAWQSSTNPSIFWTEGNAPARMSIPFISIGNAYSNFYDGWSHFTQDGVYGFNS 780
                              ****.****************************************************:**
Travis LNNMGSIYIRHVNEQSPYAITSTVRVYFKPKHVRAWVPRPPRLCAYEKSSNVNFKPTDVT 840
SEQ ID NO: 42 LNNMGSIYIRHVNEQSPHAITSTVRVYFKPKHVRAWVPRPPRLCAYEKSSNVNFKPTDVT 840
SEQ ID NO: 133 LNNMGSIYIRHVNEQSPHAITSTVRVYFKPKHVRAWVPRPPRLCAYEKSSNVNFKPTDVT 840
SEQ ID NO: 121 LNNMGSIYIRHVNEQSPHAITSTVRVYFKPKHVRAWVPRPPRLCAYEKSSNVNFKPTDVT 840
SEQ ID NO: 73 LNNMGSIYIRHVNEQSPHAITSTVRVYFKPKHVRAWVPRPPRLCAYEKSSNVNFKPTDVT 840
SEQ ID NO: 109 LNNMGSIYIRHVNEQSPHAITSTVRVYFKPKHVRAWVPRPPRLCAYEKSSNVNFKPTDVT 840
SEQ ID NO: 85 LNNMGSIYIRHVNEQSPHAITSTVRVYFKPKHVRAWVPRPPRLCAYEKSSNVNFKPTDVT 840
SEQ ID NO: 97 LNNMGSIYIRHVNEQSPHAITSTVRVYFKPKHVRAWVPRPPRLCAYEKSSNVNFKPTDVT 840
SEQ ID NO: 145 LNNMGSIYIRHVNEQSPHAITSTVRVYFKPKHVRAWVPRPPRLCAYEKSSNVNFKPTDVT 840
SEQ ID NO: 157 LNNMGSIYIRHVNEQSPHAITSTVRVYFKPKHVRAWVPRPPRLCAYEKSSNVNFKPTDVT 840
SEQ ID NO: 47 LNNMGSIYIRHVNEQSPHAITSTVRVYFKPKHVRAWVPRPPRLCAYEKSSNVNFEPTDVT 840
SEQ ID NO: 48 LNNMGSIYIRHVNEQSPHAITSTVRVYFKPKHVRAWVPRPPRLCAYEKSSNVNFEPTDVT 840
SEQ ID NO: 49 LNNMGSIYIRHVNEQSPHAITSTVRVYFKPKHVRAWVPRPPRLCAYEKSSNVNFEPTDVT 840
                              ****.*************************************************:*****
Travis TSRTSITEVPSLRPSVVNTGAFGQQSGAAYVGNYRVVNRHLATHVDWQNCVWEDYNRDLL 900
SEQ ID NO: 42 TSRTSITEVPSLRPSVVNTGAFGQQSGAAYVGNYRVVNRHLATHVDWQNCVWEDYNRDLL 900
SEQ ID NO: 133 TSRTSITEVPSLRPSVVNTGAFGQQSGAAYVGNYRVVNRHLATHVDWQNCVWEDYNRDLL 900
SEQ ID NO: 121 TSRTSITEVPSLRPSVVNTGAFGQQSGAAYVGNYRVVNRHLATHVDWQNCVWEDYNRDLL 900
SEQ ID NO: 73 TSRTSITEVPSLRPSVVNTGAFGQQSGAAYVGNYRVVNRHLATHVDWQNCVWEDYNRDLL 900
SEQ ID NO: 109 TSRTSITEVPSLRPSVVNTGAFGQQSGAAYVGNYRVVNRHLATHVDWQNCVWEDYNRDLL 900
SEQ ID NO: 85 TSRTSITEVPSLRPSVVNTGAFGQQSGAAYVGNYRVVNRHLATHVDWQNCVWEDYNRDLL 900
SEQ ID NO: 97 TSRTSITEVPSLRPSVVNTGAFGQQSGAAYVGNYRVVNRHLATHVDWQNCVWEDYNRDLL 900
SEQ ID NO: 145 TSRTSITEVPSLRPSVVNTGAFGQQSGAAYVGNYRVVNRHLATHVDWQNCVWEDYNRDLL 900
SEQ ID NO: 157 TSRTSITEVPSLRPSVVNTGAFGQQSGAAYVGNYRVVNRHLATHVDWQNCVWEDYNRDLL 900
SEQ ID NO: 47 TTRSSITEVPSLRPSLQNTGAFGQQSGAVYVGNYRVVNRHLATYNDWQNCVWEDYNRDLL 900
SEQ ID NO: 48 TTRSSITEVPSLRPSLQNTGAFGQQSGAVYVGNYRVVNRHLATYNDWQNCVWEDYNRDLL 900
SEQ ID NO: 49 TTRSSITEVPSLRPSLQNTGAFGQQSGAVYVGNYRVVNRHLATYNDWQNCVWEDYNRDLL 900
                              *:*:***********: ***********.**************: ***************
Travis VSTTTAHGCDTIARCQCTTGVYFCASRNKHYPVSFEGPGLVEVQESEYYPRRYQSHVLLA 960
SEQ ID NO: 42 VSTTTAHGCDTIARCQCTTGVYFCASRNKHYPVSFEGPGLVEVQESEYYPRRYQSHVLLA 960
SEQ ID NO: 133 VSTTTAHGCDTIARCQCTTGVYFCASRNKHYPVSFEGPGLVEVQESEYYPRRYQSHVLLA 960
SEQ ID NO: 121 VSTTTAHGCDTIARCQCTTGVYFCASRNKHYPVSFEGPGLVEVQESEYYPRRYQSHVLLA 960
SEQ ID NO: 73 VSTTTAHGCDTIARCQCTTGVYFCASRNKHYPVSFEGPGLVEVQESEYYPRRYQSHVLLA 960
SEQ ID NO: 109 VSTTTAHGCDTIARCQCTTGVYFCASRNKHYPVSFEGPGLVEVQESEYYPRRYQSHVLLA 960
SEQ ID NO: 85 VSTTTAHGCDTIARCQCTTGVYFCASRNKHYPVSFEGPGLVEVQESEYYPRRYQSHVLLA 960
SEQ ID NO: 97 VSTTTAHGCDTIARCQCTTGVYFCASRNKHYPVSFEGPGLVEVQESEYYPRRYQSHVLLA 960
SEQ ID NO: 145 VSTTTAHGCDTIARCQCTTGVYFCASRNKHYPVSFEGPGLVEVQESEYYPRRYQSHVLLA 960
SEQ ID NO: 157 VSTTTAHGCDTIARCQCTTGVYFCASRNKHYPVSYEGPGLVEVQESEYYPRRYQSHVLLA 960
SEQ ID NO: 47 VSTTTAHGCDTIARCQCTTGVYFCASKNKHYPISFEGPGLVEVQESEYYPKRYQSHVLLA 960
SEQ ID NO: 48 VSTTTAHGCDTIARCQCTTGVYFCASKNKHYPISFEGPGLVEVQESEYYPKRYQSHVLLA 960
SEQ ID NO: 49 VSTTTAHGCDTIARCQCTTGVYFCASKNKHYPISFEGPGLVEVQESEYYPKRYQSHVLLA 960
                              **************************:*****:*:***************:*********
Travis AGFSEPGDCGGILRCEHGVIGLVTMGGEGVVGFADVRDLLWLEDDAMEQGVKDYVEQLGN 1020
SEQ ID NO: 42 AGFSEPGDCGGILRCEHGVIGLVTMGGEGVVGFADVRDLLWLEDDAMEQGVKDYVEQLGN 1020
SEQ ID NO: 133 AGFSEPGDCGGILRCEHGVIGLVTMGGEGVVGFADVRDLLWLEDDAMEQGVKDYVEQLGN 1020
SEQ ID NO: 121 AGFSEPGDCGGILRCEHGVIGLVTMGGEGVVGFADVRDLLWLEDDAMEQGVKDYVEQLGN 1020
SEQ ID NO: 73 AGFSEPGDCGGILRCEHGVIGLVTMGGEGVVGFADVRDLLWLEDDAMEQGVKDYVEQLGN 1020
SEQ ID NO: 109 AGFSEPGDCGGILRCEHGVIGLVTMGGEGVVGFADVRDLLWLEDDAMEQGVKDYVEQLGN 1020
SEQ ID NO: 85 AGFSEPGDCGGILRCEHGVIGLVTMGGEGVVGFADVRDLLWLEDDAMEQGVKDYVEQLGN 1020
SEQ ID NO: 97 AGFSEPGDCGGILRCEHGVIGLVTMGGEGVVGFADVRDLLWLEDDAMEQGVKDYVEQLGN 1020
SEQ ID NO: 145 AGFSEPGDCGGILRCEHGVIGLVTMGGEGVVGFADVRDLLWLEDDAMEQGVKDYVEQLGN 1020
SEQ ID NO: 157 AGFSEPGDCGGILRCEHGVIGLVTMGGEGVVGFADVRDLLWLEDDAMEQGVKDYVEQLGN 1020
SEQ ID NO: 47 AGFSEPGDCGGILRCEHGVIGLVTMGGEGVVGFADVRDLLWLEDDAMEQGVRDYVEQLGN 1020
SEQ ID NO: 48 AGFSEPGDCGGILRCEHGVIGLVTMGGEGVVGFADVRDLLWLEDDAMEQGVRDYVEQLGN 1020
SEQ ID NO: 49 AGFSEPGDCGGILRCEHGVIGLVTMGGEGVVGFADVRDLLWLEDDAMEQGVRDYVEQLGN 1020
                              ***************************************************:********
Travis AFGSGFTNQICEQVNLLKESLVGHDSILEKSLKALVKIISALVIVVRNHDDLITVTATLA 1080
SEQ ID NO: 42 AFGSGFTNQICEQVNLLKESLVGHDSILEKSLKALVKIISALVIVVRNHDDLITVTATLA 1080
SEQ ID NO: 133 AFGSGFTNQICEQVNLLKESLVGHDSILEKSLKALVKIISALVIVVRNHDDLITVTATLA 1080
SEQ ID NO: 121 AFGSGFTNQICEQVNLLKESLVGHDSILEKSLKALVKIISALVIVVRNHDDLITVTATLA 1080
SEQ ID NO: 73 AFGSGFTNQICEQVNLLKESLVGHDSILEKSLKALVKIISALVIVVRNHDDLITVTATLA 1080
SEQ ID NO: 109 AFGSGFTNQICEQVNLLKESLVGHDSILEKSLKALVKIISALVIVVRNHDDLITVTATLA 1080
SEQ ID NO: 85 AFGSGFTNQICEQVNLLKESLVGHDSILEKSLKALVKIISALVIVVRNHDDLITVTATLA 1080
SEQ ID NO: 97 AFGSGFTNQICEQVNLLKESLVGHDSILEKSLKALVKIISALVIVVRNHDDLITVTATLA 1080
SEQ ID NO: 145 AFGSGFTNQICEQVXLLKESLVGHDSILEKSLKALVKIISALVIVVRNHDDLITVTATLA 1080
SEQ ID NO: 157 AFGSGFTNQICEQVXLLKESLVGHDSILEKSLKALVKIISALVIVVRNHDDLITVTATLA 1080
SEQ ID NO: 47 AFGSGFTNQICEQVNLLKESLVGQDSILEKSLKALVKIISALVIVVRNHDDLITVTATLA 1080
SEQ ID NO: 48 AFGSGFTNQICEQVNLLKESLVGQDSILEKSLKALVKIISALVIVVRNHDDLITVTATLA 1080
SEQ ID NO: 49 AFGSGFTNQICEQVNLLKESLVGQDSILEKSLKALVKIISALVIVVRNHDDLITVTATLA 1080
                              ************** ********:************************************
Travis LIGCTSSPWRWLKHKVSQYYGIPMAERQSNGWLKKFTEMTNACKGMEWIAIKIQKFIEWL 1140
SEQ ID NO: 42 LIGCTSSPWRWLKHKVSQYYGIPMAERQSNGWLKKFTEMTNACKGMEWIAIKIQKFIEWL 1140
SEQ ID NO: 133 LIGCTSSPWRWLKHKVSQYYGIPMAERQSNGWLKKFTEMTNACKGMEWIAIKIQKFIEWL 1140
SEQ ID NO: 121 LIGCTSSPWRWLKHKVSQYYGIPMAERQSNGWLKKFTEMTNACKGMEWIAIKIQKFIEWL 1140
SEQ ID NO: 73 LIGCTSSPWRWLKHKVSQYYGIPMAERQSNGWLKKFTEMTNACKGMEWIAIKIQKFIEWL 1140
SEQ ID NO: 109 LIGCTSSPWRWLKHKVSQYYGIPMAERQSNGWLKKFTEMTNACKGMEWIAIKIQKFIEWL 1140
SEQ ID NO: 85 LIGCTSSPWRWLKHKVSQYYGIPMAERQSNGWLKKFTEMTNACKGMEWIAIKIQKFIEWL 1140
SEQ ID NO: 97 LIGCTSSPWRWLKHKVSQYYGIPMAERQSNGWLKKFTEMTNACKGMEWIAIKIQKFIEWL 1140
SEQ ID NO: 145 LIGCTSSPWRWLKHKVSQYYGIPMAERQSNGWLKKFTEMTNACKGMEWIAIKIQKFIEWL 1140
SEQ ID NO: 157 LIGCTSSPWRWLKHKVSQYYGIPMAERQSNGWLKKFTEMTNACKGMEWIAIKIQKFIEWL 1140
SEQ ID NO: 47 LIGCTSSPWRWLKHKVSQYYGIPMAERQNNGWLKKFTEMTNACKGMEWIAIKIQKFEMGF 1140
SEQ ID NO: 48 LIGCTSSPWRWLKHKVSQYYGIPMAERQNNGWLKKFTEMTNACKGMEWIAIKIQKFVEWL 1140
SEQ ID NO: 49 LIGCTSSPWRWLKHKVSQYYGIPMAERQNNGWLKKFTEMTNACKGMEWIAIKIQKFVEWL 1140
                        ****************************.*************************** :
Travis KLKILPEVKEKHEFLNRLKQLPLLESQIATIEQSAPSQSDQEQLFSNVQYFAHYCRKYAP 1200
SEQ ID NO: 42 KLKILPEVKEKHEFLNRLKQLPLLESQIATIEQSAPSQSDQEQLFSNVQYFAHYCRKYAP 1200
SEQ ID NO: 133 KLKILPEVKEKHEFLNRLKQLPLLESQIATIEQSAPSQSDQEQLFSNVQYFAHYCRKYAP 1200
SEQ ID NO: 121 KLKILPEVKEKHEFLNRLKQLPLLESQIATIEQSAPSQSDQEQLFSNVQYFAHYCRKYAP 1200
SEQ ID NO: 73 KLKILPEVKEKHEFLNRLKQLPLLESQIATIEQSAPSQSDQEQLFSNVQYFAHYCRKYAP 1200
SEQ ID NO: 109 KLKILPEVKEKHEFLNRLKQLPLLESQIATIEQSAPSQSDQEQLFSNVQYFAHYCRKYAP 1200
SEQ ID NO: 85 KLKILPEVKEKHEFLNRLKQLPLLESQIATIEQSAPSQSDQEQLFSNVQYFAHYCRKYAP 1200
SEQ ID NO: 97 KLKILPEVKEKHEFLNRLKQLPLLESQIATIEQSAPSQSDQEQLFSNVQYFAHYCRKYAP 1200
SEQ ID NO: 145 KLKILPEVKEKHEFLNRLKQLPLLESQIATIEQSAPSQSDQEQLFSNVQYFAHYCRKYAP 1200
SEQ ID NO: 157 KLKILPEVKEKHEFLNRLKQLPLLESQIATIEQSAPSQSDQEQLFSNVQYFAHYCRKYAP 1200
SEQ ID NO: 47 KVKILPEVREKHEFLNRLKQLPLLESQIATIEQSAPSQSDQEQLFSNVQYFAHYCRKYAP 1200
SEQ ID NO: 48 KVKILPEVREKHEFLNRLKQLPLLKSQIATIEQSAPSQSDQEQLFSNVQYFAHYCRKYAP 1200
SEQ ID NO: 49 KGQILPEVREKHEFLNRLKQLPLLESQIATIEQSAPSQSDQEQLFSNVQYFAHYCRKYAP 1200
                              * :*****:***************:***********************************
Travis LYAAEAKRVFSLEKKMSNYIQFKSKCRIEPVCLLLHGSPGAGKSVATSLIGRSLAEKLNS 1260
SEQ ID NO: 42 LYAAEAKRVFSLEKKMSNYIQFKSKCRIEPVCLLLHGSPGAGKSVATSLIGRSLAEKLNS 1260
SEQ ID NO: 133 LYAAEAKRVFSLEKKMSNYIQFKSKCRIEPVCLLLHGSPGAGKSVATSLIGRSLAEKLNS 1260
SEQ ID NO: 121 LYAAEAKRVFSLEKKMSNYIQFKSKCRIEPVCLLLHGSPGAGKSVATSLIGRSLAEKLNS 1260
SEQ ID NO: 73 LYAAEAKRVFSLEKKMSNYIQFKSKCRIEPVCLLLHGSPGAGKSVATSLIGRSLAEKLNS 1260
SEQ ID NO: 109 LYAAEAKRVFSLEKKMSNYIQFKSKCRIEPVCLLLHGSPGAGKSVATSLIGRSLAEKLNS 1260
SEQ ID NO: 85 LYAAEAKRVFSLEKKMSNYIQFKSKCRIEPVCLLLHGSPGAGKSVATSLIGRSLAEKLNS 1260
SEQ ID NO: 97 LYAAEAKRVFSLEKKMSNYIQFKSKCRIEPVCLLLHGSPGAGKSVATSLIGRSLAEKLNS 1260
SEQ ID NO: 145 LYAAEAKRVFSLEKKMSNYIQFKSKCRIEPVCLLLHGSPGAGKSVATSLIGRSLAEKLNS 1260
SEQ ID NO: 157 LYAAEAKRVFSLEKKMSNYIQFKSKCRIEPVCLLLHGSPGAGKSVATSLIGRSLAEKLNS 1260
SEQ ID NO: 47 LYAAEAKRVFSLEKKMSNYIQFKSKCRIEPVCLLLHGSPGAGKSVATNLIGRSLAEKLNS 1260
SEQ ID NO: 48 LYAAEAKRVFSLEKKMSNYIQFKSKCRIEPVCLLLHGSPGAGKSVATNLIGRSLAEKLNS 1260
SEQ ID NO: 49 LYAAEAKRVFSLEKKMSNYIQFKSKCRIEPVCLLLHGSPGAGKSVATNLIGRSLAEKLNS 1260
                              ***********************************************.************
Travis SVYSLPPDPDHFDGYKQQAVVIMDDLCQNPDGKDVSLFCQMVSSVDFVPPMAALEEKGIL 1320
SEQ ID NO: 42 SVYSLPPDPDHFDGYKQQAVVIMDDLCQNPDGKDVSLFCQMVSSVDFVPPMAALEEKGIL 1320
SEQ ID NO: 133 SVYSLPPDPDHFDGYKQQAVVIMDDLCQNPDGKDVSLFCQMVSSVDFVPPMAALEEKGIL 1320
SEQ ID NO: 121 SVYSLPPDPDHFDGYKQQAVVIMDDLCQNPDGKDVSLFCQMVSSVDFVPPMAALEEKGIL 1320
SEQ ID NO: 73 SVYSLPPDPDHFDGYKQQAVVIMDDLCQNPDGKDVSLFCQMVSSVDFVPPMAALEEKGIL 1320
SEQ ID NO: 109 SVYSLPPDPDHFDGYKQQAVVIMDDLCQNPDGKDVSLFCQMVSSVDFVPPMAALEEKGIL 1320
SEQ ID NO: 85 SVYSLPPDPDHFDGYKQQAVVIMDDLCQNPDGKDVSLFCQMVSSVDFVPPMAALEEKGIL 1320
SEQ ID NO: 97 SVYSLPPDPDHFDGYKQQAVVIMDDLCQNPDGKDVSLFCQMVSSVDFVPPMAALEEKGIL 1320
SEQ ID NO: 145 SVYSLPPDPDHFDGYKQQAVVIMDDLCQNPDGKDVSLFCQMVSSVDFVPPMAALEEKGIL 1320
SEQ ID NO: 157 SVYSLPPDPDHFDGYKQQAVVIMDDLCQNPDGKDVSLFCQMVSSVDFVPPMAALEEKGIL 1320
SEQ ID NO: 47 SVYSLPPDPDHFDGYKQQAVVIMDDLCQNPDGKDVSLFCQMVSSVDFVPPMAALEEKGIL 1320
SEQ ID NO: 48 SVYSLPPDPDHFDGYKQQAVVIMDDLCQNPDGKDVSLFCQMVSSIDCAKPMAALEEKGIL 1320
SEQ ID NO: 49 SVYSLPPDPDHFDGYKQQAVVIMDDLCQNPDGKDVSLFCQMVSSVDFVPPMAALEEKGIL 1320
                              ********************************************:* . ***********
Travis FTSPFVLASTNAGSINAPTVSDSRALARRFHFDMNIEVISMYSQNGKINMPMSVKTCDEE 1380
SEQ ID NO: 42 FTSPFVLASTNAGSINAPTVSDSRALARRGHFDMNIEVISMYSQNGKINMPMSVKTCDEE 1380
SEQ ID NO: 133 FTSPFVLASTNAGSINAPTVSDSRALARRFHFDMNIEVISMYSQNGKINMPMSVKTCDEE 1380
SEQ ID NO: 121 FTSPFVLASTNAGSINAPTVSDSRALARRFHFDMNIEVISMYSQNGKINMPMSVKTCDEE 1380
SEQ ID NO: 73 FTSPFVLASTNAGSINAPTVSDSRALARRFHFDMNIEVISMYSQNGKINMPMSVKTCDEE 1380
SEQ ID NO: 109 FTSPFVLASTNAGSINAPTVSDSRALARRFHFDMNIEVISMYSQNGKINMPMSVKTCDEE 1380
SEQ ID NO: 85 FTSPFVLASTNAGSINAPTVSDSRALARRFHFDMNIEVISMYSQNGKINMPMSVKTCDEE 1380
SEQ ID NO: 97 FTSPFVLASTNAGSINAPTVSDSRALARRFHFDMNIEVISMYSQNGKINMPMSVKTCDEE 1380
SEQ ID NO: 145 FTSPFVLASTNAGSINAPTVSDSRALARRFHFDMNIEVISMYSQNGKINMPMSVKTCDEE 1380
SEQ ID NO: 157 FTSPFVLASTNAGSINAPTVSDSRALARRFHFDMNIEVISMYSQNGKINMPMSVKTCDEE 1380
SEQ ID NO: 47 FTSPFVLASTNAGSINAPTVSDSRALARRFHFDMNIEVISMYSQNGKINMPMSVKTCDEE 1380
SEQ ID NO: 48 FTSPFVLASTNAGSINAPTVSDSRALARRFHFDMNIEVISMYSQNGKINMPMSVKTCDEE 1380
SEQ ID NO: 49 FTSPFVLASTNAGSINAPTVSDSRALARRFHFDMNIEVISMYSQNGKINMPMSVKTCDEE 1380
                              ************************************************************
Travis CCPVNFKRCCPLVCGKAIQFIDRRTQVRYSLDMLVTEMFREYNHRHSVGATLEALFQGPP 1440
SEQ ID NO: 42 CCPVNFKRCCPLVCGKAIQFIDRRTQVRYSLDMLVTEMFREYNHRHSVGATLEALFQGPP 1440
SEQ ID NO: 133 CCPVNFKRCCPLVCGKAIQFIDRRTQVRYSLDMLVTEMFREYNHRHSVGATLEALFQGPP 1440
SEQ ID NO: 121 CCPVNFKRCCPLVCGKAIQFIDRRTQVRYSLDMLVTEMFREYNHRHSVGATLEALFQGPP 1440
SEQ ID NO: 73 CCPVNFKRCCPLVCGKAIQFIDRRTQVRYSLDMLVTEMFREYNHRHSVGATLEALFQGPP 1440
SEQ ID NO: 109 CCPVNFKRCCPLVCGKAIQFIDRRTQVRYSLDMLVTEMFREYNHRHSVGATLEALFQGPP 1440
SEQ ID NO: 85 CCPVNFKRCCPLVCGKAIQFIDRRTQVRYSLDMLVTEMFREYNHRHSVGATLEALFQGPP 1440
SEQ ID NO: 97 CCPVNFKRCCPLVCGKAIQFIDRRTQVRYSLDMLVTEMFREYNHRHSVGATLEALFQGPP 1440
SEQ ID NO: 145 CCPVNFKRCCPLVCGKAIQFIDRRTQVRYSLDMLVTEMFREYNHRHSVGATLEALFQGPP 1440
SEQ ID NO: 157 CCPVNFKRCCPLVCGKAIQFIDRRTQVRYSLDMLVTEMFREYNHRHSVGATLEALFQGPP 1440
SEQ ID NO: 47 CSPVNFKKCCPLVCGKAIQFIDRRTQVRYSLDMLVTEMFREYNHRHSVGATLEALFQGPP 1440
SEQ ID NO: 48 CSPVNFKKCCPLVCGKAIQFIDRRTQVRYSLDMLVTEMFREYNHRHSVGATLEALFQGPP 1440
SEQ ID NO: 49 CSPVNFKKCCPLVCGKAIQFIDRRTQVRYSLDMLVTEMFREYNHRHSVGATLEALFQGPP 1440
                              *.*****:****************************************************
Travis VIREIKISVAPETPPPPAIADLLKSVDSEAVREYCKEKGWLVPEVNSTLQIEKHVSRAFI 1500
SEQ ID NO: 42 VYREIKISVAPETPPPPAIADLLKSVDSEAVREYCKEKGWLVPEVNSTLQIEKHVSRAFI 1500
SEQ ID NO: 133 VYREIKISVAPETPPPPAIADLLKSVDSEAVREYCKEKGWLVPEVNSTLQIEKHVSRAFI 1500
SEQ ID NO: 121 VYREIKISVAPETPPPPAIADLLKSVDSEAVREYCKEKGWLVPEVNSTLQIEKHVSRAFI 1500
SEQ ID NO: 73 VYREIKISVAPETPPPPAIADLLKSVDSEAVREYCKEKGWLVPEVNSTLQIEKHVSRAFI 1500
SEQ ID NO: 109 VYREIKISVAPETPPPPAIADLLKSVDSEAVREYCKEKGWLVPEVNSTLQIEKHVSRAFI 1500
SEQ ID NO: 85 VYREIKISVAPETPPPPAIADLLKSVDSEAVREYCKEKGWLVPEVNSTLQIEKHVSRAFI 1500
SEQ ID NO: 97 VYREIKISVAPETPPPPAIADLLKSVDSEAVREYCKEKGWLVPEVNSTLQIEKHVSRAFI 1500
SEQ ID NO: 145 VYREIKISVAPETPPPPAIADLLKSVDSEAVREYCKEKGWLVPEVNSTLQIEKHVSRAFI 1500
SEQ ID NO: 157 VYREIKISVAPETPPPPAIADLLKSVDSEAVREYCKEKGWLVPEVNSTLQIEKHVSRAFI 1500
SEQ ID NO: 47 VYREIKISVAPEIPPPPAIADLLKSVDSEAVREYCKERGWLVPEINSTLQIEKHVSRAFI 1500
SEQ ID NO: 48 VYREIKISVAPEIPPPPAIADLLKSVDSEAVREYCKERGWLVPEINSTLQIEKHVSRAFI 1500
SEQ ID NO: 49 VYREIKISVAPEIPPPPAIADLLKSVDSEAVREYCKERGWLVPEINSTLQIEKHVSRAFI 1500
                              * ********** ************************:******:***************

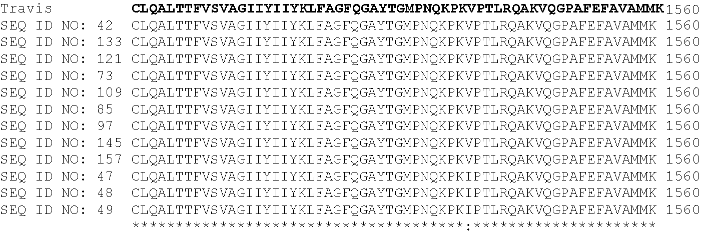

Travis RNASTVKTEYGEFTMLGIYDRWAVLPHHAKPGPTILMNDQEIGVLDAKELVDKDGTNLEL 1620
SEQ ID NO: 42 RNASTVKTEYGEFTMLGIYDRWAVLPRHAKPGPTILMNDQEIGVLDAKELVDKDGTNLEL 1620
SEQ ID NO: 133 RNASTVKTEYGEFTMLGIYDRWAVLPRHAKPGPTILMNDQEIGVLDAKELVDKDGTNLEL 1620
SEQ ID NO: 121 RNASTVKTEYGEFTMLGIYDRWAVLPRHAKPGPTILMNDQEIGVLDAKELVDKDGTNLEL 1620
SEQ ID NO: 73 RNASTVKTEYGEFTMLGIYDRWAVLPRHAKPGPTILMNDQEIGVLDAKELVDKDGTNLEL 1620
SEQ ID NO: 109 RNASTVKTEYGEFTMLGIYDRWAVLPRHAKPGPTILMNDQEIGVLDAKELVDKDGTNLEL 1620
SEQ ID NO: 85 RNASTVKTEYGEFTMLGIYDRWAVLPRHAKPGPTILMNDQEIGVLDAKELVDKDGTNLEL 1620
SEQ ID NO: 97 RNASTVKTEYGEFTMLGIYDRWAVLPRHAKPGPTILMNDQEIGVLDAKELVDKDGTNLEL 1620
SEQ ID NO: 145 RNASTVKTEYGEFTMLGIYDRWAVLPRHAKPGPTILMNDQEIGVLDAKELVDKDGTNLEL 1620
SEQ ID NO: 157 RNASTVKTEYGEFTMLGIYDRWAVLPRHAKPGPTILMNDQEIGVLDAKELVDKDGTNLEL 1620
SEQ ID NO: 47 RNASTVKTEYGEFTMLGIYDRWAVLPRHAKPGPTILMNDQEVGVVDAKELVDKDGTNLEL 1620
SEQ ID NO: 48 RNASTVKTEYGEFTMLGIYDRWAVLPRHAKPGPTILMNDQEVGVVDAKELVDKDGTNLEL 1620
SEQ ID NO: 49 RNASTVKTEYGEFTMLGIYDRWAVLPRHAKPGPTILMNDQEVGVVDAKELVDKDGTNLEL 1620
                              **************************:**************:**:***************
Travis TLLKLNRNEKFRDIRGFLAREEAEVNEAVLAINTSKFPNMYIPVGQVTDYGFLNLGGTPT 1680
SEQ ID NO: 42 TLLKLNRNEKFRDIRGFLAREEAEVNEAVLAINTSKFPNMYIPVGQVTDYGFLNLGGTPT 1680
SEQ ID NO: 133 TLLKLNRNEKFRDIRGFLAREEAEVNEAVLAINTSKFPNMYIPVGQVTDYGFLNLGGTPT 1680
SEQ ID NO: 121 TLLKLNRNEKFRDIRGFLAREEAEVNEAVLAINTSKFPNMYIPVGQVTDYGFLNLGGTPT 1680
SEQ ID NO: 73 TLLKLNRNEKFRDIRGFLAREEAEVNEAVLAINTSKFPNMYIPVGQVTDYGFLNLGGTPT 1680
SEQ ID NO: 109 TLLKLNRNEKFRDIRGFLAREEAEVNEAVLAINTSKFPNMYIPVGQVTDYGFLNLGGTPT 1680
SEQ ID NO: 85 TLLKLNRNEKFRDIRGFLAREEAEVNEAVLAINTSKFPNMYIPVGQVTDYGFLNLGGTPT 1680
SEQ ID NO: 97 TLLKLNRNEKFRDIRGFLAREEAEVNEAVLAINTSKFPNMYIPVGQVTDYGFLNLGGTPT 1680
SEQ ID NO: 145 TLLKLNRNEKFRDIRGFLAREEAEVNEAVLAINTSKFPNMYIPVGQVTDYGFLNLGGTPT 1680
SEQ ID NO: 157 TLLKLNRNEKFRDIRGFLAREEAEVNEAVLAINTSKFPNMYIPVGQVTDYGFLNLGGTPT 1680
SEQ ID NO: 47 TLLKLNRNEKFRDIRGFLTREEAEVNEAVLAINTSKFPNMYIPVGQVTDYGFLNLGGTPT 1680
SEQ ID NO: 48 TLLKLNRNEKFRDIRGFLTREEAEVNEAVLAINTSKFPNMYIPVGQVTDYGFLNLGGTPT 1680
SEQ ID NO: 49 TLLKLNRNEKFRDIRGFLTREEAEVNEAVLAINTSKFPNMYIPVGQVTDYGFLNLGGTPT 1680
                              ******************:*****************************************
Travis KRMLMYNFPTRAGQCGGVLMSTGKVLGIHVGGNGHQGFSAALLRHYFNEEQGEIEFIESS 1740
SEQ ID NO: 42 KRMLMYNFPTRAGQCGGVLMSTGKVLGIHVGGNGHQGFSAALLRHYFNEEQGEIEFIESS 1740
SEQ ID NO: 133 KRMLMYNFPTRAGQCGGVLMSTGKVLGIHVGGNGHQGFSAALLRHYFNEEQGEIEFIESS 1740
SEQ ID NO: 121 KRMLMYNFPTRAGQCGGVLMSTGKVLGIHVGGNGHQGFSAALLRHYFNEEQGEIEFIESS 1740
SEQ ID NO: 73 KRMLMYNFPTRAGQCGGVLMSTGKVLGIHVGGNGHQGFSAALLRHYFNEEQGEIEFIESS 1740
SEQ ID NO: 109 KRMLMYNFPTRAGQCGGVLMSTGKVLGIHVGGNGHQGFSAALLRHYFNEEQGEIEFIESS 1740
SEQ ID NO: 85 KRMLMYNFPTRAGQCGGVLMSTGKVLGIHVGGNGHQGFSAALLRHYFNEEQGEIEFIESS 1740
SEQ ID NO: 97 KRMLMYNFPTRAGQCGGVLMSTGKVLGIHVGGNGHQGFSAALLRHYFNEEQGEIEFIESS 1740
SEQ ID NO: 145 KRMLMYNFPTRAGQCGGVLMSTGKVLGIHVGGNGHQGFSAALLRHYFNEEQGEIEFIESS 1740
SEQ ID NO: 157 KRMLMYNFPTRAGQCGGVLMSTGKVLGIHVGGNGHQGFSAALLRHYFNEEQGEIEFIESS 1740
SEQ ID NO: 47 KRMLMYNFPTRAGQCGGVLMSTGKVLGIHVGGNGHQGFSAALLRHYFNDEQGEIEFIESS 1740
SEQ ID NO: 48 KRMLMYNFPTRAGQCGGVLMSTGKVLGIHVGGNGHQGFSAALLRHYFNDEQGEIEFIESS 1740
SEQ ID NO: 49 KRMLMYNFPTRAGQCGGVLMSTGKVLGIHVGGNGHQGFSAALLRHYFNDEQGEIEFIESS 1740
                              ************************************************:***********
Travis KDAGFPVINTPSKTKLEPSVFHQVFEGNKEPAVLRNGDPRLKVNFEEAIFSKYIGNINTH 1800
SEQ ID NO: 42 KDAGFPVINTPSKTKLEPSVFHQVFEGNKEPAVLRNGDPRLKVNFEEAIFSKYIGNINTH 1800
SEQ ID NO: 133 KDAGFPVINTPSKTKLEPSVFHQVFEGNKEPAVLRNGDPRLKVNFEEAIFSKYIGNINTH 1800
SEQ ID NO: 121 KDAGFPVINTPSKTKLEPSVFHQVFEGNKEPAVLRNGDPRLKVNFEEAIFSKYIGNINTH 1800
SEQ ID NO: 73 KDAGFPVINTPSKTKLEPSVFHQVFEGNKEPAVLRNGDPRLKVNFEEAIFSKYIGNINTH 1800
SEQ ID NO: 109 KDAGFPVINTPSKTKLEPSVFHQVFEGNKEPAVLRNGDPRLKVNFEEAIFSKYIGNINTH 1800
SEQ ID NO: 85 KDAGFPVINTPSKTKLEPSVFHQVFEGNKEPAVLRNGDPRLKVNFEEAIFSKYIGNINTH 1800
SEQ ID NO: 97 KDAGFPVINTPSKTKLEPSVFHQVFEGNKEPAVLRNGDPRLKVNFEEAIFSKYIGNINTH 1800
SEQ ID NO: 145 KDAGFPVINTPSKTKLEPSVFHQVFEGNKEPAVLRNGDPRLKVNFEEAIFSKYIGNINTH 1800
SEQ ID NO: 157 KDAGFPVINTPSKTKLEPSVFHQVFEGNKEPAVLRNGDPRLKVNFEEAIFSKYIGNINTH 1800
SEQ ID NO: 47 KEAGFPVINTPSKTKLEPSVFHHIFEGNKEPAVLRNGDPRLKANFEEAIFSKYIGNVNTH 1800
SEQ ID NO: 48 KEAGFPVINTPSKTKLEPSVFHHIFEGNKEPAVLRNGDPRLKANFEEAIFSKYIGNVNTH 1800
SEQ ID NO: 49 KEAGFPVINTPSKTKLEPSVFHHIFEGNKEPAVLRNGDPRLKANFEEAIFSKYIGNVNTH 1800
                              *:********************::******************.*************:***
Travis VDEYMLEAVDHYAGQLATLDISTEPMKLEDAVYGTEGLEALDLTTSAGYPYVAIGIKKRD 1860
SEQ ID NO: 42 VDEYMLEAVDHYAGQLATLDISTEPMKLEDAVYGTEGLEALDLTTSAGYPYVALGIKKRD 1860
SEQ ID NO: 133 VDEYMLEAVDHYAGQLATLDISTEPMKLEDAVYGTEGLEALDLTTSAGYPYVALGIKKRD 1860
SEQ ID NO: 121 VDEYMLEAVDHYAGQLATLDISTEPMKLEDAVYGTEGLEALDLTTSAGYPYVALGIKKRD 1860
SEQ ID NO: 73 VDEYMLEAVDHYAGQLATLDISTEPMKLEDAVYGTEGLEALDLTTSAGYPYVALGIKKRD 1860
SEQ ID NO: 109 VDEYMLEAVDHYAGQLATLDISTEPMKLEDAVYGTEGLEALDLTTSAGYPYVALGIKKRD 1860
SEQ ID NO: 85 VDEYMLEAVDHYAGQLATLDISTEPMKLEDAVYGTEGLEALDLTTSAGYPYVALGIKKRD 1860
SEQ ID NO: 97 VDEYMLEAVDHYAGQLATLDISTEPMKLEDAVYGTEGLEALDLTTSAGYPYVALGIKKRD 1860
SEQ ID NO: 145 VDEYMLEAVDHYAGQLATLDISTEPMKLEDAVYGTEGLEALDLTTSAGYPYVALGIKKRD 1860
SEQ ID NO: 157 VDEYMLEAVDHYAGQLATLDISTEPMKLEDAVYGTEGLEALDLTTSAGYPYVALGIKKRD 1860
SEQ ID NO: 47 VDGYMQEAVDHYAGQLATLDISTEPMKLEDAVYGTEGLEALDLTTSAGYPYVALGIKKRD 1860
SEQ ID NO: 48 VDGYMQEAIDHYAGQLATLDISTEPMKLEDAVYGTEGLEALDLTTSAGYPYVALGIKKRD 1860
SEQ ID NO: 49 VDGYMQEAIDHYAGQLATLDISTEPMKLEDAVYGTEGLEALDLTTSAGYPYVALGIKKRD 1860
                              ** ** **:********************************************:******
Travis ILSKKTKDLTKLKECMDKYGLNLPMVTYVKDELRSSEKVAKGKSRLIEASSLNDSVAMRQ 1920
SEQ ID NO: 42 ILSKKTKDLTKLKECMDKYGLNLPMVTYVKDELRSSEKVAKGKSRLIEASSLNDSVAMRQ 1920
SEQ ID NO: 133 ILSKKTKDLTKLKECMDKYGLNLPMVTYVKDELRSSEKVAKGKSRLIEASSLNDSVAMRQ 1920
SEQ ID NO: 121 ILSKKTKDLTKLKECMDKYGLNLPMVTYVKDELRSSEKVAKGKSRLIEASSLNDSVAMRQ 1920
SEQ ID NO: 73 ILSKKTKDLTKLKECMDKYGLNLPMVTYVKDELRSSEKVAKGKSRLIEASSLNDSVAMRQ 1920
SEQ ID NO: 109 ILSKKTKDLTKLKECMDKYGLNLPMVTYVKDELRSSEKVAKGKSRLIEASSLNDSVAMRQ 1920
SEQ ID NO: 85 ILSKKTKDLTKLKECMDKYGLNLPMVTYVKDELRSSEKVAKGKSRLIEASSLNDSVAMRQ 1920
SEQ ID NO: 97 ILSKKTKDLTKLKECMDKYGLNLPMVTYVKDELRSSEKVAKGKSRLIEASSLNDSVAMRQ 1920
SEQ ID NO: 145 ILSKKTKDLTKLKECMDKYGLNLPMVTYVKDELRSSEKVAKGKSRLIEASSLNDSVAMRQ 1920
SEQ ID NO: 157 ILSKKTKDLTKLKECMDKYGLNLPMVTYVKDELRSSEKVAKGKSRLIEASSLNDSVAMRQ 1920
SEQ ID NO: 47 ILSKKTKDLTKLKECMDKYGLNLPMVTYVKDELRSADKVAKGKSRLIEASSLNDSVAMRQ 1920
SEQ ID NO: 48 ILSKKTKDLTKLKECMDKYGLNLPMVTYVKDELRSADKVAKGKSRLIEASSLNDSVAMRQ 1920
SEQ ID NO: 49 ILSKKTKDLTKLKECMDKYGLNLPMVTYVKDELRSADKVAKGKSRLIEASSLNDSVAMRQ 1920
                              ***********************************::***********************
Travis TFGNLYKTFHLNPGIVTGSAVGCDPDLFWSKIPVMLDGHLIAFDYSGYDASLSPVWFACL 1980
SEQ ID NO: 42 TFGNLYKTFHLNPGIVTGSAVGCDPDLFWSKIPVMLDGHLIAFDYSGYDASLSPVWFACL 1980
SEQ ID NO: 133 TFGNLYKTFHLNPGIVTGSAVGCDPDLFWSKIPVMLDGHLIAFDYSGYDASLSPVWFACL 1980
SEQ ID NO: 121 TFGNLYKTFHLNPGIVTGSAVGCDPDLFWSKIPVMLDGHLIAFDYSGYDASLSPVWFACL 1980
SEQ ID NO: 73 TFGNLYKTFHLNPGIVTGSAVGCDPDLFWSKIPVMLDGHLIAFDYSGYDASLSPVWFACL 1980
SEQ ID NO: 109 TFGNLYKTFHLNPGIVTGSAVGCDPDLFWSKIPVMLDGHLIAFDYSGYDASLSPVWFACL 1980
SEQ ID NO: 85 TFGNLYKTFHLNPGIVTGSAVGCDPDLFWSKIPVMLDGHLIAFDYSGYDASLSPVWFACL 1980
SEQ ID NO: 97 TFGNLYKTFHLNPGIVTGSAVGCDPDLFWSKIPVMLDGHLIAFDYSGYDASLSPVWFACL 1980
SEQ ID NO: 145 TFGNLYKTFHLNPGIVTGSAVGCDPDLFWSKIPVMLDGHLIAFDYSGYDASLSPVWFACL 1980
SEQ ID NO: 157 TFGNLYKTFHLNPGIVTGSAVGCDPDLFWSKIPVMLDGHLIAFDYSGYDASLSPVWFACL 1980
SEQ ID NO: 47 TFGNLYKTFHLNPGVVTGSAVGCDPDLFWSKIPVMLDGHLIAFDYSGYDASLSPVWFACL 1980
SEQ ID NO: 48 TFGNLYKTFHLNPGVVTGSAVGCDPDLFWSKIPVMLDGHLIAFDYSGYDASLSPVWFACL 1980
SEQ ID NO: 49 TFGNLYKTFHLNPGVVTGSAVGCDPDLFWSKIPVMLDGHLIAFDYSGYDASLSPVWFACL 1980
                              **************:*********************************************
Travis KLLLEKLGYTHRETNYIDYLCNSHHLYRDKHYFVRGGMPSGCSGTSIFNSMINNIIIRTL 2040
SEQ ID NO: 42 KLLLEKLGYTHRETNYIDYLCNSHHLYRDKHYFVRGGMPSGCSGTSIFNSMINNIIIRTL 2040
SEQ ID NO: 133 KLLLEKLGYTHRETNYIDYLCNSHHLYRDKHYFVRGGMPSGCSGTSIFNSMINNIIIRTL 2040
SEQ ID NO: 121 KLLLEKLGYTHRETNYIDYLCNSHHLYRDKHYFVRGGMPSGCSGTSIFNSMINNIIIRTL 2040
SEQ ID NO: 73 KLLLEKLGYTHRETNYIDYLCNSHHLYRDKHYFVRGGMPSGCSGTSIFNSMINNIIIRTL 2040
SEQ ID NO: 109 KLLLEKLGYTHRETNYIDYLCNSHHLYRDKHYFVRGGMPSGCSGTSIFNSMINNIIIRTL 2040
SEQ ID NO: 85 KLLLEKLGYTHRETNYIDYLCNSHHLYRDKHYFVRGGMPSGCSGTSIFNSMINNIIIRTL 2040
SEQ ID NO: 97 KLLLEKLGYTHRETNYIDYLCNSHHLYRDKHYFVRGGMPSGCSGTSIFNSMINNIIIRTL 2040
SEQ ID NO: 145 KLLLEKLGYTHRETNYIDYLCNSHHLYRDKHYFVRGGMPSGCSGTSIFNSMINNIIIRTL 2040
SEQ ID NO: 157 KLLLEKLGYTHRETNYIDYLCNSHHLYRDKHYFVRGGMPSGCSGTSIFNSMINNIIIRTL 2040
SEQ ID NO: 47 KLLLEKLGYSHKETNYIDYLCNSHHLYRDKHYFVRGGMPSGCSGTSIFNSMINNIIIRTL 2040
SEQ ID NO: 48 KLLLEKLGYSHKETNYIDYLCNSHHLYRDKHYFVRGGMPSGCSGTSIFNSMINNIIIRTL 2040
SEQ ID NO: 49 KLLLEKLGYSHKETNYIDYLCNSHHLYRDKHYFVRGGMPSGCSGTSIFNSMINNIIIRTL 2040
                              *********:*:************************************************
Travis MLKVYKGIDLDQFRMIAYGDDVIASYPWPIDASLLAEAGKGYGLIMTPADKGERFNEVTW 2100
SEQ ID NO: 42 MLKVYKGIDLDQFRMIAYGDDVIASYPWPIDASLLAEAGKGYGLIMTPADKGECFNEVTW 2100
SEQ ID NO: 133 MLKVYKGIDLDQFRMIAYGDDVIASYPWPIDASLLAEAGKGYGLIMTPADKGECFNEVTW 2100
SEQ ID NO: 121 MLKVYKGIDLDQFRMIAYGDDVIASYPWPIDASLLAEAGKGYGLIMTPADKGECFNEVTW 2100
SEQ ID NO: 73 MLKVYKGIDLDQFRMIAYGDDVIASYPWPIDASLLAEAGKGYGLIMTPADKGECFNEVTW 2100
SEQ ID NO: 109 MLKVYKGIDLDQFRMIAYGDDVIASYPWPIDASLLAEAGKGYGLIMTPADKGECFNEVTW 2100
SEQ ID NO: 85 MLKVYKGIDLDQFRMIAYGDDVIASYPWPIDASLLAEAGKGYGLIMTPADKGECFNEVTW 2100
SEQ ID NO: 97 MLKVYKGIDLDQFRMIAYGDDVIASYPWPIDASLLAEAGKGYGLIMTPADKGECFNEVTW 2100
SEQ ID NO: 145 MLKVYKGIDLDQFRMIAYGDDVIASYPWPIDASLLAEAGKGYGLIMTPADKGECFNEVTW 2100
SEQ ID NO: 157 MLKVYKGIDLDQFRMIAYGDDVIASYPWPIDASLLAEAGKGYGLIMTPADKGECFNEVTW 2100
SEQ ID NO: 47 MLKVYKGIDLDQFRMIAYGDDVIASYPHPIDASLLAEAGKGYGLIMTPADKGECFNEVTW 2100
SEQ ID NO: 48 MLKVYKGIDLDQFRMIAYGDDVIASYPHPIDASLLAEAGKGYGLIMTPADKGECFNEVTW 2100
SEQ ID NO: 49 MLKVYKGIDLDQFRMIAYGDDVIASYPHPIDASLLAEAGKGYGLIMTPADKGECFNEVTW 2100
                              *************************** ************************* ******
Travis TNVTFLKRYFRADEQYPFLVHPVMPMKDIHESIRWTKDPKNTQDHVRSLCLLAWHNGEQE 2160
SEQ ID NO: 42 TNVTFLKRYFRADEQYPFLVHPVMPMKDIHESIRWTKDPKNTQDHVRSLCLLAWHNGEQE 2160
SEQ ID NO: 133 TNVTFLKRYFRADEQYPFLVHPVMPMKDIHESIRWTKDPKNTQDHVRSLCLLAWHNGEQE 2160
SEQ ID NO: 121 TNVTFLKRYFRADEQYPFLVHPVMPMKDIHESIRWTKDPKNTQDHVRSLCLLAWHNGEQE 2160
SEQ ID NO: 73 TNVTFLKRYFRADEQYPFLVHPVMPMKDIHESIRWTKDPKNTQDHVRSLCLLAWHNGEQE 2160
SEQ ID NO: 109 TNVTFLKRYFRADEQYPFLVHPVMPMKDIHESIRWTKDPKNTQDHVRSLCLLAWHNGEQE 2160
SEQ ID NO: 85 TNVTFLKRYFRADEQYPFLVHPVMPMKDIHESIRWTKDPKNTQDHVRSLCLLAWHNGEQE 2160
SEQ ID NO: 97 TNVTFLKRYFRADEQYPFLVHPVMPMKDIHESIRWTKDPKNTQDHVRSLCLLAWHNGEQE 2160
SEQ ID NO: 145 TNVTFLKRYFRADEQYPFLVHPVMPMKDIHESIRWTKDPKNTQDHVRSLCLLAWHNGEQE 2160
SEQ ID NO: 157 TNVTFLKRYFRADEQYPFLVHPVMPMKDIHESIRWTKDPKNTQDHVRSLCLLAWHNGEQE 2160
SEQ ID NO: 47 NNVTFLKRYFRADEQYPFLVHPVMPMKDIHESIRWTKDPKNTQDHVRSLCLLAWHNGERE 2160
SEQ ID NO: 48 NNVTFLKRYFRADEQYPFLVHPVMPMKDIHESIRWTKDPKNTQDHVRSLCLLAWHNGERE 2160
SEQ ID NO: 49 NNVTFLKRYFRADEQYPFLVHPVMPMKDIHESIRWTKDPKNTQDHVRSLCLLAWHNGERE 2160
                              .*********************************************************:*
Travis YEEFIRKIRSVPVGRCLTLPAFSTLRRKWLDSF 2193
SEQ ID NO: 42 YEEFIRKIRSVPVGRCLTLPAFSTLRRKWLDSF 2193
SEQ ID NO: 133 YEEFIRKIRSVPVGRCLTLPAFSTLRRKWLDSF 2193
SEQ ID NO: 121 YEEFIRKIRSVPVGRCLTLPAFSTLRRKWLDSF 2193
SEQ ID NO: 73 YEEFIRKIRSVPVGRCLTLPAFSTLRRKWLDSF 2193
SEQ ID NO: 109 YEEFIRKIRSVPVGRCLTLPAFSTLRRKWLDSF 2193
SEQ ID NO: 85 YEEFIRKIRSVPVGRCLTLPAFSTLRRKWLDSF 2193
SEQ ID NO: 97 YEEFIRKIRSVPVGRCLTLPAFSTLRRKWLDSF 2193
SEQ ID NO: 145 YEEFIRKIRSVPVGRCLTLPAFSTLRRKWLDSF 2193
SEQ ID NO: 157 YEEFIRKIRSVPVGRCLTLPAFSTLRRKWLDSF 2193
SEQ ID NO: 47 YEEFIKKIRSVPVGRCLTLPAFSTLRRKWLDSF 2193
SEQ ID NO: 48 YEEFIKKIRSVPVGRCLTLPAFSTLRRKWLDSF 2193
SEQ ID NO: 49 YEEFIKKIRSVPVGRCLTLPAFSTLRRKWLDSF 2193
                              *****:***************************
```

### Example Three: Oncolytic Activity of IVX037 Passaged in Colorectal and Ovarian Cell Lines and Tests

An exemplary bio-selected IVX-037 strain was submitted to five rounds of serial passage in human ovarian cell culture (DOV-13) and human MSS colorectal cell culture (SW480). Briefly, cancer cell monolayers were infected with the IVX-037 strain at 37° C. for Ihr, then maintenance medium (DMEM) was applied and the cultures incubated at 37° C. for 24-124 hours, until detectable CPE was evident. Infected cells and media were next used to infect monolayer cultures of the same cell type using the same infection methodology. This viral passaging process was repeated for 5 cycles to yield an IVX-037 OVO strain from the DOV-13 cells and an IVX-037 COLO strain from the SW480 cells.

### Materials and Methods

Neoplastic cell lines (2×10⁴ cells) were propagated in monolayers in 96-well plates and inoculated with 10-fold serial dilutions of stock preparations of E12 P1 vero, the IVX-037 COLO strain and the IVX037 OVO strain. Following incubation at 37° C./5% CO₂ for 5 days, the cell monolayers were examined microscopically for the presence of cytopathic effect (CPE). Fifty percent endpoint titres were calculated using the method of Karber and the mean (±SEM) with the minimum MOI (TCID50/cell) for each cell line was calculated.

Viral sequences were obtained by Sangar sequencing employing a walking strategy using conventional methodologies.

### Results

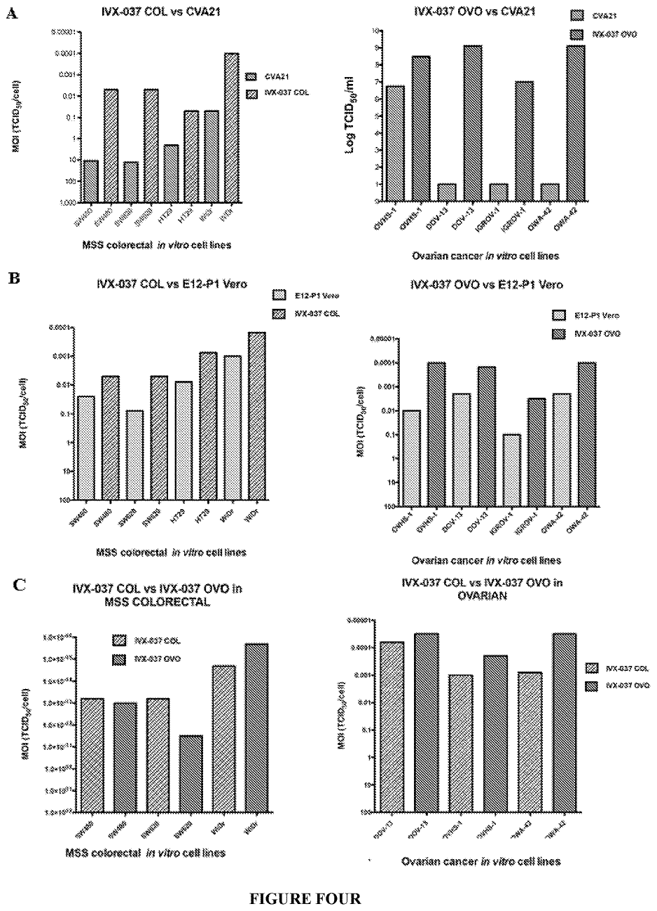

In FIG. 4A, the exemplary IVX-037 COLO strain of the invention displays significantly enhanced oncolytic activity (up to 10³ fold higher) in four human MSS-colorectal cancer cell lines compared to that by CVA21 (historical public available data). In addition, the IVX-037 OVO strain of the invention displays significantly enhanced activity (up to 10⁷ fold higher) in four high grade human ovarian cancer cell lines compared to that by CVA21 (historical public available data).

In FIG. 4B, the exemplary IVX-037 COLO strain of the invention displays significantly enhanced oncolytic activity (~10-10² fold higher) in four human MSS-colorectal cancer cell lines compared to that by the prototype E12 travis strain passaged one in Vero cells (E12 P1-Vero). While, the IVX-037 OVO strain of the invention displays significantly enhanced oncolytic activity (~10-10² fold higher) in four high grade human ovarian cancer cell lines compared to that by the prototype E12 travis strain passaged one in Vero cells (E12 P1-Vero).

In FIG. 4C, the exemplary IVX-037 COLO strain displays some enhanced oncolytic activity in 2 of 3 human MSS-colorectal cancer cell lines compared to that by the exemplary IVX-037 OVO strain. While, the IVX-037 OVO strain displays some enhanced oncolytic activity in 3 of 3 human ovarian cancer cell lines compared to that by the IVX-037 COLO strain.

Nucleotide sequences of viruses passaged in the ovarian and colorectal cancer cell lines were obtained and examples of these are shown in SEQ ID NO: 50, SEQ ID NO: 51, SEQ ID NO: 96, SEQ ID NO: 108, SEQ ID NO: 120, SEQ ID NO: 132, SEQ ID NO: 144 and SEQ ID NO: 156 respectively. Examples of the encoded amino acid sequences relating to the obtained nucleic acid sequences are shown in SEQ ID NO: 52, SEQ ID NO: 53, SEQ ID NO: 97, SEQ ID NO: 109, SEQ ID NO: 121, SEQ ID NO: 133, SEQ ID NO: 145 and SEQ ID NO: 157 respectively.

### Example Four: IVX037 Viral Strains Upregulate DDX58 (RIG-I), CD274 (PD-L1) and IFN-g Inducible Protein 10 (CXCL10) and PD-L1

### Materials and Methods

### (i) In Vitro Assay

The following protocol was followed in conducting the in vitro assay:

Seed OAW42, DOV13 or WiDr cells in 5×6-well plates in media with 2% FBS (~2.5E+05 cells/mL, 3 mLs/well) to achieve ~90-100% confluency the following day. Prepare 500 mL volume of serum-free DMEM media for cell washing and preparation of inoculum. From this media, allocate 100 mL volume for maintenance media with addition of 0.5% FBS. Prepare 10 mL of buffer RLT with addition of 100 μL of 14.3M B-ME-mercaptoethanol for cell lysis process. Buffer RLT+B-ME stable for 1 month at RT. Obtain cell count using 3 wells of 1 plate for average cell count per well and harvest 3 untreated control wells. Determine the amount of E12 virus to add per well based on number of cells/well and viral titre (if viral titre unknown, use 0.5 mL/well of p5 DOV stock diluted 1:10 in serum free media*). Remove media, wash cells once with serum-free media. Dilute virus to achieve an MOI of 10 TCID₅₀/cell in 0.5 mL of serum-free media (or as above*). Four identical plates will be set up with 3 wells of virus infected cells and 3 wells of mock infected cells per time point (serum-free media only). NB Total of ~7 mL of inoculum required for infection of 12 wells. Infect cells with 0.5 mL diluted virus or serum-free media. Incubate plates for 1 hour, gently rock plates every 15 minutes (or place on rocker) to ensure cell monolayer does not dry out. Remove inoculate and wash gently 3 times with pre-warmed serum-free media. Re-feed cells with 2-3 ml of pre-warmed media with 0.5% FBS (maintenance media) and incubate for 0, 3, 6 or 9 hrs at 37° C. NB Harvest time 0 wells immediately following re-feed step using protocol below. At each harvest time point, take photos of cell monolayers and completely remove the media (supernatant) and freeze for subsequent viral titration according to LAB-PROC 006, and potential analysis of viral RNA load according to LAB-PROC 018, LAB-PROC 031, LAB-PROC 032). NB No need to collected media from control samples. Media harvested from each virus infected well should be dispensed into 4×500 μL labelled aliquots prior to freezing. In addition to virus infected aliquots, dispense 4×500 μL aliquots of residual maintenance media and freeze for use as negative controls in RNA extractions. NB aliquoting should be performed after lysis (step 11). Directly following removal of media, lyse the cell monolayer with 350 μL buffer RLT (containing beta-ME-mercaptoethanol), collect lysate following scraping with cell scraper as per step 5.5.2-5.5.3 in LAB-PROC 025. Vortex or pipet to mix, and ensure that no cell clumps are visible before proceeding to homogenise samples using QIAshredder (add max 700 uL/shredder). Freeze samples until all samples are harvested. Use labelling nomenclature: Control wells=Dov13 C#Xhr 202007XX, Infected wells=Dov13 IVX037 #, Xhr, 202007XX. When all samples are harvested and frozen, thaw samples in 37° C. waterbath just until completely thawed and salts dissolved. If any insoluble material is visible, centrifuge for 5 min at 3,000-5,000×g and transfer supernatant to new tubes. Extract RNA from all samples (starting at step 5.8 RNA extraction and purification LAB-PROC 025). Include an extraction of a negative control (media with 0.5% FBS), and a low range positive control (control stock diluted 10⁻⁴). Total number of samples: 12 virus-infected samples (3 replicates per time point), 12 non-infected samples, negative extraction controls). Perform gene expression analysis on extracted RNA samples, include no template control (NTC), RT-PCR negative control and extracted negative control. The following TaqMan™ Gene Expression assays were used to detect levels of mRNA of CXCL10, RIG-I and CD274 relative to the levels opf the house keeping gene GUSB. TaqMan™ Gene Expression assays are designed to analyse the expression of, CXCL10, GUSB, CD274 and RIG-I gene expression using RT-qPCR. Other pre-designed TaqMan™ Gene expression assays may also be used with this assay. The TaqMan™ Gene Expression assays are designed to detect expressed mRNA (assay type ‘_m1’) and the assays HS01125296_m1 (CD274), HS00171042_m1 (CXCL10), HS99999908_m1 (GUSB) and Hs01061436_m1 (RIG-I) are selected based on the capacity to detect the human mRNA of these transcripts, but without cross-reacting with mouse, and the assay probes spans the exon in order to not detect genomic DNA. Both assays are labelled with FAM-MGB and can therefore not be multiplexed but need to be tested in single-plex assays. The TaqPath™ 1-Step Multiplex Master Mix contains components for both reverse transcription and real-time qPCR including Fast DNA polymerase, Thermostable MMLV enzyme, RNase inhibitor and buffer components. The master-mix also contains the enzyme UNG to prevent carryover between assays and contains a blend of dUTP and dTTP to enable UNG activity. The master-mix contains Mustang Purple as a passive reference dye to provide an internal reference for normalising of fluctuations in fluorescence due to changes in volume or concentration. The assay is a relative quantification gene expression assay which determines changes in the expression in the test sample relative to a reference sample (e.g. sample versus untreated control sample). The results are analysed using the comparative Ct method which compares the Ct value of the treatment sample to a control using the formula Ratio=2^{ΔCt} (where mass is the normaliser i.e. number of cells extracted or μg of RNA used as RT-qPCR template).

### (ii) In Vivo Assay

The following protocol was followed in conducting the in vivo assay:

Immune-compromised SCID-Balb/C mice were given s.c. injections of MSS-colorectal cells WiDr. Once tumours were physically palpable (≥25 mm³), mice received i.t. injections of viral dilution vehicle as a control, or an IVX-037 strain of the invention. Two mice from the control and IVX037 treatment groups were sacrificed at 24, 48 and 72 hours post-treatment and the tumours excised and placed in RNA later buffer and stored at 4° C. prior to RNA extraction and gene expression PCR analysis. The following TaqMan™ Gene Expression assays were used to detect levels of mRNA of CXCL10, RIG-I and CD274 relative to the levels of the house keeping gene GUSB in the excised tumours.

### Results

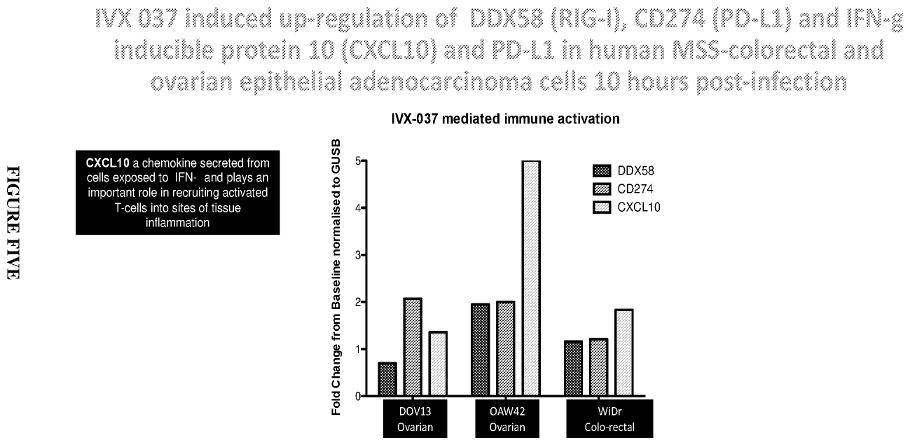

In vitro challenge of ovarian cancer cell lines DOV-13, OAW42 and human MSS-colorectal cell line WiDr with an IVX-037 OVO strain of the invention resulted in notable upregulation (1.5 to 5-fold) of mRNA encoding DDX58, CD274 and CXCL10 at 10 hours post-infection compared to control infection levels (FIG. 5).

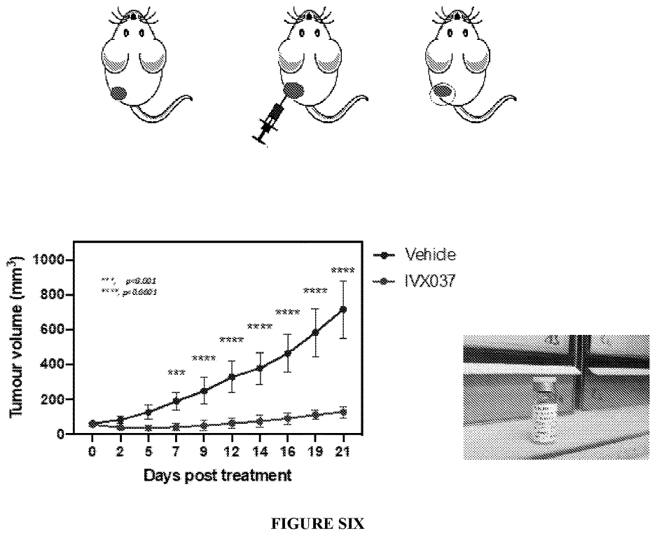

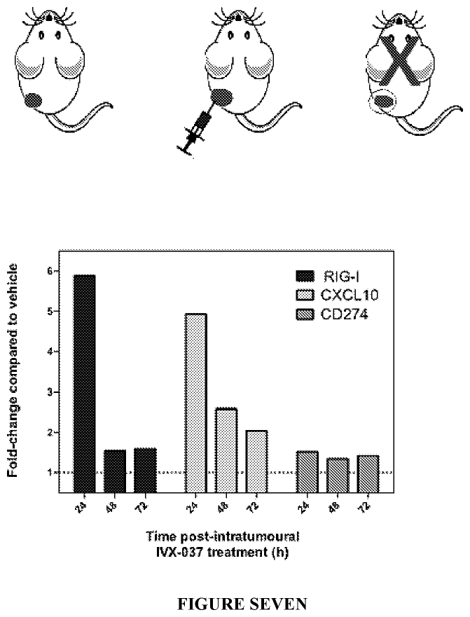

In vivo challenge of human MSS-colorectal cell xenografts in Balb-C SCID mice via a single intratumoral injection (10⁸ TCID₅₀) of an IVX-037 OVO strain of the invention yielded significant up-regulation of mRNA encoding DDX58, CD274 and CXCL10 at 24, 48 and 72 hours post infection. Most notable up-regulation was observed at 24 hr post infection, in particular the DXD-58 and CXCL10 genes (FIGS. 6 and 7).

### Example Five: Increased IVX037 Infectivity in CHO Cells Transfected with the Human FcRn Receptor

### Materials and Methods

The CHO cells were seeded into 24-well plates to a confluence of ~70-90%. The following day, transfections using the commercially available Lipofectamine 2000 with the GenEZ™ ORF clones expressing the Fcgrt (encodes for the human FcRn receptor) in the mammalian expression cloning vector pcDNA3.1+/C-(K)-DYK. The pIND plasmid without the Fcgrt gene was used as a transfection control. For the reverse transfection, cells were seeded concurrently with the lipofectamine-plasmid complex. 48 hours later, cells were infected with an IVX-037 OVO strain of the invention. After 72-hour infection, the supernatant and cells were collected, and an infectivity assay performed. Plates were scored for cytopathic effects five days later. Infectivity data was expressed as logarithmic tissue culture infectious dose 50% per mL (log 10TCID50/mL).

### Results

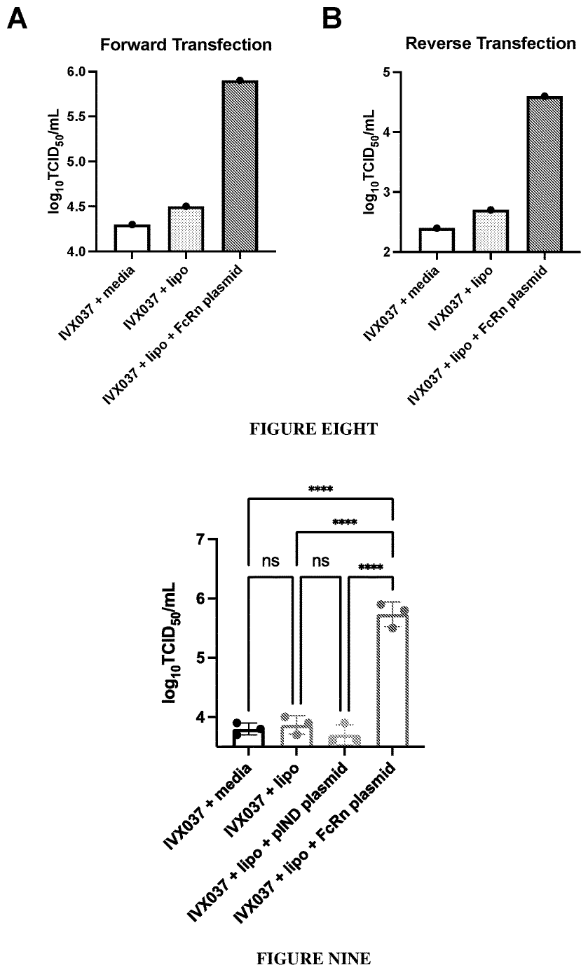

IVX037 infectivity data on CHO cells transfected with human FcRn using the forward and reverse transfection method is shown in FIG. 8. In both cases, an increase in infectivity was observed in cells expressing the human FcRn receptor.

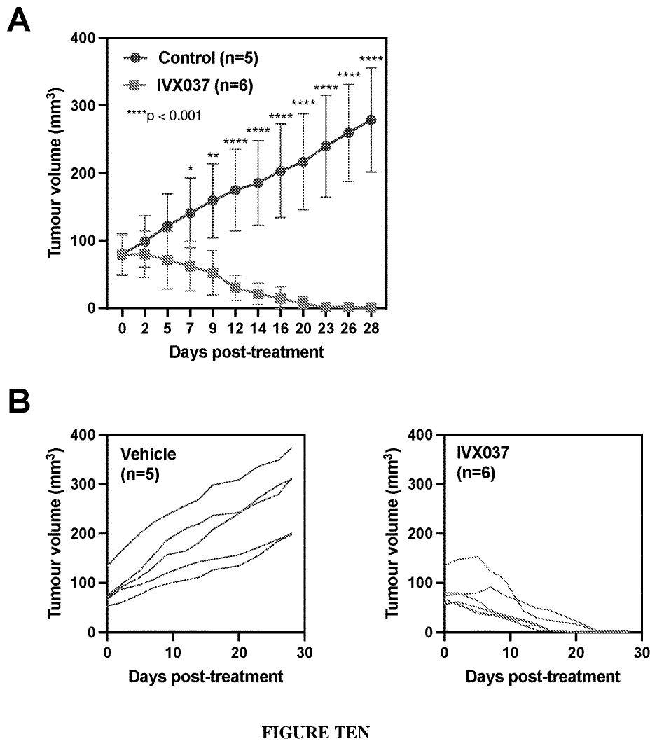

As set out in FIG. 9, infectivity data derived from CHO cells transfected with either the human FcRn plasmid or the pIND control plasmids showed a significantly higher level of infectivity in CHO cells transfected with the FcRn plasmid. The inclusion of a control plasmid confirmed that transfection with the FcRn plasmid was attributable to the transfection instead of other causes. The data here provides strong evidence that the expression of the human FcRn receptor conferred improved susceptibility in the CHO cells to an IVX037 infection. Statistical differences among the groups were determined using the one-way ANOVA.

### Example Six: Oncolytic Activity of Intratumoural IVX037 in Human Colorectal Cancer (Caco-2) Xenografts

### Materials and Methods

Immune-compromised SCID-Balb/C mice were inoculated with human colorectal cancer cells Caco-2 (2e6 cells) subcutaneously. Once tumours were physically palpable (≥25 mm3), mice received a single intratumoural injection of a control vehicle, or an IVX-037 OVO strain of the invention (1e8 TCID50/injection). Tumour volumes were measured, and animals were terminated 28 days post-treatment.

### Results

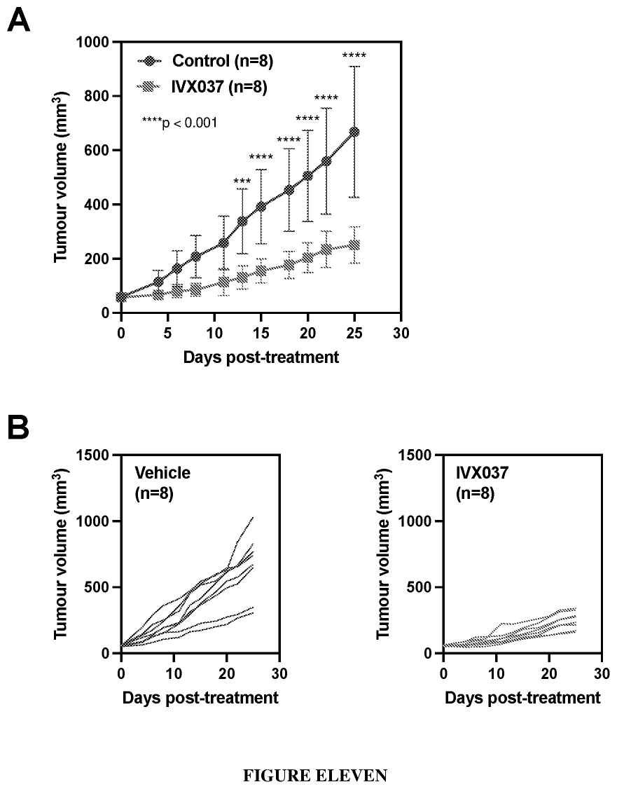

FIG. 10 indicates the average (A) and individual (B) tumour volumes of mice treated with IVX037. Strong antitumour activity was observed in the Caco-2 xenografts after one intratumoural injection of the IVX037 strain. The treatment was well tolerated, and no adverse effects were observed. Statistical differences among the treatment groups were determined using two-way ANOVA.

### Example Seven: Oncolytic Activity of Intratumoural IVX037 in Human Gastric Cancer (NCI-N87) Xenografts

### Materials and Methods

Immune-compromised SCID-Balb/C mice were inoculated with human colorectal cancer cells NCI-N87 (2e6 cells) subcutaneously. Once tumours were physically palpable (≥25 mm3), mice received a single intratumoural injection of a control vehicle, or an IVX-037 OVO strain of the invention (1e8 TCID50/injection). Tumour volumes were measured, and animals were terminated 25 days post-treatment.

### Results

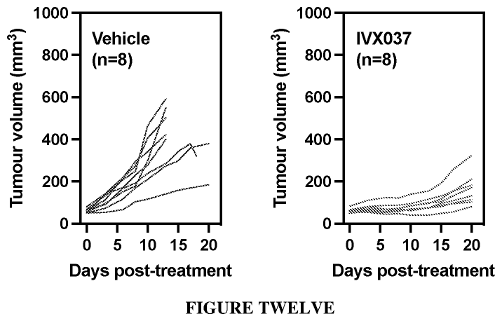

The average (A) and individual (B) tumour volumes of mice treated with the IVX037 strain are shown in FIG. 11. Strong antitumour activity was observed in the NCI-N87 xenografts after one intratumoural injection of the IVX037 strain. The treatment was well tolerated, and no adverse effects were observed. Statistical differences among the treatment groups were determined using two-way ANOVA

### Example Eight: Oncolytic Activity of Intratumoural IVX037 in Human Ovarian Cancer (IGROV-1) Xenografts

### Materials and Methods

Immune-compromised SCID-Balb/C mice were inoculated with human ovarian cancer cells IGROV-1 (2e6 cells) subcutaneously. Once tumours were physically palpable (≥25 mm3), mice received a single intratumoural injection of a control vehicle, or an IVX-037 OVO strain of the invention (1e8 TCID50/injection). Tumour volumes were measured, and animals were terminated 20 days post-treatment.

### Results

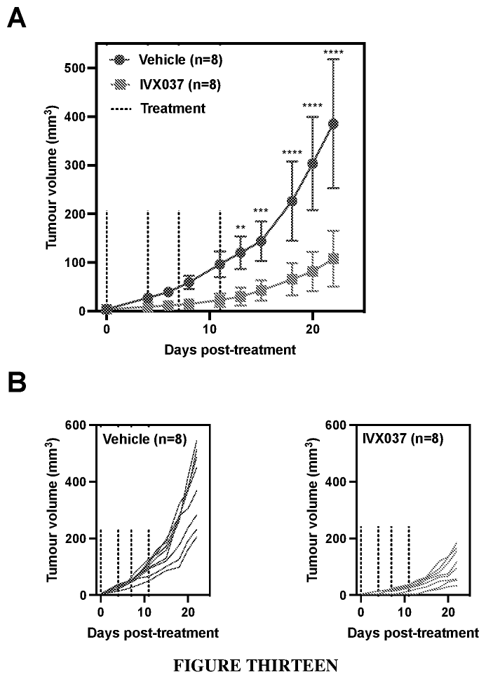

The individual tumour volumes of mice treated with the IVX037 strain are shown in FIG. 12. The IGROV-1 xenograft was more aggressive that led the tumour ulcerating. The affected animals were euthanised the data presented here are the individual volumes of each animal instead of the average. Strong antitumour activity was observed in the IGROV-1 xenografts after one intratumoural injection of the IVX037 strain and no tumour ulceration was observed in this group. The treatment was well tolerated, and no adverse effects were observed. A two-way ANOVA could not be performed with the early termination of the control animals.

### Example Nine: Oncolytic Activity of Intravenous IVX037 in Human Colorectal Cancer (WiDr) Xenografts

### Materials and Methods

Immune-compromised SCID-Balb/C mice were inoculated with human colorectal cancer cells WiDr (2e5 cells) subcutaneously. Once tumours were physically palpable (approximately 4 mm3), mice received four intravenous injections of a control vehicle, or an IVX-037 OVO strain of the invention (1e8 TCID50/injection). spaced three to four days apart. Tumour volumes were measured, and animals were terminated 22 days post-treatment.

### Results

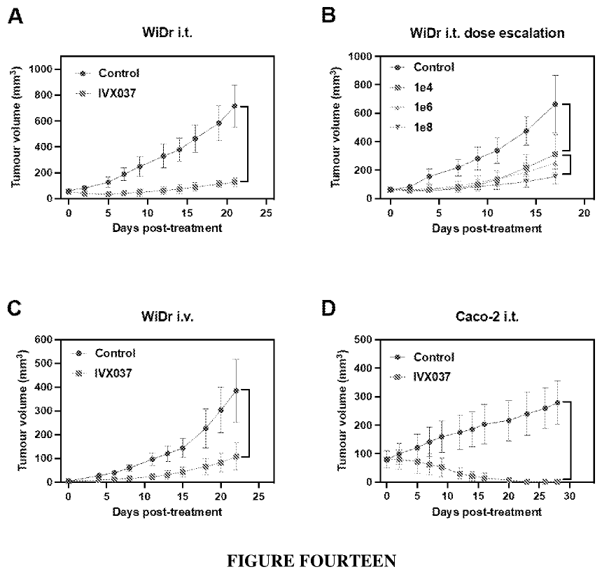

FIG. 13 shows the average (A) and individual (B) tumour volumes of mice treated with the IVX037 strain. Strong antitumour activity was observed in the WiDr xenografts after four intravenous injections of the IVX037 strain. The treatment was well tolerated, and no adverse effects were observed. Statistical differences among the treatment groups were determined using two-way ANOVA.

### Example Ten: Synthesis of IVX-037 OVO Viral Strains

The IVX-037 OVO strains of the invention belong to the Enterovirus B species, specifically wild-type echovirus 12 (E12), prototype strain Travis. To attain IVX-037 OVO material with complete manufacturing history and mitigate adventitious agent risk, the IVX-037 OVO strains were rescued from a pUC like plasmid containing the complete E12 viral sequence following transient transfection in Vero MCB LN 1595.01 cells. The E12 virus sequence inserted in the pUC-like plasmid was not genetically modified. The plasmid was synthesized at GeneArt, ThermoFisher Scientific while the E12 progeny virus rescue procedure was performed at Imm VirX. An overview of the IVX037 pre-MVSS manufacture is outlined below.

Further specifics of the IVX-037 OVO strain synthesis process our outlined in Table 1 below:

TABLE 1
IVX-037 OVO synthesis process

| | Documentation | | |
|---|---|---|---|
| Manufacturing Step | (LAB-PROC BR etc) | Sample ID in | Collected Sample ID(s) |
| Plasmid pIVX037 | QAD20AECKJC_2967406_pIVX037 | pIVX037 (3074 W) | Construct ID: 20AECKKJC |
| Containing E12 virus sequence | | sequence | Conc: 50 ng/uL |
| | | | Vector backbone: pMA |
| | | | Synthesized by GeneArt, |
| | | | ThermoFisher Scientific |
| | | | TSE-free production |
| Transfection of (0.5 ug) of | LP075 20210426 BD | Plasmid construct ID: | 1. Transfect #6A 26 Apr. 2021, |
| pIVX037 on Vero cells (MCB | Transfection A (pIVX037 | 20AECKKJC | H: 2 May 2021 |
| Lot 1595.01) cultured in 6 well | 20AEIJHC, 20EIJIC) | Gene name: pIVX037 | |
| plate using lipofection to generate | BioReliance GMP production final | Vector: pMA | |
| infectious progeny virus | report 1595.01 | Vials: | |
| Harvest of cells & supernatant | (AG48GV.912000.BUK) | Pool of 20AEIJHC & | |
| (day 7 p.i.) | | 20AEEIJIC | |
| Benzonase Endonuclease | LP076 20210504 BD Benzonase treat | Transfect #6A | 1. Transfect #6A 26 Apr. 2021, |
| digestion of residual plasmid | (transf 26Apr 6A) | 26 Apr. 2021, | (B): 4 May 2021 |
| DNA & Removal of cellular | | H: 2 May 2021 | *B-benzonase treated |
| debris by low-speed centrifugation | | | |
| Plaque-purification #1 of virus | LP077 20210505 RI Plaque Purif #1 | 1. Transfect #6A | Harvested plaque (origin: 6A |
| on Vero MCB 1595.01 (6 well | (transf 6A, 6B 26Apr) | 26 Apr. 2021, | transfection 26 Apr. 2021) |
| plate) | Harvest: day 5 p.i. (10 May 2021) | (B): 4 May 2021 | Transfect #6A 26 Apr. 2021, |
| | | *B-benzonase treated | (B): 4 May 2021 -> |
| | | | 1. 6A (B*) PP1 P1 10 May 2021 |
| Plaque-purification #2 of virus | LP077 20210510 RI Plaque Purif #2 | 1. 6A (B) PP1 P1 | Harvested plaque (origin: transfection |
| on Vero MCB 1595.01 (6 well | (transf 6A 26 Apr. 2021) | | 6A 26 Apr. 2021) |
| plate) | Harvest: day 4 p.i. (14 May 2021) | | 6A (B) PP1 P1 -> |
| | | | 1. 6A (B1) PP2 P1 14 May 2021 |
| Plaque-purification #3 of virus | LP077 20210514 RI Plaque Purif #3 | 1. 6A (B1) PP2 P1 | Harvested plaque (origin: transfection |
| on Vero MCB 1595.01 (6 well | (transf 6A 26 Apr. 2021) | 14 May 2021 | 6A 26 Apr. 2021) |
| plate) | Harvest: day 5 p.i. (19 May 2021) | | 6A (B1) PP2 P1 -> |
| | | | 1. 6A (B1) PP3 P1 19 May 2021 |
| Expansion #1 of triple plaque- | LP078 20210519 BD First expansion | 1. 6A (B1) PP3 P1 | Collected sample: |
| purified virus on Vero MCB | Vero | 19 May 2021 | 6A (B1) PP3 P1 19 May 2021 -> |
| 1595.01 cells (2 × T25 flask) | Harvest: day 4 p.i. (23 May 2021) | | Pre-pre-MVSS 24 May 2021 [6A (B1) |
| | Clarification & filtration: | | PP3 P1 exp 23/5 step 5 Apr. 2010 |
| | 24 May 2021 | | 24 May 2021] |
| Expansion #2 of triple plaque- | LP078 20210524 BD Second | Pre-pre-MVSS 24 May 2021 | 102 vials dispensed |
| purified virus on Vero MCB | expansion Vero, harvest & fill | (6A (B1) PP3 P1 | IVX037 (E12), pre-MVSS-01, (VP01) |
| 1595.01 cells (5 × T175 flask) | Harvest: day 2 p.i. (26 May 2021) | 19 May 2021 exp, | Vol 1.5 mL, DOM: 31 May 2021 |
| Clarification, filtration & fill | | H 23 May 2021, clarify & | |
| IVX037 pre-MVSS-01 | | filtered 24 May 2021) | |

Footnotes:
Bold: sample selected for further processing in next process step

### Example Eleven: IVX037 As Mediator in Immune Response and Antitumor Activity

CXCL10 is a marker of the severity of viral infection and facilitates recruitment of T cells, natural killer cells, macrophages, and dendritic cells. Evidence suggests that CXCL10 is necessary for recruitment of antitumoral T cells into melanoma tumours. Furthermore, CXCL10 signalling through the CXCR3 receptor promotes the migration of lymphocytes to dendritic cells, which was necessary for response to PD-1 blockade in a transplantable mouse model. Clinically, there is evidence that high pre-treatment CXCL9 and CXCL10 levels are correlated with response to anti-PD-(L) 1 therapy in patients with non-small-cell lung cancer, and CXCL9 and CXCL10 increase in the first few months of treatment in patients with melanoma responding to PD-1 inhibitor therapy.

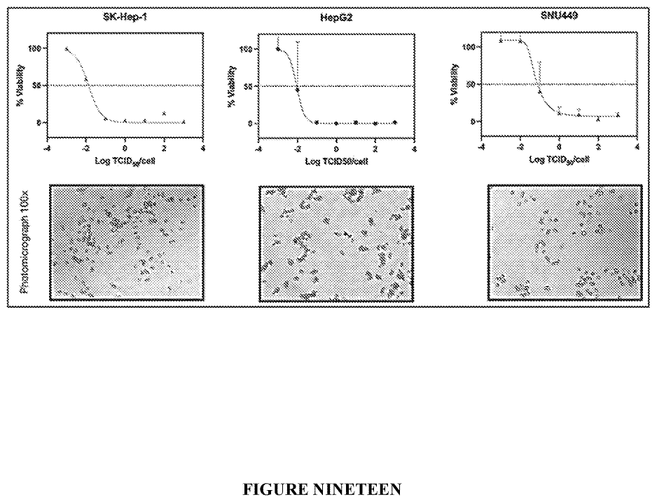

The sera of patients were quantified using a multiplex bead-based assay (The LEGENDplex™ Human Essential Immune Response Panel (13-plex), #740930, LEGENDplex™, Biolegend Inc., USA). The assay can detect 13 inflammatory cytokines/chemokines, including IL-4, IL-2, CXCL10 (IP-10), IL-1β, TNF-α, CCL2 (MCP-1), IL-17A, IL-6, IL-10, IFN-γ, IL-12p70, CXCL8 (IL-8). The staining procedures were performed as suggested by the manufacturer protocol. The flow cytometry was performed using BD LSRFortessa™ X-20 (Becton Dickinson Biosciences, San Jose, USA). Cytokine concentration was calculated based on a standard curve using BioLegend's LEGENDplex™ data analysis software provided by the manufacturer. The cytokine data were analysed at Day 1, 8, 15 and 29. Preliminary serum biomarker analysis has indicated early signs of IVX037 induction of potentially beneficial inflammatory cytokines/chemokines, such as CXCL10 (FIG. 18).

### Example Twelve: Infectivity and Oncolytic Effects of IVX037 in Hepatocellular Carcinoma (HCC) Cell Lines

A panel of 7 liver cancer cell lines with varying degrees of genetic complexity was purchased from the ATCC. They are SNU-475, C3A [HepG2/C3A, derivative of Hep G2], SNU-449, PLC/PRF/5, SNU-387, SK-HEP-1, SNU-423. Data in FIG. 19 provides a preliminary summary on the oncolytic effects of IVX037 on these liver cancer lines.

### Example Thirteen: Phase 1a Open-Label, Non-Randomized, Multi-Centre Clinical Trial of Intratumoral IVX037 in Patients with Advanced Microsatellite Stable (MSS) Colorectal, Gastroesophageal or Ovarian Cancer: Trial in Progress

### Background

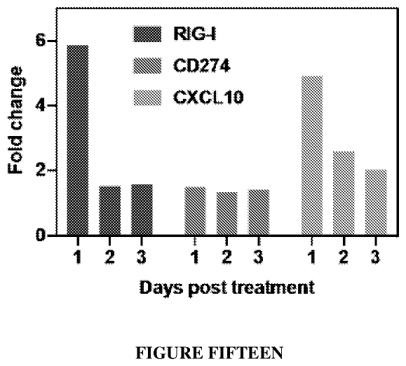

IVX037 challenge can induce selective in vitro tumour cell lytic infection via specific viral capsid cellular receptor interactions in cell cultures of human colorectal, gastric and ovarian cancers. Significant anti-tumour activity was displayed by a single intratumoral injection of IVX037 in human xenografts of microsatellite stable (MSS) colorectal (FIG. 14), gastric and ovarian cancers in SCID mice.

### In Vivo Studies

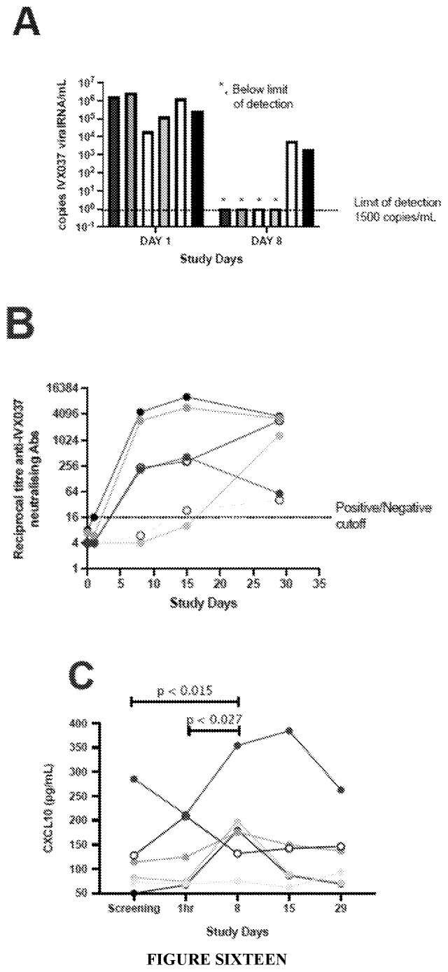

In vivo human MSS colorectal cancer xenograft studies in mice, revealed that intratumoral administration of IVX037 induced elevated levels of g-INF response genes (CXCL10, RIG-I) and up-regulated expression of a key immune-checkpoint molecule, PD-L1 (FIG. 15), indicating an inflammation phenotype within the treated tumour microenvironment (TME).

The primary pharmacodynamic studies were approved by the University of Newcastle Animal Ethics Committee and utilized in-house purified IVX037 propagated from an in-house viral seed stock. The activity of a single dose of IVX037 at 1×10⁸ TCID₅₀ administered intratumorally (IT) to female severe combined immunodeficient (SCID) mice (8 per group) bearing WiDr (an immortalized cell line of human colorectal adenocarcinoma cells) human xenografts was evaluated (TR 2020-007) 32.

The results highlighted that while tumour volumes of mice in vehicle (control) group gradually increased over three weeks post treatment, IVX037 administration reduced tumour volumes of mice in the treatment group 2 days post infection and stabilized until day 9 before slowly increasing until day 21 post infection. Overall, the average tumour volume of mice in IVX037 treated group was statistically significantly smaller compared to those in vehicle group over three weeks post treatment (FIG. 5A). Furthermore, there were no significant changes in the body weight of mice from either treatment group.

Gene profiling analysis by RT-PCR on IVX037 treated tumours revealed that expression of retinoic acid-inducible gene I (RIG-I) and C-X-C motif chemokine ligand 10 (CXCL10) increased and peaked (approximately 5-fold change) at 1 day after IVX037 infection while expression of CD274, a gene that encodes for PD-L1, increased about 1.3-fold change and remained constant over three days post infection (FIG. 15).

When tumours reached to an average size (approximately 50 mm³), mice were treated with virus IVX037 (n=8 mice) or control formulation buffer (n=6 mice) on the day 0. Mice were sacrificed at 1-, 2-, and 3-day post treatment. Tumours were collected and RNA was extracted. Expression of RIG-I, CD274 and CXCL10 was measured using RT-PCR and calculated as a fold change.

The induction of a virally inflamed TME is suggested to potentially allow increased migration of anti-tumour lymphocytes both within injected and distant lesions and elevated levels of cellular targets for immune checkpoint therapies.

Increased serum levels CXCL10 and CCL22 in melanoma patients administered with another RNA oncolytic virus, V927 in combination with pembrolizumab, were associated with responses with suggesting viral replication contributes to antitumor immunity (Silk A W, et al, 2023 Cancer Immunol Immunother; 72(6):1405-1415).

### Clinical Trial Methods

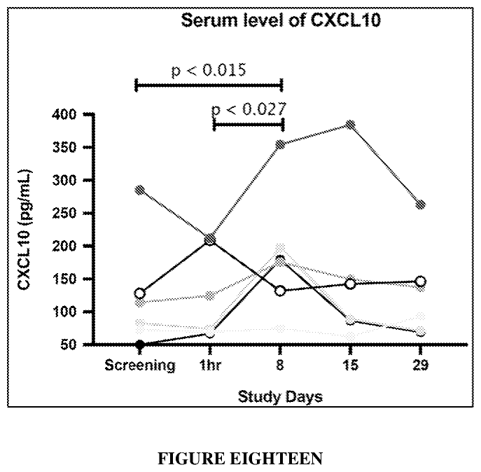

This is a Phase 1a, first-in-human, open-label, non-randomized, multi-centre clinical trial of intratumoral IVX037 in patients with advanced MSS colorectal, gastroesophageal or ovarian cancer. A general trial schema can be seen at FIG. 17.
Inclusion criteria: Patients (pts) must have one injectable tumour of liver/nodal/peritoneal disease.
Exclusion criteria: Candidate for hepatic surgery or locoregional therapy for liver or other lesions. Clinically significant ascites (Grade ≥2), continuous systemic treatment with either corticosteroid (>10 mg daily).
Intervention: Pts will be sequentially enrolled into 3 dose escalation cohorts to receive 1 (n=3 pts), 2 (n=3 pts) or up to 7 doses permitted at Investigator discretion in absence of DLTs' (n=15) of up to 3×10⁸ TCID₅₀ of IVX037 doses for Cohort 3 intratumorally, administered on Days 1, 15, 29, 43, 57, 71 and 85, as applicable.
Primary objective: to determine the feasibility, safety and tolerability of intratumoral IVX037 including the incidence of dose-limiting toxicities (DLT).
Secondary objectives: to assess the maximum tolerated dose (MTD) of IVX037, administered as either 1, 2 or 3 injections per lesion. Tumour response will be assessed using RECIST 1.1, with the first response assessment occurring at Day 50.
Exploratory objectives: Several biomarker effects of IVX037 administration in peripheral blood and tumour tissue addressing tumour infiltrating lymphocytes and cellular target expression levels for immune checkpoint therapies will be assessed.

### Results

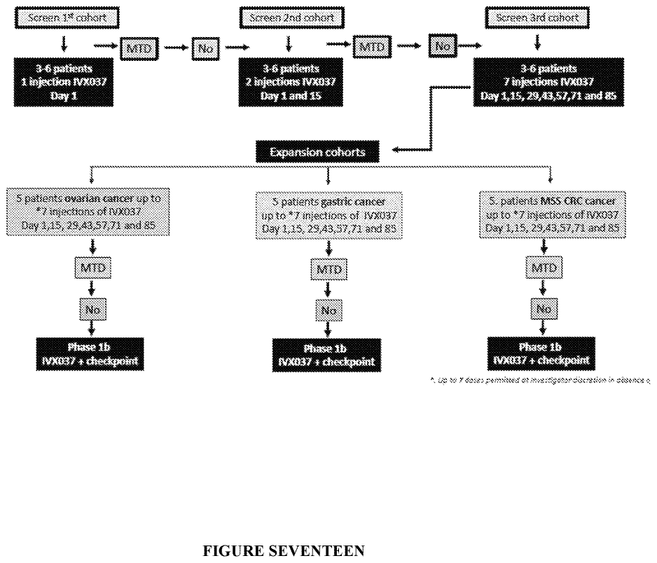

Patient recruitment commenced in April 2023, with currently 8 pts enrolled. IVX037 dosing in Cohorts 1 and 2 is complete, with Cohort 3 dosing ongoing.
To date IVX037 intralesional administration has been generally well tolerated, with all patients exhibiting some level of systemic exposure immediately following injection (FIG. 16A), with no dose-limiting toxicities observed. Mild flu-like reactions after injection (fatigue, chills, rigors, injection site discomfort) observed.
IVX037 has been successfully administered to liver, lymph node and abdominal metastases.
Currently all patients have developed serum neutralising anti-IVX037 antibodies by Day 15 post-viral administration (FIG. 16B).
Preliminary serum biomarker analysis has indicated early signs of IVX037 induction of potentially beneficial inflammatory cytokines/chemokines, such as CXCL10 (FIG. 16C).

Recruitment is ongoing and Phase 1b in combination with immune checkpoint blockade is planned.

1. A modified Picornavirus comprising change(s) in any one or more of capsid proteins: VP1, VP2, and VP3 compared to a wild-type strain of the virus, which confer enhanced binding capacity to decay accelerating factor (DAF/CD55) and/or Neonatal Fc Receptor (FcRn) compared to the wild-type strain.

2. The modified Picornavirus of claim 1, comprising said change(s) in each of the VP2 and VP3 capsid proteins or in each of the VP1, VP2 and VP3 capsid proteins.

3. (canceled)

4. The modified Picornavirus of claim 1, further comprising change(s) in any one or more of non-structural proteins 2A, 3A, 3C, and 3D compared to the wild-type strain of the virus which confer enhanced binding capacity to decay accelerating factor (DAF/CD55) compared to the wild-type strain.

5. The modified Picornavirus of claim 1, wherein the wild-type strain and the modified Picornavirus have an identical nucleotide sequence and/or an identical amino acid sequence, apart from:
(i) the change(s) in any one or more of capsid proteins VP1, VP2, and VP3; and optionally
(ii) change(s) in any one or more of non-structural proteins 2A, 3A, 3C, and 3D compared to the wild-type strain of the virus, which confer enhanced binding capacity to decay accelerating factor (DAF/CD55) compared to the wind-type strain.

6. The modified Picornavirus of claim 1, wherein the modified Picornavirus is:
(a) an Enterovirus,
(b) selected from the group consisting of Echovirus, Poliovirus, unclassified Enteroviruses, Rhinovirus, Paraechovirus, Hepatovirus, and Cardiovirus,
(c) not a Coxsackievirus,
(d) selected from the group consisting of Enterovirus B85, Coxsackievirus A9, Coxsackievirus A13, Coxsackievirus 15 or Coxsackievirus 21, or
(e) selected from the group consisting of Echovirus 1, Echovirus 3, Echovirus 6, Echovirus 7, Echovirus 9, Echovirus 11, Echovirus 12 (E12), Echovirus 12, Echovirus 13, Echovirus 14, Echovirus 15, Echovirus 17, Echovirus 25, Echovirus 26, Echovirus 29, or Echovirus 30.

7. (canceled)

8. (canceled)

9. (canceled)

10. (canceled)

11. The modified Picornavirus of claim 1, wherein:
(a) the change(s) in capsid protein VP2 comprise or consist of an asparagine to threonine change at residue 142,
(b) the change(s) in capsid protein VP3 comprise or consist of an alanine to valine change at residue 206,
(c) the change(s) in capsid protein VP2 comprise or consist of a histidine to tyrosine change at residue 154, and/or a serine to asparagine change at residue 168,
(d) the change(s) in capsid protein VP1 comprise or consist of a tyrosine to histidine change at residue 230,
(e) the change(s) in capsid protein VP1 comprise or consist of a phenylalanine to tyrosine change at residue 210, and/or
(f) the change(s) in capsid protein VP1 comprise or consist of a glutamine to arginine change at residue 132, preferably wherein the change confers enhanced binding to Neonatal Fc Receptor (FcRn).

12. (canceled)

13. (canceled)

14. (canceled)

15. (canceled)

16. (canceled)

17. (canceled)

18. The modified Picornavirus of claim 4, wherein the change(s) in the non-structural proteins 2A, 3A, 3C, and/or 3D comprise or consist of any one or more of:
a phenylalanine to tyrosine change at residue 47 of the non-structural 2A protein;
an isoleucine to tyrosine change at residue 6 of the non-structural 3A protein;
a histidine to arginine change at residue 39 of the non-structural 3C protein; and
an isoleucine to leucine change at residue 123 of the non-structural 3D protein.

19. The modified Picornavirus of claim 1, wherein the wild-type strain is Echovirus 12 strain Travis, preferably comprising or consisting of an amino acid sequence as defined in SEQ ID NO: 46.

20. (canceled)

21. The modified Picornavirus of claim 1, further comprising
(i) at least one nucleic acid sequence encoding an exogenous protein or component thereof,
preferably wherein the exogenous protein is an immunostimulatory protein, or an agent (e.g. an antibody) capable of binding to and inhibiting the biological activity of an immune checkpoint molecule or a ligand of the immune checkpoint molecule, preferably wherein:
(a) the immunostimulatory protein is selected from any one or more of: an interleukin, including but not limited to interleukin-2 (IL-2), interleukin-12 (IL-12), interleukin-15 (IL-15), interleukin-21 (IL-21), RANTES, GM-CSF, an interferon, including but not limited to IFN-γ, TNF-α, a pro-drug converting enzyme, biologically active component(s) thereof, combinations thereof; and/or
(b) the immune checkpoint molecule is selected from any one or more of: PD-1, PD-L1, PD-L2, CTLA-4, CD134, CD134L, CD137, CD137L, CD80, CD86, B7-H3, B7-H4, B7RP1, LAG-3, ICOS, TIM3, GAL9, CD28, OX-40, and TIGIT,
biologically active component(s) thereof, combinations thereof;
and/or
(ii) one or more components for expression of a tissue-specific miRNA capable of inhibiting replication of the modified Picornavirus in a tissue-specific manner.

22. (canceled)

23. (canceled)

24. (canceled)

25. (canceled)

26. (canceled)

27. (canceled)

28. (canceled)

29. The modified Picornavirus of claim 1, wherein the Picornavirus comprises:
(i) a VP1 capsid protein amino acid sequence comprising any one of SEQ ID NOs: 54-57, 74, 86, 98, 110, 122, 134, 146 or 158;
(ii) a VP2 capsid protein amino acid sequence comprising any one of SEQ ID NOs: 58-61, 75, 87, 99, 111, 123, 135, 147 or 159;
(iii) a VP3 capsid protein amino acid sequence comprising any one of SEQ ID NOs: 62, 63, 76, 88, 100, 112, 124, 136, 148 or 160;
(iv) a non-structural protein 2A sequence comprising any one of SEQ ID NOs: 64, 65, 77, 89, 101, 113, 125, 137, 149 or 161;
(v) a non-structural protein 3A sequence comprising any one of SEQ ID NOs: 66, 67, 78, 90, 102, 114, 126, 138, 150 or 162;
(vi) a non-structural protein 3C sequence comprising any one of SEQ ID NOs: 68, 69, 79, 91, 103, 115, 127, 139, 151 or 163; and/or
(vii) a non-structural protein 3D sequence comprising any one of SEQ ID NOs: 70, 71, 80, 92, 104, 116, 128, 140, 152 or 164; and/or
(viii) an amino acid sequence as defined in SEQ ID NO: 42, SEQ ID NO: 52, SEQ ID NO: 53, SEQ ID NO: 73, SEQ ID NO: 85, SEQ ID NO: 97, SEQ ID NO: 109, SEQ ID NO: 121, SEQ ID NO: 133, SEQ ID NO: 145 or SEQ ID NO: 157 or a variant thereof having at least: 80%, 85%, 90%, 95%, 96%, 97%, 98% or 99% sequence identity with SEQ ID NO: 42, SEQ ID NO: 52, SEQ ID NO: 53, SEQ ID NO: 73, SEQ ID NO: 85, SEQ ID NO: 97, SEQ ID NO: 109, SEQ ID NO: 121, SEQ ID NO: 133, SEQ ID NO: 145 or SEQ ID NO: 157.

30. (canceled)

31. The modified Picornavirus of claim 1, encoded by a nucleotide sequence comprising or consisting of an RNA or cDNA/DNA sequence defined in SEQ ID NO: 41, SEQ ID NO: 50, SEQ ID NO: 51, SEQ ID NO: 72, SEQ ID NO: 84, SEQ ID NO: 96, SEQ ID NO: 108, SEQ ID NO: 120, SEQ ID NO: 132, SEQ ID NO: 144 or SEQ ID NO: 156, or a variant thereof having at least: 80%, 85%, 90%, 95%, 96%, 97%, 98% or 99% sequence identity with an RNA or cDNA/DNA sequence defined in SEQ ID NO: 41, SEQ ID NO: 50, SEQ ID NO: 51, SEQ ID NO: 72, SEQ ID NO: 84, SEQ ID NO: 96, SEQ ID NO: 108, SEQ ID NO: 120, SEQ ID NO: 132, SEQ ID NO: 144 or SEQ ID NO: 156.

32. (canceled)

33. A pharmaceutical composition comprising any one or more of:
the modified Picornavirus of claim 1,
RNA of the modified Picornavirus of claim 1, and
complementary DNA (cDNA) encoding all or a portion of the genome of the modified Picornavirus of claim 1;
and a pharmaceutically acceptable carrier, excipient or diluent.

34. The pharmaceutical composition of claim 33 wherein the RNA of the modified Picornavirus (e.g. synthetic RNA) and/or complementary DNA (cDNA) encoding all or a portion of the genome of the modified Picornavirus is provided within a nanoparticle (e.g. a lipid nanoparticle).

35. The pharmaceutical composition of claim 33, further comprising CAR-T cells, natural Killer (NK) cells, an immunostimulatory protein, and/or an agent (e.g. an antibody) capable of binding to an immune checkpoint molecule or to a ligand of the immune checkpoint molecule, preferably wherein:
(a) the CAR-T cells are engineered to bind to any one or more of: EGFRVIII, interleukin 13Ra2, FAP, GD2, EpCam, CD133, CD70, Her2, CEA, GAP, CD5, CD38, Muc1, GPC3, BCMA, Meso, PSCA, CD33, PSMA, ROR1, Muc1, EphA2, CD123, CD19, Claudin 18.2; on the surface of a cancerous cell;
(b) the immunostimulatory protein is selected from any one or more of: an interleukin, including but not limited to interleukin-2 (IL-2), interleukin-12 (IL-12), interleukin-15 (IL-15), interleukin-21 (IL-21), RANTES, GM-CSF, an interferon, including but not limited to IFN-γ, TNF-α, a pro-drug converting enzyme, biologically active component(s) thereof, combinations thereof; and/or
(c) the immune checkpoint molecule is selected from any one or more of: PD-1, PD-L1, PD-L2, CTLA-4, CD134, CD134L, CD137, CD137L, CD80, CD86, B7-H3, B7-H4, B7RP1, LAG-3, ICOS, TIM3, GAL9, CD28, OX-40, and TIGIT, biologically active component(s) thereof, combinations thereof.

36. (canceled)

37. (canceled)

38. (canceled)

39. The pharmaceutical composition of claim 33, wherein the composition comprises:
(a) Picornaviruses at between about 10⁸ and about 10¹⁵ viral particles per mL, between about 10⁸ and 10¹² viral particles per mL, or between about 10⁸ and 10¹⁰ viral particles per mL;
(b) a particle to infectivity ratio of less than or equal to about 3000:1, less than or equal to about 300:1, in the range of about 200:1 to about 5:1, in the range of about 50:1 to about 10:1, or less than about 20:1; and/or
(c) a level of host cell DNA less than or equal to about 200 ng per mL, in the range of 1 to about 100 ng per mL, in the range of about 1 to about 50 ng per mL, or in the range of about 1 to about 10 ng per mL.

40. (canceled)

41. (canceled)

42. A method for treating cancer in a subject, the method comprising administering to the subject a therapeutically effective amount of any one or more of:
the modified Picornavirus of claim 1;
RNA of the modified Picornavirus of claim 1;
complementary DNA (cDNA) encoding all or a portion of a genome of the modified Picornavirus of claim 1; and
the pharmaceutical composition of claim 33,
wherein the cancer comprises cancerous cells which express decay accelerating factor (DAF/CD55) and/or Neonatal Fc Receptor (FcRn).

43. (canceled)

44. (canceled)

45. (canceled)

46. The method of claim 42, wherein the therapeutically effective amount is administered to the subject by parenteral, intravenous, intravesical, subcutaneous, oral, intratumoural, ocular, topical or systemic administration.

47. The method of claim 42, wherein the therapeutically effective amount is co-administered with CAR-T cells, natural killer (NK) cells, an immunostimulatory protein, and/or an agent (e.g. an antibody or a monoclonal antibody) capable of binding to an immune checkpoint molecule or to a ligand of the immune checkpoint molecule, preferably wherein:
(a) the CAR-T cells are engineered to bind to any one or more of EGFRVIII, interleukin 13Ra2, FAP, GD2, EpCam, CD133, CD70, Her2, CEA, GAP, CD5, CD38, Muc1, GPC3, BCMA, Meso, PSCA, CD33, PSMA, ROR1, Muc1, EphA2, CD123, CD19, and Claudin 18.2 on the surface of a cancerous cell;
(b) the immunostimulatory protein is selected from any one or more of an interleukin, including but not limited to interleukin-2 (IL-2), interleukin-12 (IL-12), interleukin-15 (IL-15), interleukin-21 (IL-21), RANTES, GM-CSF, an interferon, including but not limited to IFN-γ, TNF-α, a pro-drug converting enzyme, biologically active component(s) thereof, and combinations thereof; and/or
(c) the immune checkpoint molecule is selected from the group consisting of PD-1, PD-L1, PD-L2, CTLA-4, CD134, CD134L, CD137, CD137L, CD80, CD86, B7-H3, B7-H4, B7RP1, ICOS, LAG-3, TIM3, GAL9, CD28, OX-40, and TIGIT.

48. (canceled)

49. The method of claim 47, wherein the CAR-T cells, the natural killer (NK) cells, the immuno-stimulatory protein and/or the agent is/are co-administered prior to or following the administration of the therapeutically effective amount to the subject.

50. (canceled)

51. (canceled)

52. (canceled)

53. (canceled)

54. (canceled)

55. (canceled)

56. The method of claim 42, wherein the cancer is ovarian cancer, colorectal cancer, gastric cancer, liver cancer, pancreatic cancer, head and neck cancer, stomach cancer, breast cancer, sarcoma, lymphoma, or brain cancer, preferably wherein the cancer is classified as a cancer that is resistant to immune checkpoint therapy and/or wherein the subject is a human.

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)


SEQUENCE LISTING (167 sequences)

SEQ ID NO: 1 - DNA, 21 bp, synthetic construct
  source 1..21: mol_type = other DNA
gccacgtgtggagtcttgtgc

SEQ ID NO: 2 - DNA, 22 bp, synthetic construct
  source 1..22: mol_type = other DNA
ggtagtgtttattcctcgatgc

SEQ ID NO: 3 - DNA, 22 bp, synthetic construct
  source 1..22: mol_type = other DNA
cagtataggcaacgcgtatagc

SEQ ID NO: 4 - DNA, 21 bp, synthetic construct
  source 1..21: mol_type = other DNA
tctgactgtgctcgtaattgc

SEQ ID NO: 5 - DNA, 23 bp, synthetic construct
  source 1..23: mol_type = other DNA
cagcaggatgaatatacgagtgc

SEQ ID NO: 6 - DNA, 21 bp, synthetic construct
  source 1..21: mol_type = other DNA
cttctaccagacctggtcctt

SEQ ID NO: 7 - DNA, 20 bp, synthetic construct
  source 1..20: mol_type = other DNA
ttaaaacagcctgtgggttg

SEQ ID NO: 8 - DNA, 20 bp, synthetic construct
  source 1..20: mol_type = other DNA
tgaatgcggctaatcctaac

SEQ ID NO: 9 - DNA, 20 bp, synthetic construct
  source 1..20: mol_type = other DNA
aacagcagaagaatgtgggt

SEQ ID NO: 10 - DNA, 20 bp, synthetic construct
  source 1..20: mol_type = other DNA
aaagcatagtgacaaacgcc

SEQ ID NO: 11 - DNA, 20 bp, synthetic construct
  source 1..20: mol_type = other DNA
gcaaccaactgtcaactcaa

SEQ ID NO: 12 - DNA, 18 bp, synthetic construct
  source 1..18: mol_type = other DNA
aggaggccgtgaacagag

SEQ ID NO: 13 - DNA, 20 bp, synthetic construct
  source 1..20: mol_type = other DNA
ctagcatcttctggacggag

SEQ ID NO: 14 - DNA, 19 bp, synthetic construct
  source 1..19: mol_type = other DNA
cacagctcatgggtgtgac

SEQ ID NO: 15 - DNA, 22 bp, synthetic construct
  source 1..22: mol_type = other DNA
cactgtaactgctactcttgcc

SEQ ID NO: 16 - DNA, 19 bp, synthetic construct
  source 1..19: mol_type = other DNA
ggagcagggaaatcagttg

SEQ ID NO: 17 - DNA, 23 bp, synthetic construct
  source 1..23: mol_type = other DNA
gaagaacccaagtcagatattca

SEQ ID NO: 18 - DNA, 19 bp, synthetic construct
  source 1..19: mol_type = other DNA
caccatgctcggcatctat

SEQ ID NO: 19 - DNA, 20 bp, synthetic construct
  source 1..20: mol_type = other DNA
attgagagctcaaaggatgc

SEQ ID NO: 20 - DNA, 23 bp, synthetic construct
  source 1..23: mol_type = other DNA
aatcaagactcattgaagcatct

SEQ ID NO: 21 - DNA, 20 bp, synthetic construct
  source 1..20: mol_type = other DNA
actacttgctgaagctggga

SEQ ID NO: 22 - DNA, 19 bp, synthetic construct
  source 1..19: mol_type = other DNA
aaagtagtcggttccgctg

SEQ ID NO: 23 - DNA, 18 bp, synthetic construct
  source 1..18: mol_type = other DNA
accacattggcacactcc

SEQ ID NO: 24 - DNA, 19 bp, synthetic construct
  source 1..19: mol_type = other DNA
aatagtcgcgctgttgttg

SEQ ID NO: 25 - DNA, 26 bp, synthetic construct
  source 1..26: mol_type = other DNA
tatctcacctagtaatgtccttttga

SEQ ID NO: 26 - DNA, 20 bp, synthetic construct
  source 1..20: mol_type = other DNA
gtttctgctgctgttagtgc

SEQ ID NO: 27 - DNA, 20 bp, synthetic construct
  source 1..20: mol_type = other DNA
tgaaatgtgaccaaccatca

SEQ ID NO: 28 - DNA, 20 bp, synthetic construct
  source 1..20: mol_type = other DNA
tccttcaaaactgactgggt

SEQ ID NO: 29 - DNA, 21 bp, synthetic construct
  source 1..21: mol_type = other DNA
tgggtatcccgtagtattgtg

SEQ ID NO: 30 - DNA, 20 bp, synthetic construct
  source 1..20: mol_type = other DNA
gatctgggtctggtggtaag

SEQ ID NO: 31 - DNA, 20 bp, synthetic construct
  source 1..20: mol_type = other DNA
gaactgaatagccttgccac

SEQ ID NO: 32 - DNA, 20 bp, synthetic construct
  source 1..20: mol_type = other DNA
ccgatctcctgatcattcat

SEQ ID NO: 33 - DNA, 20 bp, synthetic construct
  source 1..20: mol_type = other DNA
attgccttcaaacacctgat

SEQ ID NO: 34 - DNA, 19 bp, synthetic construct
  source 1..19: mol_type = other DNA
cgggttcagatggaatgtt

SEQ ID NO: 35 - DNA, 20 bp, synthetic construct
  source 1..20: mol_type = other DNA
ccaggtaacctcattgaagc

SEQ ID NO: 36 - DNA, 19 bp, synthetic construct
  source 1..19: mol_type = other DNA
ttccgcaccgaatgcggag

SEQ ID NO: 37 - DNA, 19 bp, synthetic construct
  source 1..19: mol_type = other DNA
aaacagcctgtgggttgtc

SEQ ID NO: 38 - DNA, 18 bp, synthetic construct
  source 1..18: mol_type = other DNA
cgccatggaacaaggagt

SEQ ID NO: 39 - DNA, 32 bp, synthetic construct
  source 1..32: mol_type = other DNA
tttttttttttttttttttttttttccgcacc

SEQ ID NO: 40 - DNA, 41 bp, synthetic construct
  source 1..41: mol_type = other DNA
ataagaatgcggccgcttttttttttttttttttttttttt

SEQ ID NO: 41 - RNA, 7473 nt, Echovirus 12
  source 1..7473: mol_type = unassigned RNA
ttaaaacagcctgtgggttgtccccacccacagggcccactgggcgctagcacactggtatcccggtacctttgtgcgcctgttttatataccctcccctcagtaacttagaagttcatcacaaatgatcaatagttagctcaacaaaccagttgagcctagatcaagcacttctgttaccccgggctgagtatcaataagctgttgacacggctgaaggagaaaacgcccgttacccgaccagctacttcggagaacctagtatcaccatagaggttgcgtagcgtttcgctccgcacaaccccagtgtagatcaggtcgatgagtcaccgcgttccccacaggcgactgtggcggtggctgcgttggcggcctgcccatggggttacccatgggacgcttcaatactgacatggtgtgaagagttgactgagctagctggtagtcctccggcccctgaatgcggctaatcctaactgtggagcaagtgcccacaacccagtgggtggcttgtcgtaatgggcaactctgcagcggaaccgactactttgggtgaccgtgtttctctttattcttatattggctgcttatggtgacaatctcagagttgttaccatatagctattggtttggccaaccagtgactaacagagcaattatatatcttttcattggatttatacctataaacaacaccagttataccactttgtgttacattattggcttgaactcgaaaagatgggagcgcaagtatcaacacaaaaaactggggcacacgagactggcctgagtgctagtggaaattccatcatccactataccaacattaattactacaaagatgcggcatccaattcagcaaacaggcaggatttcacccaagaccccgggaaattcaccgagccagtgaaggatatcatgataaaatcgatgcctgcgttaaactccccaacagcagaagaatgtgggtacagcgatagagtcagatcaatcaccctggggaactctaccatcaccacgcaggagtgtgccaatgtggtggtgggatatggcacgtggcctgattatctacatgatgatgaggccactgcagaggaccaaccaacacaaccggatgtggcgacgtgtaggttttacacacttgaatccattcagtggcagaagacatcagatgggtggtggtggaagtttccggaagcattgaaagacatgggactgttcggccaaaacatgcactatcattatcttggcaggtcgggttacaccatccacgtccaatgtaacgcgtctaaattccaccagggttgtttgcttgtggtgtgtgtgcctgaagctgagatggggtgcgctacagtggcgaatgaggttaccgcagccgctctctcatctggtgaaacggcaaaatattttgcaaagactggtgcgacgggaacccacacagtgcaaagcatagtgacaaacgccggcatgggggtgggcgtgggcaatctgacaatattcccacatcagtggataaacctccgtaccaacaacagcgcgactattgtcatgccttacataaacagtgtgcccatggataacatgttcagacactacaatttcaccctaatgatcatccccttcgtcccactggacttcacagctgaggcatctacgtatgtaccaatcactgtaacagtagctcccatgtgcgccgagtacaacgggctacgcctggcctcgcatcaaggtttgccgacaatgaacacccctggtagtaaccaattcctcacatcagatgactttcaatcaccatcagccatgccacaattcgatgtcacacccgagctcaggattccaggggaggtaaagaacttaatggaaatagccgaagttgactccgtagtgcctgtgaacaatacacaggactcagtgtacaacatggatgtgtacaaaattcctgttagtggaggcaaccaactgtcaactcaagtcttcgggttccaaatgcaaccagggctaaatagtgtgttcaaaaggacattactaggtgagatactgaactactatgcacactggtctggtagtgtaaagctcacctttgtgttctgtggatcggccatggccactggcaaatttctgctggcatattcccctccaggcgcagaccccccaaagtctaggaaagaagcaatgcttggtactcacgtgatatgggatataggacttcagtcaagctgcgtcttgtgcgtaccgtggatcagccaaacacattatagattggttcagcaggatgaatatacgagtgcaggatatgtaacttgctggtatcaaactagtttggttgtcccgcccggcgctcccgtcacgtgtggagtcttgtgcttagcatcggcgtgcaatgatttctctgtgagaatgttgagagacacaccttttatcgagcaaaaacagctactccaaggagatgtggaggaggccgtgaacagagccgttgcacgggtggcggatacactacccacagggccgagaaattcagagagcatccccgcactaacagcagcagaaactgggcatacatctcaggtggttcctggggataccatgcaaacccgtcatgtgaagaattaccactccagaactgaatcgtctgttgaaaacttcttatgccgagcagcatgtgtgtatattacaaagtacaagaccaaggacagtgatccagtgcaaaggtacgccaactggcggatcaacacccgtcaaatggttcagctaaggagaaaatttgagttgtttacatacctaaggtttgatatggaagttacttttgtgatcaccagttcacaagatgatggcacgcagttggcgcaggacatgccagtattaacacaccaagtcatgtatataccacctggagggccagtgccgaatagtgttactgattttgcgtggcaatcgtctaccaaccctagcatcttctggacggagggaaacgctccagctaggatgtccatcccctttatcagtataggcaacgcgtatagcaatttctatgatggttggtcacatttcacccaagatggagtatatggttwcaactccctcaataacatgggctccatctacattaggcatgtcaatgaacaaagcccgcatgcaattacgagcacagtcagagtctatttcaaaccaaaacacgtgcgggcctgggtgccaagaccacctaggctttgtgcatatgagaaatcaagcaacgtgaatttcaaaccaacagatgtgactacctcccgaacatccatcacagaagtccctagtcttagaccatcagtggtaaatactggagctttcggtcagcaatcaggagcggcttatgtgggaaactatagagtggttaatagacacctagccactcatgttgactggcagaactgtgtgtgggaggactataacagggacctccttgtaagcaccaccacagctcatgggtgtgacaccatagccagatgccaatgcacaacaggcgtgtacttctgtgcatcgaggaataaacactacccagtcagttttgaaggaccaggtctggtagaagtccaggagagcgagtactaccccaggaggtatcaatcacacgtcttgctggctgcgggattttcagagccaggagattgtggagggattctcagatgtgagcacggtgtcatcggtctagttactatgggtggcgaaggcgttgtcggattcgctgatgtacgcgacctgttatggcttgaagatgacgccatggaacaaggagtcaaggattacgtggaacaactagggaacgcctttggttcaggattcactaaccaaatctgtgagcaggtcaacctcctaaaagagtcactggtgggtcacgattccatcctagagaagtcccttaaagccctagtgaaaatcatatcagcactagtgatagtggtgaggaaccatgatgatttgatcactgtaactgctactcttgccctcattggttgtacctcctctccatggcggtggctcaagcacaaggtgtcacaatactacgggatacccatggcagagcgccaaagcaacgggtggctcaagaagttcacagagatgaccaacgcctgcaaagggatggaatggattgccatcaaaatccaaaagttcatagagtggcttaaacttaagattttaccagaagttaaggaaaaacatgaattcctgaataggctcaagcaactcccactgttggagagtcaaatagcaaccatcgagcagagtgcaccgtcccagagcgatcaggagcagctcttttcaaatgttcagtactttgcccattattgcagaaaatatgcgcccctatacgcagctgaggcaaaaagggtgttctctcttgaaaagaaaatgagtaattacatacagttcaagtccaaatgccgtattgaacctgtatgcttgctactacatgggagccccggagcagggaaatcagttgctaccagcctcattgggcgatcactagctgaaaaattgaatagctcagtgtactccttaccaccagacccagatcactttgatggttacaagcaacaagctgtagtgatcatggacgatctgtgccaaaatccagatggaaaagatgtgtctttgttctgtcaaatggtgtctagtgtagattttgtaccaccaatggccgcgctagaggagaaaggcattttgttcacctctccattcgttctagcctcaaccaatgcaggatccatcaacgcaccaacagtttcagacagcagagccctagctagaaggttccactttgacatgaacattgaagttatctccatgtacagtcaaaatggaaaaataaacatgcccatgtcagtcaaaacatgtgatgaggaatgctgcccagtcaatttcaagagatgctgcccactggtgtgtggcaaggctattcagttcattgacagaagaacccaagtcagatattcactggatatgttagtcaccgagatgttcagggagtacaaccacaggcacagtgtgggcgccaccctcgaggctctgttccaaggtccaccggtctacagggagatcaagatcagtgttgctccagaaacacctcccccaccagcaatcgctgatttattaaaatcagtagacagtgaagctgtgagagagtactgcaaggaaaaaggctggcttgtaccggaagttaactccaccctacagattgagaagcacgtcagcagagcatttatctgtctacaagctctgaccactttcgtctcagtagctggcataatctacattatctacaagttgtttgccggctttcagggcgcgtatacggggatgccaaatcagaaacccaaagtgcccactctgagacaagctaaagtgcagggcccagcattcgagttcgctgtggcaatgatgaaaaggaacgccagcacagtgaaaacggaatatggtgagttcaccatgctcggcatctatgacagatgggcagtgctaccacgccacgccaagcctggaccgactattttgatgaatgatcaggagatcggcgtgttagacgccaaagaattagtggacaaagatgggacaaatctggagttgacccttttaaagctcaaccgcaatgagaagtttagggacatcagagggtttctggcgagagaggaagctgaagtgaatgaggctgttctggcaataaacacaagcaagttcccaaatatgtacatacccgtaggtcaagtcactgattacggtttcctgaacctggggggaacacccacaaagaggatgctcatgtacaactttccaactagagcagggcaatgtggtggggtactcatgtcaacagggaaagttcttggtatacatgtaggaggaaatggacatcaagggttctccgctgctcttcttaggcactactttaatgaggagcaaggtgaaatagagtttattgagagctcaaaggatgcaggattccctgtgatcaacacccccagcaaaacaaaactggaaccaagcgtgtttcatcaggtgtttgaaggcaataaagagccagcagtccttagaaatggtgatccgcgtcttaaagtaaactttgaagaagccatattttccaagtacatcgggaacatcaacacacatgtggatgaatacatgctcgaagcagtggatcactacgcagggcaactggctactcttgacattagcactgaacccatgaaactagaagatgcagtgtatggcactgaaggactagaggctcttgacttaacaacaagtgcagggtacccgtatgtcgcactaggcatcaaaaagagagacatcctatccaaaaagactaaagatctgactaaattgaaggaatgcatggacaagtacggtttgaacctgccgatggtgacttatgtgaaggatgaacttagatcatcagagaaagtggctaagggaaaatcaagactcattgaagcatctagtttaaatgactctgtcgcgatgaggcagacgtttggcaacttgtacaaaacattccatctgaacccgggcattgtgacaggcagtgcagttggatgtgatccggaccttttctggagtaaaatacccgtgatgttagacggccacctcatagcattcgattactccggatatgatgccagcctgagccctgtgtggttcgcttgtctaaaactgctactcgaaaaactcgggtacacgcacagagagacaaactacatcgactacctatgcaactctcaccacctgtacagagacaaacactactttgtgcgtggtggcatgccatcagggtgctccggtaccagcattttcaattcaatgatcaataacatcataatcaggacactgatgctcaaagtgtacaagggaattgacctggaccagtttagaatgattgcatatggtgatgatgtaattgcatcctacccttggcctatagacgcttcactacttgctgaagctgggaagggttatggactgattatgacaccagcagacaaaggggagtgcttcaatgaggttacctggaccaatgtcaccttcctaaagaggtactttagagctgatgagcagtatcctttcctggttcatccagttatgcccatgaaggacatccatgaatctattaggtggacaaaggatccaaagaacactcaagaccacgttcgatcgttgtgcttattggcttggcataacggagaacaagaatatgaggaattcatccgaaagatcagaagcgtcccggttgggcgctgtctgactctccccgcgttttcaactttacgcagaaaatggctggattccttttaaattagagacaatttgaaataatttaaattggcttaaccctactgtactaaccgaactagacaacggtgcagtaggggtaaattctccgcattcggtgcggaaaaaaaaaaaaaaaaaaaaaaaaaaaaaaaaaaaaaaaaaaaaaaaaaa

SEQ ID NO: 42 - protein, 2192 aa, Echovirus 12
MGAQVSTQKTGAHETGLSASGNSIIHYTNINYYKDAASNSANRQDFTQDPGKFTEPVKDIMIKSMPALNSPTAEECGYSDRVRSITLGNSTITTQECANVVVGYGTWPDYLHDDEATAEDQPTQPDVATCRFYTLESIQWQKTSDGWWWKFPEALKDMGLFGQNMHYHYLGRSGYTIHVQCNASKFHQGCLLVVCVPEAEMGCATVANEVTAAALSSGETAKYFAKTGATGTHTVQSIVTNAGMGVGVGNLTIFPHQWINLRTNNSATIVMPYINSVPMDNMFRHYNFTLMIIPFVPLDFTAEASTYVPITVTVAPMCAEYNGLRLASHQGLPTMNTPGSNQFLTSDDFQSPSAMPQFDVTPELRIPGEVKNLMEIAEVDSVVPVNNTQDSVYNMDVYKIPVSGGNQLSTQVFGFQMQPGLNSVFKRTLLGEILNYYAHWSGSVKLTFVFCGSAMATGKFLLAYSPPGADPPKSRKEAMLGTHVIWDIGLQSSCVLCVPWISQTHYRLVQQDEYTSAGYVTCWYQTSLVVPPGAPVTCGVLCLASACNDFSVRMLRDTPFIEQKQLLQGDVEEAVNRAVARVADTLPTGPRNSESIPALTAAETGHTSQVVPGDTMQTRHVKNYHSRTESSVENFLCRAACVYITKYKTKDSDPVQRYANWRINTRQMVQLRRKFELFTYLRFDMEVTFVITSSQDDGTQLAQDMPVLTHQVMYIPPGGPVPNSVTDFAWQSSTNPSIFWTEGNAPARMSIPFISIGNAYSNFYDGWSHFTQDGVYGXNSLNNMGSIYIRHVNEQSPHAITSTVRVYFKPKHVRAWVPRPPRLCAYEKSSNVNFKPTDVTTSRTSITEVPSLRPSVVNTGAFGQQSGAAYVGNYRVVNRHLATHVDWQNCVWEDYNRDLLVSTTTAHGCDTIARCQCTTGVYFCASRNKHYPVSFEGPGLVEVQESEYYPRRYQSHVLLAAGFSEPGDCGGILRCEHGVIGLVTMGGEGVVGFADVRDLLWLEDDAMEQGVKDYVEQLGNAFGSGFTNQICEQVNLLKESLVGHDSILEKSLKALVKIISALVIVVRNHDDLITVTATLALIGCTSSPWRWLKHKVSQYYGIPMAERQSNGWLKKFTEMTNACKGMEWIAIKIQKFIEWLKLKILPEVKEKHEFLNRLKQLPLLESQIATIEQSAPSQSDQEQLFSNVQYFAHYCRKYAPLYAAEAKRVFSLEKKMSNYIQFKSKCRIEPVCLLLHGSPGAGKSVATSLIGRSLAEKLNSSVYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYSQNGKINMPMSVKTCDEECCPVNFKRCCPLVCGKAIQFIDRRTQVRYSLDMLVTEMFREYNHRHSVGATLEALFQGPPVYREIKISVAPETPPPPAIADLLKSVDSEAVREYCKEKGWLVPEVNSTLQIEKHVSRAFICLQALTTFVSVAGIIYIIYKLFAGFQGAYTGMPNQKPKVPTLRQAKVQGPAFEFAVAMMKRNASTVKTEYGEFTMLGIYDRWAVLPRHAKPGPTILMNDQEIGVLDAKELVDKDGTNLELTLLKLNRNEKFRDIRGFLAREEAEVNEAVLAINTSKFPNMYIPVGQVTDYGFLNLGGTPTKRMLMYNFPTRAGQCGGVLMSTGKVLGIHVGGNGHQGFSAALLRHYFNEEQGEIEFIESSKDAGFPVINTPSKTKLEPSVFHQVFEGNKEPAVLRNGDPRLKVNFEAIFSKYIGNINTHVDEYMLEAVDHYAGQLATLDISTEPMKLEDAVYGTEGLEALDLTTSAGYPYVALGIKKRDILSKKTKDLTKLKECMDKYGLNLPMVTYVKDELRSSEKVAKGKSRLIEASSLNDSVAMRQTFGNLYKTFHLNPGIVTGSAVGCDPDLFWSKIPVMLDGHLIAFDYSGYDASLSPVWFACLKLLLEKLGYTHRETNYIDYLCNSHHLYRDKHYFVRGGMPSGCSGTSIFNSMINNIIIRTLMLKVYKGIDLDQFRMIAYGDDVIASYPWPIDASLLAEAGKGYGLIMTPADKGECFNEVTWTNVTFLKRYFRADEQYPFLVHPVMPMKDIHESIRWTKDPKNTQDHVRSLCLLAWHNGEQEYEEFIRKIRSVPVGRCLTLPAFSTLRRKWLDSF

SEQ ID NO: 43 - RNA, 6579 nt, Echovirus 12
  source 1..6579: mol_type = unassigned RNA
atgggagcgcaagtatcaacacaaaaaactggggcacacgagactggcctgagtgctagtggaaattccatcatccactataccaacattaattactacaaagatgcggcatccaattcagcaaacaggcaggatttcacccaagaccccgggaaattcaccgagccagtgaaggatatcatgataaaatcgatgcctgcgttaaactccccaacagcagaagaatgtgggtacagcgatagagtcagatcaatcaccctggggaactctaccatcaccacgcaggagtgtgccaatgtggtggtgggatatggcacgtggcctgattatctacatgatgatgaggccactgcagaggaccaaccaacacaaccggatgtggcgacgtgtaggttttacacacttgaatccattcagtggcagaagacatcagatgggtggtggtggaagtttccggaagcattgaaagacatgggactgttcggccaaaacatgcactatcattatcttggcaggtcgggttacaccatccacgtccaatgtaacgcgtctaaattccaccagggttgtttgcttgtggtgtgtgtgcctgaagctgagatggggtgcgctacagtggcgaatgaggttaccgcagccgctctctcatctggtgaaacggcaaaatattttgcaaagactggtgcgacgggaacccacacagtgcaaagcatagtgacaaacgccggcatgggggtgggcgtgggcaatctgacaatattcccacatcagtggataaacctccgtaccaacaacagcgcgactattgtcatgccttacataaacagtgtgcccatggataacatgttcagacactacaatttcaccctaatgatcatccccttcgtcccactggacttcacagctgaggcatctacgtatgtaccaatcactgtaacagtagctcccatgtgcgccgagtacaacgggctacgcctggcctcgcatcaaggtttgccgacaatgaacacccctggtagtaaccaattcctcacatcagatgactttcaatcaccatcagccatgccacaattcgatgtcacacccgagctcaggattccaggggaggtaaagaacttaatggaaatagccgaagttgactccgtagtgcctgtgaacaatacacaggactcagtgtacaacatggatgtgtacaaaattcctgttagtggaggcaaccaactgtcaactcaagtcttcgggttccaaatgcaaccagggctaaatagtgtgttcaaaaggacattactaggtgagatactgaactactatgcacactggtctggtagtgtaaagctcacctttgtgttctgtggatcggccatggccactggcaaatttctgctggcatattcccctccaggcgcagaccccccaaagtctaggaaagaagcaatgcttggtactcacgtgatatgggatataggacttcagtcaagctgcgtcttgtgcgtaccgtggatcagccaaacacattatagattggttcagcaggatgaatatacgagtgcaggatatgtaacttgctggtatcaaactagtttggttgtcccgcccggcgctcccgtcacgtgtggagtcttgtgcttagcatcggcgtgcaatgatttctctgtgagaatgttgagagacacaccttttatcgagcaaaaacagctactccaaggagatgtggaggaggccgtgaacagagccgttgcacgggtggcggatacactacccacagggccgagaaattcagagagcatccccgcactaacagcagcagaaactgggcatacatctcaggtggttcctggggataccatgcaaacccgtcatgtgaagaattaccactccagaactgaatcgtctgttgaaaacttcttatgccgagcagcatgtgtgtatattacaaagtacaagaccaaggacagtgatccagtgcaaaggtacgccaactggcggatcaacacccgtcaaatggttcagctaaggagaaaatttgagttgtttacatacctaaggtttgatatggaagttacttttgtgatcaccagttcacaagatgatggcacgcagttggcgcaggacatgccagtattaacacaccaagtcatgtatataccacctggagggccagtgccgaatagtgttactgattttgcgtggcaatcgtctaccaaccctagcatcttctggacggagggaaacgctccagctaggatgtccatcccctttatcagtataggcaacgcgtatagcaatttctatgatggttggtcacatttcacccaagatggagtatatggttacaactccctcaataacatgggctccatctacattaggcatgtcaatgaacaaagcccgcatgcaattacgagcacagtcagagtctatttcaaaccaaaacacgtgcgggcctgggtgccaagaccacctaggctttgtgcatatgagaaatcaagcaacgtgaatttcaaaccaacagatgtgactacctcccgaacatccatcacagaagtccctagtcttagaccatcagtggtaaatactggagctttcggtcagcaatcaggagcggcttatgtgggaaactatagagtggttaatagacacctagccactcatgttgactggcagaactgtgtgtgggaggactataacagggacctccttgtaagcaccaccacagctcatgggtgtgacaccatagccagatgccaatgcacaacaggcgtgtacttctgtgcatcgaggaataaacactacccagtcagttttgaaggaccaggtctggtagaagtccaggagagcgagtactaccccaggaggtatcaatcacacgtcttgctggctgcgggattttcagagccaggagattgtggagggattctcagatgtgagcacggtgtcatcggtctagttactatgggtggcgaaggcgttgtcggattcgctgatgtacgcgacctgttatggcttgaagatgacgccatggaacaaggagtcaaggattacgtggaacaactagggaacgcctttggttcaggattcactaaccaaatctgtgagcaggtcaacctcctaaaagagtcactggtgggtcacgattccatcctagagaagtcccttaaagccctagtgaaaatcatatcagcactagtgatagtggtgaggaaccatgatgatttgatcactgtaactgctactcttgccctcattggttgtacctcctctccatggcggtggctcaagcacaaggtgtcacaatactacgggatacccatggcagagcgccaaagcaacgggtggctcaagaagttcacagagatgaccaacgcctgcaaagggatggaatggattgccatcaaaatccaaaagttcatagagtggcttaaacttaagattttaccagaagttaaggaaaaacatgaattcctgaataggctcaagcaactcccactgttggagagtcaaatagcaaccatcgagcagagtgcaccgtcccagagcgatcaggagcagctcttttcaaatgttcagtactttgcccattattgcagaaaatatgcgcccctatacgcagctgaggcaaaaagggtgttctctcttgaaaagaaaatgagtaattacatacagttcaagtccaaatgccgtattgaacctgtatgcttgctactacatgggagccccggagcagggaaatcagttgctaccagcctcattgggcgatcactagctgaaaaattgaatagctcagtgtactccttaccaccagacccagatcactttgatggttacaagcaacaagctgtagtgatcatggacgatctgtgccaaaatccagatggaaaagatgtgtctttgttctgtcaaatggtgtctagtgtagattttgtaccaccaatggccgcgctagaggagaaaggcattttgttcacctctccattcgttctagcctcaaccaatgcaggatccatcaacgcaccaacagtttcagacagcagagccctagctagaaggttccactttgacatgaacattgaagttatctccatgtacagtcaaaatggaaaaataaacatgcccatgtcagtcaaaacatgtgatgaggaatgctgcccagtcaatttcaagagatgctgcccactggtgtgtggcaaggctattcagttcattgacagaagaacccaagtcagatattcactggatatgttagtcaccgagatgttcagggagtacaaccacaggcacagtgtgggcgccaccctcgaggctctgttccaaggtccaccggtctacagggagatcaagatcagtgttgctccagaaacacctcccccaccagcaatcgctgatttattaaaatcagtagacagtgaagctgtgagagagtactgcaaggaaaaaggctggcttgtaccggaagttaactccaccctacagattgagaagcacgtcagcagagcatttatctgtctacaagctctgaccactttcgtctcagtagctggcataatctacattatctacaagttgtttgccggctttcagggcgcgtatacggggatgccaaatcagaaacccaaagtgcccactctgagacaagctaaagtgcagggcccagcattcgagttcgctgtggcaatgatgaaaaggaacgccagcacagtgaaaacggaatatggtgagttcaccatgctcggcatctatgacagatgggcagtgctaccacgccacgccaagcctggaccgactattttgatgaatgatcaggagatcggcgtgttagacgccaaagaattagtggacaaagatgggacaaatctggagttgacccttttaaagctcaaccgcaatgagaagtttagggacatcagagggtttctggcgagagaggaagctgaagtgaatgaggctgttctggcaataaacacaagcaagttcccaaatatgtacatacccgtaggtcaagtcactgattacggtttcctgaacctggggggaacacccacaaagaggatgctcatgtacaactttccaactagagcagggcaatgtggtggggtactcatgtcaacagggaaagttcttggtatacatgtaggaggaaatggacatcaagggttctccgctgctcttcttaggcactactttaatgaggagcaaggtgaaatagagtttattgagagctcaaaggatgcaggattccctgtgatcaacacccccagcaaaacaaaactggaaccaagcgtgtttcatcaggtgtttgaaggcaataaagagccagcagtccttagaaatggtgatccgcgtcttaaagtaaactttgaagaagccatattttccaagtacatcgggaacatcaacacacatgtggatgaatacatgctcgaagcagtggatcactacgcagggcaactggctactcttgacattagcactgaacccatgaaactagaagatgcagtgtatggcactgaaggactagaggctcttgacttaacaacaagtgcagggtacccgtatgtcgcactaggcatcaaaaagagagacatcctatccaaaaagactaaagatctgactaaattgaaggaatgcatggacaagtacggtttgaacctgccgatggtgacttatgtgaaggatgaacttagatcatcagagaaagtggctaagggaaaatcaagactcattgaagcatctagtttaaatgactctgtcgcgatgaggcagacgtttggcaacttgtacaaaacattccatctgaacccgggcattgtgacaggcagtgcagttggatgtgatccggaccttttctggagtaaaatacccgtgatgttagacggccacctcatagcattcgattactccggatatgatgccagcctgagccctgtgtggttcgcttgtctaaaactgctactcgaaaaactcgggtacacgcacagagagacaaactacatcgactacctatgcaactctcaccacctgtacagagacaaacactactttgtgcgtggtggcatgccatcagggtgctccggtaccagcattttcaattcaatgatcaataacatcataatcaggacactgatgctcaaagtgtacaagggaattgacctggaccagtttagaatgattgcatatggtgatgatgtaattgcatcctacccttggcctatagacgcttcactacttgctgaagctgggaagggttatggactgattatgacaccagcagacaaaggggagtgcttcaatgaggttacctggaccaatgtcaccttcctaaagaggtactttagagctgatgagcagtatcctttcctggttcatccagttatgcccatgaaggacatccatgaatctattaggtggacaaaggatccaaagaacactcaagaccacgttcgatcgttgtgcttattggcttggcataacggagaacaagaatatgaggaattcatccgaaagatcagaagcgtcccggttgggcgctgtctgactctccccgcgttttcaactttacgcagaaaatggctggattccttt

SEQ ID NO: 44 - RNA, 741 nt, Echovirus 12
  source 1..741: mol_type = unassigned RNA
ttaaaacagcctgtgggttgtccccacccacagggcccactgggcgctagcacactggtatcccggtacctttgtgcgcctgttttatataccctcccctcagtaacttagaagttcatcacaaatgatcaatagttagctcaacaaaccagttgagcctagatcaagcacttctgttaccccgggctgagtatcaataagctgttgacacggctgaaggagaaaacgcccgttacccgaccagctacttcggagaacctagtatcaccatagaggttgcgtagcgtttcgctccgcacaaccccagtgtagatcaggtcgatgagtcaccgcgttccccacaggcgactgtggcggtggctgcgttggcggcctgcccatggggttacccatgggacgcttcaatactgacatggtgtgaagagttgactgagctagctggtagtcctccggcccctgaatgcggctaatcctaactgtggagcaagtgcccacaacccagtgggtggcttgtcgtaatgggcaactctgcagcggaaccgactactttgggtgaccgtgtttctctttattcttatattggctgcttatggtgacaatctcagagttgttaccatatagctattggtttggccaaccagtgactaacagagcaattatatatcttttcattggatttatacctataaacaacaccagttataccactttgtgttacattattggcttgaactcgaaaag

SEQ ID NO: 45 - RNA, 103 nt, Echovirus 12
  source 1..103: mol_type = unassigned RNA
taaattagagacaatttgaaataatttaaattggcttaaccctactgtactaaccgaactagacaacggtgcagtaggggtaaattctccgcattcggtgcgg

SEQ ID NO: 46 - protein, 2193 aa, Echovirus 12
MGAQVSTQKTGAHETGLSASGNSIIHYTNINYYKDAASNSANRQDFTQDPGKFTEPVKDIMIKSMPALNSPTAEECGYSDRVRSITLGNSTITTQECANVVVGYGTWPDYLHDDEATAEDQPTQPDVATCRFYTLESIQWQKTSDGWWWKFPEALKDMGLFGQNMHYHYLGRSGYTIHVQCNASKFHQGCLLVVCVPEAEMGCATVANEVNAAALSSGETAKHFAKTGATGTHTVQSIVTNAGMGVGVGNLTIFPHQWINLRTNNSATIVMPYINSVPMDNMFRHYNFTLMIIPFVPLDFTAEASTYVPITVTVAPMCAEYNGLRLASHQGLPTMNTPGSNQFLTSDDFQSPSAMPQFDVTPELRIPGEVKNLMEIAEVDSVVPVNNTQDSVYNMDVYKIPVSGGNQLSTQVFGFQMQPGLNSVFKRTLLGEILNYYAHWSGSVKLTFVFCGSAMALAKFLLAYSPPGADPPKSRKEAMLGTHVIWDIGLQSSCVLCVPWISQTHYRLVQQDEYTSAGYVTCWYQTSLVVPPGAPATCGVLCLASACNDFSVRMLRDTPFIEQKQLLQGDVEEAVNRAVARVADTLPTGPRNSESIPALTAAETGHTSQVVPGDTMQTRHVKNYHSRTESSVENFLCRAACVYITKYKTKDSDPVQRYANWRINTRQMVQLRRKFELFTYLRFDMEVTFVITSSQDDGTQLAQDMPVLTHQVMYIPPGGPVPNSVTDFAWQSSTNPSIFWTEGNAPARMSIPFISIGNAYSNFYDGWSHFTQDGVYGFNSLNNMGSIYIRHVNEQSPYAITSTVRVYFKPKHVRAWVPRPPRLCAYEKSSNVNFKPTDVTTSRTSITEVPSLRPSVVNTGAFGQQSGAAYVGNYRVVNRHLATHVDWQNCVWEDYNRDLLVSTTTAHGCDTIARCQCTTGVYFCASRNKHYPVSFEGPGLVEVQESEYYPRRYQSHVLLAAGFSEPGDCGGILRCEHGVIGLVTMGGEGVVGFADVRDLLWLEDDAMEQGVKDYVEQLGNAFGSGFTNQICEQVNLLKESLVGHDSILEKSLKALVKIISALVIVVRNHDDLITVTATLALIGCTSSPWRWLKHKVSQYYGIPMAERQSNGWLKKFTEMTNACKGMEWIAIKIQKFIEWLKLKILPEVKEKHEFLNRLKQLPLLESQIATIEQSAPSQSDQEQLFSNVQYFAHYCRKYAPLYAAEAKRVFSLEKKMSNYIQFKSKCRIEPVCLLLHGSPGAGKSVATSLIGRSLAEKLNSSVYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYSQNGKINMPMSVKTCDEECCPVNFKRCCPLVCGKAIQFIDRRTQVRYSLDMLVTEMFREYNHRHSVGATLEALFQGPPVIREIKISVAPETPPPPAIADLLKSVDSEAVREYCKEKGWLVPEVNSTLQIEKHVSRAFICLQALTTFVSVAGIIYIIYKLFAGFQGAYTGMPNQKPKVPTLRQAKVQGPAFEFAVAMMKRNASTVKTEYGEFTMLGIYDRWAVLPHHAKPGPTILMNDQEIGVLDAKELVDKDGTNLELTLLKLNRNEKFRDIRGFLAREEAEVNEAVLAINTSKFPNMYIPVGQVTDYGFLNLGGTPTKRMLMYNFPTRAGQCGGVLMSTGKVLGIHVGGNGHQGFSAALLRHYFNEEQGEIEFIESSKDAGFPVINTPSKTKLEPSVFHQVFEGNKEPAVLRNGDPRLKVNFEEAIFSKYIGNINTHVDEYMLEAVDHYAGQLATLDISTEPMKLEDAVYGTEGLEALDLTTSAGYPYVAIGIKKRDILSKKTKDLTKLKECMDKYGLNLPMVTYVKDELRSSEKVAKGKSRLIEASSLNDSVAMRQTFGNLYKTFHLNPGIVTGSAVGCDPDLFWSKIPVMLDGHLIAFDYSGYDASLSPVWFACLKLLLEKLGYTHRETNYIDYLCNSHHLYRDKHYFVRGGMPSGCSGTSIFNSMINNIIIRTLMLKVYKGIDLDQFRMIAYGDDVIASYPWPIDASLLAEAGKGYGLIMTPADKGECFNEVTWTNVTFLKRYFRADEQYPFLVHPVMPMKDIHESIRWTKDPKNTQDHVRSLCLLAWHNGEQEYEEFIRKIRSVPVGRCLTLPAFSTLRRKWLDSF

SEQ ID NO: 47 - protein, 2193 aa, Echovirus 12
MGAQVSTQKTGAHETGLNASGNSIIHYTNINYYKDAASNSANRQDFTQNPGKFTEPVKDIMIKSMPALNSPTAEECGYSDRVRSITLGNSTITTQECANVVVGYGVWPDYLSDDEATAEDQPTQPDVATCRFYTLESIQWQKTSDGWWWKFPEALKDMGLFGQNMHYHYLGRSGYTIHVQCNASKFHQGCLLVVCVPEAEMGCATVANEVNAAALSSGEVAKYFAKTGATGTNTVQNIVTNAGMGVGVGNLTIFPHQWINLRTNNSATIVMPYINSVPMDNMFRHYNFTLMIIPFVPLDFTAEASTYVPITVTVAPMCAEYNGLRLASHQGLPTMNTPGSNQFLTSDDFQSPSAMPQFDVTPELRIPGEVKNLMEIAEVDSVVPVNNTVDSVHNMDVYKIPVSGGNQLSTQVFGFQMQPGLNSVFKRTLLGEILNYYAHWSGSVKLTFVFCGSAMATGKFLLAYSPPGADPPKSRKEAMLGTHVIWDIGLQSSCVLCVPWISQTHYRLVQQDEYTSAGYVTCWYQTSLVVPPGAPSTCGILCLASACNDFSVRMLRDTPFIEQKQLLQGDVEEAVNRAVARVADTLPTGPRNSESIPALTAAETGHTSQVVPGDTMQTRHVKNYHSRTESSVENFLCRAACVYITKYKTKDSDPVQRYANWRINTRQMVQLRRKFELFTYLRFDMEVTFVITSSQDDGTQLAQDMPVLTHQVMYIPPGGPVPNSVTDFAWQSSTNPSIFWTEGNAPARMSIPFISIGNAYSNFYDGWSHFTQDGVYGFNSLNNMGSIYIRHVNEQSPHAITSTVRVYFKPKHVRAWVPRPPRLCAYEKSSNVNFEPTDVTTTRSSITEVPSLRPSLQNTGAFGQQSGAVYVGNYRVVNRHLATYNDWQNCVWEDYNRDLLVSTTTAHGCDTIARCQCTTGVYFCASKNKHYPISFEGPGLVEVQESEYYPKRYQSHVLLAAGFSEPGDCGGILRCEHGVIGLVTMGGEGVVGFADVRDLLWLEDDAMEQGVRDYVEQLGNAFGSGFTNQICEQVNLLKESLVGQDSILEKSLKALVKIISALVIVVRNHDDLITVTATLALIGCTSSPWRWLKHKVSQYYGIPMAERQNNGWLKKFTEMTNACKGMEWIAIKIQKFEMGFKVKILPEVREKHEFLNRLKQLPLLESQIATIEQSAPSQSDQEQLFSNVQYFAHYCRKYAPLYAAEAKRVFSLEKKMSNYIQFKSKCRIEPVCLLLHGSPGAGKSVATNLIGRSLAEKLNSSVYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYSQNGKINMPMSVKTCDEECSPVNFKKCCPLVCGKAIQFIDRRTQVRYSLDMLVTEMFREYNHRHSVGATLEALFQGPPVYREIKISVAPEIPPPPAIADLLKSVDSEAVREYCKERGWLVPEINSTLQIEKHVSRAFICLQALTTFVSVAGIIYIIYKLFAGFQGAYTGMPNQKPKIPTLRQAKVQGPAFEFAVAMMKRNASTVKTEYGEFTMLGIYDRWAVLPRHAKPGPTILMNDQEVGVVDAKELVDKDGTNLELTLLKLNRNEKFRDIRGFLTREEAEVNEAVLAINTSKFPNMYIPVGQVTDYGFLNLGGTPTKRMLMYNFPTRAGQCGGVLMSTGKVLGIHVGGNGHQGFSAALLRHYFNDEQGEIEFIESSKEAGFPVINTPSKTKLEPSVFHHIFEGNKEPAVLRNGDPRLKANFEEAIFSKYIGNVNTHVDGYMQEAIDHYAGQLATLDISTEPMKLEDAVYGTEGLEALDLTTSAGYPYVALGIKKRDILSKKTKDLTKLKECMDKYGLNLPMVTYVKDELRSADKVAKGKSRLIEASSLNDSVAMRQTFGNLYKTFHLNPGVVTGSAVGCDPDLFWSKIPVMLDGHLIAFDYSGYDASLSPVWFACLKLLLEKLGYSHKETNYIDYLCNSHHLYRDKHYFVRGGMPSGCSGTSIFNSMINNIIIRTLMLKVYKGIDLDQFRMIAYGDDVIASYPHPIDASLLAEAGKGYGLIMTPADKGECFNEVTWNNVTFLKRYFRADEQYPFLVHPVMPMKDIHESIRWTKDPKNTQDHVRSLCLLAWHNGEREYEEFIKKIRSVPVGRCLTLPAFSTLRRKWLDSF

SEQ ID NO: 48 - protein, 2133 aa, Echovirus 12
MGAQVSTQKTGAHETGLNASGNSIIHYTNINYYKDAASNSANRQDFTQDPGKFTEPVKDIMIKSMPALNSPTAEECGYSDRVRSITLGNSTITTQECANVVVGYGVWPDYLHDDEATAEDQPTQPDVATCRFYTLESIQWQKTSDGWWWKFPEALKDMGLFGQNMHYHYLGRSGYTIHVQCNASKFHQGCLLVVCVPEAEMGCATVANEVNAAALSSGEVAKYFAKTGATGTNTVQNIVTNAGMGVGVGNLTIFPHQWINLRTNNSATIVMPYINSVPMDNMFRHYNFTLMIIPFVPLDFTAEASTYVPITVTVAPMCAEYNGLRLASHQGLPTMNTPGSNQFLTSDDFQSPSAMPQFDVTPELRIPGEVKNLMEIAEVDSVVPVNNTVDSVHNMDVYKIPVSGGNQLSTQVFGFQMQPGLNSVFKRTLLGEILNYYAHWSGSVKLTFVFCGSAMATGKFLLAYSPPGADPPKSRKEAMLGTHVIWDIGLQSSCVLCVPWISQTHYRLVQQDEYTSAGYVTCWYQTSLVVPPGAPSTCGILCLASACNDFSVRMLRDTPFIEQKQLLQGDVEEAVNRAVARVADTLPTGPRNSESIPALTAAETGHTSQVVPGDTMQTRHVKNYHSRTESSVENFLCRAACVYITKYKTKDSDPVQRYANWRINTRQMVQLRRKFELFTYLRFDMEVTFVITSSQDDGTQLAQDMPVLTHQVMYIPPGGPVPNSVTDFAWQSSTNPSIFWTEGNAPARMSIPFISIGNAYSNFYDGWSHFTQDGVYGFNSLNNMGSIYIRHVNEQSPHAITSTVRVYFKPKHVRAWVPRPPRLCAYEKSSNVNFEPTDVTTTRSSITEVPSLRPSLQNTGAFGQQSGAVYVGNYRVVNRHLATYNDWQNCVWEDYNRDLLVSTTTAHGCDTIARCQCTTGVYFCASKNKHYPISFEGPGLVEVQESEYYPKRYQSHVLLAAFGSGFTNQICEQVNLLKESLVGQDSILEKSLKALVKIISALVIVVRNHDDLITVTATLALIGCTSSPWRWLKHKVSQYYGIPMAERQNNGWLKKFTEMTNACKGMEWIAIKIQKFVEWLKVKILPEVREKHEFLNRLKQLPLLKSQIATIEQSAPSQSDQEQLFSNVQYFAHYCRKYAPLYAAEAKRVFSLEKKMSNYIQFKSKCRIEPVCLLLHGSPGAGKSVATNLIGRSLAEKLNSSVYSLPPDPDHFDGYKQQAVVIMDDLCQNPDGKDVSLFCQMVSSIDCAKPMAALEEKGILFTSPFVLASTNAGSINAPTVSDSRALARRFHFDMNIEVISMYSQNGKINMPMSVKTCDEECSPVNFKKCCPLVCGKAIQFIDRRTQVRYSLDMLVTEMFREYNHRHSVGATLEALFQGPPVYREIKISVAPEIPPPPAIADLLKSVDSEAVREYCKERGWLVPEINSTLQIEKHVSRAFICLQALTTFVSVAGIIYIIYKLFAGFQGAYTGMPNQKPKIPTLRQAKVQGPAFEFAVAMMKRNASTVKTEYGEFTMLGIYDRWAVLPRHAKPGPTILMNDQEVGVVDAKELVDKDGTNLELTLLKLNRNEKFRDIRGFLTREEAEVNEAVLAINTSKFPNMYIPVGQVTDYGFLNLGGTPTKRMLMYNFPTRAGQCGGVLMSTGKVLGIHVGGNGHQGFSAALLRHYFNDEQGEIEFIESSKEAGFPVINTPSKTKLEPSVFHHIFEGNKEPAVLRNGDPRLKANFEEAIFSKYIGNVNTHVDGYMQEAIDHYAGQLATLDISTEPMKLEDAVYGTEGLEALDLTTSAGYPYVALGIKKRDILSKKTKDLTKLKECMDKYGLNLPMVTYVKDELRSADKVAKGKSRLIEASSLNDSVAMRQTFGNLYKTFHLNPGVVTGSAVGCDPDLFWSKIPVMLDGHLIAFDYSGYDASLSPVWFACLKLLLEKLGYSHKETNYIDYLCNSHHLYRDKHYFVRGGMPSGCSGTSIFNSMINNIIIRTLMLKVYKGIDLDQFRMIAYGDDVIASYPHPIDASLLAEAGKGYGLIMTPADKGECFNEVTWNNVTFLKRYFRADEQYPFLVHPVMPMKDIHESIRWTKDPKNTQDHVRSLCLLAWHNGEREYEEFIKKIRSVPVGRCLTLPAFSTLRRKWLDSF

SEQ ID NO: 49 - protein, 2193 aa, Echovirus 12
MGAQVSTQKTGAHETGLNASGNSIIHYTNINYYKDAASNSANRQDFTQDPGKFTEPVKDIMIKSMPALNSPTAEECGYSDRVRSITLGNSTITTQECANVVVGYGVWPDYLMMMKPPLRINRHKPDVATCRFYTLESIQWQKTSDGWWWKFPEALKDMGLFGQNMHYHYLGRSGYTIHVQCNASKFHQGCLLVVCVPEAEMGCATVANEVNAAALSSGEVAKYFAKTGATGTNTVQNIVTNAGMGVGVGNLTIFPHQWINLRTNNSATIVMPYINSVPMDNMFRHYNFTLMIIPFVPLDFTAEASTYVPITVTVAPMCAEYNGLRLASHQGLPTMNTPGSNQFLTSDDFQSPSAMPQFDVTPELRIPGEVKNLMEIAEVDSVVPVNNTVDSVHNMDVYKIPVSGGNQLSTQVFGFQMQPGLNSVFKRTLLGEILNYYAHWSGSVKLTFVFCGSAMATGKFLLAYSPPGADPPKSRKEAMLGTHVIWDIGLQSSCVLCVPWISQTHYRLVQQDEYTSAGYVTCWYQTSLVVPPGAPSTCGILCLASACNDFSVRMLRDTPFIEQKQLLQGDVEEAVNRAVARVADTLPTGPRNSESIPALTAAETGHTSQVVPGDTMQTRHVKNYHSRTESSVENFLCRAACVYITKYKTKDSDPVQRYANWRINTRQMVQLRRKFELFTYLRFDMEVTFVITSSQDDGTQLAQDMPVLTHQVMYIPPGGPVPNSVTDFAWQSSTNPSIFWTEGNAPARMSIPFISIGNAYSNFYDGWSHFTQDGVYGFNSLNNMGSIYIRHVNEQSPHAITSTVRVYFKPKHVRAWVPRPPRLCAYEKSSNVNFEPTDVTTTRSSITEVPSLRPSLQNTGAFGQQSGAVYVGNYRVVNRHLATYNDWQNCVWEDYNRDLLVSTTTAHGCDTIARCQCTTGVYFCASKNKHYPISFEGPGLVEVQESEYYPKRYQSHVLLAAGFSEPGDCGGILRCEHGVIGLVTMGGEGVVGFADVRDLLWLEDDAMEQGVRDYVEQLGNAFGSGFTNQICEQVNLLKESLVGQDSILEKSLKALVKIISALVIVVRNHDDLITVTATLALIGCTSSPWRWLKHKVSQYYGIPMAERQNNGWLKKFTEMTNACKGMEWIAIKIQKFVEWLKGQILPEVREKHEFLNRLKQLPLLESQIATIEQSAPSQSDQEQLFSNVQYFAHYCRKYAPLYAAEAKRVFSLEKKMSNYIQFKSKCRIEPVCLLLHGSPGAGKSVATNLIGRSLAEKLNSSVYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYSQNGKINMPMSVKTCDEECSPVNFKKCCPLVCGKAIQFIDRRTQVRYSLDMLVTEMFREYNHRHSVGATLEALFQGPPVYREIKISVAPEIPPPPAIADLLKSVDSEAVREYCKERGWLVPEINSTLQIEKHVSRAFICLQALTTFVSVAGIIYIIYKLFAGFQGAYTGMPNQKPKIPTLRQAKVQGPAFEFAVAMMKRNASTVKTEYGEFTMLGIYDRWAVLPRHAKPGPTILMNDQEVGVVDAKELVDKDGTNLELTLLKLNRNEKFRDIRGFLTREEAEVNEAVLAINTSKFPNMYIPVGQVTDYGFLNLGGTPTKRMLMYNFPTRAGQCGGVLMSTGKVLGIHVGGNGHQGFSAALLRHYFNDEQGEIEFIESSKEAGFPVINTPSKTKLEPSVFHHIFEGNKEPAVLRNGDPRLKANFEEAIFSKYIGNVNTHVDGYMQEAIDHYAGQLATLDISTEPMKLEDAVYGTEGLEALDLTTSAGYPYVALGIKKRDILSKKTKDLTKLKECMDKYGLNLPMVTYVKDELRSADKVAKGKSRLIEASSLNDSVAMRQTFGNLYKTFHLNPGVVTGSAVGCDPDLFWSKIPVMLDGHLIAFDYSGYDASLSPVWFACLKLLLEKLGYSHKETNYIDYLCNSHHLYRDKHYFVRGGMPSGCSGTSIFNSMINNIIIRTLMLKVYKGIDLDQFRMIAYGDDVIASYPHPIDASLLAEAGKGYGLIMTPADKGECFNEVTWNNVTFLKRYFRADEQYPFLVHPVMPMKDIHESIRWTKDPKNTQDHVRSLCLLAWHNGEREYEEFIKKIRSVPVGRCLTLPAFSTLRRKWLDSF

SEQ ID NO: 50 - RNA, 7474 nt, Echovirus 12
  source 1..7474: mol_type = unassigned RNA
ttaaaacagcctgtgggttgtccccacccacagggcccactgggcgctagcacactggtatcccggtacctttgtgcgcctgttttatataccctcccctcagtaacttagaagttcatcacaaatgatcaatagttagctcaacaaaccagttgagcctagatcaagcacttctgttaccccgggctgagtatcaataagctgttgacacggctgaaggagaaaacgcccgttacccgaccagctacttcggagaacctagtatcaccatagaggttgcgtagcgtttcgctccgcacaaccccagtgtagatcaggtcgatgagtcaccgcgttccccacaggcgactgtggcggtggctgcgttggcggcctgcccatggggttacccatgggacgcttcaatactgacatggtgtgaagagttgactgagctagctggtagtcctccggcccctgaatgcggctaatcctaactgtggagcaagtgcccacaacccagtgggtggcttgtcgtaatgggcaactctgcagcggaaccgactactttgggtgaccgtgtttctctttattcttatattggctgcttatggtgacaatctcagagttgttaccatatagctattggtttggccaaccagtgactaacagagcaattatatatcttttcattggatttatacctataaacaacaccagttataccactttgtgttacattattggcttgaactcgaaaagatgggagcgcaagtatcaacacaaaaaactggggcacacgagactggcctgagtgctagtggaaattccatcatccactataccaacattaattactacaaagatgcggcatccaattcagcaaacaggcaggatttcacccaagaccccgggaaattcaccgagccagtgaaggatatcatgataaaatcgatgcctgcgttaaactccccaacagcagaagaatgtgggtacagcgatagagtcagatcaatcaccctggggaactctaccatcaccacgcaggagtgtgccaatgtggtggtgggatatggcacgtggcctgattatctacatgatgatgaggccactgcagaggaccaaccaacacaaccggatgtggcgacgtgtaggttttacacacttgaatccattcagtggcagaagacatcagatgggtggtggtggaagtttccggaagcattgaaagacatgggactgttcggccaaaacatgcactatcattatcttggcaggtcgggttacaccatccacgtccaatgtaacgcgtctaaattccaccagggttgtttgcttgtggtgtgtgtgcctgaagctgagatggggtgcgctacagtggcgaatgaggttaccgcagccgctctctcatctggtgaaacggcaaaatattttgcaaagactggtgcgacgggaacccacacagtgcaaagcatagtgacaaacgccggcatgggggtgggcgtgggcaatctgacaatattcccacatcagtggataaacctccgtaccaacaacagcgcgactattgtcatgccttacataaacagtgtgcccatggataacatgttcagacactacaatttcaccctaatgatcatccccttcgtcccactggacttcacagctgaggcatctacgtatgtaccaatcactgtaacagtagctcccatgtgcgccgagtacaacgggctacgcctggcctcgcatcaaggtttgccgacaatgaacacccctggtagtaaccaattcctcacatcagatgactttcaatcaccatcagccatgccacaattcgatgtcacacccgagctcaggattccaggggaggtaaagaacttaatggaaatagccgaagttgactccgtagtgcctgtgaacaatacacaggactcagtgtacaacatggatgtgtacaaaattcctgttagtggaggcaaccaactgtcaactcaagtcttcgggttccaaatgcaaccagggctaaatagtgtgttcaaaaggacattactaggtgagatactgaactactatgcacactggtctggtagtgtaaagctcacctttgtgttctgtggatcggccatggccactggcaaatttctgctggcatattcccctccaggcgcagaccccccaaagtctaggaaagaagcaatgcttggtactcacgtgatatgggatataggacttcagtcaagctgcgtcttgtgcgtaccgtggatcagccaaacacattatagattggttcagcaggatgaatatacgagtgcaggatatgtaacttgctggtatcaaactagtttggttgtcccgcccggcgctcccgtcacgtgtggagtcttgtgcttagcatcggcgtgcaatgatttctctgtgagaatgttgagagacacaccttttatcgagcaaaaacagctactccaaggagatgtggaggaggccgtgaacagagccgttgcacgggtggcggatacactacccacagggccgagaaattcagagagcatccccgcactaacagcagcagaaactgggcatacatctcaggtggttcctggggataccatgcaaacccgtcatgtgaagaattaccactccagaactgaatcgtctgttgaaaacttcttatgccgagcagcatgtgtgtatattacaaagtacaagaccaaggacagtgatccagtgcaaaggtacgccaactggcggatcaacacccgtcaaatggttcagctaaggagaaaatttgagttgtttacatacctaaggtttgatatggaagttacttttgtgatcaccagttcacaagatgatggcacgcagttggcgcaggacatgccagtattaacacaccaagtcatgtatataccacctggagggccagtgccgaatagtgttactgattttgcgtggcaatcgtctaccaaccctagcatcttctggacggagggaaacgctccagctaggatgtccatcccctttatcagtataggcaacgcgtatagcaatttctatgatggttggtcacatttcacccaagatggagtatatggttacaactccctcaataacatgggctccatctacattaggcatgtcaatgaacaaagcccgcatgcaattacgagcacagtcagagtctatttcaaaccaaaacacgtgcgggcctgggtgccaagaccacctaggctttgtgcatatgagaaatcaagcaacgtgaatttcaaaccaacagatgtgactacctcccgaacatccatcacagaagtccctagtcttagaccatcagtggtaaatactggagctttcggtcagcaatcaggagcggcttatgtgggaaactatagagtggttaatagacacctagccactcatgttgactggcagaactgtgtgtgggaggactataacagggacctccttgtaagcaccaccacagctcatgggtgtgacaccatagccagatgccaatgcacaacaggcgtgtacttctgtgcatcgaggaataaacactacccagtcagttttgaaggaccaggtctggtagaagtccaggagagcgagtactaccccaggaggtatcaatcacacgtcttgctggctgcgggattttcagagccaggagattgtggagggattctcagatgtgagcacggtgtcatcggtctagttactatgggtggcgaaggcgttgtcggattcgctgatgtacgcgacctgttatggcttgaagatgacgccatggaacaaggagtcaaggattacgtggaacaactagggaacgcctttggttcaggattcactaaccaaatctgtgagcaggtcaacctcctaaaagagtcactggtgggtcacgattccatcctagagaagtcccttaaagccctagtgaaaatcatatcagcactagtgatagtggtgaggaaccatgatgatttgatcactgtaactgctactcttgccctcattggttgtacctcctctccatggcggtggctcaagcacaaggtgtcacaatactacgggatacccatggcagagcgccaaagcaacgggtggctcaagaagttcacagagatgaccaacgcctgcaaagggatggaatggattgccatcaaaatccaaaagttcatagagtggcttaaacttaagattttaccagaagttaaggaaaaacatgaattcctgaataggctcaagcaactcccactgttggagagtcaaatagcaaccatcgagcagagtgcaccgtcccagagcgatcaggagcagctcttttcaaatgttcagtactttgcccattattgcagaaaatatgcgcccctatacgcagctgaggcaaaaagggtgttctctcttgaaaagaaaatgagtaattacatacagttcaagtccaaatgccgtattgaacctgtatgcttgctactacatgggagccccggagcagggaaatcagttgctaccagcctcattgggcgatcactagctgaaaaattgaatagctcagtgtactccttaccaccagacccagatcactttgatggttacaagcaacaagctgtagtgatcatggacgatctgtgccaaaatccagatggaaaagatgtgtctttgttctgtcaaatggtgtctagtgtagattttgtaccaccaatggccgcgctagaggagaaaggcattttgttcacctctccattcgttctagcctcaaccaatgcaggatccatcaacgcaccaacagtttcagacagcagagccctagctagaaggttccactttgacatgaacattgaagttatctccatgtacagtcaaaatggaaaaataaacatgcccatgtcagtcaaaacatgtgatgaggaatgctgcccagtcaatttcaagagatgctgcccactggtgtgtggcaaggctattcagttcattgacagaagaacccaagtcagatattcactggatatgttagtcaccgagatgttcagggagtacaaccacaggcacagtgtgggcgccaccctcgaggctctgttccaaggtccaccggtctacagggagatcaagatcagtgttgctccagaaacacctcccccaccagcaatcgctgatttattaaaatcagtagacagtgaagctgtgagagagtactgcaaggaaaaaggctggcttgtaccggaagttaactccaccctacagattgagaagcacgtcagcagagcatttatctgtctacaagctctgaccactttcgtctcagtagctggcataatctacattatctacaagttgtttgccggctttcagggcgcgtatacggggatgccaaatcagaaacccaaagtgcccactctgagacaagctaaagtgcagggcccagcattcgagttcgctgtggcaatgatgaaaaggaacgccagcacagtgaaaacggaatatggtgagttcaccatgctcggcatctatgacagatgggcagtgctaccacgccacgccaagcctggaccgactattttgatgaatgatcaggagatcggcgtgttagacgccaaagaattagtggacaaagatgggacaaatctggagttgacccttttaaagctcaaccgcaatgagaagtttagggacatcagagggtttctggcgagagaggaagctgaagtgaatgaggctgttctggcaataaacacaagcaagttcccaaatatgtacatacccgtaggtcaagtcactgattacggtttcctgaacctggggggaacacccacaaagaggatgctcatgtacaactttccaactagagcagggcaatgtggtggggtactcatgtcaacagggaaagttcttggtatacatgtaggaggaaatggacatcaagggttctccgctgctcttcttaggcactactttaatgaggagcaaggtgaaatagagtttattgagagctcaaaggatgcaggattccctgtgatcaacacccccagcaaaacaaaactggaaccaagcgtgtttcatcaggtgtttgaaggcaataaagagccagcagtccttagaaatggtgatccgcgtcttaaagtaaactttgaagaagccatattttccaagtacatcgggaacatcaacacacatgtggatgaatacatgctcgaagcagtggatcactacgcagggcaactggctactcttgacattagcactgaacccatgaaactagaagatgcagtgtatggcactgaaggactagaggctcttgacttaacaacaagtgcagggtacccgtatgtcgcactaggcatcaaaaagagagacatcctatccaaaaagactaaagatctgactaaattgaaggaatgcatggacaagtacggtttgaacctgccgatggtgacttatgtgaaggatgaacttagatcatcagagaaagtggctaagggaaaatcaagactcattgaagcatctagtttaaatgactctgtcgcgatgaggcagacgtttggcaacttgtacaaaacattccatctgaacccgggcattgtgacaggcagtgcagttggatgtgatccggaccttttctggagtaaaatacccgtgatgttagacggccacctcatagcattcgattactccggatatgatgccagcctgagccctgtgtggttcgcttgtctaaaactgctactcgaaaaactcgggtacacgcacagagagacaaactacatcgactacctatgcaactctcaccacctgtacagagacaaacactactttgtgcgtggtggcatgccatcagggtgctccggtaccagcattttcaattcaatgatcaataacatcataatcaggacactgatgctcaaagtgtacaagggaattgacctggaccagtttagaatgattgcatatggtgatgatgtaattgcatcctacccttggcctatagacgcttcactacttgctgaagctgggaagggttatggactgattatgacaccagcagacaaaggggagtgcttcaatgaggttacctggaccaatgtcaccttcctaaagaggtactttagagctgatgagcagtatcctttcctggttcatccagttatgcccatgaaggacatccatgaatctattaggtggacaaaggatccaaagaacactcaagaccacgttcgatcgttgtgcttattggcttggcataacggagaacaagaatatgaggaattcatccgaaagatcagaagcgtcccggttgggcgctgtctgactctccccgcgttttcaactttacgcagaaaatggctggattccttttaaattagagacaatttgaaataatttaaattggcttaaccctactgtactaaccgaactagacaacggtgcagtaggggtaaattctccgcattcggtgcggaaaaaaaaaaaaaaaaaaaaaaaaaaaaaaaaaaaaaaaaaaaaaaaaaaa

SEQ ID NO: 51 - RNA, 7473 nt, Echovirus 12
  source 1..7473: mol_type = unassigned RNA
ttaaaacagcctgtgggttgtccccacccacagggcccactgggcgctagcacactggtatcccggtacctttgtgcgcctgttttatataccctcccctcagtaacttagaagttcatcacaaatgatcaatagttagctcaacaaaccagttgagcctagatcaagcacttctgttaccccgggctgagtatcaataagctgttgacacggctgaaggagaaaacgcccgttacccgaccagctacttcggagaacctagtatcaccatagaggttgcgtagcgtttcgctccgcacaaccccagtgtagatcaggtcgatgagtcaccgcgttccccacaggcgactgtggcggtggctgcgttggcggcctgcccatggggttacccatgggacgcttcaatactgacatggtgtgaagagttgactgagctagctggtagtcctccggcccctgaatgcggctaatcctaactgtggagcaagtgcccacaacccagtgggtggcttgtcgtaatgggcaactctgcagcggaaccgactactttgggtgaccgtgtttctctttattcttatattggctgcttatggtgacaatctcagagttgttaccatatagctattggtttggccaaccagtgactaacagagcaattatatatcttttcattggatttatacctataaacaacaccagttataccactttgtgttacattattggcttgaactcgaaaagatgggagcgcaagtatcaacacaaaaaactggggcacacgagactggcctgagtgctagtggaaattccatcatccactataccaacattaattactacaaagatgcggcatccaattcagcaaacaggcaggatttcacccaagaccccgggaaattcaccgagccagtgaaggatatcatgataaaatcgatgcctgcgttaaactccccaacagcagaagaatgtgggtacagcgatagagtcagatcaatcaccctggggaactctaccatcaccacgcaggagtgtgccaatgtggtggtgggatatggcacgtggcctgattatctacatgatgatgaggccactgcagaggaccaaccaacacaaccggatgtggcgacgtgtaggttttacacacttgaatccattcagtggcagaagacatcagatgggtggtggtggaagtttccggaagcattgaaagacatgggactgttcggccaaaacatgcactatcattatcttggcaggtcgggttacaccatccacgtccaatgtaacgcgtctaaattccaccagggttgtttgcttgtggtgtgtgtgcctgaagctgagatggggtgcgctacagtggcgaatgaggttaccgcagccgctctctcatctggtgaaacggcaaaatattttgcaaagactggtgcgacgggaacccacacagtgcaaagcatagtgacaaacgccggcatgggggtgggcgtgggcaatctgacaatattcccacatcagtggataaacctccgtaccaacaacagcgcgactattgtcatgccttacataaacagtgtgcccatggataacatgttcagacactacaatttcaccctaatgatcatccccttcgtcccactggacttcacagctgaggcatctacgtatgtaccaatcactgtaacagtagctcccatgtgcgccgagtacaacgggctacgcctggcctcgcatcaaggtttgccgacaatgaacacccctggtagtaaccaattcctcacatcagatgactttcaatcaccatcagccatgccacaattcgatgtcacacccgagctcaggattccaggggaggtaaagaacttaatggaaatagccgaagttgactccgtagtgcctgtgaacaatacacaggactcagtgtacaacatggatgtgtacaaaattcctgttagtggaggcaaccaactgtcaactcaagtcttcgggttccaaatgcaaccagggctaaatagtgtgttcaaaaggacattactaggtgagatactgaactactatgcacactggtctggtagtgtaaagctcacctttgtgttctgtggatcggccatggccactggcaaatttctgctggcatattcccctccaggcgcagaccccccaaagtctaggaaagaagcaatgcttggtactcacgtgatatgggatataggacttcagtcaagctgcgtcttgtgcgtaccgtggatcagccaaacacattatagattggttcagcaggatgaatatacgagtgcaggatatgtaacttgctggtatcaaactagtttggttgtcccgcccggcgctcccgtcacgtgtggagtcttgtgcttagcatcggcgtgcaatgatttctctgtgagaatgttgagagacacaccttttatcgagcaaaaacagctactccaaggagatgtggaggaggccgtgaacagagccgttgcacgggtggcggatacactacccacagggccgagaaattcagagagcatccccgcactaacagcagcagaaactgggcatacatctcaggtggttcctggggataccatgcaaacccgtcatgtgaagaattaccactccagaactgaatcgtctgttgaaaacttcttatgccgagcagcatgtgtgtatattacaaagtacaagaccaaggacagtgatccagtgcaaaggtacgccaactggcggatcaacacccgtcaaatggttcagctaaggagaaaatttgagttgtttacatacctaaggtttgatatggaagttacttttgtgatcaccagttcacaagatgatggcacgcggttggcgcaggacatgccagtattaacacaccaagtcatgtatataccacctggagggccagtgccgaatagtgttactgattttgcgtggcaatcgtctaccaaccctagcatcttctggacggagggaaacgctccagctaggatgtccatcccctttatcagtataggcaacgcgtatagcaatttctatgatggttggtcacatttcacccaagatggagtatatggtttcaactccctcaataacatgggctccatctacattaggcatgtcaatgaacaaagcccgcatgcaattacgagcacagtcagagtctatttcaaaccaaaacacgtgcgggcctgggtgccaagaccacctaggctttgtgcatatgagaaatcaagcaacgtgaatttcaaaccaacagatgtgactacctcccgaacatccatcacagaagtccctagtcttagaccatcagtggtaaatactggagctttcggtcagcaatcaggagcggcttatgtgggaaactatagagtggttaatagacacctagccactcatgttgactggcagaactgtgtgtgggaggactataacagggacctccttgtaagcaccaccacagctcatgggtgtgacaccatagccagatgccaatgcacaacaggcgtgtacttctgtgcatcgaggaataaacactacccagtcagttwtgaaggaccaggtctggtagaagtccaggagagcgagtactaccccaggaggtatcaatcacacgtcttgctggctgcgggattttcagagccaggagattgtggagggattctcagatgtgagcacggtgtcatcggtctagttactatgggtggcgaaggcgttgtcggattcgctgatgtacgcgacctgttatggcttgaagatgacgccatggaacaaggagtcaaggattacgtggaacaactagggaacgcctttggttcaggattcactaaccaaatctgtgagcaggtcaayctcctaaaagagtcactggtgggtcacgattccatcctagagaagtcccttaaagccctagtgaaaatcatatcagcactagtgatagtggtgaggaaccatgatgatttgatcactgtaactgctactcttgccctcattggttgtacctcctctccatggcggtggctcaagcacaaggtgtcacaatactacgggatacccatggcagagcgccaaagcaacgggtggctcaagaagttcacagagatgaccaacgcctgcaaagggatggaatggattgccatcaaaatccaaaagttcatagagtggcttaaacttaagattttaccagaagttaaggaaaaacatgaattcctgaataggctcaagcaactcccactgttggagagtcaaatagcaaccatcgagcagagtgcaccgtcccagagcgatcaggagcagctcttttcaaatgttcagtactttgcccattattgcagaaaatatgcgcccctatacgcagctgaggcaaaaagggtgttctctcttgaaaagaaaatgagtaattacatacagttcaagtccaaatgccgtattgaacctgtatgcttgctactacatgggagccccggagcagggaaatcagttgctaccagcctcattgggcgatcactagctgaaaaattgaatagctcagtgtactccttaccaccagacccagatcactttgatggttacaagcaacaagctgtagtgatcatggacgatctgtgccaaaatccagatggaaaagatgtgtctttgttctgtcaaatggtgtctagtgtagattttgtaccaccaatggccgcgctagaggagaaaggcattttgttcacctctccattcgttctagcctcaaccaatgcaggatccatcaacgcaccaacagtttcagacagcagagccctagctagaaggttccactttgacatgaacattgaagttatctccatgtacagtcaaaatggaaaaataaacatgcccatgtcagtcaaaacatgtgatgaggaatgctgcccagtcaatttcaagagatgctgcccactggtgtgtggcaaggctattcagttcattgacagaagaacccaagtcagatattcactggatatgttagtcaccgagatgttcagggagtacaaccacaggcacagtgtgggcgccaccctcgaggctctgttccaaggtccaccggtctacagggagatcaagatcagtgttgctccagaaacacctcccccaccagcaatcgctgatttattaaaatcagtagacagtgaagctgtgagagagtactgcaaggaaaaaggctggcttgtaccggaagttaactccaccctacagattgagaagcacgtcagcagagcatttatctgtctacaagctctgaccactttcgtctcagtagctggcataatctacattatctacaagttgtttgccggctttcagggcgcgtatacggggatgccaaatcagaaacccaaagtgcccactctgagacaagctaaagtgcagggcccagcattcgagttcgctgtggcaatgatgaaaaggaacgccagcacagtgaaaacggaatatggtgagttcaccatgctcggcatctatgacagatgggcagtgctaccacgccacgccaagcctggaccgactattttgatgaatgatcaggagatcggcgtgttagacgccaaagaattagtggacaaagatgggacaaatctggagttgacccttttaaagctcaaccgcaatgagaagtttagggacatcagagggtttctggcgagagaggaagctgaagtgaatgaggctgttctggcaataaacacaagcaagttcccaaatatgtacatacccgtaggtcaagtcactgattacggtttcctgaacctggggggaacacccacaaagaggatgctcatgtacaactttccaactagagcagggcaatgtggtggggtactcatgtcaacagggaaagttcttggtatacatgtaggaggaaatggacatcaagggttctccgctgctcttcttaggcactactttaatgaggagcaaggtgaaatagagtttattgagagctcaaaggatgcaggattccctgtgatcaacacccccagcaaaacaaaactggaaccaagcgtgtttcatcaggtgtttgaaggcaataaagagccagcagtccttagaaatggtgatccgcgtcttaaagtaaactttgaagaagccatattttccaagtacatcgggaacatcaacacacatgtggatgaatacatgctcgaagcagtggatcactacgcagggcaactggctactcttgacattagcactgaacccatgaaactagaagatgcagtgtatggcactgaaggactagaggctcttgacttaacaacaagtgcagggtacccgtatgtcgcactaggcatcaaaaagagagacatcctatccaaaaagactaaagatctgactaaattgaaggaatgcatggacaagtacggtttgaacctgccgatggtgacttatgtgaaggatgaacttagatcatcagagaaagtggctaagggaaaatcaagactcattgaagcatctagtttaaatgactctgtcgcgatgaggcagacgtttggcaacttgtacaaaacattccatctgaacccgggcattgtgacaggcagtgcagttggatgtgatccggaccttttctggagtaaaatacccgtgatgttagacggccacctcatagcattcgattactccggatatgatgccagcctgagccctgtgtggttcgcttgtctaaaactgctactcgaaaaactcgggtacacgcacagagagacaaactacatcgactacctatgcaactctcaccacctgtacagagacaaacactactttgtgcgtggtggcatgccatcagggtgctccggtaccagcattttcaattcaatgatcaataacatcataatcaggacactgatgctcaaagtgtacaagggaattgacctggaccagtttagaatgattgcatatggtgatgatgtaattgcatcctacccttggcctatagacgcttcactacttgctgaagctgggaagggttatggactgattatgacaccagcagacaaaggggagtgcttcaatgaggttacctggaccaatgtcaccttcctaaagaggtactttagagctgatgagcagtatcctttcctggttcatccagttatgcccatgaaggacatccatgaatctattaggtggacaaaggatccaaagaacactcaagaccacgttcgatcgttgtgcttattggcttggcataacggagaacaagaatatgaggaattcatccgaaagatcagaagcgtcccggttgggcgctgtctgactctccccgcgttttcaactttacgcagaaaatggctggattccttttaaattagagacaatttgaaataatttaaattggcttaaccctactgtactaaccgaactagacaacggtgcagtaggggtaaattctccgcattcggtgcggaaaaaaaaaaaaaaaaaaaaaaaaaaaaaaaaaaaaaaaaaaaaaaaaaa

SEQ ID NO: 52 - protein, 2193 aa, Echovirus 12
MGAQVSTQKTGAHETGLSASGNSIIHYTNINYYKDAASNSANRQDFTQDPGKFTEPVKDIMIKSMPALNSPTAEECGYSDRVRSITLGNSTITTQECANVVVGYGTWPDYLHDDEATAEDQPTQPDVATCRFYTLESIQWQKTSDGWWWKFPEALKDMGLFGQNMHYHYLGRSGYTIHVQCNASKFHQGCLLVVCVPEAEMGCATVANEVTAAALSSGETAKYFAKTGATGTHTVQSIVTNAGMGVGVGNLTIFPHQWINLRTNNSATIVMPYINSVPMDNMFRHYNFTLMIIPFVPLDFTAEASTYVPITVTVAPMCAEYNGLRLASHQGLPTMNTPGSNQFLTSDDFQSPSAMPQFDVTPELRIPGEVKNLMEIAEVDSVVPVNNTQDSVYNMDVYKIPVSGGNQLSTQVFGFQMQPGLNSVFKRTLLGEILNYYAHWSGSVKLTFVFCGSAMATGKFLLAYSPPGADPPKSRKEAMLGTHVIWDIGLQSSCVLCVPWISQTHYRLVQQDEYTSAGYVTCWYQTSLVVPPGAPVTCGVLCLASACNDFSVRMLRDTPFIEQKQLLQGDVEEAVNRAVARVADTLPTGPRNSESIPALTAAETGHTSQVVPGDTMQTRHVKNYHSRTESSVENFLCRAACVYITKYKTKDSDPVQRYANWRINTRQMVQLRRKFELFTYLRFDMEVTFVITSSQDDGTQLAQDMPVLTHQVMYIPPGGPVPNSVTDFAWQSSTNPSIFWTEGNAPARMSIPFISIGNAYSNFYDGWSHFTQDGVYGYNSLNNMGSIYIRHVNEQSPHAITSTVRVYFKPKHVRAWVPRPPRLCAYEKSSNVNFKPTDVTTSRTSITEVPSLRPSVVNTGAFGQQSGAAYVGNYRVVNRHLATHVDWQNCVWEDYNRDLLVSTTTAHGCDTIARCQCTTGVYFCASRNKHYPVSFEGPGLVEVQESEYYPRRYQSHVLLAAGFSEPGDCGGILRCEHGVIGLVTMGGEGVVGFADVRDLLWLEDDAMEQGVKDYVEQLGNAFGSGFTNQICEQVNLLKESLVGHDSILEKSLKALVKIISALVIVVRNHDDLITVTATLALIGCTSSPWRWLKHKVSQYYGIPMAERQSNGWLKKFTEMTNACKGMEWIAIKIQKFIEWLKLKILPEVKEKHEFLNRLKQLPLLESQIATIEQSAPSQSDQEQLFSNVQYFAHYCRKYAPLYAAEAKRVFSLEKKMSNYIQFKSKCRIEPVCLLLHGSPGAGKSVATSLIGRSLAEKLNSSVYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYSQNGKINMPMSVKTCDEECCPVNFKRCCPLVCGKAIQFIDRRTQVRYSLDMLVTEMFREYNHRHSVGATLEALFQGPPVYREIKISVAPETPPPPAIADLLKSVDSEAVREYCKEKGWLVPEVNSTLQIEKHVSRAFICLQALTTFVSVAGIIYIIYKLFAGFQGAYTGMPNQKPKVPTLRQAKVQGPAFEFAVAMMKRNASTVKTEYGEFTMLGIYDRWAVLPRHAKPGPTILMNDQEIGVLDAKELVDKDGTNLELTLLKLNRNEKFRDIRGFLAREEAEVNEAVLAINTSKFPNMYIPVGQVTDYGFLNLGGTPTKRMLMYNFPTRAGQCGGVLMSTGKVLGIHVGGNGHQGFSAALLRHYFNEEQGEIEFIESSKDAGFPVINTPSKTKLEPSVFHQVFEGNKEPAVLRNGDPRLKVNFEEAIFSKYIGNINTHVDEYMLEAVDHYAGQLATLDISTEPMKLEDAVYGTEGLEALDLTTSAGYPYVALGIKKRDILSKKTKDLTKLKECMDKYGLNLPMVTYVKDELRSSEKVAKGKSRLIEASSLNDSVAMRQTFGNLYKTFHLNPGIVTGSAVGCDPDLFWSKIPVMLDGHLIAFDYSGYDASLSPVWFACLKLLLEKLGYTHRETNYIDYLCNSHHLYRDKHYFVRGGMPSGCSGTSIFNSMINNIIIRTLMLKVYKGIDLDQFRMIAYGDDVIASYPWPIDASLLAEAGKGYGLIMTPADKGECFNEVTWTNVTFLKRYFRADEQYPFLVHPVMPMKDIHESIRWTKDPKNTQDHVRSLCLLAWHNGEQEYEEFIRKIRSVPVGRCLTLPAFSTLRRKWLDSF

SEQ ID NO: 53 - protein, 2193 aa, Echovirus 12
MGAQVSTQKTGAHETGLSASGNSIIHYTNINYYKDAASNSANRQDFTQDPGKFTEPVKDIMIKSMPALNSPTAEECGYSDRVRSITLGNSTITTQECANVVVGYGTWPDYLHDDEATAEDQPTQPDVATCRFYTLESIQWQKTSDGWWWKFPEALKDMGLFGQNMHYHYLGRSGYTIHVQCNASKFHQGCLLVVCVPEAEMGCATVANEVTAAALSSGETAKYFAKTGATGTHTVQSIVTNAGMGVGVGNLTIFPHQWINLRTNNSATIVMPYINSVPMDNMFRHYNFTLMIIPFVPLDFTAEASTYVPITVTVAPMCAEYNGLRLASHQGLPTMNTPGSNQFLTSDDFQSPSAMPQFDVTPELRIPGEVKNLMEIAEVDSVVPVNNTQDSVYNMDVYKIPVSGGNQLSTQVFGFQMQPGLNSVFKRTLLGEILNYYAHWSGSVKLTFVFCGSAMATGKFLLAYSPPGADPPKSRKEAMLGTHVIWDIGLQSSCVLCVPWISQTHYRLVQQDEYTSAGYVTCWYQTSLVVPPGAPVTCGVLCLASACNDFSVRMLRDTPFIEQKQLLQGDVEEAVNRAVARVADTLPTGPRNSESIPALTAAETGHTSQVVPGDTMQTRHVKNYHSRTESSVENFLCRAACVYITKYKTKDSDPVQRYANWRINTRQMVQLRRKFELFTYLRFDMEVTFVITSSQDDGTRLAQDMPVLTHQVMYIPPGGPVPNSVTDFAWQSSTNPSIFWTEGNAPARMSIPFISIGNAYSNFYDGWSHFTQDGVYGFNSLNNMGSIYIRHVNEQSPHAITSTVRVYFKPKHVRAWVPRPPRLCAYEKSSNVNFKPTDVTTSRTSITEVPSLRPSVVNTGAFGQQSGAAYVGNYRVVNRHLATHVDWQNCVWEDYNRDLLVSTTTAHGCDTIARCQCTTGVYFCASRNKHYPVSFEGPGLVEVQESEYYPRRYQSHVLLAAGFSEPGDCGGILRCEHGVIGLVTMGGEGVVGFADVRDLLWLEDDAMEQGVKDYVEQLGNAFGSGFTNQICEQVNLLKESLVGHDSILEKSLKALVKIISALVIVVRNHDDLITVTATLALIGCTSSPWRWLKHKVSQYYGIPMAERQSNGWLKKFTEMTNACKGMEWIAIKIQKFIEWLKLKILPEVKEKHEFLNRLKQLPLLESQIATIEQSAPSQSDQEQLFSNVQYFAHYCRKYAPLYAAEAKRVFSLEKKMSNYIQFKSKCRIEPVCLLLHGSPGAGKSVATSLIGRSLAEKLNSSVYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYSQNGKINMPMSVKTCDEECCPVNFKRCCPLVCGKAIQFIDRRTQVRYSLDMLVTEMFREYNHRHSVGATLEALFQGPPVYREIKISVAPETPPPPAIADLLKSVDSEAVREYCKEKGWLVPEVNSTLQIEKHVSRAFICLQALTTFVSVAGIIYIIYKLFAGFQGAYTGMPNQKPKVPTLRQAKVQGPAFEFAVAMMKRNASTVKTEYGEFTMLGIYDRWAVLPRHAKPGPTILMNDQEIGVLDAKELVDKDGTNLELTLLKLNRNEKFRDIRGFLAREEAEVNEAVLAINTSKFPNMYIPVGQVTDYGFLNLGGTPTKRMLMYNFPTRAGQCGGVLMSTGKVLGIHVGGNGHQGFSAALLRHYFNEEQGEIEFIESSKDAGFPVINTPSKTKLEPSVFHQVFEGNKEPAVLRNGDPRLKVNFEEAIFSKYIGNINTHVDEYMLEAVDHYAGQLATLDISTEPMKLEDAVYGTEGLEALDLTTSAGYPYVALGIKKRDILSKKTKDLTKLKECMDKYGLNLPMVTYVKDELRSSEKVAKGKSRLIEASSLNDSVAMRQTFGNLYKTFHLNPGIVTGSAVGCDPDLFWSKIPVMLDGHLIAFDYSGYDASLSPVWFACLKLLLEKLGYTHRETNYIDYLCNSHHLYRDKHYFVRGGMPSGCSGTSIFNSMINNIIIRTLMLKVYKGIDLDQFRMIAYGDDVIASYPWPIDASLLAEAGKGYGLIMTPADKGECFNEVTWTNVTFLKRYFRADEQYPFLVHPVMPMKDIHESIRWTKDPKNTQDHVRSLCLLAWHNGEQEYEEFIRKIRSVPVGRCLTLPAFSTLRRKWLDSF

SEQ ID NO: 54 - protein, 7 aa, Echovirus 12
DGTRLAQ

SEQ ID NO: 55 - protein, 7 aa, Echovirus 12
VYGYNSL

SEQ ID NO: 56 - protein, 7 aa, Echovirus 12
QSPHAIT

SEQ ID NO: 57 - protein, 320 aa, Echovirus 12
GDVEEAVNRAVARVADTLPTGPRNSESIPALTAAETGHTSQVVPGDTMQTRHVKNYHSRTESSVENFLCRAACVYITKYKTKDSDPVQRYANWRINTRQMVQLRRKFELFTYLRFDMEVTFVITSSQDDGTQLAQDMPVLTHQVMYIPPGGPVPNSVTDFAWQSSTNPSIFWTEGNAPARMSIPFISIGNAYSNFYDGWSHFTQDGVYGYNSLNNMGSIYIRHVNEQSPHAITSTVRVYFKPKHVRAWVPRPPRLCAYEKSSNVNFKPTDVTTSRTSITEVPSLRPSVVNTGAFGQQSGAAYVGNYRVVNRHLATHVDWQ

SEQ ID NO: 58 - protein, 7 aa, Echovirus 12
NEVTAAA

SEQ ID NO: 59 - protein, 7 aa, Echovirus 12
TAKYFAK

SEQ ID NO: 60 - protein, 7 aa, Echovirus 12
TVQNIVT

SEQ ID NO: 61 - protein, 261 aa, Echovirus 12
SPTAEECGYSDRVRSITLGNSTITTQECANVVVGYGTWPDYLHDDEATAEDQPTQPDVATCRFYTLESIQWQKTSDGWWWKFPEALKDMGLFGQNMHYHYLGRSGYTIHVQCNASKFHQGCLLVVCVPEAEMGCATVANEVTAAALSSGETAKYFAKTGATGTHTVQSIVTNAGMGVGVGNLTIFPHQWINLRTNNSATIVMPYINSVPMDNMFRHYNFTLMIIPFVPLDFTAEASTYVPITVTVAPMCAEYNGLRLASHQ

SEQ ID NO: 62 - protein, 7 aa, Echovirus 12
GAPVTCG

SEQ ID NO: 63 - protein, 238 aa, Echovirus 12
GLPTMNTPGSNQFLTSDDFQSPSAMPQFDVTPELRIPGEVKNLMEIAEVDSVVPVNNTQDSVYNMDVYKIPVSGGNQLSTQVFGFQMQPGLNSVFKRTLLGEILNYYAHWSGSVKLTFVFCGSAMATGKFLLAYSPPGADPPKSRKEAMLGTHVIWDIGLQSSCVLCVPWISQTHYRLVQQDEYTSAGYVTCWYQTSLVVPPGAPVTCGVLCLASACNDFSVRMLRDTPFIEQKQLLQ

SEQ ID NO: 64 - protein, 7 aa, Echovirus 12
PVSYEGP

SEQ ID NO: 65 - protein, 121 aa, Echovirus 12
NCVWEDYNRDLLVSTTTAHGCDTIARCQCTTGVYFCASRNKHYPVSFEGPGLVEVQESEYYPRRYQSHVLLAAGFSEPGDCGGILRCEHGVIGLVTMGGEGVVGFADVRDLLWLEDDAMEQ

SEQ ID NO: 66 - protein, 7 aa, Echovirus 12
PPVYREI

SEQ ID NO: 67 - protein, 90 aa, Echovirus 12
QGPPVYREIKISVAPETPPPPAIADLLKSVDSEAVREYCKEKGWLVPEVNSTLQIEKHVSRAFICLQALTTFVSVAGIIYIIYKLFAGFQ

SEQ ID NO: 68 - protein, 6 aa, Echovirus 12
LPRHAK

SEQ ID NO: 69 - protein, 183 aa, Echovirus 12
GPAFEFAVAMMKRNASTVKTEYGEFTMLGIYDRWAVLPRHAKPGPTILMNDQEIGVLDAKELVDKDGTNLELTLLKLNRNEKFRDIRGFLAREEAEVNEAVLAINTSKFPNMYIPVGQVTDYGFLNLGGTPTKRMLMYNFPTRAGQCGGVLMSTGKVLGIHVGGNGHQGFSAALLRHYFNEEQ

SEQ ID NO: 70 - protein, 7 aa, Echovirus 12
YVALGIK

SEQ ID NO: 71 - protein, 462 aa, Echovirus 12
GEIEFIESSKDAGFPVINTPSKTKLEPSVFHQVFEGNKEPAVLRNGDPRLKVNFEEAIFSKYIGNINTHVDEYMLEAVDHYAGQLATLDISTEPMKLEDAVYGTEGLEALDLTTSAGYPYVALGIKKRDILSKKTKDLTKLKECMDKYGLNLPMVTYVKDELRSSEKVAKGKSRLIEASSLNDSVAMRQTFGNLYKTFHLNPGIVTGSAVGCDPDLFWSKIPVMLDGHLIAFDYSGYDASLSPVWFACLKLLLEKLGYTHRETNYIDYLCNSHHLYRDKHYFVRGGMPSGCSGTSIFNSMINNIIIRTLMLKVYKGIDLDQFRMIAYGDDVIASYPWPIDASLLAEAGKGYGLIMTPADKGECFNEVTWTNVTFLKRYFRADEQYPFLVHPVMPMKDIHESIRWTKDPKNTQDHVRSLCLLAWHNGEQEYEEFIRKIRSVPVGRCLTLPAFSTLRRKWLDSF

SEQ ID NO: 72 - RNA, 7473 nt, Echovirus 12
  source 1..7473: mol_type = unassigned RNA
ttaaaacagcctgtgggttgtccccacccacagggcccactgggcgctagcacactggtatcccggtacctttgtgcgcctgttttatataccctcccctcagtaacttagaagttcatcacaaatgatcaatagttagctcaacaaaccagttgagcctagatcaagcacttctgttaccccgggctgagtatcaataagctgttgacacggctgaaggagaaaacgcccgttacccgaccagctacttcggagaacctagtatcaccatagaggttgcgtagcgtttcgctccgcacaaccccagtgtagatcaggtcgatgagtcaccgcgttccccacaggcgactgtggcggtggctgcgttggcggcctgcccatggggttacccatgggacgcttcaatactgacatggtgtgaagagttgactgagctagctggtagtcctccggcccctgaatgcggctaatcctaactgtggagcaagtgcccacaacccagtgggtggcttgtcgtaatgggcaactctgcagcggaaccgactactttgggtgaccgtgtttctctttattcttatattggctgcttatggtgacaatctcagagttgttaccatatagctattggtttggccaaccagtgactaacagagcaattatatatcttttcattggatttatacctataaacaacaccagttataccactttgtgttacattattggcttgaactcgaaaagatgggagcgcaagtatcaacacaaaaaactggggcacacgagactggcctgagtgctagtggaaattccatcatccactataccaacattaattactacaaagatgcggcatccaattcagcaaacaggcaggatttcacccaagaccccgggaaattcaccgagccagtgaaggatatcatgataaaatcgatgcctgcgttaaactccccaacagcagaagaatgtgggtacagcgatagagtcagatcaatcaccctggggaactctaccatcaccacgcaggagtgtgccaatgtggtggtgggatatggcacgtggcctgattatctacatgatgatgaggccactgcagaggaccaaccaacacaaccggatgtggcgacgtgtaggttttacacacttgaatccattcagtggcagaagacatcagatgggtggtggtggaagtttccggaagcattgaaagacatgggactgttcggccaaaacatgcactatcattatcttggcaggtcgggttacaccatccacgtccaatgtaacgcgtctaaattccaccagggttgtttgcttgtggtgtgtgtgcctgaagctgagatggggtgcgctacagtggcgaatgaggttaccgcagccgctctctcatctggtgaaacggcaaaatattttgcaaagactggtgcgacgggaacccacacagtgcaaagcatagtgacaaacgccggcatgggggtgggcgtgggcaatctgacaatattcccacatcagtggataaacctccgtaccaacaacagcgcgactattgtcatgccttacataaacagtgtgcccatggataacatgttcagacactacaatttcaccctaatgatcatccccttcgtcccactggacttcacagctgaggcatctacgtatgtaccaatcactgtaacagtagctcccatgtgcgccgagtacaacgggctacgcctggcctcgcatcaaggtttgccgacaatgaacacccctggtagtaaccaattcctcacatcagatgactttcaatcaccatcagccatgccacaattcgatgtcacacccgagctcaggattccaggggaggtaaagaacttaatggaaatagccgaagttgactccgtagtgcctgtgaacaatacacaggactcagtgtacaacatggatgtgtacaaaattcctgttagtggaggcaaccaactgtcaactcaagtcttcgggttccaaatgcaaccagggctaaatagtgtgttcaaaaggacattactaggtgagatactgaactactatgcacactggtctggtagtgtaaagctcacctttgtgttctgtggatcggccatggccactggcaaatttctgctggcatattcccctccaggcgcagaccccccaaagtctaggaaagaagcaatgcttggtactcacgtgatatgggatataggacttcagtcaagctgcgtcttgtgcgtaccgtggatcagccaaacacattatagattggttcagcaggatgaatatacgagtgcaggatatgtaacttgctggtatcaaactagtttggttgtcccgcccggcgctcccgtcacgtgtggagtcttgtgcttagcatcggcgtgcaatgatttctctgtgagaatgttgagagacacaccttttatcgagcaaaaacagctactccaaggagatgtggaggaggccgtgaacagagccgttgcacgggtggcggatacactacccacagggccgagaaattcagagagcatccccgcactaacagcagcagaaactgggcatacatctcaggtggttcctggggataccatgcaaacccgtcatgtgaagaattaccactccagaactgaatcgtctgttgaaaacttcttatgccgagcagcatgtgtgtatattacaaagtacaagaccaaggacagtgatccagtgcaaaggtacgccaactggcggatcaacacccgtcaaatggttcagctaaggagaaaatttgagttgtttacatacctaaggtttgatatggaagttacttttgtgatcaccagttcacaagatgatggcacgcagttggcgcaggacatgccagtattaacacaccaagtcatgtatataccacctggagggccagtgccgaatagtgttactgattttgcgtggcaatcgtctaccaaccctagcatcttctggacggagggaaacgctccagctaggatgtccatcccctttatcagtataggcaacgcgtatagcaatttctatgatggttggtcacatttcacccaagatggagtatatggttacaactccctcaataacatgggctccatctacattaggcatgtcaatgaacaaagcccgcatgcaattacgagcacagtcagagtctatttcaaaccaaaacacgtgcgggcctgggtgccaagaccacctaggctttgtgcatatgagaaatcaagcaacgtgaatttcaaaccaacagatgtgactacctcccgaacatccatcacagaagtccctagtcttagaccatcagtggtaaatactggagctttcggtcagcaatcaggagcggcttatgtgggaaactatagagtggttaatagacacctagccactcatgttgactggcagaactgtgtgtgggaggactataacagggacctccttgtaagcaccaccacagctcatgggtgtgacaccatagccagatgccaatgcacaacaggcgtgtacttctgtgcatcgaggaataaacactacccagtcagttttgaaggaccaggtctggtagaagtccaggagagcgagtactaccccaggaggtatcaatcacacgtcttgctggctgcgggattttcagagccaggagattgtggagggattctcagatgtgagcacggtgtcatcggtctagttactatgggtggcgaaggcgttgtcggattcgctgatgtacgcgacctgttatggcttgaagatgacgccatggaacaaggagtcaaggattacgtggaacaactagggaacgcctttggttcaggattcactaaccaaatctgtgagcaggtcaacctcctaaaagagtcactggtgggtcacgattccatcctagagaagtcccttaaagccctagtgaaaatcatatcagcactagtgatagtggtgaggaaccatgatgatttgatcactgtaactgctactcttgccctcattggttgtacctcctctccatggcggtggctcaagcacaaggtgtcacaatactacgggatacccatggcagagcgccaaagcaacgggtggctcaagaagttcacagagatgaccaacgcctgcaaagggatggaatggattgccatcaaaatccaaaagttcatagagtggcttaaacttaagattttaccagaagttaaggaaaaacatgaattcctgaataggctcaagcaactcccactgttggagagtcaaatagcaaccatcgagcagagtgcaccgtcccagagcgatcaggagcagctcttttcaaatgttcagtactttgcccattattgcagaaaatatgcgcccctatacgcagctgaggcaaaaagggtgttctctcttgaaaagaaaatgagtaattacatacagttcaagtccaaatgccgtattgaacctgtatgcttgctactacatgggagccccggagcagggaaatcagttgctaccagcctcattgggcgatcactagctgaaaaattgaatagctcagtgtactccttaccaccagacccagatcactttgatggttacaagcaacaagctgtagtgatcatggacgatctgtgccaaaatccagatggaaaagatgtgtctttgttctgtcaaatggtgtctagtgtagattttgtaccaccaatggccgcgctagaggagaaaggcattttgttcacctctccattcgttctagcctcaaccaatgcaggatccatcaacgcaccaacagtttcagacagcagagccctagctagaaggttccactttgacatgaacattgaagttatctccatgtacagtcaaaatggaaaaataaacatgcccatgtcagtcaaaacatgtgatgaggaatgctgcccagtcaatttcaagagatgctgcccactggtgtgtggcaaggctattcagttcattgacagaagaacccaagtcagatattcactggatatgttagtcaccgagatgttcagggagtacaaccacaggcacagtgtgggcgccaccctcgaggctctgttccaaggtccaccggtctacagggagatcaagatcagtgttgctccagaaacacctcccccaccagcaatcgctgatttattaaaatcagtagacagtgaagctgtgagagagtactgcaaggaaaaaggctggcttgtaccggaagttaactccaccctacagattgagaagcacgtcagcagagcatttatctgtctacaagctctgaccactttcgtctcagtagctggcataatctacattatctacaagttgtttgccggctttcagggcgcgtatacggggatgccaaatcagaaacccaaagtgcccactctgagacaagctaaagtgcagggcccagcattcgagttcgctgtggcaatgatgaaaaggaacgccagcacagtgaaaacggaatatggtgagttcaccatgctcggcatctatgacagatgggcagtgctaccacgccacgccaagcctggaccgactattttgatgaatgatcaggagatcggcgtgttagacgccaaagaattagtggacaaagatgggacaaatctggagttgacccttttaaagctcaaccgcaatgagaagtttagggacatcagagggtttctggcgagagaggaagctgaagtgaatgaggctgttctggcaataaacacaagcaagttcccaaatatgtacatacccgtaggtcaagtcactgattacggtttcctgaacctggggggaacacccacaaagaggatgctcatgtacaactttccaactagagcagggcaatgtggtggggtactcatgtcaacagggaaagttcttggtatacatgtaggaggaaatggacatcaagggttctccgctgctcttcttaggcactactttaatgaggagcaaggtgaaatagagtttattgagagctcaaaggatgcaggattccctgtgatcaacacccccagcaaaacaaaactggaaccaagcgtgtttcatcaggtgtttgaaggcaataaagagccagcagtccttagaaatggtgatccgcgtcttaaagtaaactttgaagaagccatattttccaagtacatcgggaacatcaacacacatgtggatgaatacatgctcgaagcagtggatcactacgcagggcaactggctactcttgacattagcactgaacccatgaaactagaagatgcagtgtatggcactgaaggactagaggctcttgacttaacaacaagtgcagggtacccgtatgtcgcactaggcatcaaaaagagagacatcctatccaaaaagactaaagatctgactaaattgaaggaatgcatggacaagtacggtttgaacctgccgatggtgacttatgtgaaggatgaacttagatcatcagagaaagtggctaagggaaaatcaagactcattgaagcatctagtttaaatgactctgtcgcgatgaggcagacgtttggcaacttgtacaaaacattccatctgaacccgggcattgtgacaggcagtgcagttggatgtgatccggaccttttctggagtaaaatacccgtgatgttagacggccacctcatagcattcgattactccggatatgatgccagcctgagccctgtgtggttcgcttgtctaaaactgctactcgaaaaactcgggtacacgcacagagagacaaactacatcgactacctatgcaactctcaccacctgtacagagacaaacactactttgtgcgtggtggcatgccatcagggtgctccggtaccagcattttcaattcaatgatcaataacatcataatcaggacactgatgctcaaagtgtacaagggaattgacctggaccagtttagaatgattgcatatggtgatgatgtaattgcatcctacccttggcctatagacgcttcactacttgctgaagctgggaagggttatggactgattatgacaccagcagacaaaggggagtgcttcaatgaggttacctggaccaatgtcaccttcctaaagaggtactttagagctgatgagcagtatcctttcctggttcatccagttatgcccatgaaggacatccatgaatctattaggtggacaaaggatccaaagaacactcaagaccacgttcgatcgttgtgcttattggcttggcataacggagaacaagaatatgaggaattcatccgaaagatcagaagcgtcccggttgggcgctgtctgactctccccgcgttttcaactttacgcagaaaatggctggattccttttaaattagagacaatttgaaataatttaaattggcttaaccctactgtactaaccgaactagacaacggtgcagtaggggtaaattctccgcattcggtgcggaaaaaaaaaaaaaaaaaaaaaaaaaaaaaaaaaaaaaaaaaaaaaaaaaa

SEQ ID NO: 73 - protein, 2193 aa, Echovirus 12; identical to SEQ ID NO: 52

SEQ ID NO: 74 - protein, 320 aa, Echovirus 12; identical to SEQ ID NO: 57

SEQ ID NO: 75 - protein, 261 aa, Echovirus 12; identical to SEQ ID NO: 61

SEQ ID NO: 76 - protein, 238 aa, Echovirus 12; identical to SEQ ID NO: 63

SEQ ID NO: 77 - protein, 121 aa, Echovirus 12; identical to SEQ ID NO: 65

SEQ ID NO: 78 - protein, 90 aa, Echovirus 12; identical to SEQ ID NO: 67

SEQ ID NO: 79 - protein, 183 aa, Echovirus 12; identical to SEQ ID NO: 69

SEQ ID NO: 80 - protein, 462 aa, Echovirus 12; identical to SEQ ID NO: 71

SEQ ID NO: 81 - RNA, 6579 nt, Echovirus 12; identical to SEQ ID NO: 43
  source 1..6579: mol_type = unassigned RNA

SEQ ID NO: 82 - RNA, 741 nt, Echovirus 12; identical to SEQ ID NO: 44
  source 1..741: mol_type = unassigned RNA

SEQ ID NO: 83 - RNA, 103 nt, Echovirus 12; identical to SEQ ID NO: 45
  source 1..103: mol_type = unassigned RNA

SEQ ID NO: 84 - RNA, 7474 nt, Echovirus 12
  source 1..7474: mol_type = unassigned RNA
ttaaaacagcctgtgggttgtccccacccacagggcccactgggcgctagcacactggtatcccggtacctttgtgcgcctgttttatataccctcccctcagtaacttagaagttcatcacaaatgatcaatagttagctcaacaaaccagttgagcctagatcaagcacttctgttaccccgggctgagtatcaataagctgttgacacggctgaaggagaaaacgcccgttacccgaccagctacttcggagaacctagtatcaccatagaggttgcgtagcgtttcgctccgcacaaccccagtgtagatcaggtcgatgagtcaccgcgttccccacaggcgactgtggcggtggctgcgttggcggcctgcccatggggttacccatgggacgcttcaatactgacatggtgtgaagagttgactgagctagctggtagtcctccggcccctgaatgcggctaatcctaactgtggagcaagtgcccacaacccagtgggtggcttgtcgtaatgggcaactctgcagcggaaccgactactttgggtgaccgtgtttctctttattcttatattggctgcttatggtgacaatctcagagttgttaccatatagctattggtttggccaaccagtgactaacagagcaattatatatcttttcattggatttatacctataaacaacaccagttataccactttgtgttacattattggcttgaactcgaaaagatgggagcgcaagtatcaacacaaaaaactggggcacacgagactggcctgagtgctagtggaaattccatcatccactataccaacattaattactacaaagatgcggcatccaattcagcaaacaggcaggatttcacccaagaccccgggaaattcaccgagccagtgaaggatatcatgataaaatcgatgcctgcgttaaactccccaacagcagaagaatgtgggtacagcgatagagtcagatcaatcaccctggggaactctaccatcaccacgcaggagtgtgccaatgtggtggtgggatatggcacgtggcctgattatctacatgatgatgaggccactgcagaggaccaaccaacacaaccggatgtggcgacgtgtaggttttacacacttgaatccattcagtggcagaagacatcagatgggtggtggtggaagtttccggaagcattgaaagacatgggactgttcggccaaaacatgcactatcattatcttggcaggtcgggttacaccatccacgtccaatgtaacgcgtctaaattccaccagggttgtttgcttgtggtgtgtgtgcctgaagctgagatggggtgcgctacagtggcgaatgaggttaccgcagccgctctctcatctggtgaaacggcaaaatattttgcaaagactggtgcgacgggaacccacacagtgcaaagcatagtgacaaacgccggcatgggggtgggcgtgggcaatctgacaatattcccacatcagtggataaacctccgtaccaacaacagcgcgactattgtcatgccttacataaacagtgtgcccatggataacatgttcagacactacaatttcaccctaatgatcatccccttcgtcccactggacttcacagctgaggcatctacgtatgtaccaatcactgtaacagtagctcccatgtgcgccgagtacaacgggctacgcctggcctcgcatcaaggtttgccgacaatgaacacccctggtagtaaccaattcctcacatcagatgactttcaatcaccatcagccatgccacaattcgatgtcacacccgagctcaggattccaggggaggtaaagaacttaatggaaatagccgaagttgactccgtagtgcctgtgaacaatacacaggactcagtgtacaacatggatgtgtacaaaattcctgttagtggaggcaaccaactgtcaactcaagtcttcgggttccaaatgcaaccagggctaaatagtgtgttcaaaaggacattactaggtgagatactgaactactatgcacactggtctggtagtgtaaagctcacctttgtgttctgtggatcggccatggccactggcaaatttctgctggcatattcccctccaggcgcagaccccccaaagtctaggaaagaagcaatgcttggtactcacgtgatatgggatataggacttcagtcaagctgcgtcttgtgcgtaccgtggatcagccaaacacattatagattggttcagcaggatgaatatacgagtgcaggatatgtaacttgctggtatcaaactagtttggttgtcccgcccggcgctcccgtcacgtgtggagtcttgtgcttagcatcggcgtgcaatgatttctctgtgagaatgttgagagacacaccttttatcgagcaaaaacagctactccaaggagatgtggaggaggccgtgaacagagccgttgcacgggtggcggatacactacccacagggccgagaaattcagagagcatccccgcactaacagcagcagaaactgggcatacatctcaggtggttcctggggataccatgcaaacccgtcatgtgaagaattaccactccagaactgaatcgtctgttgaaaacttcttatgccgagcagcatgtgtgtatattacaaagtacaagaccaaggacagtgatccagtgcaaaggtacgccaactggcggatcaacacccgtcaaatggttcagctaaggagaaaatttgagttgtttacatacctaaggtttgatatggaagttacttttgtgatcaccagttcacaagatgatggcacgcagttggcgcaggacatgccagtattaacacaccaagtcatgtatataccacctggagggccagtgccgaatagtgttactgattttgcgtggcaatcgtctaccaaccctagcatcttctggacggagggaaacgctccagctaggatgtccatcccctttatcagtataggcaacgcgtatagcaatttctatgatggttggtcacatttcacccaagatggagtatatggtttcaactccctcaataacatgggctccatctacattaggcatgtcaatgaacaaagcccgcatgcaattacgagcacagtcagagtctatttcaaaccaaaacacgtgcgggcctgggtgccaagaccacctaggctttgtgcatatgagaaatcaagcaacgtgaatttcaaaccaacagatgtgactacctcccgaacatccatcacagaagtccctagtcttagaccatcagtggtaaatactggagctttcggtcagcaatcaggagcggcttatgtgggaaactatagagtggttaatagacacctagccactcatgttgactggcagaactgtgtgtgggaggactataacagggacctccttgtaagcaccaccacagctcatgggtgtgacaccatagccagatgccaatgcacaacaggcgtgtacttctgtgcatcgaggaataaacactacccagtcagttttgaaggaccaggtctggtagaagtccaggagagcgagtactaccccaggaggtatcaatcacacgtcttgctggctgcgggattttcagagccaggagattgtggagggattctcagatgtgagcacggtgtcatcggtctagttactatgggtggcgaaggcgttgtcggattcgctgatgtacgcgacctgttatggcttgaagatgacgccatggaacaaggagtcaaggattacgtggaacaactagggaacgcctttggttcaggattcactaaccaaatctgtgagcaggtcaacctcctaaaagagtcactggtgggtcacgattccatcctagagaagtcccttaaagccctagtgaaaatcatatcagcactagtgatagtggtgaggaaccatgatgatttgatcactgtaactgctactcttgccctcattggttgtacctcctctccatggcggtggctcaagcacaaggtgtcacaatactacgggatacccatggcagagcgccaaagcaacgggtggctcaagaagttcacagagatgaccaacgcctgcaaagggatggaatggattgccatcaaaatccaaaagttcatagagtggcttaaacttaagattttaccagaagttaaggaaaaacatgaattcctgaataggctcaagcaactcccactgttggagagtcaaatagcaaccatcgagcagagtgcaccgtcccagagcgatcaggagcagctcttttcaaatgttcagtactttgcccattattgcagaaaatatgcgcccctatacgcagctgaggcaaaaagggtgttctctcttgaaaagaaaatgagtaattacatacagttcaagtccaaatgccgtattgaacctgtatgcttgctactacatgggagccccggagcagggaaatcagttgctaccagcctcattgggcgatcactagctgaaaaattgaatagctcagtgtactccttaccaccagacccagatcactttgatggttacaagcaacaagctgtagtgatcatggacgatctgtgccaaaatccagatggaaaagatgtgtctttgttctgtcaaatggtgtctagtgtagattttgtaccaccaatggccgcgctagaggagaaaggcattttgttcacctctccattcgttctagcctcaaccaatgcaggatccatcaacgcaccaacagtttcagacagcagagccctagctagaaggttccactttgacatgaacattgaagttatctccatgtacagtcaaaatggaaaaataaacatgcccatgtcagtcaaaacatgtgatgaggaatgctgcccagtcaatttcaagagatgctgcccactggtgtgtggcaaggctattcagttcattgacagaagaacccaagtcagatattcactggatatgttagtcaccgagatgttcagggagtacaaccacaggcacagtgtgggcgccaccctcgaggctctgttccaaggtccaccggtctacagggagatcaagatcagtgttgctccagaaacacctcccccaccagcaatcgctgatttattaaaatcagtagacagtgaagctgtgagagagtactgcaaggaaaaaggctggcttgtaccggaagttaactccaccctacagattgagaagcacgtcagcagagcatttatctgtctacaagctctgaccactttcgtctcagtagctggcataatctacattatctacaagttgtttgccggctttcagggcgcgtatacggggatgccaaatcagaaacccaaagtgcccactctgagacaagctaaagtgcagggcccagcattcgagttcgctgtggcaatgatgaaaaggaacgccagcacagtgaaaacggaatatggtgagttcaccatgctcggcatctatgacagatgggcagtgctaccacgccacgccaagcctggaccgactattttgatgaatgatcaggagatcggcgtgttagacgccaaagaattagtggacaaagatgggacaaatctggagttgacccttttaaagctcaaccgcaatgagaagtttagggacatcagagggtttctggcgagagaggaagctgaagtgaatgaggctgttctggcaataaacacaagcaagttcccaaatatgtacatacccgtaggtcaagtcactgattacggtttcctgaacctggggggaacacccacaaagaggatgctcatgtacaactttccaactagagcagggcaatgtggtggggtactcatgtcaacagggaaagttcttggtatacatgtaggaggaaatggacatcaagggttctccgctgctcttcttaggcactactttaatgaggagcaaggtgaaatagagtttattgagagctcaaaggatgcaggattccctgtgatcaacacccccagcaaaacaaaactggaaccaagcgtgtttcatcaggtgtttgaaggcaataaagagccagcagtccttagaaatggtgatccgcgtcttaaagtaaactttgaagaagccatattttccaagtacatcgggaacatcaacacacatgtggatgaatacatgctcgaagcagtggatcactacgcagggcaactggctactcttgacattagcactgaacccatgaaactagaagatgcagtgtatggcactgaaggactagaggctcttgacttaacaacaagtgcagggtacccgtatgtcgcactaggcatcaaaaagagagacatcctatccaaaaagactaaagatctgactaaattgaaggaatgcatggacaagtacggtttgaacctgccgatggtgacttatgtgaaggatgaacttagatcatcagagaaagtggctaagggaaaatcaagactcattgaagcatctagtttaaatgactctgtcgcgatgaggcagacgtttggcaacttgtacaaaacattccatctgaacccgggcattgtgacaggcagtgcagttggatgtgatccggaccttttctggagtaaaatacccgtgatgttagacggccacctcatagcattcgattactccggatatgatgccagcctgagccctgtgtggttcgcttgtctaaaactgctactcgaaaaactcgggtacacgcacagagagacaaactacatcgactacctatgcaactctcaccacctgtacagagacaaacactactttgtgcgtggtggcatgccatcagggtgctccggtaccagcattttcaattcaatgatcaataacatcataatcaggacactgatgctcaaagtgtacaagggaattgacctggaccagtttagaatgattgcatatggtgatgatgtaattgcatcctacccttggcctatagacgcttcactacttgctgaagctgggaagggttatggactgattatgacaccagcagacaaaggggagtgcttcaatgaggttacctggaccaatgtcaccttcctaaagaggtactttagagctgatgagcagtatcctttcctggttcatccagttatgcccatgaaggacatccatgaatctattaggtggacaaaggatccaaagaacactcaagaccacgttcgatcgttgtgcttattggcttggcataacggagaacaagaatatgaggaattcatccgaaagatcagaagcgtcccggttgggcgctgtctgactctccccgcgttttcaactttacgcagaaaatggctggattccttttaaattagagacaatttgaaataatttaaattggcttaaccctactgtactaaccgaactagacaacggtgcagtaggggtaaattctccgcattcggtgcggaaaaaaaaaaaaaaaaaaaaaaaaaaaaaaaaaaaaaaaaaaaaaaaaaaa

SEQ ID NO: 85 - protein, 2193 aa, Echovirus 12
MGAQVSTQKTGAHETGLSASGNSIIHYTNINYYKDAASNSANRQDFTQDPGKFTEPVKDIMIKSMPALNSPTAEECGYSDRVRSITLGNSTITTQECANVVVGYGTWPDYLHDDEATAEDQPTQPDVATCRFYTLESIQWQKTSDGWWWKFPEALKDMGLFGQNMHYHYLGRSGYTIHVQCNASKFHQGCLLVVCVPEAEMGCATVANEVTAAALSSGETAKYFAKTGATGTHTVQSIVTNAGMGVGVGNLTIFPHQWINLRTNNSATIVMPYINSVPMDNMFRHYNFTLMIIPFVPLDFTAEASTYVPITVTVAPMCAEYNGLRLASHQGLPTMNTPGSNQFLTSDDFQSPSAMPQFDVTPELRIPGEVKNLMEIAEVDSVVPVNNTQDSVYNMDVYKIPVSGGNQLSTQVFGFQMQPGLNSVFKRTLLGEILNYYAHWSGSVKLTFVFCGSAMATGKFLLAYSPPGADPPKSRKEAMLGTHVIWDIGLQSSCVLCVPWISQTHYRLVQQDEYTSAGYVTCWYQTSLVVPPGAPVTCGVLCLASACNDFSVRMLRDTPFIEQKQLLQGDVEEAVNRAVARVADTLPTGPRNSESIPALTAAETGHTSQVVPGDTMQTRHVKNYHSRTESSVENFLCRAACVYITKYKTKDSDPVQRYANWRINTRQMVQLRRKFELFTYLRFDMEVTFVITSSQDDGTQLAQDMPVLTHQVMYIPPGGPVPNSVTDFAWQSSTNPSIFWTEGNAPARMSIPFISIGNAYSNFYDGWSHFTQDGVYGFNSLNNMGSIYIRHVNEQSPHAITSTVRVYFKPKHVRAWVPRPPRLCAYEKSSNVNFKPTDVTTSRTSITEVPSLRPSVVNTGAFGQQSGAAYVGNYRVVNRHLATHVDWQNCVWEDYNRDLLVSTTTAHGCDTIARCQCTTGVYFCASRNKHYPVSFEGPGLVEVQESEYYPRRYQSHVLLAAGFSEPGDCGGILRCEHGVIGLVTMGGEGVVGFADVRDLLWLEDDAMEQGVKDYVEQLGNAFGSGFTNQICEQVNLLKESLVGHDSILEKSLKALVKIISALVIVVRNHDDLITVTATLALIGCTSSPWRWLKHKVSQYYGIPMAERQSNGWLKKFTEMTNACKGMEWIAIKIQKFIEWLKLKILPEVKEKHEFLNRLKQLPLLESQIATIEQSAPSQSDQEQLFSNVQYFAHYCRKYAPLYAAEAKRVFSLEKKMSNYIQFKSKCRIEPVCLLLHGSPGAGKSVATSLIGRSLAEKLNSSVYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYSQNGKINMPMSVKTCDEECCPVNFKRCCPLVCGKAIQFIDRRTQVRYSLDMLVTEMFREYNHRHSVGATLEALFQGPPVYREIKISVAPETPPPPAIADLLKSVDSEAVREYCKEKGWLVPEVNSTLQIEKHVSRAFICLQALTTFVSVAGIIYIIYKLFAGFQGAYTGMPNQKPKVPTLRQAKVQGPAFEFAVAMMKRNASTVKTEYGEFTMLGIYDRWAVLPRHAKPGPTILMNDQEIGVLDAKELVDKDGTNLELTLLKLNRNEKFRDIRGFLAREEAEVNEAVLAINTSKFPNMYIPVGQVTDYGFLNLGGTPTKRMLMYNFPTRAGQCGGVLMSTGKVLGIHVGGNGHQGFSAALLRHYFNEEQGEIEFIESSKDAGFPVINTPSKTKLEPSVFHQVFEGNKEPAVLRNGDPRLKVNFEEAIFSKYIGNINTHVDEYMLEAVDHYAGQLATLDISTEPMKLEDAVYGTEGLEALDLTTSAGYPYVALGIKKRDILSKKTKDLTKLKECMDKYGLNLPMVTYVKDELRSSEKVAKGKSRLIEASSLNDSVAMRQTFGNLYKTFHLNPGIVTGSAVGCDPDLFWSKIPVMLDGHLIAFDYSGYDASLSPVWFACLKLLLEKLGYTHRETNYIDYLCNSHHLYRDKHYFVRGGMPSGCSGTSIFNSMINNIIIRTLMLKVYKGIDLDQFRMIAYGDDVIASYPWPIDASLLAEAGKGYGLIMTPADKGECFNEVTWTNVTFLKRYFRADEQYPFLVHPVMPMKDIHESIRWTKDPKNTQDHVRSLCLLAWHNGEQEYEEFIRKIRSVPVGRCLTLPAFSTLRRKWLDSF

SEQ ID NO: 86 - protein, 320 aa, Echovirus 12
GDVEEAVNRAVARVADTLPTGPRNSESIPALTAAETGHTSQVVPGDTMQTRHVKNYHSRTESSVENFLCRAACVYITKYKTKDSDPVQRYANWRINTRQMVQLRRKFELFTYLRFDMEVTFVITSSQDDGTQLAQDMPVLTHQVMYIPPGGPVPNSVTDFAWQSSTNPSIFWTEGNAPARMSIPFISIGNAYSNFYDGWSHFTQDGVYGFNSLNNMGSIYIRHVNEQSPHAITSTVRVYFKPKHVRAWVPRPPRLCAYEKSSNVNFKPTDVTTSRTSITEVPSLRPSVVNTGAFGQQSGAAYVGNYRVVNRHLATHVDWQ

SEQ ID NO: 87 - protein, 291 aa, Echovirus 12
SPTAEECGYSDRVRSITLGNSTITTQECANVVVGYGTWPDYLHDDEATAEDQPTQPDVATCRFYTLESIQWQKTSDGWWWKFPEALKDMGLFGQNMHYHYLGRSGYTIHVQCNASKFHQGCLLVVCVPEAEMGCATVANEVTAAALSSGETAKYFAKTGATGTHTVQSIVTNAGMGVGVGNLTIFPHQWINLRTNNSATIVMPYINSVPMDNMFRHYNFTLMIIPFVPLDFTAEASTYVPITVTVAPMCAEYNGLRLASHQGLPTMNTPGSNQFLTSDDFQSPSAMPQFDV

SEQ ID NO: 88 - protein, 238 aa, Echovirus 12; identical to SEQ ID NO: 63

SEQ ID NO: 89 - protein, 121 aa, Echovirus 12; identical to SEQ ID NO: 65

SEQ ID NO: 90 - protein, 90 aa, Echovirus 12; identical to SEQ ID NO: 67

SEQ ID NO: 91 - protein, 183 aa, Echovirus 12; identical to SEQ ID NO: 69

SEQ ID NO: 92 - protein, 462 aa, Echovirus 12; identical to SEQ ID NO: 71

SEQ ID NO: 93 - RNA, 6579 nt, Echovirus 12
  source 1..6579: mol_type = unassigned RNA
atgggagcgcaagtatcaacacaaaaaactggggcacacgagactggcctgagtgctagtggaaattccatcatccactataccaacattaattactacaaagatgcggcatccaattcagcaaacaggcaggatttcacccaagaccccgggaaattcaccgagccagtgaaggatatcatgataaaatcgatgcctgcgttaaactccccaacagcagaagaatgtgggtacagcgatagagtcagatcaatcaccctggggaactctaccatcaccacgcaggagtgtgccaatgtggtggtgggatatggcacgtggcctgattatctacatgatgatgaggccactgcagaggaccaaccaacacaaccggatgtggcgacgtgtaggttttacacacttgaatccattcagtggcagaagacatcagatgggtggtggtggaagtttccggaagcattgaaagacatgggactgttcggccaaaacatgcactatcattatcttggcaggtcgggttacaccatccacgtccaatgtaacgcgtctaaattccaccagggttgtttgcttgtggtgtgtgtgcctgaagctgagatggggtgcgctacagtggcgaatgaggttaccgcagccgctctctcatctggtgaaacggcaaaatattttgcaaagactggtgcgacgggaacccacacagtgcaaagcatagtgacaaacgccggcatgggggtgggcgtgggcaatctgacaatattcccacatcagtggataaacctccgtaccaacaacagcgcgactattgtcatgccttacataaacagtgtgcccatggataacatgttcagacactacaatttcaccctaatgatcatccccttcgtcccactggacttcacagctgaggcatctacgtatgtaccaatcactgtaacagtagctcccatgtgcgccgagtacaacgggctacgcctggcctcgcatcaaggtttgccgacaatgaacacccctggtagtaaccaattcctcacatcagatgactttcaatcaccatcagccatgccacaattcgatgtcacacccgagctcaggattccaggggaggtaaagaacttaatggaaatagccgaagttgactccgtagtgcctgtgaacaatacacaggactcagtgtacaacatggatgtgtacaaaattcctgttagtggaggcaaccaactgtcaactcaagtcttcgggttccaaatgcaaccagggctaaatagtgtgttcaaaaggacattactaggtgagatactgaactactatgcacactggtctggtagtgtaaagctcacctttgtgttctgtggatcggccatggccactggcaaatttctgctggcatattcccctccaggcgcagaccccccaaagtctaggaaagaagcaatgcttggtactcacgtgatatgggatataggacttcagtcaagctgcgtcttgtgcgtaccgtggatcagccaaacacattatagattggttcagcaggatgaatatacgagtgcaggatatgtaacttgctggtatcaaactagtttggttgtcccgcccggcgctcccgtcacgtgtggagtcttgtgcttagcatcggcgtgcaatgatttctctgtgagaatgttgagagacacaccttttatcgagcaaaaacagctactccaaggagatgtggaggaggccgtgaacagagccgttgcacgggtggcggatacactacccacagggccgagaaattcagagagcatccccgcactaacagcagcagaaactgggcatacatctcaggtggttcctggggataccatgcaaacccgtcatgtgaagaattaccactccagaactgaatcgtctgttgaaaacttcttatgccgagcagcatgtgtgtatattacaaagtacaagaccaaggacagtgatccagtgcaaaggtacgccaactggcggatcaacacccgtcaaatggttcagctaaggagaaaatttgagttgtttacatacctaaggtttgatatggaagttacttttgtgatcaccagttcacaagatgatggcacgcagttggcgcaggacatgccagtattaacacaccaagtcatgtatataccacctggagggccagtgccgaatagtgttactgattttgcgtggcaatcgtctaccaaccctagcatcttctggacggagggaaacgctccagctaggatgtccatcccctttatcagtataggcaacgcgtatagcaatttctatgatggttggtcacatttcacccaagatggagtatatggtttcaactccctcaataacatgggctccatctacattaggcatgtcaatgaacaaagcccgcatgcaattacgagcacagtcagagtctatttcaaaccaaaacacgtgcgggcctgggtgccaagaccacctaggctttgtgcatatgagaaatcaagcaacgtgaatttcaaaccaacagatgtgactacctcccgaacatccatcacagaagtccctagtcttagaccatcagtggtaaatactggagctttcggtcagcaatcaggagcggcttatgtgggaaactatagagtggttaatagacacctagccactcatgttgactggcagaactgtgtgtgggaggactataacagggacctccttgtaagcaccaccacagctcatgggtgtgacaccatagccagatgccaatgcacaacaggcgtgtacttctgtgcatcgaggaataaacactacccagtcagttttgaaggaccaggtctggtagaagtccaggagagcgagtactaccccaggaggtatcaatcacacgtcttgctggctgcgggattttcagagccaggagattgtggagggattctcagatgtgagcacggtgtcatcggtctagttactatgggtggcgaaggcgttgtcggattcgctgatgtacgcgacctgttatggcttgaagatgacgccatggaacaaggagtcaaggattacgtggaacaactagggaacgcctttggttcaggattcactaaccaaatctgtgagcaggtcaacctcctaaaagagtcactggtgggtcacgattccatcctagagaagtcccttaaagccctagtgaaaatcatatcagcactagtgatagtggtgaggaaccatgatgatttgatcactgtaactgctactcttgccctcattggttgtacctcctctccatggcggtggctcaagcacaaggtgtcacaatactacgggatacccatggcagagcgccaaagcaacgggtggctcaagaagttcacagagatgaccaacgcctgcaaagggatggaatggattgccatcaaaatccaaaagttcatagagtggcttaaacttaagattttaccagaagttaaggaaaaacatgaattcctgaataggctcaagcaactcccactgttggagagtcaaatagcaaccatcgagcagagtgcaccgtcccagagcgatcaggagcagctcttttcaaatgttcagtactttgcccattattgcagaaaatatgcgcccctatacgcagctgaggcaaaaagggtgttctctcttgaaaagaaaatgagtaattacatacagttcaagtccaaatgccgtattgaacctgtatgcttgctactacatgggagccccggagcagggaaatcagttgctaccagcctcattgggcgatcactagctgaaaaattgaatagctcagtgtactccttaccaccagacccagatcactttgatggttacaagcaacaagctgtagtgatcatggacgatctgtgccaaaatccagatggaaaagatgtgtctttgttctgtcaaatggtgtctagtgtagattttgtaccaccaatggccgcgctagaggagaaaggcattttgttcacctctccattcgttctagcctcaaccaatgcaggatccatcaacgcaccaacagtttcagacagcagagccctagctagaaggttccactttgacatgaacattgaagttatctccatgtacagtcaaaatggaaaaataaacatgcccatgtcagtcaaaacatgtgatgaggaatgctgcccagtcaatttcaagagatgctgcccactggtgtgtggcaaggctattcagttcattgacagaagaacccaagtcagatattcactggatatgttagtcaccgagatgttcagggagtacaaccacaggcacagtgtgggcgccaccctcgaggctctgttccaaggtccaccggtctacagggagatcaagatcagtgttgctccagaaacacctcccccaccagcaatcgctgatttattaaaatcagtagacagtgaagctgtgagagagtactgcaaggaaaaaggctggcttgtaccggaagttaactccaccctacagattgagaagcacgtcagcagagcatttatctgtctacaagctctgaccactttcgtctcagtagctggcataatctacattatctacaagttgtttgccggctttcagggcgcgtatacggggatgccaaatcagaaacccaaagtgcccactctgagacaagctaaagtgcagggcccagcattcgagttcgctgtggcaatgatgaaaaggaacgccagcacagtgaaaacggaatatggtgagttcaccatgctcggcatctatgacagatgggcagtgctaccacgccacgccaagcctggaccgactattttgatgaatgatcaggagatcggcgtgttagacgccaaagaattagtggacaaagatgggacaaatctggagttgacccttttaaagctcaaccgcaatgagaagtttagggacatcagagggtttctggcgagagaggaagctgaagtgaatgaggctgttctggcaataaacacaagcaagttcccaaatatgtacatacccgtaggtcaagtcactgattacggtttcctgaacctggggggaacacccacaaagaggatgctcatgtacaactttccaactagagcagggcaatgtggtggggtactcatgtcaacagggaaagttcttggtatacatgtaggaggaaatggacatcaagggttctccgctgctcttcttaggcactactttaatgaggagcaaggtgaaatagagtttattgagagctcaaaggatgcaggattccctgtgatcaacacccccagcaaaacaaaactggaaccaagcgtgtttcatcaggtgtttgaaggcaataaagagccagcagtccttagaaatggtgatccgcgtcttaaagtaaactttgaagaagccatattttccaagtacatcgggaacatcaacacacatgtggatgaatacatgctcgaagcagtggatcactacgcagggcaactggctactcttgacattagcactgaacccatgaaactagaagatgcagtgtatggcactgaaggactagaggctcttgacttaacaacaagtgcagggtacccgtatgtcgcactaggcatcaaaaagagagacatcctatccaaaaagactaaagatctgactaaattgaaggaatgcatggacaagtacggtttgaacctgccgatggtgacttatgtgaaggatgaacttagatcatcagagaaagtggctaagggaaaatcaagactcattgaagcatctagtttaaatgactctgtcgcgatgaggcagacgtttggcaacttgtacaaaacattccatctgaacccgggcattgtgacaggcagtgcagttggatgtgatccggaccttttctggagtaaaatacccgtgatgttagacggccacctcatagcattcgattactccggatatgatgccagcctgagccctgtgtggttcgcttgtctaaaactgctactcgaaaaactcgggtacacgcacagagagacaaactacatcgactacctatgcaactctcaccacctgtacagagacaaacactactttgtgcgtggtggcatgccatcagggtgctccggtaccagcattttcaattcaatgatcaataacatcataatcaggacactgatgctcaaagtgtacaagggaattgacctggaccagtttagaatgattgcatatggtgatgatgtaattgcatcctacccttggcctatagacgcttcactacttgctgaagctgggaagggttatggactgattatgacaccagcagacaaaggggagtgcttcaatgaggttacctggaccaatgtcaccttcctaaagaggtactttagagctgatgagcagtatcctttcctggttcatccagttatgcccatgaaggacatccatgaatctattaggtggacaaaggatccaaagaacactcaagaccacgttcgatcgttgtgcttattggcttggcataacggagaacaagaatatgaggaattcatccgaaagatcagaagcgtcccggttgggcgctgtctgactctccccgcgttttcaactttacgcagaaaatggctggattccttt

SEQ ID NO: 94 - RNA, 741 nt, Echovirus 12; identical to SEQ ID NO: 44
  source 1..741: mol_type = unassigned RNA

SEQ ID NO: 95 - RNA, 103 nt, Echovirus 12; identical to SEQ ID NO: 45
  source 1..103: mol_type = unassigned RNA

SEQ ID NO: 96 - RNA, 7403 nt, Echovirus 12
  source 1..7403: mol_type = unassigned RNA
ttgtgcgcctgttttatataccctcccctcagtaacttagaagttcatcacaaatgatcaatagttagctcaacaaaccagttgagcctagatcaagcacttctgttaccccgggctgagtatcaataagctgttgacacggctgaaggagaaaacgcccgttacccgaccagctacttcggagaacctagtatcaccatagaggttgcgtagcgtttcgctccgcacaaccccagtgtagatcaggtcgatgagtcaccgcgttccccacaggcgactgtggcggtggctgcgttggcggcctgcccatggggttacccatgggacgcttcaatactgacatggtgtgaagagttgactgagctagctggtagtcctccggcccctgaatgcggctaatcctaactgtggagcaagtgcccacaacccagtgggtggcttgtcgtaatgggcaactctgcagcggaaccgactactttgggtgaccgtgtttctctttattcttatattggctgcttatggtgacaatctcagagttgttaccatatagctattggtttggccaaccagtgactaacagagcaattatatatcttttcattggatttatacctataaacaacaccagttataccactttgtgttacattattggcttgaactcgaaaagatgggagcgcaagtatcaacacaaaaaactggggcacacgagactggcctgagtgctagtggaaattccatcatccactataccaacattaattactacaaagatgcggcatccaattcagcaaacaggcaggatttcacccaagaccccgggaaattcaccgagccagtgaaggatatcatgataaaatcgatgcctgcgttaaactccccaacagcagaagaatgtgggtacagcgatagagtcagatcaatcaccctggggaactctaccatcaccacgcaggagtgtgccaatgtggtggtgggatatggcacgtggcctgattatctacatgatgatgaggccactgcagaggaccaaccaacacaaccggatgtggcgacgtgtaggttttacacacttgaatccattcagtggcagaagacatcagatgggtggtggtggaagtttccggaagcattgaaagacatgggactgttcggccaaaacatgcactatcattatcttggcaggtcgggttacaccatccacgtccaatgtaacgcgtctaaattccaccagggttgtttgcttgtggtgtgtgtgcctgaagctgagatggggtgcgctacagtggcgaatgaggttaccgcagccgctctctcatctggtgaaacggcaaaatattttgcaaagactggtgcgacgggaacccacacagtgcaaagcatagtgacaaacgccggcatgggggtgggcgtgggcaatctgacaatattcccacatcagtggataaacctccgtaccaacaacagcgcgactattgtcatgccttacataaacagtgtgcccatggataacatgttcagacactacaatttcaccctaatgatcatccccttcgtcccactggacttcacagctgaggcatctacgtatgtaccaatcactgtaacagtagctcccatgtgcgccgagtacaacgggctacgcctggcctcgcatcaaggtttgccgacaatgaacacccctggtagtaaccaattcctcacatcagatgactttcaatcaccatcagccatgccacaattcgatgtcacacccgagctcaggattccaggggaggtaaagaacttaatggaaatagccgaagttgactccgtagtgcctgtgaacaatacacaggactcagtgtacaacatggatgtgtacaaaattcctgttagtggaggcaaccaactgtcaactcaagtcttcgggttccaaatgcaaccagggctaaatagtgtgttcaaaaggacattactaggtgagatactgaactactatgcacactggtctggtagtgtaaagctcacctttgtgttctgtggatcggccatggccactggcaaatttctgctggcatattcccctccaggcgcagaccccccaaagtctaggaaagaagcaatgcttggtactcacgtgatatgggatataggacttcagtcaagctgcgtcttgtgcgtaccgtggatcagccaaacacattatagattggttcagcaggatgaatatacgagtgcaggatatgtaacttgctggtatcaaactagtttggttgtcccgcccggcgctcccgtcacgtgtggagtcttgtgcttagcatcggcgtgcaatgatttctctgtgagaatgttgagagacacaccttttatcgagcaaaaacagctactccaaggagatgtggaggaggccgtgaacagagccgttgcacgggtggcggatacactacccacagggccgagaaattcagagagcatccccgcactaacagcagcagaaactgggcatacatctcaggtggttcctggggataccatgcaaacccgtcatgtgaagaattaccactccagaactgaatcgtctgttgaaaacttcttatgccgagcagcatgtgtgtatattacaaagtacaagaccaaggacagtgatccagtgcaaaggtacgccaactggcggatcaacacccgtcaaatggttcagctaaggagaaaatttgagttgtttacatacctaaggtttgatatggaagttacttttgtgatcaccagttcacaagatgatggcacgcagttggcgcaggacatgccagtattaacacaccaagtcatgtatataccacctggagggccagtgccgaatagtgttactgattttgcgtggcaatcgtctaccaaccctagcatcttctggacggagggaaacgctccagctaggatgtccatcccctttatcagtataggcaacgcgtatagcaatttctatgatggttggtcacatttcacccaagatggagtatatggtttcaactccctcaataacatgggctccatctacattaggcatgtcaatgaacaaagcccgcatgcaattacgagcacagtcagagtctatttcaaaccaaaacacgtgcgggcctgggtgccaagaccacctaggctttgtgcatatgagaaatcaagcaacgtgaatttcaaaccaacagatgtgactacctcccgaacatccatcacagaagtccctagtcttagaccatcagtggtaaatactggagctttcggtcagcaatcaggagcggcttatgtgggaaactatagagtggttaatagacacctagccactcatgttgactggcagaactgtgtgtgggaggactataacagggacctccttgtaagcaccaccacagctcatgggtgtgacaccatagccagatgccaatgcacaacaggcgtgtacttctgtgcatcgaggaataaacactacccagtcagttttgaaggaccaggtctggtagaagtccaggagagcgagtactaccccaggaggtatcaatcacacgtcttgctggctgcgggattttcagagccaggagattgtggagggattctcagatgtgagcacggtgtcatcggtctagttactatgggtggcgaaggcgttgtcggattcgctgatgtacgcgacctgttatggcttgaagatgacgccatggaacaaggagtcaaggattacgtggaacaactagggaacgcctttggttcaggattcactaaccaaatctgtgagcaggtcaacctcctaaaagagtcactggtgggtcacgattccatcctagagaagtcccttaaagccctagtgaaaatcatatcagcactagtgatagtggtgaggaaccatgatgatttgatcactgtaactgctactcttgccctcattggttgtacctcctctccatggcggtggctcaagcacaaggtgtcacaatactacgggatacccatggcagagcgccaaagcaacgggtggctcaagaagttcacagagatgaccaacgcctgcaaagggatggaatggattgccatcaaaatccaaaagttcatagagtggcttaaacttaagattttaccagaagttaaggaaaaacatgaattcctgaataggctcaagcaactcccactgttggagagtcaaatagcaaccatcgagcagagtgcaccgtcccagagcgatcaggagcagctcttttcaaatgttcagtactttgcccattattgcagaaaatatgcgcccctatacgcagctgaggcaaaaagggtgttctctcttgaaaagaaaatgagtaattacatacagttcaagtccaaatgccgtattgaacctgtatgcttgctactacatgggagccccggagcagggaaatcagttgctaccagcctcattgggcgatcactagctgaaaaattgaatagctcagtgtactccttaccaccagacccagatcactttgatggttacaagcaacaagctgtagtgatcatggacgatctgtgccaaaatccagatggaaaagatgtgtctttgttctgtcaaatggtgtctagtgtagattttgtaccaccaatggccgcgctagaggagaaaggcattttgttcacctctccattcgttctagcctcaaccaatgcaggatccatcaacgcaccaacagtttcagacagcagagccctagctagaaggttccactttgacatgaacattgaagttatctccatgtacagtcaaaatggaaaaataaacatgcccatgtcagtcaaaacatgtgatgaggaatgctgcccagtcaatttcaagagatgctgcccactggtgtgtggcaaggctattcagttcattgacagaagaacccaagtcagatattcactggatatgttagtcaccgagatgttcagggagtacaaccacaggcacagtgtgggcgccaccctcgaggctctgttccaaggtccaccggtctacagggagatcaagatcagtgttgctccagaaacacctcccccaccagcaatcgctgatttattaaaatcagtagacagtgaagctgtgagagagtactgcaaggaaaaaggctggcttgtaccggaagttaactccaccctacagattgagaagcacgtcagcagagcatttatctgtctacaagctctgaccactttcgtctcagtagctggcataatctacattatctacaagttgtttgccggctttcagggcgcgtatacggggatgccaaatcagaaacccaaagtgcccactctgagacaagctaaagtgcagggcccagcattcgagttcgctgtggcaatgatgaaaaggaacgccagcacagtgaaaacggaatatggtgagttcaccatgctcggcatctatgacagatgggcagtgctaccacgccacgccaagcctggaccgactattttgatgaatgatcaggagatcggcgtgttagacgccaaagaattagtggacaaagatgggacaaatctggagttgacccttttaaagctcaaccgcaatgagaagtttagggacatcagagggtttctggcgagagaggaagctgaagtgaatgaggctgttctggcaataaacacaagcaagttcccaaatatgtacatacccgtaggtcaagtcactgattacggtttcctgaacctggggggaacacccacaaagaggatgctcatgtacaactttccaactagagcagggcaatgtggtggggtactcatgtcaacagggaaagttcttggtatacatgtaggaggaaatggacatcaagggttctccgctgctcttcttaggcactactttaatgaggagcaaggtgaaatagagtttattgagagctcaaaggatgcaggattccctgtgatcaacacccccagcaaaacaaaactggaaccaagcgtgtttcatcaggtgtttgaaggcaataaagagccagcagtccttagaaatggtgatccgcgtcttaaagtaaactttgaagaagccatattttccaagtacatcgggaacatcaacacacatgtggatgaatacatgctcgaagcagtggatcactacgcagggcaactggctactcttgacattagcactgaacccatgaaactagaagatgcagtgtatggcactgaaggactagaggctcttgacttaacaacaagtgcagggtacccgtatgtcgcactaggcatcaaaaagagagacatcctatccaaaaagactaaagatctgactaaattgaaggaatgcatggacaagtacggtttgaacctgccgatggtgacttatgtgaaggatgaacttagatcatcagagaaagtggctaagggaaaatcaagactcattgaagcatctagtttaaatgactctgtcgcgatgaggcagacgtttggcaacttgtacaaaacattccatctgaacccgggcattgtgacaggcagtgcagttggatgtgatccggaccttttctggagtaaaatacccgtgatgttagacggccacctcatagcattcgattactccggatatgatgccagcctgagccctgtgtggttcgcttgtctaaaactgctactcgaaaaactcgggtacacgcacagagagacaaactacatcgactacctatgcaactctcaccacctgtacagagacaaacactactttgtgcgtggtggcatgccatcagggtgctccggtaccagcattttcaattcaatgatcaataacatcataatcaggacactgatgctcaaagtgtacaagggaattgacctggaccagtttagaatgattgcatatggtgatgatgtaattgcatcctacccttggcctatagacgcttcactacttgctgaagctgggaagggttatggactgattatgacaccagcagacaaaggggagtgcttcaatgaggttacctggaccaatgtcaccttcctaaagaggtactttagagctgatgagcagtatcctttcctggttcatccagttatgcccatgaaggacatccatgaatctattaggtggacaaaggatccaaagaacactcaagaccacgttcgatcgttgtgcttattggcttggcataacggagaacaagaatatgaggaattcatccgaaagatcagaagcgtcccggttgggcgctgtctgactctccccgcgttttcaactttacgcagaaaatggctggattccttttaaattagagacaatttgaaataatttaaattggcttaaccctactgtactaaccgaactagacaacggtgcagtaggggtaaattctccgcattcggtgcggaaaaaaaaaaaaaaaaaaaaaaaaaaaaaaaaaaaaaaaaaaaaaaaaaaa

SEQ ID NO: 97 - protein, 2193 aa, Echovirus 12; identical to SEQ ID NO: 85

SEQ ID NO: 98 - protein, 320 aa, Echovirus 12; identical to SEQ ID NO: 86

SEQ ID NO: 99 - protein, 291 aa, Echovirus 12; identical to SEQ ID NO: 87

SEQ ID NO: 100 - protein, 238 aa, Echovirus 12; identical to SEQ ID NO: 63

SEQ ID NO: 101 - protein, 121 aa, Echovirus 12; identical to SEQ ID NO: 65

SEQ ID NO: 102 - protein, 90 aa, Echovirus 12; identical to SEQ ID NO: 67

SEQ ID NO: 103 - protein, 183 aa, Echovirus 12; identical to SEQ ID NO: 69

SEQ ID NO: 104 - protein, 462 aa, Echovirus 12; identical to SEQ ID NO: 71

SEQ ID NO: 105 - RNA, 6579 nt, Echovirus 12; identical to SEQ ID NO: 93
  source 1..6579: mol_type = unassigned RNA

SEQ ID NO: 106 - RNA, 741 nt, Echovirus 12; identical to SEQ ID NO: 44
  source 1..741: mol_type = unassigned RNA

SEQ ID NO: 107 - RNA, 103 nt, Echovirus 12; identical to SEQ ID NO: 45
  source 1..103: mol_type = unassigned RNA

SEQ ID NO: 108 - RNA, 7474 nt, Echovirus 12; identical to SEQ ID NO: 50
  source 1..7474: mol_type = unassigned RNA

SEQ ID NO: 109 - protein, 2193 aa, Echovirus 12; identical to SEQ ID NO: 52

SEQ ID NO: 110 - protein, 320 aa, Echovirus 12; identical to SEQ ID NO: 57

SEQ ID NO: 111 - protein, 291 aa, Echovirus 12; identical to SEQ ID NO: 87

SEQ ID NO: 112 - protein, 238 aa, Echovirus 12; identical to SEQ ID NO: 63

SEQ ID NO: 113 - protein, 121 aa, Echovirus 12; identical to SEQ ID NO: 65

SEQ ID NO: 114 - protein, 90 aa, Echovirus 12; identical to SEQ ID NO: 67

SEQ ID NO: 115 - protein, 183 aa, Echovirus 12; identical to SEQ ID NO: 69

SEQ ID NO: 116 - protein, 462 aa, Echovirus 12; identical to SEQ ID NO: 71

SEQ ID NO: 117 - RNA, 6579 nt, Echovirus 12; identical to SEQ ID NO: 43
  source 1..6579: mol_type = unassigned RNA

SEQ ID NO: 118 - RNA, 741 nt, Echovirus 12; identical to SEQ ID NO: 44
  source 1..741: mol_type = unassigned RNA

SEQ ID NO: 119 - RNA, 103 nt, Echovirus 12; identical to SEQ ID NO: 45
  source 1..103: mol_type = unassigned RNA

SEQ ID NO: 120 - RNA, 7474 nt, Echovirus 12
  source 1..7474: mol_type = unassigned RNA
ttaaaacagcctgtgggttgtccccacccacagggcccactgggcgctagcacactggtatcccggtacctttgtgcgcctgttttatataccctcccctcagtaacttagaagttcatcacaaatgatcaatagttagctcaacaaaccagttgagcctagatcaagcacttctgttaccccgggctgagtatcaataagctgttgacacggctgaaggagaaaacgcccgttacccgaccagctacttcggagaacctagtatcaccatagaggttgcgtagcgtttcgctccgcacaaccccagtgtagatcaggtcgatgagtcaccgcgttccccacaggcgactgtggcggtggctgcgttggcggcctgcccatggggttacccatgggacgcttcaatactgacatggtgtgaagagttgactgagctagctggtagtcctccggcccctgaatgcggctaatcctaactgtggagcaagtgcccacaacccagtgggtggcttgtcgtaatgggcaactctgcagcggaaccgactactttgggtgaccgtgtttctctttattcttatattggctgcttatggtgacaatctcagagttgttaccatatagctattggtttggccaaccagtgactaacagagcaattatatatcttttcattggatttatacctataaacaacaccagttataccactttgtgttacattattggcttgaactcgaaaagatgggagcgcaagtatcaacacaaaaaactggggcacacgagactggcctgagtgctagtggaaattccatcatccactataccaacattaattactacaaagatgcggcatccaattcagcaaacaggcaggatttcacccaagaccccgggaaattcaccgagccagtgaaggatatcatgataaaatcgatgcctgcgttaaactccccaacagcagaagaatgtgggtacagcgatagagtcagatcaatcaccctggggaactctaccatcaccacgcaggagtgtgccaatgtggtggtgggatatggcacgtggcctgattatctacatgatgatgaggccactgcagaggaccaaccaacacaaccggatgtggcgacgtgtaggttttacacacttgaatccattcagtggcagaagacatcagatgggtggtggtggaagtttccggaagcattgaaagacatgggactgttcggccaaaacatgcactatcattatcttggcaggtcgggttacaccatccacgtccaatgtaacgcgtctaaattccaccagggttgtttgcttgtggtgtgtgtgcctgaagctgagatggggtgcgctacagtggcgaatgaggttaccgcagccgctctctcatctggtgaaacggcaaaatattttgcaaagactggtgcgacgggaacccacacagtgcaaaacatagtgacaaacgccggcatgggggtgggcgtgggcaatctgacaatattcccacatcagtggataaacctccgtaccaacaacagcgcgactattgtcatgccttacataaacagtgtgcccatggataacatgttcagacactacaatttcaccctaatgatcatccccttcgtcccactggacttcacagctgaggcatctacgtatgtaccaatcactgtaacagtagctcccatgtgcgccgagtacaacgggctacgcctggcctcgcatcaaggtttgccgacaatgaacacccctggtagtaaccaattcctcacatcagatgactttcaatcaccatcagccatgccacaattcgatgtcacacccgagctcaggattccaggggaggtaaagaacttaatggaaatagccgaagttgactccgtagtgcctgtgaacaatacacaggactcagtgtacaacatggatgtgtacaaaattcctgttagtggaggcaaccaactgtcaactcaagtcttcgggttccaaatgcaaccagggctaaatagtgtgttcaaaaggacattactaggtgagatactgaactactatgcacactggtctggtagtgtaaagctcacctttgtgttctgtggatcggccatggccactggcaaatttctgctggcatattcccctccaggcgcagaccccccaaagtctaggaaagaagcaatgcttggtactcacgtgatatgggatataggacttcagtcaagctgcgtcttgtgcgtaccgtggatcagccaaacacattatagattggttcagcaggatgaatatacgagtgcaggatatgtaacttgctggtatcaaactagtttggttgtcccgcccggcgctcccgtcacgtgtggagtcttgtgcttagcatcggcgtgcaatgatttctctgtgagaatgttgagagacacaccttttatcgagcaaaaacagctactccaaggagatgtggaggaggccgtgaacagagccgttgcacgggtggcggatacactacccacagggccgagaaattcagagagcatccccgcactaacagcagcagaaactgggcatacatctcaggtggttcctggggataccatgcaaacccgtcatgtgaagaattaccactccagaactgaatcgtctgttgaaaacttcttatgccgagcagcatgtgtgtatattacaaagtacaagaccaaggacagtgatccagtgcaaaggtacgccaactggcggatcaacacccgtcaaatggttcagctaaggagaaaatttgagttgtttacatacctaaggtttgatatggaagttacttttgtgatcaccagttcacaagatgatggcacgcagttggcgcaggacatgccagtattaacacaccaagtcatgtatataccacctggagggccagtgccgaatagtgttactgattttgcgtggcaatcgtctaccaaccctagcatcttctggacggagggaaacgctccagctaggatgtccatcccctttatcagtataggcaacgcgtatagcaatttctatgatggttggtcacatttcacccaagatggagtatatggttacaactccctcaataacatgggctccatctacattaggcatgtcaatgaacaaagcccgcatgcaattacgagcacagtcagagtctatttcaaaccaaaacacgtgcgggcctgggtgccaagaccacctaggctttgtgcatatgagaaatcaagcaacgtgaatttcaaaccaacagatgtgactacctcccgaacatccatcacagaagtccctagtcttagaccatcagtggtaaatactggagctttcggtcagcaatcaggagcggcttatgtgggaaactatagagtggttaatagacacctagccactcatgttgactggcagaactgtgtgtgggaggactataacagggacctccttgtaagcaccaccacagctcatgggtgtgacaccatagccagatgccaatgcacaacaggcgtgtacttctgtgcatcgaggaataaacactacccagtcagttttgaaggaccaggtctggtagaagtccaggagagcgagtactaccccaggaggtatcaatcacacgtcttgctggctgcgggattttcagagccaggagattgtggagggattctcagatgtgagcacggtgtcatcggtctagttactatgggtggcgaaggcgttgtcggattcgctgatgtacgcgacctgttatggcttgaagatgacgccatggaacaaggagtcaaggattacgtggaacaactagggaacgcctttggttcaggattcactaaccaaatctgtgagcaggtcaacctcctaaaagagtcactggtgggtcacgattccatcctagagaagtcccttaaagccctagtgaaaatcatatcagcactagtgatagtggtgaggaaccatgatgatttgatcactgtaactgctactcttgccctcattggttgtacctcctctccatggcggtggctcaagcacaaggtgtcacaatactacgggatacccatggcagagcgccaaagcaacgggtggctcaagaagttcacagagatgaccaacgcctgcaaagggatggaatggattgccatcaaaatccaaaagttcatagagtggcttaaacttaagattttaccagaagttaaggaaaaacatgaattcctgaataggctcaagcaactcccactgttggagagtcaaatagcaaccatcgagcagagtgcaccgtcccagagcgatcaggagcagctcttttcaaatgttcagtactttgcccattattgcagaaaatatgcgcccctatacgcagctgaggcaaaaagggtgttctctcttgaaaagaaaatgagtaattacatacagttcaagtccaaatgccgtattgaacctgtatgcttgctactacatgggagccccggagcagggaaatcagttgctaccagcctcattgggcgatcactagctgaaaaattgaatagctcagtgtactccttaccaccagacccagatcactttgatggttacaagcaacaagctgtagtgatcatggacgatctgtgccaaaatccagatggaaaagatgtgtctttgttctgtcaaatggtgtctagtgtagattttgtaccaccaatggccgcgctagaggagaaaggcattttgttcacctctccattcgttctagcctcaaccaatgcaggatccatcaacgcaccaacagtttcagacagcagagccctagctagaaggttccactttgacatgaacattgaagttatctccatgtacagtcaaaatggaaaaataaacatgcccatgtcagtcaaaacatgtgatgaggaatgctgcccagtcaatttcaagagatgctgcccactggtgtgtggcaaggctattcagttcattgacagaagaacccaagtcagatattcactggatatgttagtcaccgagatgttcagggagtacaaccacaggcacagtgtgggcgccaccctcgaggctctgttccaaggtccaccggtctacagggagatcaagatcagtgttgctccagaaacacctcccccaccagcaatcgctgatttattaaaatcagtagacagtgaagctgtgagagagtactgcaaggaaaaaggctggcttgtaccggaagttaactccaccctacagattgagaagcacgtcagcagagcatttatctgtctacaagctctgaccactttcgtctcagtagctggcataatctacattatctacaagttgtttgccggctttcagggcgcgtatacggggatgccaaatcagaaacccaaagtgcccactctgagacaagctaaagtgcagggcccagcattcgagttcgctgtggcaatgatgaaaaggaacgccagcacagtgaaaacggaatatggtgagttcaccatgctcggcatctatgacagatgggcagtgctaccacgccacgccaagcctggaccgactattttgatgaatgatcaggagatcggcgtgttagacgccaaagaattagtggacaaagatgggacaaatctggagttgacccttttaaagctcaaccgcaatgagaagtttagggacatcagagggtttctggcgagagaggaagctgaagtgaatgaggctgttctggcaataaacacaagcaagttcccaaatatgtacatacccgtaggtcaagtcactgattacggtttcctgaacctggggggaacacccacaaagaggatgctcatgtacaactttccaactagagcagggcaatgtggtggggtactcatgtcaacagggaaagttcttggtatacatgtaggaggaaatggacatcaagggttctccgctgctcttcttaggcactactttaatgaggagcaaggtgaaatagagtttattgagagctcaaaggatgcaggattccctgtgatcaacacccccagcaaaacaaaactggaaccaagcgtgtttcatcaggtgtttgaaggcaataaagagccagcagtccttagaaatggtgatccgcgtcttaaagtaaactttgaagaagccatattttccaagtacatcgggaacatcaacacacatgtggatgaatacatgctcgaagcagtggatcactacgcagggcaactggctactcttgacattagcactgaacccatgaaactagaagatgcagtgtatggcactgaaggactagaggctcttgacttaacaacaagtgcagggtacccgtatgtcgcactaggcatcaaaaagagagacatcctatccaaaaagactaaagatctgactaaattgaaggaatgcatggacaagtacggtttgaacctgccgatggtgacttatgtgaaggatgaacttagatcatcagagaaagtggctaagggaaaatcaagactcattgaagcatctagtttaaatgactctgtcgcgatgaggcagacgtttggcaacttgtacaaaacattccatctgaacccgggcattgtgacaggcagtgcagttggatgtgatccggaccttttctggagtaaaatacccgtgatgttagacggccacctcatagcattcgattactccggatatgatgccagcctgagccctgtgtggttcgcttgtctaaaactgctactcgaaaaactcgggtacacgcacagagagacaaactacatcgactacctatgcaactctcaccacctgtacagagacaaacactactttgtgcgtggtggcatgccatcagggtgctccggtaccagcattttcaattcaatgatcaataacatcataatcaggacactgatgctcaaagtgtacaagggaattgacctggaccagtttagaatgattgcatatggtgatgatgtaattgcatcctacccttggcctatagacgcttcactacttgctgaagctgggaagggttatggactgattatgacaccagcagacaaaggggagtgcttcaatgaggttacctggaccaatgtcaccttcctaaagaggtactttagagctgatgagcagtatcctttcctggttcatccagttatgcccatgaaggacatccatgaatctattaggtggacaaaggatccaaagaacactcaagaccacgttcgatcgttgtgcttattggcttggcataacggagaacaagaatatgaggaattcatccgaaagatcagaagcgtcccggttgggcgctgtctgactctccccgcgttttcaactttacgcagaaaatggctggattccttttaaattagagacaatttgaaataatttaaattggcttaaccctactgtactaaccgaactagacaacggtgcagtaggggtaaattctccgcattcggtgcggaaaaaaaaaaaaaaaaaaaaaaaaaaaaaaaaaaaaaaaaaaaaaaaaaaa

SEQ ID NO: 121 - protein, 2193 aa, Echovirus 12
MGAQVSTQKTGAHETGLSASGNSIIHYTNINYYKDAASNSANRQDFTQDPGKFTEPVKDIMIKSMPALNSPTAEECGYSDRVRSITLGNSTITTQECANVVVGYGTWPDYLHDDEATAEDQPTQPDVATCRFYTLESIQWQKTSDGWWWKFPEALKDMGLFGQNMHYHYLGRSGYTIHVQCNASKFHQGCLLVVCVPEAEMGCATVANEVTAAALSSGETAKYFAKTGATGTHTVQNIVTNAGMGVGVGNLTIFPHQWINLRTNNSATIVMPYINSVPMDNMFRHYNFTLMIIPFVPLDFTAEASTYVPITVTVAPMCAEYNGLRLASHQGLPTMNTPGSNQFLTSDDFQSPSAMPQFDVTPELRIPGEVKNLMEIAEVDSVVPVNNTQDSVYNMDVYKIPVSGGNQLSTQVFGFQMQPGLNSVFKRTLLGEILNYYAHWSGSVKLTFVFCGSAMATGKFLLAYSPPGADPPKSRKEAMLGTHVIWDIGLQSSCVLCVPWISQTHYRLVQQDEYTSAGYVTCWYQTSLVVPPGAPVTCGVLCLASACNDFSVRMLRDTPFIEQKQLLQGDVEEAVNRAVARVADTLPTGPRNSESIPALTAAETGHTSQVVPGDTMQTRHVKNYHSRTESSVENFLCRAACVYITKYKTKDSDPVQRYANWRINTRQMVQLRRKFELFTYLRFDMEVTFVITSSQDDGTQLAQDMPVLTHQVMYIPPGGPVPNSVTDFAWQSSTNPSIFWTEGNAPARMSIPFISIGNAYSNFYDGWSHFTQDGVYGYNSLNNMGSIYIRHVNEQSPHAITSTVRVYFKPKHVRAWVPRPPRLCAYEKSSNVNFKPTDVTTSRTSITEVPSLRPSVVNTGAFGQQSGAAYVGNYRVVNRHLATHVDWQNCVWEDYNRDLLVSTTTAHGCDTIARCQCTTGVYFCASRNKHYPVSFEGPGLVEVQESEYYPRRYQSHVLLAAGFSEPGDCGGILRCEHGVIGLVTMGGEGVVGFADVRDLLWLEDDAMEQGVKDYVEQLGNAFGSGFTNQICEQVNLLKESLVGHDSILEKSLKALVKIISALVIVVRNHDDLITVTATLALIGCTSSPWRWLKHKVSQYYGIPMAERQSNGWLKKFTEMTNACKGMEWIAIKIQKFIEWLKLKILPEVKEKHEFLNRLKQLPLLESQIATIEQSAPSQSDQEQLFSNVQYFAHYCRKYAPLYAAEAKRVFSLEKKMSNYIQFKSKCRIEPVCLLLHGSPGAGKSVATSLIGRSLAEKLNSSVYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYSQNGKINMPMSVKTCDEECCPVNFKRCCPLVCGKAIQFIDRRTQVRYSLDMLVTEMFREYNHRHSVGATLEALFQGPPVYREIKISVAPETPPPPAIADLLKSVDSEAVREYCKEKGWLVPEVNSTLQIEKHVSRAFICLQALTTFVSVAGIIYIIYKLFAGFQGAYTGMPNQKPKVPTLRQAKVQGPAFEFAVAMMKRNASTVKTEYGEFTMLGIYDRWAVLPRHAKPGPTILMNDQEIGVLDAKELVDKDGTNLELTLLKLNRNEKFRDIRGFLAREEAEVNEAVLAINTSKFPNMYIPVGQVTDYGFLNLGGTPTKRMLMYNFPTRAGQCGGVLMSTGKVLGIHVGGNGHQGFSAALLRHYFNEEQGEIEFIESSKDAGFPVINTPSKTKLEPSVFHQVFEGNKEPAVLRNGDPRLKVNFEEAIFSKYIGNINTHVDEYMLEAVDHYAGQLATLDISTEPMKLEDAVYGTEGLEALDLTTSAGYPYVALGIKKRDILSKKTKDLTKLKECMDKYGLNLPMVTYVKDELRSSEKVAKGKSRLIEASSLNDSVAMRQTFGNLYKTFHLNPGIVTGSAVGCDPDLFWSKIPVMLDGHLIAFDYSGYDASLSPVWFACLKLLLEKLGYTHRETNYIDYLCNSHHLYRDKHYFVRGGMPSGCSGTSIFNSMINNIIIRTLMLKVYKGIDLDQFRMIAYGDDVIASYPWPIDASLLAEAGKGYGLIMTPADKGECFNEVTWTNVTFLKRYFRADEQYPFLVHPVMPMKDIHESIRWTKDPKNTQDHVRSLCLLAWHNGEQEYEEFIRKIRSVPVGRCLTLPAFSTLRRKWLDSF

SEQ ID NO: 122 - protein, 320 aa, Echovirus 12; identical to SEQ ID NO: 57

SEQ ID NO: 123 - protein, 291 aa, Echovirus 12
SPTAEECGYSDRVRSITLGNSTITTQECANVVVGYGTWPDYLHDDEATAEDQPTQPDVATCRFYTLESIQWQKTSDGWWWKFPEALKDMGLFGQNMHYHYLGRSGYTIHVQCNASKFHQGCLLVVCVPEAEMGCATVANEVTAAALSSGETAKYFAKTGATGTHTVQNIVTNAGMGVGVGNLTIFPHQWINLRTNNSATIVMPYINSVPMDNMFRHYNFTLMIIPFVPLDFTAEASTYVPITVTVAPMCAEYNGLRLASHQGLPTMNTPGSNQFLTSDDFQSPSAMPQFDV

SEQ ID NO: 124 - protein, 238 aa, Echovirus 12; identical to SEQ ID NO: 63

SEQ ID NO: 125 - protein, 121 aa, Echovirus 12; identical to SEQ ID NO: 65

SEQ ID NO: 126 - protein, 90 aa, Echovirus 12; identical to SEQ ID NO: 67

SEQ ID NO: 127 - protein, 183 aa, Echovirus 12; identical to SEQ ID NO: 69

SEQ ID NO: 128 - protein, 462 aa, Echovirus 12; identical to SEQ ID NO: 71

SEQ ID NO: 129 - RNA, 6579 nt, Echovirus 12
  source 1..6579: mol_type = unassigned RNA
atgggagcgcaagtatcaacacaaaaaactggggcacacgagactggcctgagtgctagtggaaattccatcatccactataccaacattaattactacaaagatgcggcatccaattcagcaaacaggcaggatttcacccaagaccccgggaaattcaccgagccagtgaaggatatcatgataaaatcgatgcctgcgttaaactccccaacagcagaagaatgtgggtacagcgatagagtcagatcaatcaccctggggaactctaccatcaccacgcaggagtgtgccaatgtggtggtgggatatggcacgtggcctgattatctacatgatgatgaggccactgcagaggaccaaccaacacaaccggatgtggcgacgtgtaggttttacacacttgaatccattcagtggcagaagacatcagatgggtggtggtggaagtttccggaagcattgaaagacatgggactgttcggccaaaacatgcactatcattatcttggcaggtcgggttacaccatccacgtccaatgtaacgcgtctaaattccaccagggttgtttgcttgtggtgtgtgtgcctgaagctgagatggggtgcgctacagtggcgaatgaggttaccgcagccgctctctcatctggtgaaacggcaaaatattttgcaaagactggtgcgacgggaacccacacagtgcaaaacatagtgacaaacgccggcatgggggtgggcgtgggcaatctgacaatattcccacatcagtggataaacctccgtaccaacaacagcgcgactattgtcatgccttacataaacagtgtgcccatggataacatgttcagacactacaatttcaccctaatgatcatccccttcgtcccactggacttcacagctgaggcatctacgtatgtaccaatcactgtaacagtagctcccatgtgcgccgagtacaacgggctacgcctggcctcgcatcaaggtttgccgacaatgaacacccctggtagtaaccaattcctcacatcagatgactttcaatcaccatcagccatgccacaattcgatgtcacacccgagctcaggattccaggggaggtaaagaacttaatggaaatagccgaagttgactccgtagtgcctgtgaacaatacacaggactcagtgtacaacatggatgtgtacaaaattcctgttagtggaggcaaccaactgtcaactcaagtcttcgggttccaaatgcaaccagggctaaatagtgtgttcaaaaggacattactaggtgagatactgaactactatgcacactggtctggtagtgtaaagctcacctttgtgttctgtggatcggccatggccactggcaaatttctgctggcatattcccctccaggcgcagaccccccaaagtctaggaaagaagcaatgcttggtactcacgtgatatgggatataggacttcagtcaagctgcgtcttgtgcgtaccgtggatcagccaaacacattatagattggttcagcaggatgaatatacgagtgcaggatatgtaacttgctggtatcaaactagtttggttgtcccgcccggcgctcccgtcacgtgtggagtcttgtgcttagcatcggcgtgcaatgatttctctgtgagaatgttgagagacacaccttttatcgagcaaaaacagctactccaaggagatgtggaggaggccgtgaacagagccgttgcacgggtggcggatacactacccacagggccgagaaattcagagagcatccccgcactaacagcagcagaaactgggcatacatctcaggtggttcctggggataccatgcaaacccgtcatgtgaagaattaccactccagaactgaatcgtctgttgaaaacttcttatgccgagcagcatgtgtgtatattacaaagtacaagaccaaggacagtgatccagtgcaaaggtacgccaactggcggatcaacacccgtcaaatggttcagctaaggagaaaatttgagttgtttacatacctaaggtttgatatggaagttacttttgtgatcaccagttcacaagatgatggcacgcagttggcgcaggacatgccagtattaacacaccaagtcatgtatataccacctggagggccagtgccgaatagtgttactgattttgcgtggcaatcgtctaccaaccctagcatcttctggacggagggaaacgctccagctaggatgtccatcccctttatcagtataggcaacgcgtatagcaatttctatgatggttggtcacatttcacccaagatggagtatatggttacaactccctcaataacatgggctccatctacattaggcatgtcaatgaacaaagcccgcatgcaattacgagcacagtcagagtctatttcaaaccaaaacacgtgcgggcctgggtgccaagaccacctaggctttgtgcatatgagaaatcaagcaacgtgaatttcaaaccaacagatgtgactacctcccgaacatccatcacagaagtccctagtcttagaccatcagtggtaaatactggagctttcggtcagcaatcaggagcggcttatgtgggaaactatagagtggttaatagacacctagccactcatgttgactggcagaactgtgtgtgggaggactataacagggacctccttgtaagcaccaccacagctcatgggtgtgacaccatagccagatgccaatgcacaacaggcgtgtacttctgtgcatcgaggaataaacactacccagtcagttttgaaggaccaggtctggtagaagtccaggagagcgagtactaccccaggaggtatcaatcacacgtcttgctggctgcgggattttcagagccaggagattgtggagggattctcagatgtgagcacggtgtcatcggtctagttactatgggtggcgaaggcgttgtcggattcgctgatgtacgcgacctgttatggcttgaagatgacgccatggaacaaggagtcaaggattacgtggaacaactagggaacgcctttggttcaggattcactaaccaaatctgtgagcaggtcaacctcctaaaagagtcactggtgggtcacgattccatcctagagaagtcccttaaagccctagtgaaaatcatatcagcactagtgatagtggtgaggaaccatgatgatttgatcactgtaactgctactcttgccctcattggttgtacctcctctccatggcggtggctcaagcacaaggtgtcacaatactacgggatacccatggcagagcgccaaagcaacgggtggctcaagaagttcacagagatgaccaacgcctgcaaagggatggaatggattgccatcaaaatccaaaagttcatagagtggcttaaacttaagattttaccagaagttaaggaaaaacatgaattcctgaataggctcaagcaactcccactgttggagagtcaaatagcaaccatcgagcagagtgcaccgtcccagagcgatcaggagcagctcttttcaaatgttcagtactttgcccattattgcagaaaatatgcgcccctatacgcagctgaggcaaaaagggtgttctctcttgaaaagaaaatgagtaattacatacagttcaagtccaaatgccgtattgaacctgtatgcttgctactacatgggagccccggagcagggaaatcagttgctaccagcctcattgggcgatcactagctgaaaaattgaatagctcagtgtactccttaccaccagacccagatcactttgatggttacaagcaacaagctgtagtgatcatggacgatctgtgccaaaatccagatggaaaagatgtgtctttgttctgtcaaatggtgtctagtgtagattttgtaccaccaatggccgcgctagaggagaaaggcattttgttcacctctccattcgttctagcctcaaccaatgcaggatccatcaacgcaccaacagtttcagacagcagagccctagctagaaggttccactttgacatgaacattgaagttatctccatgtacagtcaaaatggaaaaataaacatgcccatgtcagtcaaaacatgtgatgaggaatgctgcccagtcaatttcaagagatgctgcccactggtgtgtggcaaggctattcagttcattgacagaagaacccaagtcagatattcactggatatgttagtcaccgagatgttcagggagtacaaccacaggcacagtgtgggcgccaccctcgaggctctgttccaaggtccaccggtctacagggagatcaagatcagtgttgctccagaaacacctcccccaccagcaatcgctgatttattaaaatcagtagacagtgaagctgtgagagagtactgcaaggaaaaaggctggcttgtaccggaagttaactccaccctacagattgagaagcacgtcagcagagcatttatctgtctacaagctctgaccactttcgtctcagtagctggcataatctacattatctacaagttgtttgccggctttcagggcgcgtatacggggatgccaaatcagaaacccaaagtgcccactctgagacaagctaaagtgcagggcccagcattcgagttcgctgtggcaatgatgaaaaggaacgccagcacagtgaaaacggaatatggtgagttcaccatgctcggcatctatgacagatgggcagtgctaccacgccacgccaagcctggaccgactattttgatgaatgatcaggagatcggcgtgttagacgccaaagaattagtggacaaagatgggacaaatctggagttgacccttttaaagctcaaccgcaatgagaagtttagggacatcagagggtttctggcgagagaggaagctgaagtgaatgaggctgttctggcaataaacacaagcaagttcccaaatatgtacatacccgtaggtcaagtcactgattacggtttcctgaacctggggggaacacccacaaagaggatgctcatgtacaactttccaactagagcagggcaatgtggtggggtactcatgtcaacagggaaagttcttggtatacatgtaggaggaaatggacatcaagggttctccgctgctcttcttaggcactactttaatgaggagcaaggtgaaatagagtttattgagagctcaaaggatgcaggattccctgtgatcaacacccccagcaaaacaaaactggaaccaagcgtgtttcatcaggtgtttgaaggcaataaagagccagcagtccttagaaatggtgatccgcgtcttaaagtaaactttgaagaagccatattttccaagtacatcgggaacatcaacacacatgtggatgaatacatgctcgaagcagtggatcactacgcagggcaactggctactcttgacattagcactgaacccatgaaactagaagatgcagtgtatggcactgaaggactagaggctcttgacttaacaacaagtgcagggtacccgtatgtcgcactaggcatcaaaaagagagacatcctatccaaaaagactaaagatctgactaaattgaaggaatgcatggacaagtacggtttgaacctgccgatggtgacttatgtgaaggatgaacttagatcatcagagaaagtggctaagggaaaatcaagactcattgaagcatctagtttaaatgactctgtcgcgatgaggcagacgtttggcaacttgtacaaaacattccatctgaacccgggcattgtgacaggcagtgcagttggatgtgatccggaccttttctggagtaaaatacccgtgatgttagacggccacctcatagcattcgattactccggatatgatgccagcctgagccctgtgtggttcgcttgtctaaaactgctactcgaaaaactcgggtacacgcacagagagacaaactacatcgactacctatgcaactctcaccacctgtacagagacaaacactactttgtgcgtggtggcatgccatcagggtgctccggtaccagcattttcaattcaatgatcaataacatcataatcaggacactgatgctcaaagtgtacaagggaattgacctggaccagtttagaatgattgcatatggtgatgatgtaattgcatcctacccttggcctatagacgcttcactacttgctgaagctgggaagggttatggactgattatgacaccagcagacaaaggggagtgcttcaatgaggttacctggaccaatgtcaccttcctaaagaggtactttagagctgatgagcagtatcctttcctggttcatccagttatgcccatgaaggacatccatgaatctattaggtggacaaaggatccaaagaacactcaagaccacgttcgatcgttgtgcttattggcttggcataacggagaacaagaatatgaggaattcatccgaaagatcagaagcgtcccggttgggcgctgtctgactctccccgcgttttcaactttacgcagaaaatggctggattccttt

SEQ ID NO: 130 - RNA, 741 nt, Echovirus 12; identical to SEQ ID NO: 44
  source 1..741: mol_type = unassigned RNA

SEQ ID NO: 131 - RNA, 103 nt, Echovirus 12; identical to SEQ ID NO: 45
  source 1..103: mol_type = unassigned RNA

SEQ ID NO: 132 - RNA, 6579 nt, Echovirus 12
  source 1..6579: mol_type = unassigned RNA
atgggagcgcaagtatcaacacaaaaaactggggcacacgagactggcctgagtgctagtggaaattccatcatccactataccaacattaattactacaaagatgcggcatccaattcagcaaacaggcaggatttcacccaagaccccgggaaattcaccgagccagtgaaggatatcatgataaaatcgatgcctgcgttaaactccccaacagcagaagaatgtgggtacagcgatagagtcagatcaatcaccctggggaactctaccatcaccacgcaggagtgtgccaatgtggtggtgggatatggcacgtggcctgattatctacatgatgatgaggccactgcagaggaccaaccaacacaaccggatgtggcgacgtgtaggttttacacacttgaatccattcagtggcagaagacatcagatgggtggtggtggaagtttccggaagcattgaaagacatgggactgttcggccaaaacatgcactatcattatcttggcaggtcgggttacaccatccacgtccaatgtaacgcgtctaaattccaccagggttgtttgcttgtggtgtgtgtgcctgaagctgagatggggtgcgctacagtggcgaatgaggttaccgcagccgctctctcatctggtgaaacggcaaaatattttgcaaagactggtgcgacgggaacccacacagtgcaaaacatagtgacaaacgccggcatgggggtgggcgtgggcaatctgacaatattcccacatcagtggataaacctccgtaccaacaacagcgcgactattgtcatgccttacataaacagtgtgcccatggataacatgttcagacactacaatttcaccctaatgatcatccccttcgtcccactggacttcacagctgaggcatctacgtatgtaccaatcactgtaacagtagctcccatgtgcgccgagtacaacgggctacgcctggcctcgcatcaaggtttgccgacaatgaacacccctggtagtaaccaattcctcacatcagatgactttcaatcaccatcagccatgccacaattcgatgtcacacccgagctcaggattccaggggaggtaaagaacttaatggaaatagccgaagttgactccgtagtgcctgtgaacaatacacaggactcagtgtacaacatggatgtgtacaaaattcctgttagtggaggcaaccaactgtcaactcaagtcttcgggttccaaatgcaaccagggctaaatagtgtgttcaaaaggacattactaggtgagatactgaactactatgcacactggtctggtagtgtaaagctcacctttgtgttctgtggatcggccatggccactggcaaatttctgctggcatattcccctccaggcgcagaccccccaaagtctaggaaagaagcaatgcttggtactcacgtgatatgggatataggacttcagtcaagctgcgtcttgtgcgtaccgtggatcagccaaacacattatagattggttcagcaggatgaatatacgagtgcaggatatgtaacttgctggtatcaaactagtttggttgtcccgcccggcgctcccgtcacgtgtggagtcttgtgcttagcatcggcgtgcaatgatttctctgtgagaatgttgagagacacaccttttatcgagcaaaaacagctactccaaggagatgtggaggaggccgtgaacagagccgttgcacgggtggcggatacactacccacagggccgagaaattcagagagcatccccgcactaacagcagcagaaactgggcatacatctcaggtggttcctggggataccatgcaaacccgtcatgtgaagaattaccactccagaactgaatcgtctgttgaaaacttcttatgccgagcagcatgtgtgtatattacaaagtacaagaccaaggacagtgatccagtgcaaaggtacgccaactggcggatcaacacccgtcaaatggttcagctaaggagaaaatttgagttgtttacatacctaaggtttgatatggaagttacttttgtgatcaccagttcacaagatgatggcacgcagttggcgcaggacatgccagtattaacacaccaagtcatgtatataccacctggagggccagtgccgaatagtgttactgattttgcgtggcaatcgtctaccaaccctagcatcttctggacggagggaaacgctccagctaggatgtccatcccctttatcagtataggcaacgcgtatagcaatttctatgatggttggtcacatttcacccaagatggagtatatggtttcaactccctcaataacatgggctccatctacattaggcatgtcaatgaacaaagcccgcatgcaattacgagcacagtcagagtctatttcaaaccaaaacacgtgcgggcctgggtgccaagaccacctaggctttgtgcatatgagaaatcaagcaacgtgaatttcaaaccaacagatgtgactacctcccgaacatccatcacagaagtccctagtcttagaccatcagtggtaaatactggagctttcggtcagcaatcaggagcggcttatgtgggaaactatagagtggttaatagacacctagccactcatgttgactggcagaactgtgtgtgggaggactataacagggacctccttgtaagcaccaccacagctcatgggtgtgacaccatagccagatgccaatgcacaacaggcgtgtacttctgtgcatcgaggaataaacactacccagtcagttttgaaggaccaggtctggtagaagtccaggagagcgagtactaccccaggaggtatcaatcacacgtcttgctggctgcgggattttcagagccaggagattgtggagggattctcagatgtgagcacggtgtcatcggtctagttactatgggtggcgaaggcgttgtcggattcgctgatgtacgcgacctgttatggcttgaagatgacgccatggaacaaggagtcaaggattacgtggaacaactagggaacgcctttggttcaggattcactaaccaaatctgtgagcaggtcaacctcctaaaagagtcactggtgggtcacgattccatcctagagaagtcccttaaagccctagtgaaaatcatatcagcactagtgatagtggtgaggaaccatgatgatttgatcactgtaactgctactcttgccctcattggttgtacctcctctccatggcggtggctcaagcacaaggtgtcacaatactacgggatacccatggcagagcgccaaagcaacgggtggctcaagaagttcacagagatgaccaacgcctgcaaagggatggaatggattgccatcaaaatccaaaagttcatagagtggcttaaacttaagattttaccagaagttaaggaaaaacatgaattcctgaataggctcaagcaactcccactgttggagagtcaaatagcaaccatcgagcagagtgcaccgtcccagagcgatcaggagcagctcttttcaaatgttcagtactttgcccattattgcagaaaatatgcgcccctatacgcagctgaggcaaaaagggtgttctctcttgaaaagaaaatgagtaattacatacagttcaagtccaaatgccgtattgaacctgtatgcttgctactacatgggagccccggagcagggaaatcagttgctaccagcctcattgggcgatcactagctgaaaaattgaatagctcagtgtactccttaccaccagacccagatcactttgatggttacaagcaacaagctgtagtgatcatggacgatctgtgccaaaatccagatggaaaagatgtgtctttgttctgtcaaatggtgtctagtgtagattttgtaccaccaatggccgcgctagaggagaaaggcattttgttcacctctccattcgttctagcctcaaccaatgcaggatccatcaacgcaccaacagtttcagacagcagagccctagctagaaggttccactttgacatgaacattgaagttatctccatgtacagtcaaaatggaaaaataaacatgcccatgtcagtcaaaacatgtgatgaggaatgctgcccagtcaatttcaagagatgctgcccactggtgtgtggcaaggctattcagttcattgacagaagaacccaagtcagatattcactggatatgttagtcaccgagatgttcagggagtacaaccacaggcacagtgtgggcgccaccctcgaggctctgttccaaggtccaccggtctacagggagatcaagatcagtgttgctccagaaacacctcccccaccagcaatcgctgatttattaaaatcagtagacagtgaagctgtgagagagtactgcaaggaaaaaggctggcttgtaccggaagttaactccaccctacagattgagaagcacgtcagcagagcatttatctgtctacaagctctgaccactttcgtctcagtagctggcataatctacattatctacaagttgtttgccggctttcagggcgcgtatacggggatgccaaatcagaaacccaaagtgcccactctgagacaagctaaagtgcagggcccagcattcgagttcgctgtggcaatgatgaaaaggaacgccagcacagtgaaaacggaatatggtgagttcaccatgctcggcatctatgacagatgggcagtgctaccacgccacgccaagcctggaccgactattttgatgaatgatcaggagatcggcgtgttagacgccaaagaattagtggacaaagatgggacaaatctggagttgacccttttaaagctcaaccgcaatgagaagtttagggacatcagagggtttctggcgagagaggaagctgaagtgaatgaggctgttctggcaataaacacaagcaagttcccaaatatgtacatacccgtaggtcaagtcactgattacggtttcctgaacctggggggaacacccacaaagaggatgctcatgtacaactttccaactagagcagggcaatgtggtggggtactcatgtcaacagggaaagttcttggtatacatgtaggaggaaatggacatcaagggttctccgctgctcttcttaggcactactttaatgaggagcaaggtgaaatagagtttattgagagctcaaaggatgcaggattccctgtgatcaacacccccagcaaaacaaaactggaaccaagcgtgtttcatcaggtgtttgaaggcaataaagagccagcagtccttagaaatggtgatccgcgtcttaaagtaaactttgaagaagccatattttccaagtacatcgggaacatcaacacacatgtggatgaatacatgctcgaagcagtggatcactacgcagggcaactggctactcttgacattagcactgaacccatgaaactagaagatgcagtgtatggcactgaaggactagaggctcttgacttaacaacaagtgcagggtacccgtatgtcgcactaggcatcaaaaagagagacatcctatccaaaaagactaaagatctgactaaattgaaggaatgcatggacaagtacggtttgaacctgccgatggtgacttatgtgaaggatgaacttagatcatcagagaaagtggctaagggaaaatcaagactcattgaagcatctagtttaaatgactctgtcgcgatgaggcagacgtttggcaacttgtacaaaacattccatctgaacccgggcattgtgacaggcagtgcagttggatgtgatccggaccttttctggagtaaaatacccgtgatgttagacggccacctcatagcattcgattactccggatatgatgccagcctgagccctgtgtggttcgcttgtctaaaactgctactcgaaaaactcgggtacacgcacagagagacaaactacatcgactacctatgcaactctcaccacctgtacagagacaaacactactttgtgcgtggtggcatgccatcagggtgctccggtaccagcattttcaattcaatgatcaataacatcataatcaggacactgatgctcaaagtgtacaagggaattgacctggaccagtttagaatgattgcatatggtgatgatgtaattgcatcctacccttggcctatagacgcttcactacttgctgaagctgggaagggttatggactgattatgacaccagcagacaaaggggagtgcttcaatgaggttacctggaccaatgtcaccttcctaaagaggtactttagagctgatgagcagtatcctttcctggttcatccagttatgcccatgaaggacatccatgaatctattaggtggacaaaggatccaaagaacactcaagaccacgttcgatcgttgtgcttattggcttggcataacggagaacaagaatatgaggaattcatccgaaagatcagaagcgtcccggttgggcgctgtctgactctccccgcgttttcaactttacgcagaaaatggctggattccttt

SEQ ID NO: 133 - protein, 2193 aa, Echovirus 12
MGAQVSTQKTGAHETGLSASGNSIIHYTNINYYKDAASNSANRQDFTQDPGKFTEPVKDIMIKSMPALNSPTAEECGYSDRVRSITLGNSTITTQECANVVVGYGTWPDYLHDDEATAEDQPTQPDVATCRFYTLESIQWQKTSDGWWWKFPEALKDMGLFGQNMHYHYLGRSGYTIHVQCNASKFHQGCLLVVCVPEAEMGCATVANEVTAAALSSGETAKYFAKTGATGTHTVQNIVTNAGMGVGVGNLTIFPHQWINLRTNNSATIVMPYINSVPMDNMFRHYNFTLMIIPFVPLDFTAEASTYVPITVTVAPMCAEYNGLRLASHQGLPTMNTPGSNQFLTSDDFQSPSAMPQFDVTPELRIPGEVKNLMEIAEVDSVVPVNNTQDSVYNMDVYKIPVSGGNQLSTQVFGFQMQPGLNSVFKRTLLGEILNYYAHWSGSVKLTFVFCGSAMATGKFLLAYSPPGADPPKSRKEAMLGTHVIWDIGLQSSCVLCVPWISQTHYRLVQQDEYTSAGYVTCWYQTSLVVPPGAPVTCGVLCLASACNDFSVRMLRDTPFIEQKQLLQGDVEEAVNRAVARVADTLPTGPRNSESIPALTAAETGHTSQVVPGDTMQTRHVKNYHSRTESSVENFLCRAACVYITKYKTKDSDPVQRYANWRINTRQMVQLRRKFELFTYLRFDMEVTFVITSSQDDGTQLAQDMPVLTHQVMYIPPGGPVPNSVTDFAWQSSTNPSIFWTEGNAPARMSIPFISIGNAYSNFYDGWSHFTQDGVYGFNSLNNMGSIYIRHVNEQSPHAITSTVRVYFKPKHVRAWVPRPPRLCAYEKSSNVNFKPTDVTTSRTSITEVPSLRPSVVNTGAFGQQSGAAYVGNYRVVNRHLATHVDWQNCVWEDYNRDLLVSTTTAHGCDTIARCQCTTGVYFCASRNKHYPVSFEGPGLVEVQESEYYPRRYQSHVLLAAGFSEPGDCGGILRCEHGVIGLVTMGGEGVVGFADVRDLLWLEDDAMEQGVKDYVEQLGNAFGSGFTNQICEQVNLLKESLVGHDSILEKSLKALVKIISALVIVVRNHDDLITVTATLALIGCTSSPWRWLKHKVSQYYGIPMAERQSNGWLKKFTEMTNACKGMEWIAIKIQKFIEWLKLKILPEVKEKHEFLNRLKQLPLLESQIATIEQSAPSQSDQEQLFSNVQYFAHYCRKYAPLYAAEAKRVFSLEKKMSNYIQFKSKCRIEPVCLLLHGSPGAGKSVATSLIGRSLAEKLNSSVYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYSQNGKINMPMSVKTCDEECCPVNFKRCCPLVCGKAIQFIDRRTQVRYSLDMLVTEMFREYNHRHSVGATLEALFQGPPVYREIKISVAPETPPPPAIADLLKSVDSEAVREYCKEKGWLVPEVNSTLQIEKHVSRAFICLQALTTFVSVAGIIYIIYKLFAGFQGAYTGMPNQKPKVPTLRQAKVQGPAFEFAVAMMKRNASTVKTEYGEFTMLGIYDRWAVLPRHAKPGPTILMNDQEIGVLDAKELVDKDGTNLELTLLKLNRNEKFRDIRGFLAREEAEVNEAVLAINTSKFPNMYIPVGQVTDYGFLNLGGTPTKRMLMYNFPTRAGQCGGVLMSTGKVLGIHVGGNGHQGFSAALLRHYFNEEQGEIEFIESSKDAGFPVINTPSKTKLEPSVFHQVFEGNKEPAVLRNGDPRLKVNFEEAIFSKYIGNINTHVDEYMLEAVDHYAGQLATLDISTEPMKLEDAVYGTEGLEALDLTTSAGYPYVALGIKKRDILSKKTKDLTKLKECMDKYGLNLPMVTYVKDELRSSEKVAKGKSRLIEASSLNDSVAMRQTFGNLYKTFHLNPGIVTGSAVGCDPDLFWSKIPVMLDGHLIAFDYSGYDASLSPVWFACLKLLLEKLGYTHRETNYIDYLCNSHHLYRDKHYFVRGGMPSGCSGTSIFNSMINNIIIRTLMLKVYKGIDLDQFRMIAYGDDVIASYPWPIDASLLAEAGKGYGLIMTPADKGECFNEVTWTNVTFLKRYFRADEQYPFLVHPVMPMKDIHESIRWTKDPKNTQDHVRSLCLLAWHNGEQEYEEFIRKIRSVPVGRCLTLPAFSTLRRKWLDSF

SEQ ID NO: 134 - protein, 320 aa, Echovirus 12; identical to SEQ ID NO: 86

SEQ ID NO: 135 - protein, 291 aa, Echovirus 12; identical to SEQ ID NO: 123

SEQ ID NO: 136 - protein, 238 aa, Echovirus 12; identical to SEQ ID NO: 63

SEQ ID NO: 137 - protein, 121 aa, Echovirus 12; identical to SEQ ID NO: 65

SEQ ID NO: 138 - protein, 90 aa, Echovirus 12; identical to SEQ ID NO: 67

SEQ ID NO: 139 - protein, 183 aa, Echovirus 12; identical to SEQ ID NO: 69

SEQ ID NO: 140 - protein, 462 aa, Echovirus 12; identical to SEQ ID NO: 71

SEQ ID NO: 141 - RNA, 7320 nt, Echovirus 12
  source 1..7320: mol_type = unassigned RNA
ttaaaacagcctgtgggttgtccccacccacagggcccactgggcgctagcacactggtatcccggtacctttgtgcgcctgttttatataccctcccctcagtaacttagaagttcatcacaaatgatcaatagttagctcaacaaaccagttgagcctagatcaagcacttctgttaccccgggctgagtatcaataagctgttgacacggctgaaggagaaaacgcccgttacccgaccagctacttcggagaacctagtatcaccatagaggttgcgtagcgtttcgctccgcacaaccccagtgtagatcaggtcgatgagtcaccgcgttccccacaggcgactgtggcggtggctgcgttggcggcctgcccatggggttacccatgggacgcttcaatactgacatggtgtgaagagttgactgagctagctggtagtcctccggcccctgaatgcggctaatcctaactgtggagcaagtgcccacaacccagtgggtggcttgtcgtaatgggcaactctgcagcggaaccgactactttgggtgaccgtgtttctctttattcttatattggctgcttatggtgacaatctcagagttgttaccatatagctattggtttggccaaccagtgactaacagagcaattatatatcttttcattggatttatacctataaacaacaccagttataccactttgtgttacattattggcttgaactcgaaaagatgggagcgcaagtatcaacacaaaaaactggggcacacgagactggcctgagtgctagtggaaattccatcatccactataccaacattaattactacaaagatgcggcatccaattcagcaaacaggcaggatttcacccaagaccccgggaaattcaccgagccagtgaaggatatcatgataaaatcgatgcctgcgttaaactccccaacagcagaagaatgtgggtacagcgatagagtcagatcaatcaccctggggaactctaccatcaccacgcaggagtgtgccaatgtggtggtgggatatggcacgtggcctgattatctacatgatgatgaggccactgcagaggaccaaccaacacaaccggatgtggcgacgtgtaggttttacacacttgaatccattcagtggcagaagacatcagatgggtggtggtggaagtttccggaagcattgaaagacatgggactgttcggccaaaacatgcactatcattatcttggcaggtcgggttacaccatccacgtccaatgtaacgcgtctaaattccaccagggttgtttgcttgtggtgtgtgtgcctgaagctgagatggggtgcgctacagtggcgaatgaggttaccgcagccgctctctcatctggtgaaacggcaaaatattttgcaaagactggtgcgacgggaacccacacagtgcaaaacatagtgacaaacgccggcatgggggtgggcgtgggcaatctgacaatattcccacatcagtggataaacctccgtaccaacaacagcgcgactattgtcatgccttacataaacagtgtgcccatggataacatgttcagacactacaatttcaccctaatgatcatccccttcgtcccactggacttcacagctgaggcatctacgtatgtaccaatcactgtaacagtagctcccatgtgcgccgagtacaacgggctacgcctggcctcgcatcaaggtttgccgacaatgaacacccctggtagtaaccaattcctcacatcagatgactttcaatcaccatcagccatgccacaattcgatgtcacacccgagctcaggattccaggggaggtaaagaacttaatggaaatagccgaagttgactccgtagtgcctgtgaacaatacacaggactcagtgtacaacatggatgtgtacaaaattcctgttagtggaggcaaccaactgtcaactcaagtcttcgggttccaaatgcaaccagggctaaatagtgtgttcaaaaggacattactaggtgagatactgaactactatgcacactggtctggtagtgtaaagctcacctttgtgttctgtggatcggccatggccactggcaaatttctgctggcatattcccctccaggcgcagaccccccaaagtctaggaaagaagcaatgcttggtactcacgtgatatgggatataggacttcagtcaagctgcgtcttgtgcgtaccgtggatcagccaaacacattatagattggttcagcaggatgaatatacgagtgcaggatatgtaacttgctggtatcaaactagtttggttgtcccgcccggcgctcccgtcacgtgtggagtcttgtgcttagcatcggcgtgcaatgatttctctgtgagaatgttgagagacacaccttttatcgagcaaaaacagctactccaaggagatgtggaggaggccgtgaacagagccgttgcacgggtggcggatacactacccacagggccgagaaattcagagagcatccccgcactaacagcagcagaaactgggcatacatctcaggtggttcctggggataccatgcaaacccgtcatgtgaagaattaccactccagaactgaatcgtctgttgaaaacttcttatgccgagcagcatgtgtgtatattacaaagtacaagaccaaggacagtgatccagtgcaaaggtacgccaactggcggatcaacacccgtcaaatggttcagctaaggagaaaatttgagttgtttacatacctaaggtttgatatggaagttacttttgtgatcaccagttcacaagatgatggcacgcagttggcgcaggacatgccagtattaacacaccaagtcatgtatataccacctggagggccagtgccgaatagtgttactgattttgcgtggcaatcgtctaccaaccctagcatcttctggacggagggaaacgctccagctaggatgtccatcccctttatcagtataggcaacgcgtatagcaatttctatgatggttggtcacatttcacccaagatggagtatatggtttcaactccctcaataacatgggctccatctacattaggcatgtcaatgaacaaagcccgcatgcaattacgagcacagtcagagtctatttcaaaccaaaacacgtgcgggcctgggtgccaagaccacctaggctttgtgcatatgagaaatcaagcaacgtgaatttcaaaccaacagatgtgactacctcccgaacatccatcacagaagtccctagtcttagaccatcagtggtaaatactggagctttcggtcagcaatcaggagcggcttatgtgggaaactatagagtggttaatagacacctagccactcatgttgactggcagaactgtgtgtgggaggactataacagggacctccttgtaagcaccaccacagctcatgggtgtgacaccatagccagatgccaatgcacaacaggcgtgtacttctgtgcatcgaggaataaacactacccagtcagttttgaaggaccaggtctggtagaagtccaggagagcgagtactaccccaggaggtatcaatcacacgtcttgctggctgcgggattttcagagccaggagattgtggagggattctcagatgtgagcacggtgtcatcggtctagttactatgggtggcgaaggcgttgtcggattcgctgatgtacgcgacctgttatggcttgaagatgacgccatggaacaaggagtcaaggattacgtggaacaactagggaacgcctttggttcaggattcactaaccaaatctgtgagcaggtcaacctcctaaaagagtcactggtgggtcacgattccatcctagagaagtcccttaaagccctagtgaaaatcatatcagcactagtgatagtggtgaggaaccatgatgatttgatcactgtaactgctactcttgccctcattggttgtacctcctctccatggcggtggctcaagcacaaggtgtcacaatactacgggatacccatggcagagcgccaaagcaacgggtggctcaagaagttcacagagatgaccaacgcctgcaaagggatggaatggattgccatcaaaatccaaaagttcatagagtggcttaaacttaagattttaccagaagttaaggaaaaacatgaattcctgaataggctcaagcaactcccactgttggagagtcaaatagcaaccatcgagcagagtgcaccgtcccagagcgatcaggagcagctcttttcaaatgttcagtactttgcccattattgcagaaaatatgcgcccctatacgcagctgaggcaaaaagggtgttctctcttgaaaagaaaatgagtaattacatacagttcaagtccaaatgccgtattgaacctgtatgcttgctactacatgggagccccggagcagggaaatcagttgctaccagcctcattgggcgatcactagctgaaaaattgaatagctcagtgtactccttaccaccagacccagatcactttgatggttacaagcaacaagctgtagtgatcatggacgatctgtgccaaaatccagatggaaaagatgtgtctttgttctgtcaaatggtgtctagtgtagattttgtaccaccaatggccgcgctagaggagaaaggcattttgttcacctctccattcgttctagcctcaaccaatgcaggatccatcaacgcaccaacagtttcagacagcagagccctagctagaaggttccactttgacatgaacattgaagttatctccatgtacagtcaaaatggaaaaataaacatgcccatgtcagtcaaaacatgtgatgaggaatgctgcccagtcaatttcaagagatgctgcccactggtgtgtggcaaggctattcagttcattgacagaagaacccaagtcagatattcactggatatgttagtcaccgagatgttcagggagtacaaccacaggcacagtgtgggcgccaccctcgaggctctgttccaaggtccaccggtctacagggagatcaagatcagtgttgctccagaaacacctcccccaccagcaatcgctgatttattaaaatcagtagacagtgaagctgtgagagagtactgcaaggaaaaaggctggcttgtaccggaagttaactccaccctacagattgagaagcacgtcagcagagcatttatctgtctacaagctctgaccactttcgtctcagtagctggcataatctacattatctacaagttgtttgccggctttcagggcgcgtatacggggatgccaaatcagaaacccaaagtgcccactctgagacaagctaaagtgcagggcccagcattcgagttcgctgtggcaatgatgaaaaggaacgccagcacagtgaaaacggaatatggtgagttcaccatgctcggcatctatgacagatgggcagtgctaccacgccacgccaagcctggaccgactattttgatgaatgatcaggagatcggcgtgttagacgccaaagaattagtggacaaagatgggacaaatctggagttgacccttttaaagctcaaccgcaatgagaagtttagggacatcagagggtttctggcgagagaggaagctgaagtgaatgaggctgttctggcaataaacacaagcaagttcccaaatatgtacatacccgtaggtcaagtcactgattacggtttcctgaacctggggggaacacccacaaagaggatgctcatgtacaactttccaactagagcagggcaatgtggtggggtactcatgtcaacagggaaagttcttggtatacatgtaggaggaaatggacatcaagggttctccgctgctcttcttaggcactactttaatgaggagcaaggtgaaatagagtttattgagagctcaaaggatgcaggattccctgtgatcaacacccccagcaaaacaaaactggaaccaagcgtgtttcatcaggtgtttgaaggcaataaagagccagcagtccttagaaatggtgatccgcgtcttaaagtaaactttgaagaagccatattttccaagtacatcgggaacatcaacacacatgtggatgaatacatgctcgaagcagtggatcactacgcagggcaactggctactcttgacattagcactgaacccatgaaactagaagatgcagtgtatggcactgaaggactagaggctcttgacttaacaacaagtgcagggtacccgtatgtcgcactaggcatcaaaaagagagacatcctatccaaaaagactaaagatctgactaaattgaaggaatgcatggacaagtacggtttgaacctgccgatggtgacttatgtgaaggatgaacttagatcatcagagaaagtggctaagggaaaatcaagactcattgaagcatctagtttaaatgactctgtcgcgatgaggcagacgtttggcaacttgtacaaaacattccatctgaacccgggcattgtgacaggcagtgcagttggatgtgatccggaccttttctggagtaaaatacccgtgatgttagacggccacctcatagcattcgattactccggatatgatgccagcctgagccctgtgtggttcgcttgtctaaaactgctactcgaaaaactcgggtacacgcacagagagacaaactacatcgactacctatgcaactctcaccacctgtacagagacaaacactactttgtgcgtggtggcatgccatcagggtgctccggtaccagcattttcaattcaatgatcaataacatcataatcaggacactgatgctcaaagtgtacaagggaattgacctggaccagtttagaatgattgcatatggtgatgatgtaattgcatcctacccttggcctatagacgcttcactacttgctgaagctgggaagggttatggactgattatgacaccagcagacaaaggggagtgcttcaatgaggttacctggaccaatgtcaccttcctaaagaggtactttagagctgatgagcagtatcctttcctggttcatccagttatgcccatgaaggacatccatgaatctattaggtggacaaaggatccaaagaacactcaagaccacgttcgatcgttgtgcttattggcttggcataacggagaacaagaatatgaggaattcatccgaaagatcagaagcgtcccggttgggcgctgtctgactctccccgcgttttcaactttacgcagaaaatggctggattccttt

SEQ ID NO: 142 - RNA, 741 nt, Echovirus 12; identical to SEQ ID NO: 44
  source 1..741: mol_type = unassigned RNA

SEQ ID NO: 143 - RNA, 103 nt, Echovirus 12; identical to SEQ ID NO: 45
  source 1..103: mol_type = unassigned RNA

SEQ ID NO: 144 - RNA, 7473 nt, Echovirus 12
  source 1..7473: mol_type = unassigned RNA
ttaaaacagcctgtgggttgtccccacccacagggcccactgggcgctagcacactggtatcccggtacctttgtgcgcctgttttatataccctcccctcagtaacttagaagttcatcacaaatgatcaatagttagctcaacaaaccagttgagcctagatcaagcacttctgttaccccgggctgagtatcaataagctgttgacacggctgaaggagaaaacgcccgttacccgaccagctacttcggagaacctagtatcaccatagaggttgcgtagcgtttcgctccgcacaaccccagtgtagatcaggtcgatgagtcaccgcgttccccacaggcgactgtggcggtggctgcgttggcggcctgcccatggggttacccatgggacgcttcaatactgacatggtgtgaagagttgactgagctagctggtagtcctccggcccctgaatgcggctaatcctaactgtggagcaagtgcccacaacccagtgggtggcttgtcgtaatgggcaactctgcagcggaaccgactactttgggtgaccgtgtttctctttattcttatattggctgcttatggtgacaatctcagagttgttaccatatagctattggtttggccaaccagtgactaacagagcaattatatatcttttcattggatttatacctataaacaacaccagttataccactttgtgttacattattggcttgaactcgaaaagatgggagcgcaagtatcaacacaaaaaactggggcacacgagactggcctgagtgctagtggaaattccatcatccactataccaacattaattactacaaagatgcggcatccaattcagcaaacaggcaggatttcacccaagaccccgggaaattcaccgagccagtgaaggatatcatgataaaatcgatgcctgcgttaaactccccaacagcagaagaatgtgggtacagcgatagagtcagatcaatcaccctggggaactctaccatcaccacgcaggagtgtgccaatgtggtggtgggatatggcacgtggcctgattatctacatgatgatgaggccactgcagaggaccaaccaacacaaccggatgtggcgacgtgtaggttttacacacttgaatccattcagtggcagaagacatcagatgggtggtggtggaagtttccggaagcattgaaagacatgggactgttcggccaaaacatgcactatcattatcttggcaggtcgggttacaccatccacgtccaatgtaacgcgtctaaattccaccagggttgtttgcttgtggtgtgtgtgcctgaagctgagatggggtgcgctacagtggcgaatgaggttaccgcagccgctctctcatctggtgaaacggcaaaatattttgcaaagactggtgcgacgggaacccacacagtgcaaagcatagtgacaaacgccggcatgggggtgggcgtgggcaatctgacaatattcccacatcagtggataaacctccgtaccaacaacagcgcgactattgtcatgccttacataaacagtgtgcccatggataacatgttcagacactacaatttcaccctaatgatcatccccttcgtcccactggacttcacagctgaggcatctacgtatgtaccaatcactgtaacagtagctcccatgtgcgccgagtacaacgggctacgcctggcctcgcatcaaggtttgccgacaatgaacacccctggtagtaaccaattcctcacatcagatgactttcaatcaccatcagccatgccacaattcgatgtcacacccgagctcaggattccaggggaggtaaagaacttaatggaaatagccgaagttgactccgtagtgcctgtgaacaatacacaggactcagtgtacaacatggatgtgtacaaaattcctgttagtggaggcaaccaactgtcaactcaagtcttcgggttccaaatgcaaccagggctaaatagtgtgttcaaaaggacattactaggtgagatactgaactactatgcacactggtctggtagtgtaaagctcacctttgtgttctgtggatcggccatggccactggcaaatttctgctggcatattcccctccaggcgcagaccccccaaagtctaggaaagaagcaatgcttggtactcacgtgatatgggatataggacttcagtcaagctgcgtcttgtgcgtaccgtggatcagccaaacacattatagattggttcagcaggatgaatatacgagtgcaggatatgtaacttgctggtatcaaactagtttggttgtcccgcccggcgctcccgtcacgtgtggagtcttgtgcttagcatcggcgtgcaatgatttctctgtgagaatgttgagagacacaccttttatcgagcaaaaacagctactccaaggagatgtggaggaggccgtgaacagagccgttgcacgggtggcggatacactacccacagggccgagaaattcagagagcatccccgcactaacagcagcagaaactgggcatacatctcaggtggttcctggggataccatgcaaacccgtcatgtgaagaattaccactccagaactgaatcgtctgttgaaaacttcttatgccgagcagcatgtgtgtatattacaaagtacaagaccaaggacagtgatccagtgcaaaggtacgccaactggcggatcaacacccgtcaaatggttcagctaaggagaaaatttgagttgtttacatacctaaggtttgatatggaagttacttttgtgatcaccagttcacaagatgatggcacgcggttggcgcaggacatgccagtattaacacaccaagtcatgtatataccacctggagggccagtgccgaatagtgttactgattttgcgtggcaatcgtctaccaaccctagcatcttctggacggagggaaacgctccagctaggatgtccatcccctttatcagtataggcaacgcgtatagcaatttctatgatggttggtcacatttcacccaagatggagtatatggtttcaactccctcaataacatgggctccatctacattaggcatgtcaatgaacaaagcccgcatgcaattacgagcacagtcagagtctatttcaaaccaaaacacgtgcgggcctgggtgccaagaccacctaggctttgtgcatatgagaaatcaagcaacgtgaatttcaaaccaacagatgtgactacctcccgaacatccatcacagaagtccctagtcttagaccatcagtggtaaatactggagctttcggtcagcaatcaggagcggcttatgtgggaaactatagagtggttaatagacacctagccactcatgttgactggcagaactgtgtgtgggaggactataacagggacctccttgtaagcaccaccacagctcatgggtgtgacaccatagccagatgccaatgcacaacaggcgtgtacttctgtgcatcgaggaataaacactacccagtcagttttgaaggaccaggtctggtagaagtccaggagagcgagtactaccccaggaggtatcaatcacacgtcttgctggctgcgggattttcagagccaggagattgtggagggattctcagatgtgagcacggtgtcatcggtctagttactatgggtggcgaaggcgttgtcggattcgctgatgtacgcgacctgttatggcttgaagatgacgccatggaacaaggagtcaaggattacgtggaacaactagggaacgcctttggttcaggattcactaaccaaatctgtgagcaggtcaayctcctaaaagagtcactggtgggtcacgattccatcctagagaagtcccttaaagccctagtgaaaatcatatcagcactagtgatagtggtgaggaaccatgatgatttgatcactgtaactgctactcttgccctcattggttgtacctcctctccatggcggtggctcaagcacaaggtgtcacaatactacgggatacccatggcagagcgccaaagcaacgggtggctcaagaagttcacagagatgaccaacgcctgcaaagggatggaatggattgccatcaaaatccaaaagttcatagagtggcttaaacttaagattttaccagaagttaaggaaaaacatgaattcctgaataggctcaagcaactcccactgttggagagtcaaatagcaaccatcgagcagagtgcaccgtcccagagcgatcaggagcagctcttttcaaatgttcagtactttgcccattattgcagaaaatatgcgcccctatacgcagctgaggcaaaaagggtgttctctcttgaaaagaaaatgagtaattacatacagttcaagtccaaatgccgtattgaacctgtatgcttgctactacatgggagccccggagcagggaaatcagttgctaccagcctcattgggcgatcactagctgaaaaattgaatagctcagtgtactccttaccaccagacccagatcactttgatggttacaagcaacaagctgtagtgatcatggacgatctgtgccaaaatccagatggaaaagatgtgtctttgttctgtcaaatggtgtctagtgtagattttgtaccaccaatggccgcgctagaggagaaaggcattttgttcacctctccattcgttctagcctcaaccaatgcaggatccatcaacgcaccaacagtttcagacagcagagccctagctagaaggttccactttgacatgaacattgaagttatctccatgtacagtcaaaatggaaaaataaacatgcccatgtcagtcaaaacatgtgatgaggaatgctgcccagtcaatttcaagagatgctgcccactggtgtgtggcaaggctattcagttcattgacagaagaacccaagtcagatattcactggatatgttagtcaccgagatgttcagggagtacaaccacaggcacagtgtgggcgccaccctcgaggctctgttccaaggtccaccggtctacagggagatcaagatcagtgttgctccagaaacacctcccccaccagcaatcgctgatttattaaaatcagtagacagtgaagctgtgagagagtactgcaaggaaaaaggctggcttgtaccggaagttaactccaccctacagattgagaagcacgtcagcagagcatttatctgtctacaagctctgaccactttcgtctcagtagctggcataatctacattatctacaagttgtttgccggctttcagggcgcgtatacggggatgccaaatcagaaacccaaagtgcccactctgagacaagctaaagtgcagggcccagcattcgagttcgctgtggcaatgatgaaaaggaacgccagcacagtgaaaacggaatatggtgagttcaccatgctcggcatctatgacagatgggcagtgctaccacgccacgccaagcctggaccgactattttgatgaatgatcaggagatcggcgtgttagacgccaaagaattagtggacaaagatgggacaaatctggagttgacccttttaaagctcaaccgcaatgagaagtttagggacatcagagggtttctggcgagagaggaagctgaagtgaatgaggctgttctggcaataaacacaagcaagttcccaaatatgtacatacccgtaggtcaagtcactgattacggtttcctgaacctggggggaacacccacaaagaggatgctcatgtacaactttccaactagagcagggcaatgtggtggggtactcatgtcaacagggaaagttcttggtatacatgtaggaggaaatggacatcaagggttctccgctgctcttcttaggcactactttaatgaggagcaaggtgaaatagagtttattgagagctcaaaggatgcaggattccctgtgatcaacacccccagcaaaacaaaactggaaccaagcgtgtttcatcaggtgtttgaaggcaataaagagccagcagtccttagaaatggtgatccgcgtcttaaagtaaactttgaagaagccatattttccaagtacatcgggaacatcaacacacatgtggatgaatacatgctcgaagcagtggatcactacgcagggcaactggctactcttgacattagcactgaacccatgaaactagaagatgcagtgtatggcactgaaggactagaggctcttgacttaacaacaagtgcagggtacccgtatgtcgcactaggcatcaaaaagagagacatcctatccaaaaagactaaagatctgactaaattgaaggaatgcatggacaagtacggtttgaacctgccgatggtgacttatgtgaaggatgaacttagatcatcagagaaagtggctaagggaaaatcaagactcattgaagcatctagtttaaatgactctgtcgcgatgaggcagacgtttggcaacttgtacaaaacattccatctgaacccgggcattgtgacaggcagtgcagttggatgtgatccggaccttttctggagtaaaatacccgtgatgttagacggccacctcatagcattcgattactccggatatgatgccagcctgagccctgtgtggttcgcttgtctaaaactgctactcgaaaaactcgggtacacgcacagagagacaaactacatcgactacctatgcaactctcaccacctgtacagagacaaacactactttgtgcgtggtggcatgccatcagggtgctccggtaccagcattttcaattcaatgatcaataacatcataatcaggacactgatgctcaaagtgtacaagggaattgacctggaccagtttagaatgattgcatatggtgatgatgtaattgcatcctacccttggcctatagacgcttcactacttgctgaagctgggaagggttatggactgattatgacaccagcagacaaaggggagtgcttcaatgaggttacctggaccaatgtcaccttcctaaagaggtactttagagctgatgagcagtatcctttcctggttcatccagttatgcccatgaaggacatccatgaatctattaggtggacaaaggatccaaagaacactcaagaccacgttcgatcgttgtgcttattggcttggcataacggagaacaagaatatgaggaattcatccgaaagatcagaagcgtcccggttgggcgctgtctgactctccccgcgttttcaactttacgcagaaaatggctggattccttttaaattagagacaatttgaaataatttaaattggcttaaccctactgtactaaccgaactagacaacggtgcagtaggggtaaattctccgcattcggtgcggaaaaaaaaaaaaaaaaaaaaaaaaaaaaaaaaaaaaaaaaaaaaaaaaaa

SEQ ID NO: 145 - protein, 2193 aa, Echovirus 12
MGAQVSTQKTGAHETGLSASGNSIIHYTNINYYKDAASNSANRQDFTQDPGKFTEPVKDIMIKSMPALNSPTAEECGYSDRVRSITLGNSTITTQECANVVVGYGTWPDYLHDDEATAEDQPTQPDVATCRFYTLESIQWQKTSDGWWWKFPEALKDMGLFGQNMHYHYLGRSGYTIHVQCNASKFHQGCLLVVCVPEAEMGCATVANEVTAAALSSGETAKYFAKTGATGTHTVQSIVTNAGMGVGVGNLTIFPHQWINLRTNNSATIVMPYINSVPMDNMFRHYNFTLMIIPFVPLDFTAEASTYVPITVTVAPMCAEYNGLRLASHQGLPTMNTPGSNQFLTSDDFQSPSAMPQFDVTPELRIPGEVKNLMEIAEVDSVVPVNNTQDSVYNMDVYKIPVSGGNQLSTQVFGFQMQPGLNSVFKRTLLGEILNYYAHWSGSVKLTFVFCGSAMATGKFLLAYSPPGADPPKSRKEAMLGTHVIWDIGLQSSCVLCVPWISQTHYRLVQQDEYTSAGYVTCWYQTSLVVPPGAPVTCGVLCLASACNDFSVRMLRDTPFIEQKQLLQGDVEEAVNRAVARVADTLPTGPRNSESIPALTAAETGHTSQVVPGDTMQTRHVKNYHSRTESSVENFLCRAACVYITKYKTKDSDPVQRYANWRINTRQMVQLRRKFELFTYLRFDMEVTFVITSSQDDGTRLAQDMPVLTHQVMYIPPGGPVPNSVTDFAWQSSTNPSIFWTEGNAPARMSIPFISIGNAYSNFYDGWSHFTQDGVYGFNSLNNMGSIYIRHVNEQSPHAITSTVRVYFKPKHVRAWVPRPPRLCAYEKSSNVNFKPTDVTTSRTSITEVPSLRPSVVNTGAFGQQSGAAYVGNYRVVNRHLATHVDWQNCVWEDYNRDLLVSTTTAHGCDTIARCQCTTGVYFCASRNKHYPVSFEGPGLVEVQESEYYPRRYQSHVLLAAGFSEPGDCGGILRCEHGVIGLVTMGGEGVVGFADVRDLLWLEDDAMEQGVKDYVEQLGNAFGSGFTNQICEQVXLLKESLVGHDSILEKSLKALVKIISALVIVVRNHDDLITVTATLALIGCTSSPWRWLKHKVSQYYGIPMAERQSNGWLKKFTEMTNACKGMEWIAIKIQKFIEWLKLKILPEVKEKHEFLNRLKQLPLLESQIATIEQSAPSQSDQEQLFSNVQYFAHYCRKYAPLYAAEAKRVFSLEKKMSNYIQFKSKCRIEPVCLLLHGSPGAGKSVATSLIGRSLAEKLNSSVYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYSQNGKINMPMSVKTCDEECCPVNFKRCCPLVCGKAIQFIDRRTQVRYSLDMLVTEMFREYNHRHSVGATLEALFQGPPVYREIKISVAPETPPPPAIADLLKSVDSEAVREYCKEKGWLVPEVNSTLQIEKHVSRAFICLQALTTFVSVAGIIYIIYKLFAGFQGAYTGMPNQKPKVPTLRQAKVQGPAFEFAVAMMKRNASTVKTEYGEFTMLGIYDRWAVLPRHAKPGPTILMNDQEIGVLDAKELVDKDGTNLELTLLKLNRNEKFRDIRGFLAREEAEVNEAVLAINTSKFPNMYIPVGQVTDYGFLNLGGTPTKRMLMYNFPTRAGQCGGVLMSTGKVLGIHVGGNGHQGFSAALLRHYFNEEQGEIEFIESSKDAGFPVINTPSKTKLEPSVFHQVFEGNKEPAVLRNGDPRLKVNFEEAIFSKYIGNINTHVDEYMLEAVDHYAGQLATLDISTEPMKLEDAVYGTEGLEALDLTTSAGYPYVALGIKKRDILSKKTKDLTKLKECMDKYGLNLPMVTYVKDELRSSEKVAKGKSRLIEASSLNDSVAMRQTFGNLYKTFHLNPGIVTGSAVGCDPDLFWSKIPVMLDGHLIAFDYSGYDASLSPVWFACLKLLLEKLGYTHRETNYIDYLCNSHHLYRDKHYFVRGGMPSGCSGTSIFNSMINNIIIRTLMLKVYKGIDLDQFRMIAYGDDVIASYPWPIDASLLAEAGKGYGLIMTPADKGECFNEVTWTNVTFLKRYFRADEQYPFLVHPVMPMKDIHESIRWTKDPKNTQDHVRSLCLLAWHNGEQEYEEFIRKIRSVPVGRCLTLPAFSTLRRKWLDSF

SEQ ID NO: 146 - protein, 320 aa, Echovirus 12
GDVEEAVNRAVARVADTLPTGPRNSESIPALTAAETGHTSQVVPGDTMQTRHVKNYHSRTESSVENFLCRAACVYITKYKTKDSDPVQRYANWRINTRQMVQLRRKFELFTYLRFDMEVTFVITSSQDDGTRLAQDMPVLTHQVMYIPPGGPVPNSVTDFAWQSSTNPSIFWTEGNAPARMSIPFISIGNAYSNFYDGWSHFTQDGVYGFNSLNNMGSIYIRHVNEQSPHAITSTVRVYFKPKHVRAWVPRPPRLCAYEKSSNVNFKPTDVTTSRTSITEVPSLRPSVVNTGAFGQQSGAAYVGNYRVVNRHLATHVDWQ

SEQ ID NO: 147 - protein, 291 aa, Echovirus 12; identical to SEQ ID NO: 87

SEQ ID NO: 148 - protein, 238 aa, Echovirus 12; identical to SEQ ID NO: 63

SEQ ID NO: 149 - protein, 121 aa, Echovirus 12; identical to SEQ ID NO: 65

SEQ ID NO: 150 - protein, 90 aa, Echovirus 12; identical to SEQ ID NO: 67

SEQ ID NO: 151 - protein, 183 aa, Echovirus 12; identical to SEQ ID NO: 69

SEQ ID NO: 152 - protein, 462 aa, Echovirus 12; identical to SEQ ID NO: 71

SEQ ID NO: 153 - RNA, 6579 nt, Echovirus 12
  source 1..6579: mol_type = unassigned RNA
atgggagcgcaagtatcaacacaaaaaactggggcacacgagactggcctgagtgctagtggaaattccatcatccactataccaacattaattactacaaagatgcggcatccaattcagcaaacaggcaggatttcacccaagaccccgggaaattcaccgagccagtgaaggatatcatgataaaatcgatgcctgcgttaaactccccaacagcagaagaatgtgggtacagcgatagagtcagatcaatcaccctggggaactctaccatcaccacgcaggagtgtgccaatgtggtggtgggatatggcacgtggcctgattatctacatgatgatgaggccactgcagaggaccaaccaacacaaccggatgtggcgacgtgtaggttttacacacttgaatccattcagtggcagaagacatcagatgggtggtggtggaagtttccggaagcattgaaagacatgggactgttcggccaaaacatgcactatcattatcttggcaggtcgggttacaccatccacgtccaatgtaacgcgtctaaattccaccagggttgtttgcttgtggtgtgtgtgcctgaagctgagatggggtgcgctacagtggcgaatgaggttaccgcagccgctctctcatctggtgaaacggcaaaatattttgcaaagactggtgcgacgggaacccacacagtgcaaagcatagtgacaaacgccggcatgggggtgggcgtgggcaatctgacaatattcccacatcagtggataaacctccgtaccaacaacagcgcgactattgtcatgccttacataaacagtgtgcccatggataacatgttcagacactacaatttcaccctaatgatcatccccttcgtcccactggacttcacagctgaggcatctacgtatgtaccaatcactgtaacagtagctcccatgtgcgccgagtacaacgggctacgcctggcctcgcatcaaggtttgccgacaatgaacacccctggtagtaaccaattcctcacatcagatgactttcaatcaccatcagccatgccacaattcgatgtcacacccgagctcaggattccaggggaggtaaagaacttaatggaaatagccgaagttgactccgtagtgcctgtgaacaatacacaggactcagtgtacaacatggatgtgtacaaaattcctgttagtggaggcaaccaactgtcaactcaagtcttcgggttccaaatgcaaccagggctaaatagtgtgttcaaaaggacattactaggtgagatactgaactactatgcacactggtctggtagtgtaaagctcacctttgtgttctgtggatcggccatggccactggcaaatttctgctggcatattcccctccaggcgcagaccccccaaagtctaggaaagaagcaatgcttggtactcacgtgatatgggatataggacttcagtcaagctgcgtcttgtgcgtaccgtggatcagccaaacacattatagattggttcagcaggatgaatatacgagtgcaggatatgtaacttgctggtatcaaactagtttggttgtcccgcccggcgctcccgtcacgtgtggagtcttgtgcttagcatcggcgtgcaatgatttctctgtgagaatgttgagagacacaccttttatcgagcaaaaacagctactccaaggagatgtggaggaggccgtgaacagagccgttgcacgggtggcggatacactacccacagggccgagaaattcagagagcatccccgcactaacagcagcagaaactgggcatacatctcaggtggttcctggggataccatgcaaacccgtcatgtgaagaattaccactccagaactgaatcgtctgttgaaaacttcttatgccgagcagcatgtgtgtatattacaaagtacaagaccaaggacagtgatccagtgcaaaggtacgccaactggcggatcaacacccgtcaaatggttcagctaaggagaaaatttgagttgtttacatacctaaggtttgatatggaagttacttttgtgatcaccagttcacaagatgatggcacgcggttggcgcaggacatgccagtattaacacaccaagtcatgtatataccacctggagggccagtgccgaatagtgttactgattttgcgtggcaatcgtctaccaaccctagcatcttctggacggagggaaacgctccagctaggatgtccatcccctttatcagtataggcaacgcgtatagcaatttctatgatggttggtcacatttcacccaagatggagtatatggtttcaactccctcaataacatgggctccatctacattaggcatgtcaatgaacaaagcccgcatgcaattacgagcacagtcagagtctatttcaaaccaaaacacgtgcgggcctgggtgccaagaccacctaggctttgtgcatatgagaaatcaagcaacgtgaatttcaaaccaacagatgtgactacctcccgaacatccatcacagaagtccctagtcttagaccatcagtggtaaatactggagctttcggtcagcaatcaggagcggcttatgtgggaaactatagagtggttaatagacacctagccactcatgttgactggcagaactgtgtgtgggaggactataacagggacctccttgtaagcaccaccacagctcatgggtgtgacaccatagccagatgccaatgcacaacaggcgtgtacttctgtgcatcgaggaataaacactacccagtcagttttgaaggaccaggtctggtagaagtccaggagagcgagtactaccccaggaggtatcaatcacacgtcttgctggctgcgggattttcagagccaggagattgtggagggattctcagatgtgagcacggtgtcatcggtctagttactatgggtggcgaaggcgttgtcggattcgctgatgtacgcgacctgttatggcttgaagatgacgccatggaacaaggagtcaaggattacgtggaacaactagggaacgcctttggttcaggattcactaaccaaatctgtgagcaggtcaayctcctaaaagagtcactggtgggtcacgattccatcctagagaagtcccttaaagccctagtgaaaatcatatcagcactagtgatagtggtgaggaaccatgatgatttgatcactgtaactgctactcttgccctcattggttgtacctcctctccatggcggtggctcaagcacaaggtgtcacaatactacgggatacccatggcagagcgccaaagcaacgggtggctcaagaagttcacagagatgaccaacgcctgcaaagggatggaatggattgccatcaaaatccaaaagttcatagagtggcttaaacttaagattttaccagaagttaaggaaaaacatgaattcctgaataggctcaagcaactcccactgttggagagtcaaatagcaaccatcgagcagagtgcaccgtcccagagcgatcaggagcagctcttttcaaatgttcagtactttgcccattattgcagaaaatatgcgcccctatacgcagctgaggcaaaaagggtgttctctcttgaaaagaaaatgagtaattacatacagttcaagtccaaatgccgtattgaacctgtatgcttgctactacatgggagccccggagcagggaaatcagttgctaccagcctcattgggcgatcactagctgaaaaattgaatagctcagtgtactccttaccaccagacccagatcactttgatggttacaagcaacaagctgtagtgatcatggacgatctgtgccaaaatccagatggaaaagatgtgtctttgttctgtcaaatggtgtctagtgtagattttgtaccaccaatggccgcgctagaggagaaaggcattttgttcacctctccattcgttctagcctcaaccaatgcaggatccatcaacgcaccaacagtttcagacagcagagccctagctagaaggttccactttgacatgaacattgaagttatctccatgtacagtcaaaatggaaaaataaacatgcccatgtcagtcaaaacatgtgatgaggaatgctgcccagtcaatttcaagagatgctgcccactggtgtgtggcaaggctattcagttcattgacagaagaacccaagtcagatattcactggatatgttagtcaccgagatgttcagggagtacaaccacaggcacagtgtgggcgccaccctcgaggctctgttccaaggtccaccggtctacagggagatcaagatcagtgttgctccagaaacacctcccccaccagcaatcgctgatttattaaaatcagtagacagtgaagctgtgagagagtactgcaaggaaaaaggctggcttgtaccggaagttaactccaccctacagattgagaagcacgtcagcagagcatttatctgtctacaagctctgaccactttcgtctcagtagctggcataatctacattatctacaagttgtttgccggctttcagggcgcgtatacggggatgccaaatcagaaacccaaagtgcccactctgagacaagctaaagtgcagggcccagcattcgagttcgctgtggcaatgatgaaaaggaacgccagcacagtgaaaacggaatatggtgagttcaccatgctcggcatctatgacagatgggcagtgctaccacgccacgccaagcctggaccgactattttgatgaatgatcaggagatcggcgtgttagacgccaaagaattagtggacaaagatgggacaaatctggagttgacccttttaaagctcaaccgcaatgagaagtttagggacatcagagggtttctggcgagagaggaagctgaagtgaatgaggctgttctggcaataaacacaagcaagttcccaaatatgtacatacccgtaggtcaagtcactgattacggtttcctgaacctggggggaacacccacaaagaggatgctcatgtacaactttccaactagagcagggcaatgtggtggggtactcatgtcaacagggaaagttcttggtatacatgtaggaggaaatggacatcaagggttctccgctgctcttcttaggcactactttaatgaggagcaaggtgaaatagagtttattgagagctcaaaggatgcaggattccctgtgatcaacacccccagcaaaacaaaactggaaccaagcgtgtttcatcaggtgtttgaaggcaataaagagccagcagtccttagaaatggtgatccgcgtcttaaagtaaactttgaagaagccatattttccaagtacatcgggaacatcaacacacatgtggatgaatacatgctcgaagcagtggatcactacgcagggcaactggctactcttgacattagcactgaacccatgaaactagaagatgcagtgtatggcactgaaggactagaggctcttgacttaacaacaagtgcagggtacccgtatgtcgcactaggcatcaaaaagagagacatcctatccaaaaagactaaagatctgactaaattgaaggaatgcatggacaagtacggtttgaacctgccgatggtgacttatgtgaaggatgaacttagatcatcagagaaagtggctaagggaaaatcaagactcattgaagcatctagtttaaatgactctgtcgcgatgaggcagacgtttggcaacttgtacaaaacattccatctgaacccgggcattgtgacaggcagtgcagttggatgtgatccggaccttttctggagtaaaatacccgtgatgttagacggccacctcatagcattcgattactccggatatgatgccagcctgagccctgtgtggttcgcttgtctaaaactgctactcgaaaaactcgggtacacgcacagagagacaaactacatcgactacctatgcaactctcaccacctgtacagagacaaacactactttgtgcgtggtggcatgccatcagggtgctccggtaccagcattttcaattcaatgatcaataacatcataatcaggacactgatgctcaaagtgtacaagggaattgacctggaccagtttagaatgattgcatatggtgatgatgtaattgcatcctacccttggcctatagacgcttcactacttgctgaagctgggaagggttatggactgattatgacaccagcagacaaaggggagtgcttcaatgaggttacctggaccaatgtcaccttcctaaagaggtactttagagctgatgagcagtatcctttcctggttcatccagttatgcccatgaaggacatccatgaatctattaggtggacaaaggatccaaagaacactcaagaccacgttcgatcgttgtgcttattggcttggcataacggagaacaagaatatgaggaattcatccgaaagatcagaagcgtcccggttgggcgctgtctgactctccccgcgttttcaactttacgcagaaaatggctggattccttt

SEQ ID NO: 154 - RNA, 741 nt, Echovirus 12; identical to SEQ ID NO: 44
  source 1..741: mol_type = unassigned RNA

SEQ ID NO: 155 - RNA, 103 nt, Echovirus 12; identical to SEQ ID NO: 45
  source 1..103: mol_type = unassigned RNA

SEQ ID NO: 156 - RNA, 7473 nt, Echovirus 12
  source 1..7473: mol_type = unassigned RNA
ttaaaacagcctgtgggttgtccccacccacagggcccactgggcgctagcacactggtatcccggtacctttgtgcgcctgttttatataccctcccctcagtaacttagaagttcatcacaaatgatcaatagttagctcaacaaaccagttgagcctagatcaagcacttctgttaccccgggctgagtatcaataagctgttgacacggctgaaggagaaaacgcccgttacccgaccagctacttcggagaacctagtatcaccatagaggttgcgtagcgtttcgctccgcacaaccccagtgtagatcaggtcgatgagtcaccgcgttccccacaggcgactgtggcggtggctgcgttggcggcctgcccatggggttacccatgggacgcttcaatactgacatggtgtgaagagttgactgagctagctggtagtcctccggcccctgaatgcggctaatcctaactgtggagcaagtgcccacaacccagtgggtggcttgtcgtaatgggcaactctgcagcggaaccgactactttgggtgaccgtgtttctctttattcttatattggctgcttatggtgacaatctcagagttgttaccatatagctattggtttggccaaccagtgactaacagagcaattatatatcttttcattggatttatacctataaacaacaccagttataccactttgtgttacattattggcttgaactcgaaaagatgggagcgcaagtatcaacacaaaaaactggggcacacgagactggcctgagtgctagtggaaattccatcatccactataccaacattaattactacaaagatgcggcatccaattcagcaaacaggcaggatttcacccaagaccccgggaaattcaccgagccagtgaaggatatcatgataaaatcgatgcctgcgttaaactccccaacagcagaagaatgtgggtacagcgatagagtcagatcaatcaccctggggaactctaccatcaccacgcaggagtgtgccaatgtggtggtgggatatggcacgtggcctgattatctacatgatgatgaggccactgcagaggaccaaccaacacaaccggatgtggcgacgtgtaggttttacacacttgaatccattcagtggcagaagacatcagatgggtggtggtggaagtttccggaagcattgaaagacatgggactgttcggccaaaacatgcactatcattatcttggcaggtcgggttacaccatccacgtccaatgtaacgcgtctaaattccaccagggttgtttgcttgtggtgtgtgtgcctgaagctgagatggggtgcgctacagtggcgaatgaggttaccgcagccgctctctcatctggtgaaacggcaaaatattttgcaaagactggtgcgacgggaacccacacagtgcaaagcatagtgacaaacgccggcatgggggtgggcgtgggcaatctgacaatattcccacatcagtggataaacctccgtaccaacaacagcgcgactattgtcatgccttacataaacagtgtgcccatggataacatgttcagacactacaatttcaccctaatgatcatccccttcgtcccactggacttcacagctgaggcatctacgtatgtaccaatcactgtaacagtagctcccatgtgcgccgagtacaacgggctacgcctggcctcgcatcaaggtttgccgacaatgaacacccctggtagtaaccaattcctcacatcagatgactttcaatcaccatcagccatgccacaattcgatgtcacacccgagctcaggattccaggggaggtaaagaacttaatggaaatagccgaagttgactccgtagtgcctgtgaacaatacacaggactcagtgtacaacatggatgtgtacaaaattcctgttagtggaggcaaccaactgtcaactcaagtcttcgggttccaaatgcaaccagggctaaatagtgtgttcaaaaggacattactaggtgagatactgaactactatgcacactggtctggtagtgtaaagctcacctttgtgttctgtggatcggccatggccactggcaaatttctgctggcatattcccctccaggcgcagaccccccaaagtctaggaaagaagcaatgcttggtactcacgtgatatgggatataggacttcagtcaagctgcgtcttgtgcgtaccgtggatcagccaaacacattatagattggttcagcaggatgaatatacgagtgcaggatatgtaacttgctggtatcaaactagtttggttgtcccgcccggcgctcccgtcacgtgtggagtcttgtgcttagcatcggcgtgcaatgatttctctgtgagaatgttgagagacacaccttttatcgagcaaaaacagctactccaaggagatgtggaggaggccgtgaacagagccgttgcacgggtggcggatacactacccacagggccgagaaattcagagagcatccccgcactaacagcagcagaaactgggcatacatctcaggtggttcctggggataccatgcaaacccgtcatgtgaagaattaccactccagaactgaatcgtctgttgaaaacttcttatgccgagcagcatgtgtgtatattacaaagtacaagaccaaggacagtgatccagtgcaaaggtacgccaactggcggatcaacacccgtcaaatggttcagctaaggagaaaatttgagttgtttacatacctaaggtttgatatggaagttacttttgtgatcaccagttcacaagatgatggcacgcggttggcgcaggacatgccagtattaacacaccaagtcatgtatataccacctggagggccagtgccgaatagtgttactgattttgcgtggcaatcgtctaccaaccctagcatcttctggacggagggaaacgctccagctaggatgtccatcccctttatcagtataggcaacgcgtatagcaatttctatgatggttggtcacatttcacccaagatggagtatatggtttcaactccctcaataacatgggctccatctacattaggcatgtcaatgaacaaagcccgcatgcaattacgagcacagtcagagtctatttcaaaccaaaacacgtgcgggcctgggtgccaagaccacctaggctttgtgcatatgagaaatcaagcaacgtgaatttcaaaccaacagatgtgactacctcccgaacatccatcacagaagtccctagtcttagaccatcagtggtaaatactggagctttcggtcagcaatcaggagcggcttatgtgggaaactatagagtggttaatagacacctagccactcatgttgactggcagaactgtgtgtgggaggactataacagggacctccttgtaagcaccaccacagctcatgggtgtgacaccatagccagatgccaatgcacaacaggcgtgtacttctgtgcatcgaggaataaacactacccagtcagttatgaaggaccaggtctggtagaagtccaggagagcgagtactaccccaggaggtatcaatcacacgtcttgctggctgcgggattttcagagccaggagattgtggagggattctcagatgtgagcacggtgtcatcggtctagttactatgggtggcgaaggcgttgtcggattcgctgatgtacgcgacctgttatggcttgaagatgacgccatggaacaaggagtcaaggattacgtggaacaactagggaacgcctttggttcaggattcactaaccaaatctgtgagcaggtcaayctcctaaaagagtcactggtgggtcacgattccatcctagagaagtcccttaaagccctagtgaaaatcatatcagcactagtgatagtggtgaggaaccatgatgatttgatcactgtaactgctactcttgccctcattggttgtacctcctctccatggcggtggctcaagcacaaggtgtcacaatactacgggatacccatggcagagcgccaaagcaacgggtggctcaagaagttcacagagatgaccaacgcctgcaaagggatggaatggattgccatcaaaatccaaaagttcatagagtggcttaaacttaagattttaccagaagttaaggaaaaacatgaattcctgaataggctcaagcaactcccactgttggagagtcaaatagcaaccatcgagcagagtgcaccgtcccagagcgatcaggagcagctcttttcaaatgttcagtactttgcccattattgcagaaaatatgcgcccctatacgcagctgaggcaaaaagggtgttctctcttgaaaagaaaatgagtaattacatacagttcaagtccaaatgccgtattgaacctgtatgcttgctactacatgggagccccggagcagggaaatcagttgctaccagcctcattgggcgatcactagctgaaaaattgaatagctcagtgtactccttaccaccagacccagatcactttgatggttacaagcaacaagctgtagtgatcatggacgatctgtgccaaaatccagatggaaaagatgtgtctttgttctgtcaaatggtgtctagtgtagattttgtaccaccaatggccgcgctagaggagaaaggcattttgttcacctctccattcgttctagcctcaaccaatgcaggatccatcaacgcaccaacagtttcagacagcagagccctagctagaaggttccactttgacatgaacattgaagttatctccatgtacagtcaaaatggaaaaataaacatgcccatgtcagtcaaaacatgtgatgaggaatgctgcccagtcaatttcaagagatgctgcccactggtgtgtggcaaggctattcagttcattgacagaagaacccaagtcagatattcactggatatgttagtcaccgagatgttcagggagtacaaccacaggcacagtgtgggcgccaccctcgaggctctgttccaaggtccaccggtctacagggagatcaagatcagtgttgctccagaaacacctcccccaccagcaatcgctgatttattaaaatcagtagacagtgaagctgtgagagagtactgcaaggaaaaaggctggcttgtaccggaagttaactccaccctacagattgagaagcacgtcagcagagcatttatctgtctacaagctctgaccactttcgtctcagtagctggcataatctacattatctacaagttgtttgccggctttcagggcgcgtatacggggatgccaaatcagaaacccaaagtgcccactctgagacaagctaaagtgcagggcccagcattcgagttcgctgtggcaatgatgaaaaggaacgccagcacagtgaaaacggaatatggtgagttcaccatgctcggcatctatgacagatgggcagtgctaccacgccacgccaagcctggaccgactattttgatgaatgatcaggagatcggcgtgttagacgccaaagaattagtggacaaagatgggacaaatctggagttgacccttttaaagctcaaccgcaatgagaagtttagggacatcagagggtttctggcgagagaggaagctgaagtgaatgaggctgttctggcaataaacacaagcaagttcccaaatatgtacatacccgtaggtcaagtcactgattacggtttcctgaacctggggggaacacccacaaagaggatgctcatgtacaactttccaactagagcagggcaatgtggtggggtactcatgtcaacagggaaagttcttggtatacatgtaggaggaaatggacatcaagggttctccgctgctcttcttaggcactactttaatgaggagcaaggtgaaatagagtttattgagagctcaaaggatgcaggattccctgtgatcaacacccccagcaaaacaaaactggaaccaagcgtgtttcatcaggtgtttgaaggcaataaagagccagcagtccttagaaatggtgatccgcgtcttaaagtaaactttgaagaagccatattttccaagtacatcgggaacatcaacacacatgtggatgaatacatgctcgaagcagtggatcactacgcagggcaactggctactcttgacattagcactgaacccatgaaactagaagatgcagtgtatggcactgaaggactagaggctcttgacttaacaacaagtgcagggtacccgtatgtcgcactaggcatcaaaaagagagacatcctatccaaaaagactaaagatctgactaaattgaaggaatgcatggacaagtacggtttgaacctgccgatggtgacttatgtgaaggatgaacttagatcatcagagaaagtggctaagggaaaatcaagactcattgaagcatctagtttaaatgactctgtcgcgatgaggcagacgtttggcaacttgtacaaaacattccatctgaacccgggcattgtgacaggcagtgcagttggatgtgatccggaccttttctggagtaaaatacccgtgatgttagacggccacctcatagcattcgattactccggatatgatgccagcctgagccctgtgtggttcgcttgtctaaaactgctactcgaaaaactcgggtacacgcacagagagacaaactacatcgactacctatgcaactctcaccacctgtacagagacaaacactactttgtgcgtggtggcatgccatcagggtgctccggtaccagcattttcaattcaatgatcaataacatcataatcaggacactgatgctcaaagtgtacaagggaattgacctggaccagtttagaatgattgcatatggtgatgatgtaattgcatcctacccttggcctatagacgcttcactacttgctgaagctgggaagggttatggactgattatgacaccagcagacaaaggggagtgcttcaatgaggttacctggaccaatgtcaccttcctaaagaggtactttagagctgatgagcagtatcctttcctggttcatccagttatgcccatgaaggacatccatgaatctattaggtggacaaaggatccaaagaacactcaagaccacgttcgatcgttgtgcttattggcttggcataacggagaacaagaatatgaggaattcatccgaaagatcagaagcgtcccggttgggcgctgtctgactctccccgcgttttcaactttacgcagaaaatggctggattccttttaaattagagacaatttgaaataatttaaattggcttaaccctactgtactaaccgaactagacaacggtgcagtaggggtaaattctccgcattcggtgcggaaaaaaaaaaaaaaaaaaaaaaaaaaaaaaaaaaaaaaaaaaaaaaaaaa

SEQ ID NO: 157 - protein, 2193 aa, Echovirus 12
MGAQVSTQKTGAHETGLSASGNSIIHYTNINYYKDAASNSANRQDFTQDPGKFTEPVKDIMIKSMPALNSPTAEECGYSDRVRSITLGNSTITTQECANVVVGYGTWPDYLHDDEATAEDQPTQPDVATCRFYTLESIQWQKTSDGWWWKFPEALKDMGLFGQNMHYHYLGRSGYTIHVQCNASKFHQGCLLVVCVPEAEMGCATVANEVTAAALSSGETAKYFAKTGATGTHTVQSIVTNAGMGVGVGNLTIFPHQWINLRTNNSATIVMPYINSVPMDNMFRHYNFTLMIIPFVPLDFTAEASTYVPITVTVAPMCAEYNGLRLASHQGLPTMNTPGSNQFLTSDDFQSPSAMPQFDVTPELRIPGEVKNLMEIAEVDSVVPVNNTQDSVYNMDVYKIPVSGGNQLSTQVFGFQMQPGLNSVFKRTLLGEILNYYAHWSGSVKLTFVFCGSAMATGKFLLAYSPPGADPPKSRKEAMLGTHVIWDIGLQSSCVLCVPWISQTHYRLVQQDEYTSAGYVTCWYQTSLVVPPGAPVTCGVLCLASACNDFSVRMLRDTPFIEQKQLLQGDVEEAVNRAVARVADTLPTGPRNSESIPALTAAETGHTSQVVPGDTMQTRHVKNYHSRTESSVENFLCRAACVYITKYKTKDSDPVQRYANWRINTRQMVQLRRKFELFTYLRFDMEVTFVITSSQDDGTRLAQDMPVLTHQVMYIPPGGPVPNSVTDFAWQSSTNPSIFWTEGNAPARMSIPFISIGNAYSNFYDGWSHFTQDGVYGFNSLNNMGSIYIRHVNEQSPHAITSTVRVYFKPKHVRAWVPRPPRLCAYEKSSNVNFKPTDVTTSRTSITEVPSLRPSVVNTGAFGQQSGAAYVGNYRVVNRHLATHVDWQNCVWEDYNRDLLVSTTTAHGCDTIARCQCTTGVYFCASRNKHYPVSYEGPGLVEVQESEYYPRRYQSHVLLAAGFSEPGDCGGILRCEHGVIGLVTMGGEGVVGFADVRDLLWLEDDAMEQGVKDYVEQLGNAFGSGFTNQICEQVXLLKESLVGHDSILEKSLKALVKIISALVIVVRNHDDLITVTATLALIGCTSSPWRWLKHKVSQYYGIPMAERQSNGWLKKFTEMTNACKGMEWIAIKIQKFIEWLKLKILPEVKEKHEFLNRLKQLPLLESQIATIEQSAPSQSDQEQLFSNVQYFAHYCRKYAPLYAAEAKRVFSLEKKMSNYIQFKSKCRIEPVCLLLHGSPGAGKSVATSLIGRSLAEKLNSSVYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYSQNGKINMPMSVKTCDEECCPVNFKRCCPLVCGKAIQFIDRRTQVRYSLDMLVTEMFREYNHRHSVGATLEALFQGPPVYREIKISVAPETPPPPAIADLLKSVDSEAVREYCKEKGWLVPEVNSTLQIEKHVSRAFICLQALTTFVSVAGIIYIIYKLFAGFQGAYTGMPNQKPKVPTLRQAKVQGPAFEFAVAMMKRNASTVKTEYGEFTMLGIYDRWAVLPRHAKPGPTILMNDQEIGVLDAKELVDKDGTNLELTLLKLNRNEKFRDIRGFLAREEAEVNEAVLAINTSKFPNMYIPVGQVTDYGFLNLGGTPTKRMLMYNFPTRAGQCGGVLMSTGKVLGIHVGGNGHQGFSAALLRHYFNEEQGEIEFIESSKDAGFPVINTPSKTKLEPSVFHQVFEGNKEPAVLRNGDPRLKVNFEEAIFSKYIGNINTHVDEYMLEAVDHYAGQLATLDISTEPMKLEDAVYGTEGLEALDLTTSAGYPYVALGIKKRDILSKKTKDLTKLKECMDKYGLNLPMVTYVKDELRSSEKVAKGKSRLIEASSLNDSVAMRQTFGNLYKTFHLNPGIVTGSAVGCDPDLFWSKIPVMLDGHLIAFDYSGYDASLSPVWFACLKLLLEKLGYTHRETNYIDYLCNSHHLYRDKHYFVRGGMPSGCSGTSIFNSMINNIIIRTLMLKVYKGIDLDQFRMIAYGDDVIASYPWPIDASLLAEAGKGYGLIMTPADKGECFNEVTWTNVTFLKRYFRADEQYPFLVHPVMPMKDIHESIRWTKDPKNTQDHVRSLCLLAWHNGEQEYEEFIRKIRSVPVGRCLTLPAFSTLRRKWLDSF

SEQ ID NO: 158 - protein, 320 aa, Echovirus 12; identical to SEQ ID NO: 146

SEQ ID NO: 159 - protein, 291 aa, Echovirus 12; identical to SEQ ID NO: 87

SEQ ID NO: 160 - protein, 238 aa, Echovirus 12; identical to SEQ ID NO: 63

SEQ ID NO: 161 - protein, 121 aa, Echovirus 12
NCVWEDYNRDLLVSTTTAHGCDTIARCQCTTGVYFCASRNKHYPVSYEGPGLVEVQESEYYPRRYQSHVLLAAGFSEPGDCGGILRCEHGVIGLVTMGGEGVVGFADVRDLLWLEDDAMEQ

SEQ ID NO: 162 - protein, 90 aa, Echovirus 12; identical to SEQ ID NO: 67

SEQ ID NO: 163 - protein, 183 aa, Echovirus 12; identical to SEQ ID NO: 69

SEQ ID NO: 164 - protein, 462 aa, Echovirus 12; identical to SEQ ID NO: 71

SEQ ID NO: 165 - RNA, 6579 nt, Echovirus 12
  source 1..6579: mol_type = unassigned RNA
atgggagcgcaagtatcaacacaaaaaactggggcacacgagactggcctgagtgctagtggaaattccatcatccactataccaacattaattactacaaagatgcggcatccaattcagcaaacaggcaggatttcacccaagaccccgggaaattcaccgagccagtgaaggatatcatgataaaatcgatgcctgcgttaaactccccaacagcagaagaatgtgggtacagcgatagagtcagatcaatcaccctggggaactctaccatcaccacgcaggagtgtgccaatgtggtggtgggatatggcacgtggcctgattatctacatgatgatgaggccactgcagaggaccaaccaacacaaccggatgtggcgacgtgtaggttttacacacttgaatccattcagtggcagaagacatcagatgggtggtggtggaagtttccggaagcattgaaagacatgggactgttcggccaaaacatgcactatcattatcttggcaggtcgggttacaccatccacgtccaatgtaacgcgtctaaattccaccagggttgtttgcttgtggtgtgtgtgcctgaagctgagatggggtgcgctacagtggcgaatgaggttaccgcagccgctctctcatctggtgaaacggcaaaatattttgcaaagactggtgcgacgggaacccacacagtgcaaagcatagtgacaaacgccggcatgggggtgggcgtgggcaatctgacaatattcccacatcagtggataaacctccgtaccaacaacagcgcgactattgtcatgccttacataaacagtgtgcccatggataacatgttcagacactacaatttcaccctaatgatcatccccttcgtcccactggacttcacagctgaggcatctacgtatgtaccaatcactgtaacagtagctcccatgtgcgccgagtacaacgggctacgcctggcctcgcatcaaggtttgccgacaatgaacacccctggtagtaaccaattcctcacatcagatgactttcaatcaccatcagccatgccacaattcgatgtcacacccgagctcaggattccaggggaggtaaagaacttaatggaaatagccgaagttgactccgtagtgcctgtgaacaatacacaggactcagtgtacaacatggatgtgtacaaaattcctgttagtggaggcaaccaactgtcaactcaagtcttcgggttccaaatgcaaccagggctaaatagtgtgttcaaaaggacattactaggtgagatactgaactactatgcacactggtctggtagtgtaaagctcacctttgtgttctgtggatcggccatggccactggcaaatttctgctggcatattcccctccaggcgcagaccccccaaagtctaggaaagaagcaatgcttggtactcacgtgatatgggatataggacttcagtcaagctgcgtcttgtgcgtaccgtggatcagccaaacacattatagattggttcagcaggatgaatatacgagtgcaggatatgtaacttgctggtatcaaactagtttggttgtcccgcccggcgctcccgtcacgtgtggagtcttgtgcttagcatcggcgtgcaatgatttctctgtgagaatgttgagagacacaccttttatcgagcaaaaacagctactccaaggagatgtggaggaggccgtgaacagagccgttgcacgggtggcggatacactacccacagggccgagaaattcagagagcatccccgcactaacagcagcagaaactgggcatacatctcaggtggttcctggggataccatgcaaacccgtcatgtgaagaattaccactccagaactgaatcgtctgttgaaaacttcttatgccgagcagcatgtgtgtatattacaaagtacaagaccaaggacagtgatccagtgcaaaggtacgccaactggcggatcaacacccgtcaaatggttcagctaaggagaaaatttgagttgtttacatacctaaggtttgatatggaagttacttttgtgatcaccagttcacaagatgatggcacgcggttggcgcaggacatgccagtattaacacaccaagtcatgtatataccacctggagggccagtgccgaatagtgttactgattttgcgtggcaatcgtctaccaaccctagcatcttctggacggagggaaacgctccagctaggatgtccatcccctttatcagtataggcaacgcgtatagcaatttctatgatggttggtcacatttcacccaagatggagtatatggtttcaactccctcaataacatgggctccatctacattaggcatgtcaatgaacaaagcccgcatgcaattacgagcacagtcagagtctatttcaaaccaaaacacgtgcgggcctgggtgccaagaccacctaggctttgtgcatatgagaaatcaagcaacgtgaatttcaaaccaacagatgtgactacctcccgaacatccatcacagaagtccctagtcttagaccatcagtggtaaatactggagctttcggtcagcaatcaggagcggcttatgtgggaaactatagagtggttaatagacacctagccactcatgttgactggcagaactgtgtgtgggaggactataacagggacctccttgtaagcaccaccacagctcatgggtgtgacaccatagccagatgccaatgcacaacaggcgtgtacttctgtgcatcgaggaataaacactacccagtcagttatgaaggaccaggtctggtagaagtccaggagagcgagtactaccccaggaggtatcaatcacacgtcttgctggctgcgggattttcagagccaggagattgtggagggattctcagatgtgagcacggtgtcatcggtctagttactatgggtggcgaaggcgttgtcggattcgctgatgtacgcgacctgttatggcttgaagatgacgccatggaacaaggagtcaaggattacgtggaacaactagggaacgcctttggttcaggattcactaaccaaatctgtgagcaggtcaayctcctaaaagagtcactggtgggtcacgattccatcctagagaagtcccttaaagccctagtgaaaatcatatcagcactagtgatagtggtgaggaaccatgatgatttgatcactgtaactgctactcttgccctcattggttgtacctcctctccatggcggtggctcaagcacaaggtgtcacaatactacgggatacccatggcagagcgccaaagcaacgggtggctcaagaagttcacagagatgaccaacgcctgcaaagggatggaatggattgccatcaaaatccaaaagttcatagagtggcttaaacttaagattttaccagaagttaaggaaaaacatgaattcctgaataggctcaagcaactcccactgttggagagtcaaatagcaaccatcgagcagagtgcaccgtcccagagcgatcaggagcagctcttttcaaatgttcagtactttgcccattattgcagaaaatatgcgcccctatacgcagctgaggcaaaaagggtgttctctcttgaaaagaaaatgagtaattacatacagttcaagtccaaatgccgtattgaacctgtatgcttgctactacatgggagccccggagcagggaaatcagttgctaccagcctcattgggcgatcactagctgaaaaattgaatagctcagtgtactccttaccaccagacccagatcactttgatggttacaagcaacaagctgtagtgatcatggacgatctgtgccaaaatccagatggaaaagatgtgtctttgttctgtcaaatggtgtctagtgtagattttgtaccaccaatggccgcgctagaggagaaaggcattttgttcacctctccattcgttctagcctcaaccaatgcaggatccatcaacgcaccaacagtttcagacagcagagccctagctagaaggttccactttgacatgaacattgaagttatctccatgtacagtcaaaatggaaaaataaacatgcccatgtcagtcaaaacatgtgatgaggaatgctgcccagtcaatttcaagagatgctgcccactggtgtgtggcaaggctattcagttcattgacagaagaacccaagtcagatattcactggatatgttagtcaccgagatgttcagggagtacaaccacaggcacagtgtgggcgccaccctcgaggctctgttccaaggtccaccggtctacagggagatcaagatcagtgttgctccagaaacacctcccccaccagcaatcgctgatttattaaaatcagtagacagtgaagctgtgagagagtactgcaaggaaaaaggctggcttgtaccggaagttaactccaccctacagattgagaagcacgtcagcagagcatttatctgtctacaagctctgaccactttcgtctcagtagctggcataatctacattatctacaagttgtttgccggctttcagggcgcgtatacggggatgccaaatcagaaacccaaagtgcccactctgagacaagctaaagtgcagggcccagcattcgagttcgctgtggcaatgatgaaaaggaacgccagcacagtgaaaacggaatatggtgagttcaccatgctcggcatctatgacagatgggcagtgctaccacgccacgccaagcctggaccgactattttgatgaatgatcaggagatcggcgtgttagacgccaaagaattagtggacaaagatgggacaaatctggagttgacccttttaaagctcaaccgcaatgagaagtttagggacatcagagggtttctggcgagagaggaagctgaagtgaatgaggctgttctggcaataaacacaagcaagttcccaaatatgtacatacccgtaggtcaagtcactgattacggtttcctgaacctggggggaacacccacaaagaggatgctcatgtacaactttccaactagagcagggcaatgtggtggggtactcatgtcaacagggaaagttcttggtatacatgtaggaggaaatggacatcaagggttctccgctgctcttcttaggcactactttaatgaggagcaaggtgaaatagagtttattgagagctcaaaggatgcaggattccctgtgatcaacacccccagcaaaacaaaactggaaccaagcgtgtttcatcaggtgtttgaaggcaataaagagccagcagtccttagaaatggtgatccgcgtcttaaagtaaactttgaagaagccatattttccaagtacatcgggaacatcaacacacatgtggatgaatacatgctcgaagcagtggatcactacgcagggcaactggctactcttgacattagcactgaacccatgaaactagaagatgcagtgtatggcactgaaggactagaggctcttgacttaacaacaagtgcagggtacccgtatgtcgcactaggcatcaaaaagagagacatcctatccaaaaagactaaagatctgactaaattgaaggaatgcatggacaagtacggtttgaacctgccgatggtgacttatgtgaaggatgaacttagatcatcagagaaagtggctaagggaaaatcaagactcattgaagcatctagtttaaatgactctgtcgcgatgaggcagacgtttggcaacttgtacaaaacattccatctgaacccgggcattgtgacaggcagtgcagttggatgtgatccggaccttttctggagtaaaatacccgtgatgttagacggccacctcatagcattcgattactccggatatgatgccagcctgagccctgtgtggttcgcttgtctaaaactgctactcgaaaaactcgggtacacgcacagagagacaaactacatcgactacctatgcaactctcaccacctgtacagagacaaacactactttgtgcgtggtggcatgccatcagggtgctccggtaccagcattttcaattcaatgatcaataacatcataatcaggacactgatgctcaaagtgtacaagggaattgacctggaccagtttagaatgattgcatatggtgatgatgtaattgcatcctacccttggcctatagacgcttcactacttgctgaagctgggaagggttatggactgattatgacaccagcagacaaaggggagtgcttcaatgaggttacctggaccaatgtcaccttcctaaagaggtactttagagctgatgagcagtatcctttcctggttcatccagttatgcccatgaaggacatccatgaatctattaggtggacaaaggatccaaagaacactcaagaccacgttcgatcgttgtgcttattggcttggcataacggagaacaagaatatgaggaattcatccgaaagatcagaagcgtcccggttgggcgctgtctgactctccccgcgttttcaactttacgcagaaaatggctggattccttt

SEQ ID NO: 166 - RNA, 741 nt, Echovirus 12; identical to SEQ ID NO: 44
  source 1..741: mol_type = unassigned RNA

SEQ ID NO: 167 - RNA, 103 nt, Echovirus 12; identical to SEQ ID NO: 45
  source 1..103: mol_type = unassigned RNA